(19) United States
(12) Patent Application Publication
(10) Pub. No.: US 2016/0319034 A1
(43) Pub. Date: Nov. 3, 2016

(54) MET-BINDING AGENTS AND USES THEREOF

(71) Applicant: ONCOMED PHARMACEUTICALS, INC., Redwood City, CA (US)

(72) Inventors: Austin L. GURNEY, San Francisco, CA (US); Ming-Hong XIE, Foster City, CA (US); Christopher John BOND, San Mateo, CA (US)

(21) Appl. No.: 14/776,262

(22) PCT Filed: Mar. 12, 2014
(86) PCT No.: PCT/US14/24258

§ 371 Date: Sep. 14, 2015

Related U.S. Application Data
(60) Provisional application No. 61/783,552, filed on Mar. 14, 2013.
(60) Provisional application No. 61/829,477, filed on May 31, 2013.
(60) Provisional application No. 61/898,851, filed on Nov. 1, 2013.

(51) Int. Cl. C07K 16/40; C07K 14/705; C07K 16/30; G01N 33/574; A61K 45/06; A61K 38/17; C12Q 1/68; C07K 16/28; A61K 39/395

(52) U.S. Cl. CPC C07K 16/40; C07K 16/28; C07K 14/705; C07K 16/3023; C07K 16/3015; C07K 16/303; A61K 39/39558; A61K 45/06; A61K 38/177; C12Q 1/6886; G01N 33/5748; C07K 2319/30; C07K 2317/31; C07K 2317/92; C07K 2317/565; C07K 2317/34; C07K 2317/76; C07K 2317/77; C07K 2317/75; C07K 2317/56; C07K 2317/14; C07K 2317/515; C07K 2317/51; C07K 2317/526; C07K 2317/52; A61K 2039/505

(57) ABSTRACT

The present invention relates to binding agents that specifically bind human MET, binding agents that specifically bind one or more components of the WNT pathway, bispecific agents that bind both human MET and one or more components of the WNT pathway, and methods of using the agents for treating diseases such as cancer.

### CROSS-REFERENCE TO RELATED APPLICATIONS

This application claims priority benefit of U.S. Provisional Application No. 61/783,552, filed Mar. 14, 2013; U.S. Provisional Application No. 61/829,477, filed May 31, 2013; and U.S. Provisional Application No. 61/898,851, filed Nov. 1, 2013, each of which is hereby incorporated by reference herein in its entirety.

### FIELD OF THE INVENTION

The present invention generally relates to antibodies, bispecific agents, and other binding agents that bind MET, one or more components of the WNT pathway, or both MET and one or more components of the WNT pathway, particularly bispecific agents that bind both MET and one or more WNT proteins, as well as to methods of using the binding agents for the treatment of diseases such as cancer.

### BACKGROUND OF THE INVENTION

Cancer is one of the leading causes of death in the developed world, with over one million people diagnosed with cancer and 500,000 deaths per year in the United States alone. Overall it is estimated that more than 1 in 3 people will develop some form of cancer during their lifetime. There are more than 200 different types of cancer, four of which—breast, lung, colorectal, and prostate—account for almost half of all new cases (Siegel et al., 2011, CA: A Cancer J Clin. 61:212-236).

Signaling pathways normally connect extracellular signals to the nucleus leading to expression of genes that directly or indirectly control cell growth, differentiation, survival, and death. In a wide variety of cancers, signaling pathways are dysregulated and may be linked to tumor initiation and/or progression. Signaling pathways implicated in human oncogenesis include, but are not limited to, the WNT pathway, the Ras-Raf-MEK-ERK or MAPK pathway, the PI3K-AKT pathway, the MET/HGF pathway, the CDKN2A/CDK4 pathway, the Bcl-2/TP53 pathway, and the NOTCH pathway.

The MET/HGF (hepatocyte growth factor) pathway has been shown to play a critical role in early embryonic development. However, in adult tissues the MET pathway is tightly controlled and primarily quiescent in its growth signaling program, except in processes such as wound repair. Dysregulation of the MET pathway may lead to cell proliferation, protection from apoptosis, angiogenesis, invasion, and metastasis. MET may be dysregulated by a variety of different mechanisms including protein over-expression, constitutive activation, ligand-dependent activation, gene amplification, gene mutation, and/or MET modifications (e.g., phosphorylation). The MET pathway has been shown to be dysregulated in many tumor types, including but not limited to, lung, colorectal, breast, liver, gastric, pancreatic, and brain.

The WNT signaling pathway is one of several critical regulators of embryonic pattern formation, post-embryonic tissue maintenance, and stem cell biology. More specifically, WNT signaling plays an important role in the generation of cell polarity and cell fate specification including self-renewal by stem cell populations. Unregulated activation of the WNT pathway is associated with numerous human cancers where it is believed the activation can alter the developmental fate of cells. The activation of the WNT pathway may maintain tumor cells in an undifferentiated state and/or lead to uncontrolled proliferation. Thus, carcinogenesis can proceed by overtaking homeostatic mechanisms that control normal development and tissue repair (reviewed in Reya & Clevers, 2005, Nature, 434:843-50; Beachy et al., 2004, Nature, 432:324-31).

The MET pathway and the WNT pathway have both been identified as potential targets for cancer therapy. It is one of the objectives of the present invention to provide improved molecules for cancer treatment, particularly bispecific agents that specifically bind human MET and one or more WNT components of the WNT pathway (i.e., WNT proteins). Another objective of the invention is to use these novel bispecific agents to modulate the MET pathway and the WNT pathway and inhibit tumor growth.

### SUMMARY OF THE INVENTION

The present invention provides binding agents, such as antibodies, soluble receptors, or bispecific agents that bind MET, one or more components of the WNT pathway, or both MET and one or more components of the WNT pathway, as well as compositions, such as pharmaceutical compositions, comprising the binding agents. Binding agents that bind MET, bind one or more components of the WNT pathway, or bind both MET and one or more components of the WNT pathway, and pharmaceutical compositions of such binding agents, are also provided. In certain embodiments, the binding agents are novel polypeptides, such as antibodies, antibody fragments, and other polypeptides related to such antibodies. In certain embodiments, the binding agents are novel polypeptides, such as soluble receptors and other polypeptides related to such soluble receptors. In certain embodiments, the binding agents are antibodies that specifically bind human MET. In some embodiments, the binding agents are antibodies that specifically bind one or more human WNT proteins. In some embodiments, the binding agents are antibodies that specifically bind one or more human Frizzled (FZD) proteins. In some embodiments, the binding agents are soluble FZD receptors that specifically bind one or more human WNT proteins. In some embodiments, the binding agents are bispecific agents that specifically bind human MET and one or more components of the WNT pathway. In some embodiments, the binding agents are bispecific agents that specifically bind human MET and one or more human WNT proteins. In some embodiments, the binding agents are bispecific molecules that specifically bind human MET and one or more human FZD proteins. The invention further provides methods of inhibiting the growth of a tumor by administering the binding agents to a subject with a tumor. The invention further provides methods of treating cancer by administering the binding agents to a subject in need thereof. In some embodiments, the methods of treating cancer or inhibiting tumor growth comprise targeting cancer stem cells with the binding agents. In certain embodiments, the methods comprise reducing the frequency of cancer stem cells in a tumor, reducing the number of cancer stem cells in a tumor, reducing the tumorigenicity of a tumor, and/or reducing the tumorigenicity of a tumor by reducing the number or frequency of cancer stem cells in the tumor.

In one aspect, the invention provides a binding agent, such as an antibody, that specifically binds human MET. In some embodiments, the binding agent inhibits binding of MET to hepatocyte growth factor. In some embodiments, the binding agent inhibits activation of MET. In certain embodiments, the binding agent (e.g., a bispecific agent) specifically binds one or more components of the human WNT pathway in addition to binding human MET. In certain embodiments, the binding agent (e.g., a bispecific agent) specifically binds one or more human FZD proteins in addition to binding human MET. In certain embodiments, the binding agent (e.g., a bispecific agent) specifically binds one or more human WNT proteins in addition to binding human MET.

In certain embodiments, the binding agent specifically binds the extracellular domain of human MET. In some embodiments, the binding agent specifically binds the Sema domain of human MET. In some embodiments, the binding agent specifically binds within the Sema domain of human MET. In some embodiments, the binding agent specifically binds the PSI (plexin-semaphorin-integrin) domain of human MET. In some embodiments, the binding agent specifically binds an IPT (immunoglobulin-like fold, plexin, transcription factor) domain of human MET. In some embodiments, the binding agent specifically binds one or more of the IPT repeat domains of human MET. In some embodiments, the binding agent specifically binds the Sema domain, the PSI domain, and/or one or more of the IPT repeat domains of human MET.

In some embodiments, the binding agent specifically binds within amino acids 25-932 of human MET (SEQ ID NO:93). In some embodiments, the binding agent specifically binds within amino acids 25-836 of human MET (SEQ ID NO:93). In some embodiments, the binding agent specifically binds within amino acids 25-515 of human MET (SEQ ID NO:93). In some embodiments, the binding agent specifically binds within amino acids 50-130 of human MET (SEQ ID NO:93). In some embodiments, the binding agent specifically binds within amino acids 70-110 of human MET (SEQ ID NO:93). In some embodiments, the binding agent specifically binds an epitope that comprises amino acids 97-101 of human MET (SEQ ID NO:93). In some embodiments, the binding agent specifically binds an epitope that comprises the glutamine at position 99 of SEQ ID NO:93. In some embodiments, the binding agent specifically binds within amino acids 519-562 of human MET (SEQ ID NO:93). In some embodiments, the binding agent specifically binds within amino acids 563-950 of human MET (SEQ ID NO:93). In some embodiments, the binding agent specifically binds within amino acids 563-836 of human MET (SEQ ID NO:93). In some embodiments, the binding agent specifically binds within amino acids 563-656 of human MET (SEQ ID NO:93). In some embodiments, the binding agent specifically binds within amino acids 657-740 of human MET (SEQ ID NO:93). In some embodiments, the binding agent specifically binds within amino acids 741-855 of human MET (SEQ ID NO:93). In some embodiments, the binding agent specifically binds within amino acids 856-950 of human MET (SEQ ID NO:93).

In some embodiments, the binding agent is an antibody that specifically binds human MET. In some embodiments, the MET-binding agent is an antibody that comprises a heavy chain CDR1 comprising ASYAWS (SEQ ID NO:1), a heavy chain CDR2 comprising YISYSGGTDYNPSLKS (SEQ ID NO:2), and a heavy chain CDR3 comprising KGAY (SEQ ID NO:3); and a light chain CDR1 comprising SASSSVSSSYLY (SEQ ID NO:4), a light chain CDR2 comprising STSNLAS (SEQ ID NO:5), and a light chain CDR3 comprising HQWSSYPYT (SEQ ID NO:6).

In certain embodiments, the MET-binding agent is an antibody that comprises a heavy chain variable region having at least about 80% sequence identity to SEQ ID NO:7; and/or a light chain variable region having at least about 80% sequence identity to SEQ ID NO:8. In certain embodiments, the binding agent comprises a heavy chain variable region having at least about 90% sequence identity to SEQ ID NO:7; and/or a light chain variable region having at least about 90% sequence identity to SEQ ID NO:8. In certain embodiments, the binding agent comprises a heavy chain variable region having at least about 95% sequence identity to SEQ ID NO:7; and/or a light chain variable region having at least about 95% sequence identity to SEQ ID NO:8. In certain embodiments, the binding agent is an antibody that comprises a heavy chain variable region of SEQ ID NO:7, and/or a light chain variable region of SEQ ID NO:8.

In certain embodiments, the MET-binding agent is an antibody that comprises a heavy chain variable region having at least about 80% sequence identity to SEQ ID NO:94; and/or a light chain variable region having at least about 80% sequence identity to SEQ ID NO:95. In certain embodiments, the binding agent comprises a heavy chain variable region having at least about 90% sequence identity to SEQ ID NO:94; and/or a light chain variable region having at least about 90% sequence identity to SEQ ID NO:95. In certain embodiments, the binding agent comprises a heavy chain variable region having at least about 95% sequence identity to SEQ ID NO:94; and/or a light chain variable region having at least about 95% sequence identity to SEQ ID NO:95. In certain embodiments, the binding agent is an antibody that comprises a heavy chain variable region of SEQ ID NO:94, and/or a light chain variable region of SEQ ID NO:95. In certain embodiments, the binding agent is an antibody that comprises a heavy chain variable region of SEQ ID NO:94 and a light chain variable region of SEQ ID NO:95.

In some embodiments, the MET-binding agent is an antibody that comprises a heavy chain of SEQ ID NO:9, SEQ ID NO:10, SEQ ID NO:12, SEQ ID NO:13, SEQ ID NO:88, SEQ ID NO:96, SEQ ID NO:97, SEQ ID NO:99, SEQ ID NO:100, SEQ ID NO:108, SEQ ID NO:110, SEQ ID NO:111, or SEQ ID NO:112; and/or a light chain of SEQ ID NO:11, SEQ ID NO:14, SEQ ID NO:98, or SEQ ID NO:101.

In some embodiments, the binding agent is antibody 73R009. In some embodiments, the binding agent is a variant of antibody 73R009. In some embodiments, the binding agent is a monovalent version of 73R009. In some embodiments, the binding agent is a humanized version of antibody 73R009. In some embodiments, the binding agent is antibody 73R009 H12L7, also referred to as 73R010. In some embodiments, the binding agent is a variant of antibody 73R010 comprising a substitution of a cysteine residue with a serine residue to reduce undesirable disulfide bond formation. Reference to antibody “73R010”, includes the variant with a cysteine substitution.

In another aspect, the invention provides a binding agent that is a bispecific agent, wherein the bispecific agent specifically binds human MET. In some embodiments, the bispecific agent specifically binds human MET and a second target. In some embodiments the bispecific agent binds human MET and one or more components of the human WNT pathway. In some embodiments, the bispecific agent binds both human MET and one or more human WNT proteins. In some embodiments, the bispecific agent is a bispecific antibody. In some embodiments, the bispecific antibody binds both human MET and one or more components of the human WNT pathway. In some embodiments, the bispecific antibody binds both human MET and one or more human WNT proteins. In some embodiments, the bispecific antibody binds both human MET and one or more human FZD proteins. In certain embodiments, the bispecific antibody comprises two identical light chains. In certain embodiments, the bispecific antibody is an IgG antibody. In certain embodiments, the bispecific antibody is an IgG1 antibody. In certain embodiments, the bispecific antibody is an IgG2 antibody.

In another aspect, the invention provides a bispecific agent that comprises a first arm that comprises a first binding site and a second arm that comprises a second binding site. In some embodiments, the first binding site comprises a first antigen-binding site from a first antibody and the second binding site comprises a second antibody-binding site from a second antibody. In some embodiments, the first binding site comprises an antigen-binding site from an antibody and the second binding site comprises a binding site that is not from an antibody. In some embodiments, the first arm comprises a monovalent antibody and the second arm comprises a soluble receptor.

In some embodiments, the bispecific agent comprises: a first binding site that specifically binds human MET, and a second binding site that specifically binds one or more components of the WNT pathway. In some embodiments, the bispecific agent comprises a first binding site that specifically binds human MET, and a second binding site that specifically binds one or more components of the WNT pathway, wherein the first binding site comprises a heavy chain CDR1 comprising ASYAWS (SEQ ID NO:1), a heavy chain CDR2 comprising YISYSGGTDYNPSLKS (SEQ ID NO:2), and a heavy chain CDR3 comprising KGAY (SEQ ID NO:3). In some embodiments, the bispecific agent further comprises: a light chain CDR1 comprising SASSSVSSSYLY (SEQ ID NO:4), a light chain CDR2 comprising STSNLAS (SEQ ID NO:5), and a light chain CDR3 comprising HQWSSYPYT (SEQ ID NO:6). In some embodiments, the bispecific agent comprises: a first binding site that specifically binds human MET, wherein the first binding site comprises (a) a heavy chain CDR1 comprising ASYAWS (SEQ ID NO:1), a heavy chain CDR2 comprising YISYSGGTDYNPSLKS (SEQ ID NO:2), and a heavy chain CDR3 comprising KGAY (SEQ ID NO:3), and a light chain CDR1 comp′ sing SASSSVSSSYLY (SEQ ID NO:4), a light chain CDR2 comprising STSNLAS (SEQ ID NO:5), and a light chain CDR3 comprising HQWSSYPYT (SEQ ID NO:6).

In some embodiments, the bispecific agent comprises: a first binding site that specifically binds human MET, and a second binding site that specifically binds one or more components of the WNT pathway. In some embodiments, the bispecific agent comprises a first binding site that specifically binds human MET, and a second binding site that specifically binds one or more components of the WNT pathway, wherein the first binding site comprises a heavy chain CDR1 comprising GYTFTSYWLH (SEQ ID NO:78), a heavy chain CDR2 comprising GMIDPSNSDTRFNPNFKD (SEQ ID NO:79), and a heavy chain CDR3 comprising TYGSYVSPLDY (SEQ ID NO:81), SYGSYVSPLDY (SEQ ID NO:82), ATYGSYVSPLDY (SEQ ID NO:83), or XYGSYVSPLDY (SEQ ID NO:80), wherein X is not R; and a light chain CDR1 comprising KSSQSLLYTSSQKNYLA (SEQ ID NO:84), a light chain CDR2 comprising WASTRES (SEQ ID NO:85), and a light chain CDR3 comprising QQYYAYPWT (SEQ ID NO:86).

In some embodiments, the bispecific agent comprises a first binding site that specifically binds human MET, and a second binding site that specifically binds one or more components of the WNT pathway, wherein the first binding site comprises a first antigen-binding site from a first antibody, and the second binding site comprises a second antigen-binding site from a second antibody. Thus, in some embodiments, the bispecific agent is a bispecific antibody. In some embodiments, the second binding site specifically binds one or more human WNT proteins. In some embodiments, the one or more WNT proteins is selected from the group consisting of: WNT1, WNT2, WNT2b, WNT3, WNT3a, WNT7a, WNT7b, WNT8a, WNT8b, WNT10a, and WNT10b. In some embodiments, the second binding site specifically binds one or more Frizzled (FZD) proteins. In some embodiments, the one or more FZD proteins is selected from the group consisting of: FZD1, FZD2, FZD3, FZD4, FZD5, FZD6, FZD7, FZD8, FZD9, and FZD10. In some embodiments, the one or more FZD proteins is selected from the group consisting of: FZD1, FZD2, FZD5, FZD7, and FZD8.

In some embodiments, the bispecific agent comprises a first binding site that specifically binds human MET, and a second binding site that specifically binds one or more components of the WNT pathway, wherein the second binding site comprises a soluble receptor. In some embodiments, the soluble receptor comprises an extracellular domain (ECD) of a human FZD protein. In some embodiments, the soluble receptor comprises a fragment of an ECD of a human FZD protein. In some embodiments, the soluble receptor comprises a Fri domain of a human FZD protein. In some embodiments, the soluble receptor comprises a Fri domain of human FZD1, the Fri domain of human FZD2, the Fri domain of human FZD3, the Fr′ domain of human FZD4, the Fri domain of human FZD5, the Fri domain of human FZD6, the Fri domain of human FZD7, the Fri domain of human FZD8, the Fri domain of human FZD9, or the Fri domain of human FZD10. In some embodiments, the soluble receptor comprises the Fri domain of human FZD8. In some embodiments, the soluble receptor comprises a Fri domain of a human FZD protein that comprises a sequence selected from the group consisting of: SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, and SEQ ID NO:31. In some embodiments, the soluble receptor comprises a minimal core Fri domain of a human FZD protein that comprises a sequence selected from the group consisting of: SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, and SEQ ID NO:41. In some embodiments, the soluble receptor comprises a Fri domain of a human FZD protein of SEQ ID NO:28, SEQ ID NO:29, or SEQ ID NO:39. In some embodiments, the Fri domain of a human FZD protein is directly linked to a heterologous polypeptide (i.e., a non-FZD polypeptide). In some embodiments, the Fri domain of a human FZD protein is connected to a heterologous polypeptide by a linker. In some embodiments, the heterologous polypeptide comprises a human Fc region. In some embodiments, the heterologous polypeptide comprises: SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:42, SEQ ID NO:43, SEQ ID NO:91, or SEQ ID NO:92. In some embodiments, the soluble receptor comprises: (a) a first polypeptide of SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, or SEQ ID NO:41; and (b) a second polypeptide of SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:42, SEQ ID NO:43, SEQ ID NO:91, or SEQ ID NO:92, wherein the first polypeptide is directly linked to the second polypeptide. In some embodiments, the soluble receptor comprises: (a) a first polypeptide of SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, or SEQ ID NO:41; and (b) a second polypeptide of SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:42, SEQ ID NO:43, SEQ ID NO:91, or SEQ ID NO:92, wherein the first polypeptide is connected to the second polypeptide by a linker. In some embodiments, the soluble receptor comprises a first polypeptide comprising SEQ ID NO:28. In some embodiments, the soluble receptor comprises a first polypeptide of SEQ ID NO:28. In some embodiments, the soluble receptor comprises a first polypeptide of SEQ ID NO:28, and a second polypeptide of SEQ ID NO:44, SEQ ID NO:45, SEQ ID NO:46, SEQ ID NO:47, SEQ ID NO:48, SEQ ID NO:49, SEQ ID NO:50, SEQ ID NO:51, or SEQ ID NO:52. In some embodiments, the soluble receptor comprises a first polypeptide comprising SEQ ID NO:29. In some embodiments, the soluble receptor comprises a first polypeptide of SEQ ID NO:29. In some embodiments, the soluble receptor comprises a first polypeptide of SEQ ID NO:29, and a second polypeptide SEQ ID NO:44, SEQ ID NO:45, SEQ ID NO:46, SEQ ID NO:47, or SEQ ID NO:48, SEQ ID NO:49, SEQ ID NO:50, SEQ ID NO:51, or SEQ ID NO:52. In some embodiments, the soluble receptor comprises SEQ ID NO:52 or SEQ ID NO:50. In some embodiments, the soluble receptor comprises SEQ ID NO:52.

In some embodiments, the bispecific agent comprises a first arm that specifically binds human MET, and a second arm that specifically binds one or more components of the WNT pathway, wherein the first arm comprises a heavy chain CDR1 comprising ASYAWS (SEQ ID NO:1), a heavy chain CDR2 comprising YISYSGGTDYNPSLKS (SEQ ID NO:2), and a heavy chain CDR3 comprising KGAY (SEQ ID NO:3), and a light chain CDR1 comprising SASSSVSSSYLY (SEQ ID NO:4), a light chain CDR2 comprising STSNLAS (SEQ ID NO:5), and a light chain CDR3 comprising HQWSSYPYT (SEQ ID NO:6), and wherein the second arm comprises SEQ ID NO:56 or SEQ ID NO:87.

In some embodiments, a bispecific agent comprises a first binding site that specifically binds human MET, and a second binding site that specifically binds one or more components of the WNT pathway, wherein the first binding site comprises a heavy chain variable region having at least about 90% sequence identity to SEQ ID NO:7 and a light chain variable region having at least about 90% sequence identity to SEQ ID NO:8. In some embodiments, the first antigen-binding site comprises a heavy chain variable region having at least about 95% sequence identity to SEQ ID NO:7 and a light chain variable region have at least about 95% sequence identity to SEQ ID NO:8. In some embodiments, the first antigen-binding site comprises a heavy chain variable region of SEQ ID NO:7 and a light chain variable region of SEQ ID NO:8.

In some embodiments, a bispecific agent comprises a first arm that specifically binds human MET, and a second arm that specifically binds one or more components of the WNT pathway, wherein the first arm comprises a heavy chain variable region having at least about 90% sequence identity to SEQ ID NO:7 and a light chain variable region having at least about 90% sequence identity to SEQ ID NO:8, and wherein the second arm comprises SEQ ID NO:56 or SEQ ID NO:87.

In some embodiments, a bispecific agent comprises a first binding site that specifically binds human MET, and a second binding site that specifically binds one or more components of the WNT pathway, wherein the first binding site comprises a heavy chain variable region having at least about 90% sequence identity to SEQ ID NO:94 and a light chain variable region having at least about 90% sequence identity to SEQ ID NO:95. In some embodiments, the first antigen-binding site comprises a heavy chain variable region having at least about 95% sequence identity to SEQ ID NO:94 and a light chain variable region have at least about 95% sequence identity to SEQ ID NO:95. In some embodiments, the first antigen-binding site comprises a heavy chain variable region of SEQ ID NO:94 and a light chain variable region of SEQ ID NO:95.

In some embodiments, a bispecific agent comprises a first arm that specifically binds human MET, and a second arm that specifically binds one or more components of the WNT pathway, wherein the first arm comprises a heavy chain variable region having at least about 90% sequence identity to SEQ ID NO:94 and a light chain variable region having at least about 90% sequence identity to SEQ ID NO:95, and wherein the second arm comprises SEQ ID NO:56 or SEQ ID NO:87.

In some embodiments, the bispecific agent comprises (a) a first binding site that binds human MET with a K_{D} between about 0.1 nM and about 1.0 nM and (b) a second binding site that specifically binds one or more components of the human WNT pathway with a K_{D} between about 0.1 nM and about 20 nM.

In certain embodiments of each of the aforementioned aspects, as well as other aspects and/or embodiments described elsewhere herein, the binding agent is isolated. In certain embodiments of each of the aforementioned aspects, as well as other aspects and/or embodiments described elsewhere herein, the binding agent is substantially pure.

In another aspect, the invention provides a polypeptide selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:55, SEQ ID NO:56, SEQ ID NO:87, SEQ ID NO:88, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:108, SEQ ID NO:110, SEQ ID NO:111, and SEQ ID NO:112. In some embodiments, the polypeptide is isolated. In certain embodiments, the polypeptide is substantially pure. In certain embodiments, the polypeptide is an antibody or part of an antibody, such as an antibody fragment. In some embodiments, the polypeptide is a soluble receptor or a fragment of a soluble receptor. In some embodiments, the polypeptide is a fusion protein.

The invention further provides cells that comprise the bispecific agents, antibodies, soluble receptors, and/or polypeptides described herein. The invention further provides cells that produce the bispecific agents, antibodies, soluble receptors, and/or polypeptides described herein. In some embodiments, the cell is a prokaryotic cell. In some embodiments, the cell is an eukaryotic cell.

In another aspect, the invention provides isolated polynucleotide molecules comprising a polynucleotide that encodes the binding agents and/or polypeptides of each of the aforementioned aspects, as well as other aspects and/or embodiments described herein. In some embodiments, the polynucleotide comprises a polynucleotide sequence that encodes a sequence selected from the group consisting of: SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:55, SEQ ID NO:56, SEQ ID NO:87, SEQ ID NO:88, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:108, SEQ ID NO:110, SEQ ID NO:111, and SEQ ID NO:112. In some embodiments, the polynucleotide comprises a sequence selected from the group consisting of: SEQ ID NO:15, SEQ ID NO:16, SEQ ID NO:17, SEQ ID NO:18, SEQ ID NO:19, SEQ ID NO:20, SEQ ID NO:89, SEQ ID NO:90, SEQ ID NO:102, SEQ ID NO:103, SEQ ID NO:104, SEQ ID NO:105, SEQ ID NO:106, SEQ ID NO:107, and SEQ ID NO:109.

The invention further provides expression vectors that comprise the polynucleotides, as well as cells that comprise the expression vectors and/or the polynucleotides. In some embodiments, the cell is a prokaryotic cell. In some embodiments, the cell is an eukaryotic cell.

Pharmaceutical compositions comprising a binding agent, a bispecific agent, an antibody, a soluble receptor, and/or a polypeptide described herein and a pharmaceutically acceptable carrier are further provided.

In another aspect, the invention provides methods of using the binding agents, bispecific agents, antibodies, soluble receptors, and/or polypeptides described herein. In some embodiments, a method of inhibiting growth of a tumor comprises contacting the tumor with an effective amount of a bispecific agent or antibody described herein. In some embodiments, a method of inhibiting growth of a tumor in a subject comprises administering to the subject a therapeutically effective amount of a bispecific agent or antibody described herein. In some embodiments, a method of reducing the tumorigenicity of a tumor in a subject by reducing the frequency of cancer stem cells in the tumor comprises administering to the subject a therapeutically effective amount of a bispecific agent or antibody described herein. In some embodiments, a method of reducing the frequency of cancer stem cells in a tumor in a subject comprises administering to the subject a therapeutically effective amount of a bispecific agent or antibody described herein. In some embodiments, a method of inhibiting epithelial-mesenchymal transition (EMT) in a tumor in a subject comprises administering to the subject a therapeutically effective amount of a bispecific agent or antibody described herein. In some embodiments, the tumor is selected from the group consisting of colorectal tumor, colon tumor, ovarian tumor, pancreatic tumor, lung tumor, liver tumor, breast tumor, kidney tumor, prostate tumor, gastrointestinal tumor, melanoma, cervical tumor, bladder tumor, glioblastoma, and head and neck tumor.

In some embodiments, a method of treating cancer in a subject comprises administering to the subject a therapeutically effective amount of a bispecific agent or antibody described herein. The invention also provides a bispecific agent or antibody for use in a method of treating cancer, wherein the bispecific agent or antibody is an agent or antibody described herein. The invention also provides the use of a bispecific agent or antibody described herein for the manufacture of a medicament for the treatment of cancer. In some embodiments, the cancer is selected from the group consisting of colorectal cancer, colon cancer, ovarian cancer, pancreatic cancer, lung cancer, liver cancer, breast cancer, kidney cancer, prostate cancer, gastrointestinal cancer, melanoma, cervical cancer, bladder cancer, glioblastoma, and head and neck cancer. In some embodiments, a method further comprises administering at least one additional therapeutic agent.

The invention also provides a bispecific agent or antibody for use in a method of treating cancer, wherein the bispecific agent or antibody is an agent or antibody described herein. The invention also provides the use of a bispecific agent or antibody described herein for the manufacture of a medicament for the treatment of cancer.

Also provided are methods of treatment comprising administering to a subject (e.g., a human) an effective amount of a binding agent, a bispecific agent, an antibody, a soluble receptor, or a polypeptide described herein, wherein the binding agent, a bispecific agent, an antibody, a soluble receptor, or a polypeptide is part of a pharmaceutical composition.

In another aspect, the invention provides a method of identifying a human subject or selecting a human subject for treatment with a binding agent, a bispecific agent, an antibody, a soluble receptor, or a polypeptide described herein. In some embodiments, the method comprises determining if the subject has a tumor that has an elevated expression level of MET as compared to a reference sample or to a pre-determined level. In some embodiments, the method comprises identifying a subject for treatment or selecting a subject for treatment if the tumor has an elevated level of MET expression.

Where aspects or embodiments of the invention are described in terms of a Markush group or other grouping of alternatives, the present invention encompasses not only the entire group listed as a whole, but also each member of the group individually and all possible subgroups of the main group, and also the main group absent one or more of the group members. The present invention also envisages the explicit exclusion of one or more of any of the group members in the claimed invention.

### BRIEF DESCRIPTIONS OF THE DRAWINGS

FIG. 1A. Inhibition of binding of hepatocyte growth factor to human MET. Cells treated with only HGF were used as a positive control and untreated transfected cells were used as a negative control.

FIG. 1B. Inhibition of binding of hepatocyte growth factor to human MET. HEK-293T cells were transiently transfected with a human MET construct and then subsequently mixed with anti-MET antibody 5D5 and hepatocyte growth factor (HGF). Specific binding is indicated by the presence of signal within the box overlay on each FACS plot. The percent binding is shown underneath each FACS plot. The percent inhibition of binding as compared to the percent binding of the average of the two positive controls in shown underneath each FACS plot.

FIG. 1C. Inhibition of binding of hepatocyte growth factor to human MET, HEK-293T cells were transiently transfected with a human MET construct and then subsequently mixed with a monovalent version of anti-MET antibody 73R009 and hepatocyte growth factor (HGF). Specific binding is indicated by the presence of signal within the box overlay on each FACS plot. The percent binding is shown underneath each FACS plot. The percent inhibition of binding as compared to the percent binding of the average of the two positive controls in shown underneath each FACS plot.

FIG. 1D. Inhibition of binding of hepatocyte growth factor to human MET. HEK-293T cells were transiently transfected with a human MET construct and then subsequently mixed with anti-MET/FZD8-Fc bispecific agent 315B06 and hepatocyte growth factor (HGF). Specific binding is indicated by the presence of signal within the box overlay on each FACS plot. The percent binding is shown underneath each FACS plot. The percent inhibition of binding as compared to the percent binding of the average of the two positive controls in shown underneath each FACS plot.

FIG. 2. Inhibition of MET activity induced by hepatocyte growth factor. A549 cells were pre-treated for one hour with a monovalent version of anti-MET antibody 73R009, anti-MET/FZD8 bispecific agent 5D5/FZD8-Fc, or anti-MET/FZD8-Fc bispecific agent 315B06 and then stimulated with human hepatocyte growth factor. Cell lysates were analyzed by Western blotting.

FIG. 3. Inhibition of WNT signaling. A 8×TCF-luciferase reporter assay was used to measure WNT signaling in STF-293 cells. STF-293 cells were treated with anti-MET/FZD8-Fc bispecific agent 315B06 (-♦-) or control binding agents monovalent anti-MET antibody 5D5/FLAG (-X-) and monovalent FZD8-Fc/FLAG (-▴-). Cells were exposed to medium containing WNT3a L cell-conditioned medium (- [symbol] -), or control medium from cells not over-expressing WNT3a (--).

FIG. 4A. Inhibition of OMP-LU45 lung tumor growth. LU45 lung tumor cells were injected subcutaneously into NOD/SCID mice. Mice were treated with a control antibody (--), monovalent anti-MET antibody 5D5 (-□-), monovalent FZD8-Fc/FLAG (-▴-), bivalent FZD8-Fc (54F28) (-♦-), or anti-MET/FZD8-Fc bispecific agent (-▾-). Data is shown as tumor volume (mm³) over days post treatment.

FIG. 4B. Inhibition of OMP-LU45 lung tumor growth. LU45 lung tumor cells were injected subcutaneously into NOD/SCID mice. Mice were treated with a control antibody (--), monovalent anti-MET antibody 5D5 (-□-), monovalent FZD8-Fc/FLAG (-▴-), bivalent FZD8-Fc (54F28) or anti-MET/FZD8-Fc bispecific agent (-▾-) in combination with taxol. Data is shown as tumor volume (mm³) over days post treatment.

FIG. 5. Epitope mapping of 315B07. HEK-293T cells were transiently transfected with a sema constructs and then subsequently mixed with anti-MET/FZD8-Fc bispecific agent 315107 or anti-MET/FZD8-Fc bispecific agent 5D5/FZD8-Fc. Specific binding was determined by FACS analyses.

FIG. 6. Ribbon structure of MET showing the predicted epitope of anti-MET/FZD8-Fc bispecific agent 315B07 and the predicted epitope of anti-MET antibody 5D5.

FIG. 7. Inhibition of MMTV-Wnt1 breast tumor growth. MMTV-Wnt1 tumor cells were injected subcutaneously into NOD/SCID mice. Mice were treated with a control antibody (-▪-), bivalent FZD8-Fc (54F28) (-▾-), anti-MET/FZD8-Fc bispecific agent 315B07 at 30 mg/kg (--), or anti-MET/FZD8-Fc bispecific agent 315B07 at 60 mg/kg (-Δ-). Data is shown as tumor volume (mm³) over days post treatment.

FIG. 8. Inhibition of KP4 pancreatic tumor growth. KP4 pancreatic tumor cells were injected subcutaneously into NOD/SCID mice. Mice were treated with a control antibody (-▪-), anti-FZD antibody 18R5 (-▾-), monovalent FZD8-Fc/FLAG (-Δ-), monovalent anti-MET antibody 5D5/FLAG (--), or anti-MET/FZD8-Fc bispecific agent (-◯-). Data is shown as tumor volume (mm³) over days post treatment.

FIG. 9. Inhibition of OMP-LIV1 hepatocellular cancer tumor growth. OMP-LIV1 hepatocellular cancer (HCC) tumor cells were injected subcutaneously into NOD/SCID mice. Mice were treated with sorafenib (-▪-), monovalent anti-MET/FLAG in combination with sorafenib (-▾-), monovalent FZD8-Fc/FLAG in combination with sorafenib (--), or anti-MET/FZD8-Fc bispecific agent 315B07 in combination with sorafenib (-Δ-). Data is shown as tumor volume (mm³) over days post treatment.

### DETAILED DESCRIPTION OF THE INVENTION

The present invention provides novel binding agents that bind MET, bind one or more components of the WNT pathway, or bind both MET and one or more components of the WNT pathway. The phrase “components of the WNT pathway” as used herein, generally refers to one or more WNT proteins and/or one or more FZD proteins. Related polypeptides and polynucleotides, compositions comprising the binding agents, and methods of making the binding agents are also provided. Methods of using the novel binding agents, such as methods of inhibiting tumor growth, methods of treating cancer, methods of reducing tumorigenicity of a tumor, methods of reducing the frequency of cancer stem cells in a tumor, methods of inhibiting EMT, methods of inhibiting angiogenesis, and/or methods of identifying and/or selecting subjects for treatment, are further provided.

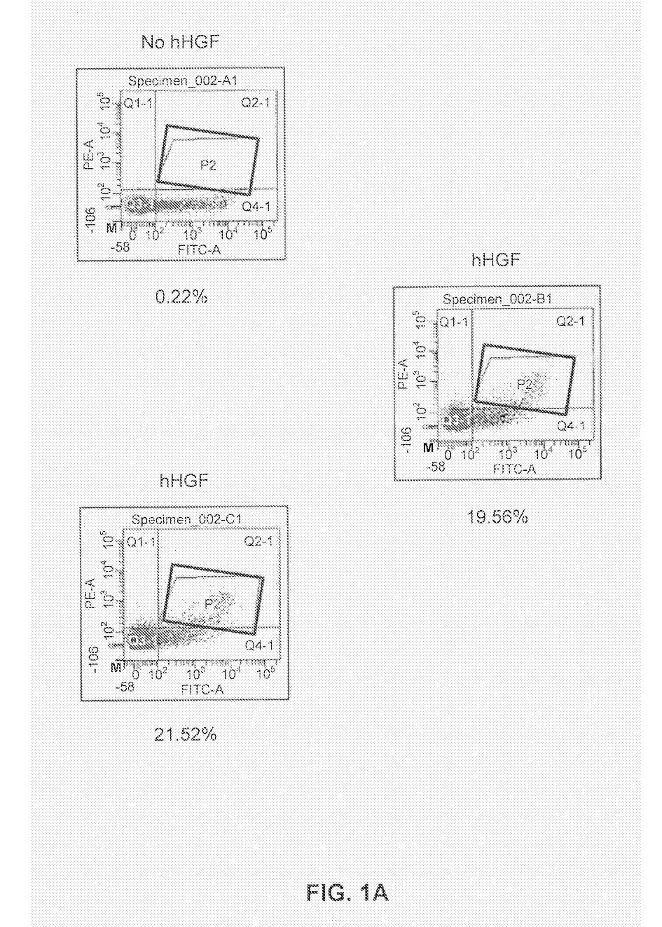

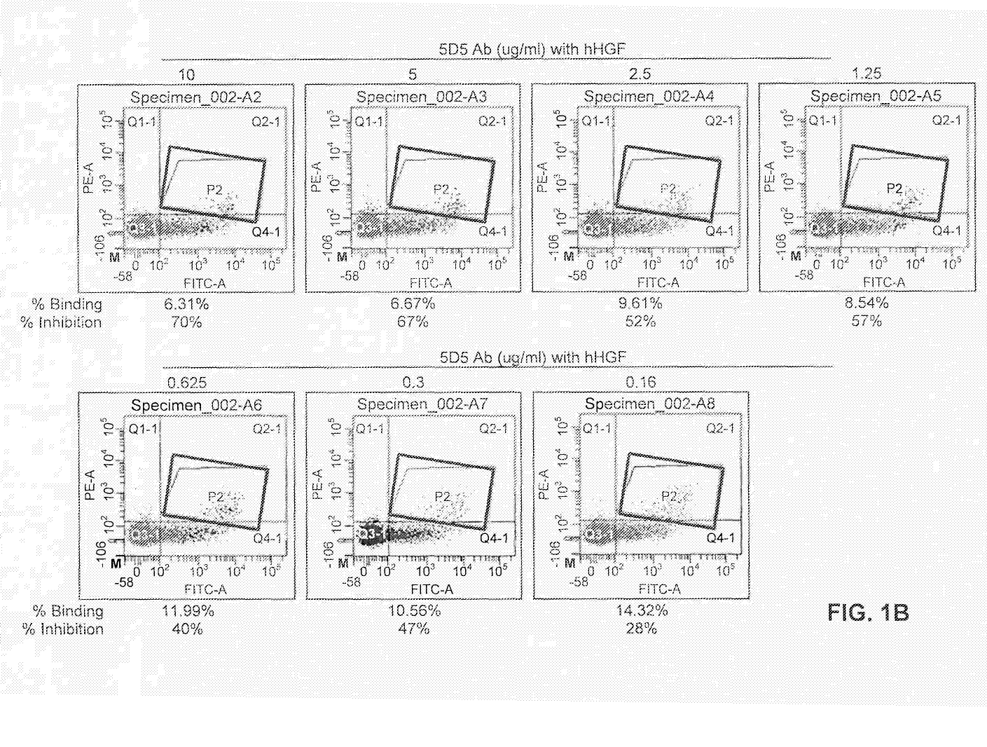

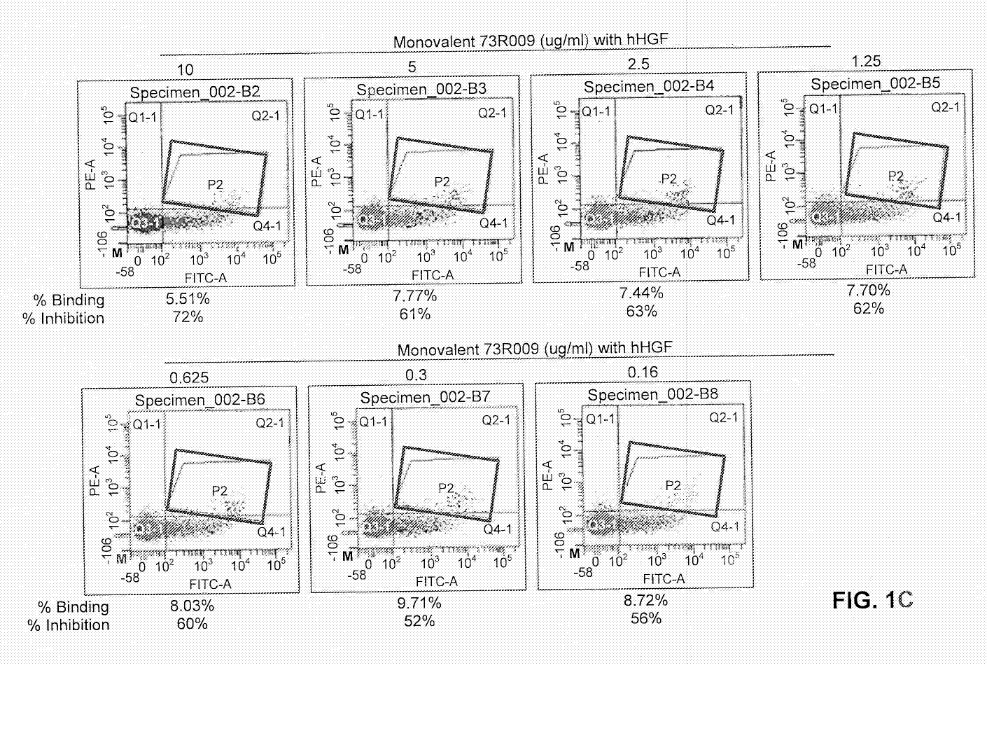

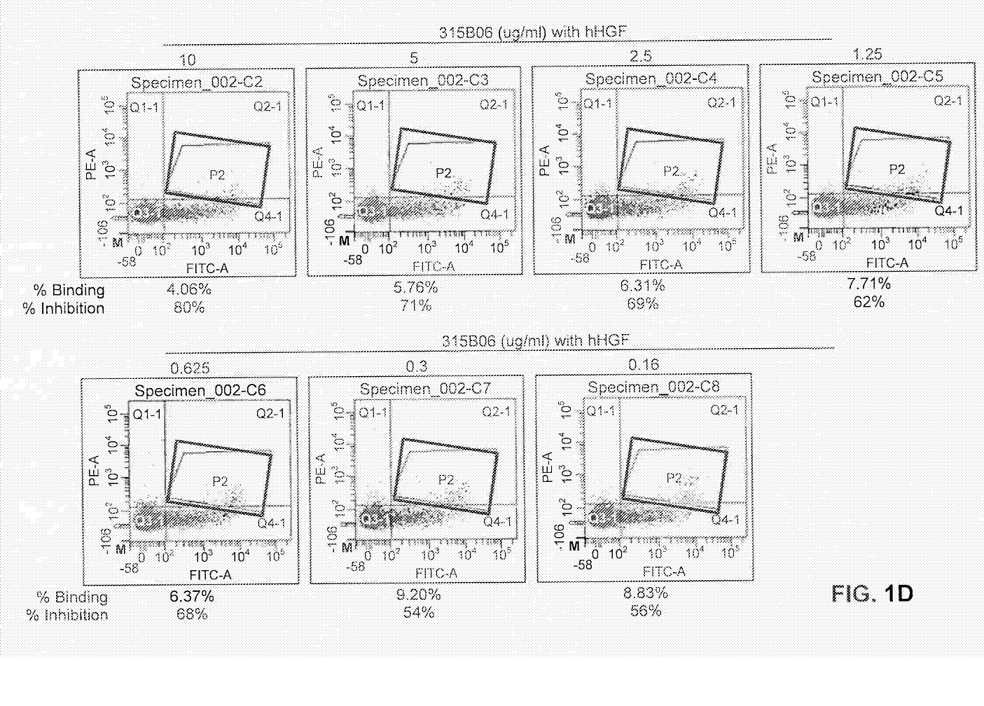

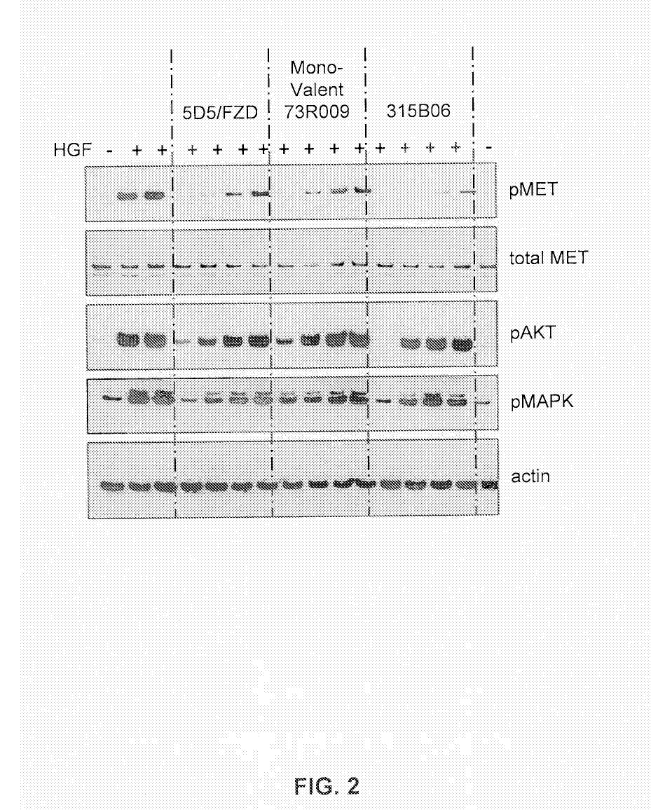

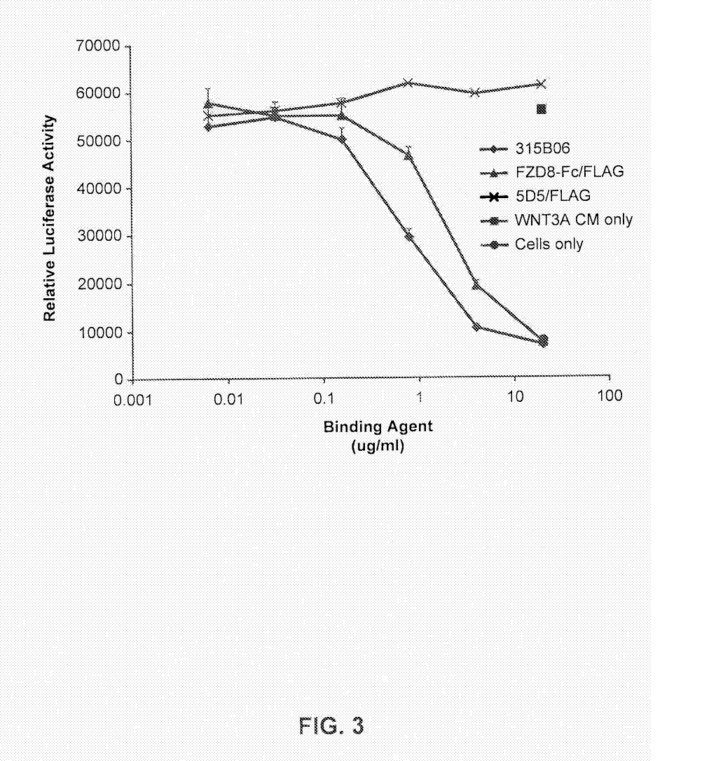

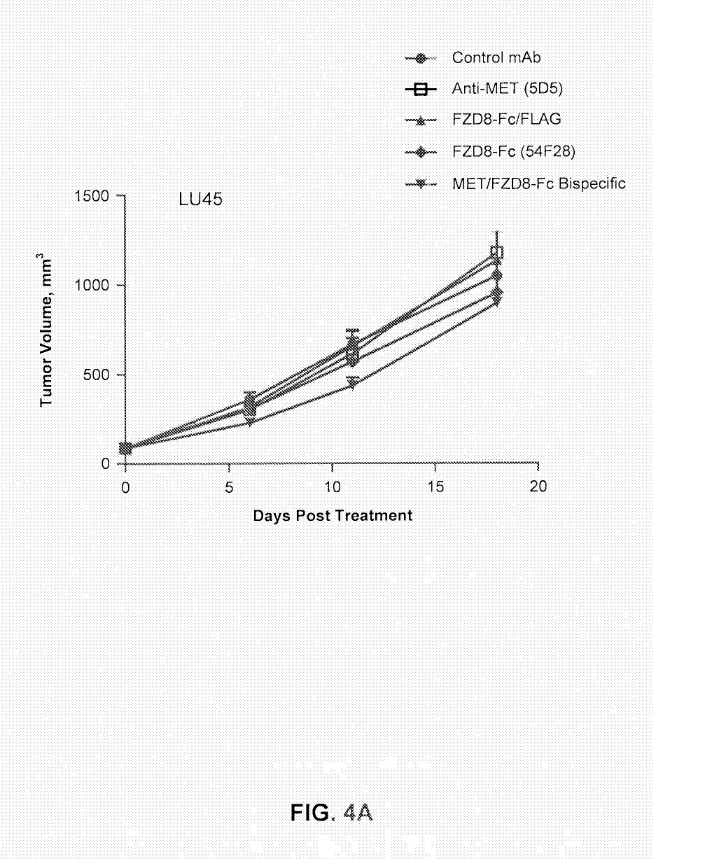

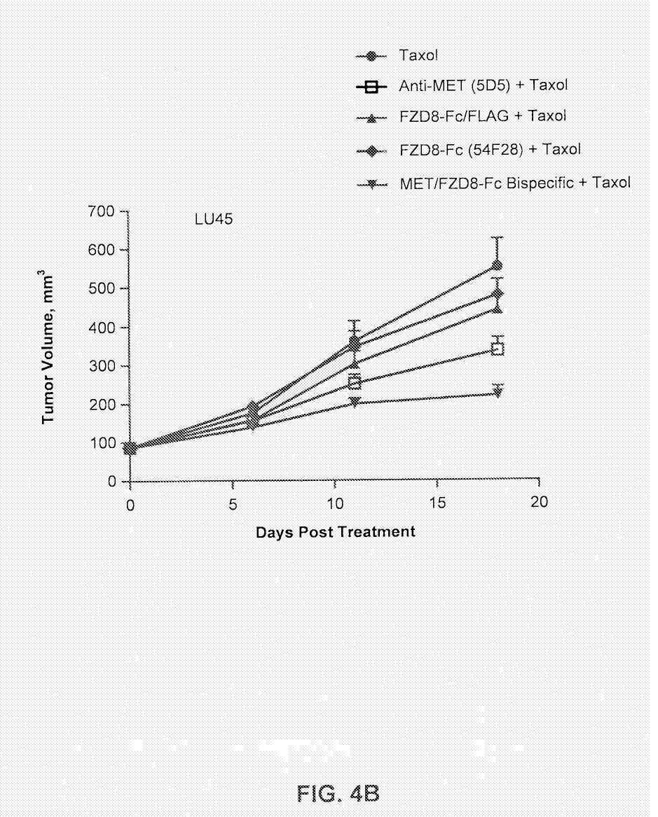

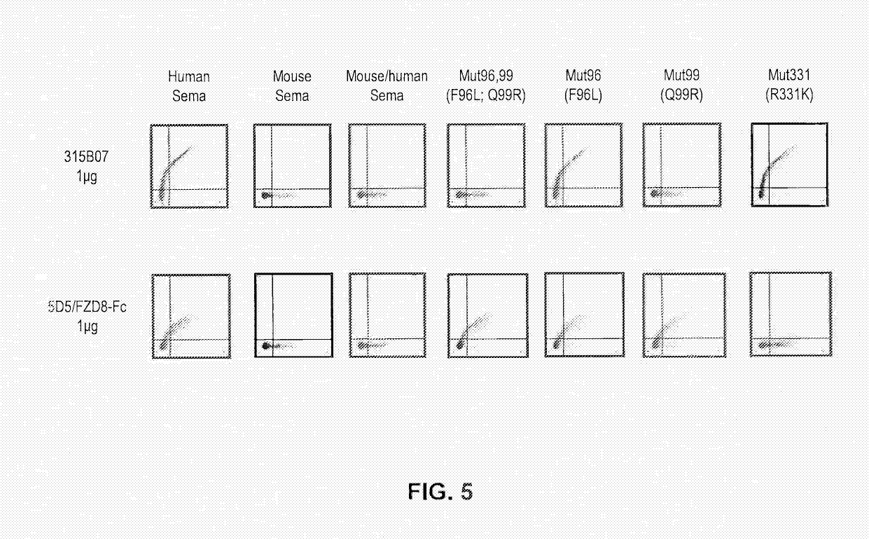

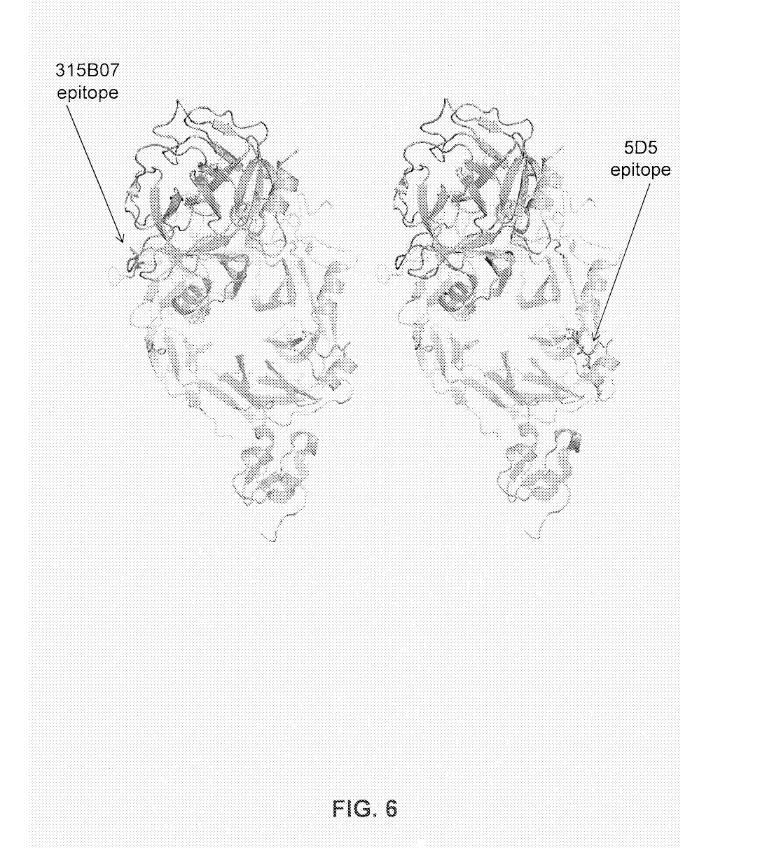

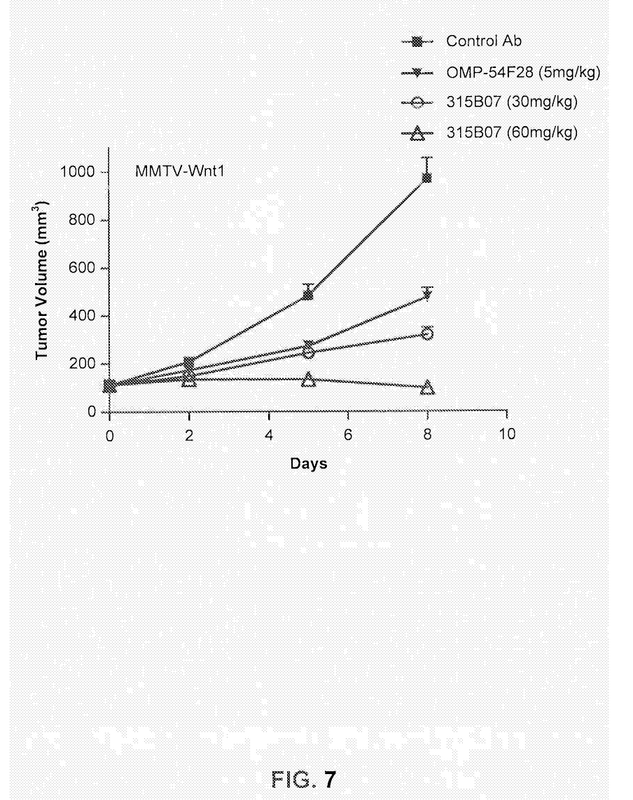

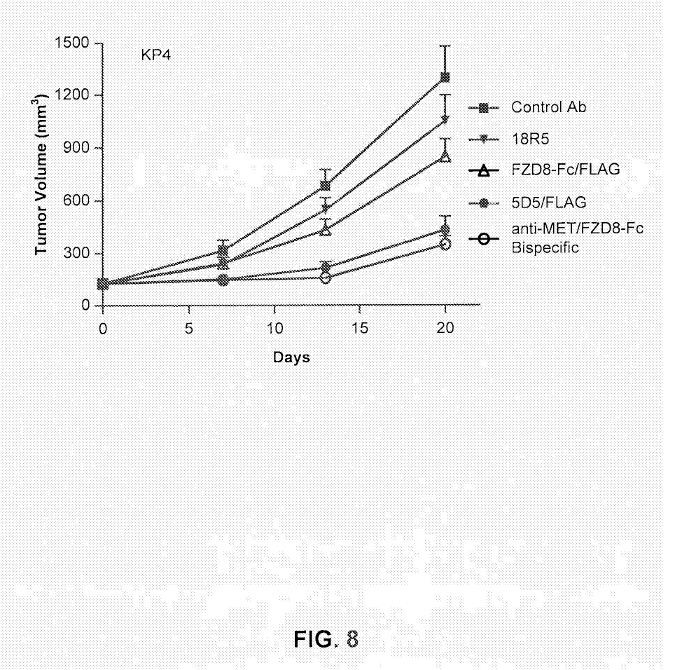

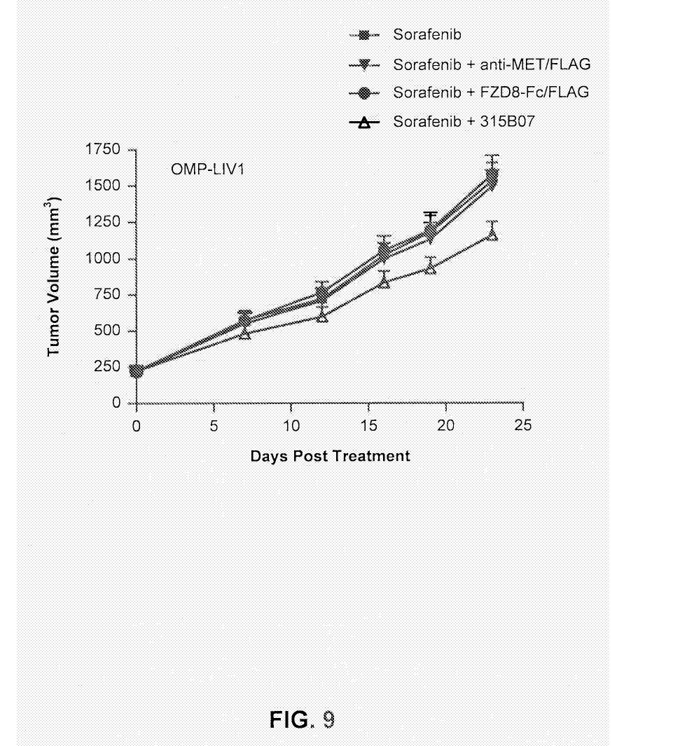

A monoclonal antibody that specifically binds human MET has been identified, antibody 73R009. A humanized version of antibody 73R009 was generated, antibody 73R010. The antibody 73R009 has a binding affinity for human MET of about 1.1 nM and does not bind mouse MIT. A monovalent version of the antibody has been generated and has a binding affinity for human MET of 1.4 nM. Bispecific agents that specifically bind human MET and one or more human WNT proteins have been produced, 315B06, 315B07, and 315B09. Bispecific agent 315B06 has a binding affinity for human MET of 1.8 nM and does not bind mouse MET. Bispecific agent 315B07 has a binding affinity for human MET of about 125 pM and does not bind mouse MET. Bispecific agent 315B06 inhibits binding of human hepatocyte growth factor (HGF) to human MET (Example 2, FIG. 1). Bispecific agent 315B06 inhibits HGF-induced MET activity (Example 3, FIG. 2). Bispecific agent 315B06 inhibits WNT pathway signaling (Example 4, FIG. 3). The epitope on human MET for the bispecific agent 315B07 was determined and shown to be distinct from the epitope of monovalent anti-MET antibody 5D5 (Example 7, FIGS. 5 and 6). A bispecific agent comprising an anti-MET antibody and a FZD8-Fc inhibited growth of a human lung tumor when combined with taxol (Example 5, FIG. 4). A bispecific agent comprising an anti-MET antibody and a FZD8-Fc inhibited growth of a breast tumor (Example 8. FIG. 7), of a human pancreatic tumor (Example 9, FIG. 8), and of a human liver tumor (Example 10, FIG. 9).

### I. DEFINITIONS

To facilitate an understanding of the present invention, a number of terms and phrases are defined below.

The term “antibody” as used herein refers to an immunoglobulin molecule that recognizes and specifically binds a target, such as a protein, polypeptide, peptide, carbohydrate, polynucleotide, lipid, or combinations of the foregoing, through at least one antigen-binding site within the variable region of the immunoglobulin molecule. As used herein, the term encompasses intact polyclonal antibodies, intact monoclonal antibodies, single chain antibodies, antibody fragments (such as Fab, Fab′, F(ab′)2, and Fv fragments), single chain Fv (scFv) antibodies, multispecific antibodies such as bispecific antibodies, monospecific antibodies, monovalent antibodies, chimeric antibodies, humanized antibodies, human antibodies, fusion proteins comprising an antigen-binding site of an antibody, and any other modified immunoglobulin molecule comprising an antigen-binding site as long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1, and IgA2), based on the identity of their heavy chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, single chain antibodies, and multispecific antibodies formed from antibody fragments. “Antibody fragment” as used herein comprises at least one antigen-binding site or epitope-binding site.

The term “variable region” of an antibody refers to the variable region of an antibody light chain, or the variable region of an antibody heavy chain, either alone or in combination. The variable region of a heavy or light chain generally consist of four framework regions (FR) connected by three complementarity determining regions (CDRs), also known as “hypervariable regions”. The CDRs in each chain are held together in close proximity by the framework regions and, with the CDRs from the other chain, contribute to the formation of the antigen-binding site(s) of the antibody. There are at least two techniques for determining CDRs: (1) an approach based on cross-species sequence variability (i.e., Kabat et al., 1991, Sequences of Proteins of Immunological Interest, 5th Edition, National Institutes of Health, Bethesda, Md.), and (2) an approach based on crystallographic studies of antigen-antibody complexes (Al-Lazikani et al., 1997, J. Mol. Biol., 273:927-948). In addition, combinations of these two approaches are sometimes used in the art to determine CDRs.

The term “monoclonal antibody” as used herein refers to a homogeneous antibody population involved in the highly specific recognition and binding of a single antigenic determinant or epitope. This is in contrast to polyclonal antibodies that typically include a mixture of different antibodies directed against a variety of different antigenic determinants. The term “monoclonal antibody” encompasses both intact and full-length monoclonal antibodies as well as antibody fragments (e.g., Fab, Fab′, F(ab′)2, Fv), single chain (scFv) antibodies, fusion proteins comprising an antibody portion, and any other modified immunoglobulin molecule comprising an antigen-binding site. Furthermore, “monoclonal antibody” refers to such antibodies made by any number of techniques, including but not limited to, hybridoma production, phage selection, recombinant expression, and transgenic animals.

The term “humanized antibody” as used herein refers to forms of non-human (e.g., murine) antibodies that are specific immunoglobulin chains, chimeric immunoglobulins, or fragments thereof that contain minimal non-human sequences. Typically, humanized antibodies are human immunoglobulins in which residues of the CDRs are replaced by residues from the CDRs of a non-human species (e.g., mouse, rat, rabbit, or hamster) that have the desired specificity, affinity, and/or binding capability (Jones et al., 1986, Nature, 321:522-525; Riechmann et al., 1988, Nature, 332:323-327; Verhoeyen et al., 1988, Science, 239:1534-1536). In some instances, the Fv framework region residues of a human immunoglobulin are replaced with the corresponding residues in an antibody from a non-human species that has the desired specificity, affinity, and/or binding capability. The humanized antibody can be farther modified by the substitution of additional residues either in the Fv framework region and/or within the replaced non-human residues to refine and optimize antibody specificity, affinity, and/or binding capability. In general, the humanized antibody will comprise substantially all of at least one, and typically two, variable domains containing all or substantially all of the CDRs that correspond to the non-human immunoglobulin whereas all or substantially all of the framework regions are those of a human immunoglobulin consensus sequence. The humanized antibody can also comprise at least a portion of an immunoglobulin constant region or domain (Fc), typically that of a human immunoglobulin. Methods used to generate humanized antibodies are well known in the art.

The term “human antibody” as used herein refers to an antibody produced by a human or an antibody having an amino acid sequence corresponding to an antibody produced by a human. A human antibody may be made using any of the techniques known in the art. This definition of a human antibody specifically excludes a humanized antibody comprising non-human CDRs.

The term “chimeric antibody” as used herein refers to an antibody wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and/or binding capability, while the constant regions correspond to sequences in antibodies derived from another species (usually human).

The phrase “affinity-matured antibody” as used herein refers to an antibody with one or more alterations in one or more CDRs thereof that result in an improvement in the affinity of the antibody for antigen, compared to a parent antibody that does not possess those alterations(s). The definition also includes alterations in non-CDR residues made in conjunction with alterations to CDR residues. Preferred affinity-matured antibodies will have nanomolar or even picomolar affinities for the target antigen. Affinity-matured antibodies are produced by procedures known in the art. For example, Marks et al., 1992, Bio/Technology 10:779-783, describes affinity maturation by VH and VL domain shuffling. Random mutagenesis of CDR and/or framework residues is described by Barbas et al., 1994, PNAS, 91:3809-3813; Schier et al., 1995, Gene, 169:147-155; Yelton et al., 1995, 1 Immunol. 155:1994-2004; Jackson et al., 1995, J. Immunol., 154:3310-9; and Hawkins et al., 1992, J. Mol. Biol., 226:889-896. Site-directed mutagenesis may also be used to obtain affinity-matured antibodies.

The terms “epitope” and “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5 or 8-10 amino acids in a unique spatial conformation.

The terms “heteromultimeric molecule” or “heteromultimer” or “heteromultimeric complex” or “heteromultimeric polypeptide” are used interchangeably herein to refer to a molecule comprising at least a first polypeptide and a second polypeptide, wherein the second polypeptide differs in amino acid sequence from the first polypeptide by at least one amino acid residue. The heteromultimeric molecule can comprise a “heterodimer” or “heterodimeric agent” formed by the first and second polypeptide or can form higher order tertiary structures where additional polypeptides are present.

The terms “antagonist” and “antagonistic” as used herein refer to any molecule that partially or fully blocks, inhibits, reduces, or neutralizes a biological activity of a target and/or signaling pathway (e.g., the WNT pathway or MET pathway). The term “antagonist” is used herein to include any molecule that partially or fully blocks, inhibits, reduces, or neutralizes the activity of a protein. Suitable antagonist molecules specifically include, but are not limited to, antagonist antibodies, antibody fragments, soluble receptors, or fragments of soluble receptors.

The terms “modulation” and “modulate” as used herein refer to a change or an alteration in a biological activity. Modulation includes, but is not limited to, stimulating or inhibiting an activity. Modulation may be an increase or a decrease in activity (e.g., a decrease in pathway signaling), a change in binding characteristics, or any other change in the biological, functional, or immunological properties associated with the activity of a protein, pathway, or other biological point of interest.

The terms “selectively binds” or “specifically binds” mean that a binding agent or an antibody reacts or associates more frequently, more rapidly, with greater duration, with greater affinity, or with some combination of the above to the epitope, protein, or target molecule than with alternative substances, including unrelated or related proteins. In certain embodiments “specifically hinds” means, for instance, that an antibody binds a protein with a K_{D} of about 0.1 mM or less, but more usually less than about 1 μM. In certain embodiments, “specifically binds” means that an antibody binds a target at times with a K_{D} of at least about 0.1 μM or less, at other times at least about 0.01 μM or less, and at other times at least about 1 nM or less. Because of the sequence identity between homologous proteins in different species, specific binding can include an antibody that recognizes a protein in more than one species (e.g., human MET and mouse MET). Likewise, because of homology within certain regions of polypeptide sequences of different proteins, specific binding can include an antibody (or other polypeptide or binding agent) that recognizes more than one protein (e.g., human WNT1 and human WNT7). It is understood that, in certain embodiments, an antibody or binding agent that specifically binds a first target may or may not specifically bind a second target. As such, “specific binding” does not necessarily require (although it can include) exclusive binding, i.e. binding to a single target. Thus, a binding agent may, in certain embodiments, specifically hind more than one target. In certain embodiments, multiple targets may be bound by the same binding site on the agent or antibody. For example, an antibody may, in certain instances, comprise two identical antigen-binding sites, each of which specifically binds the same epitope on two or more proteins. In certain alternative embodiments, an antibody may be bispecific or multispecific and comprise at least two antigen-binding sites with differing specificities. By way of non-limiting example, a bispecific agent may comprise one binding site that recognizes a target on one protein (e.g., human MET) and further comprise a second, different binding site that recognizes a different target on a second protein (e.g., a human WNT protein). Generally, but not necessarily, reference to binding means specific binding.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. It is understood that, because the polypeptides of this invention may be based upon antibodies, in certain embodiments, the polypeptides can occur as single chains or associated chains (e.g., dimers).

The terms “polynucleotide” and “nucleic acid” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

“Conditions of high stringency” may be identified by those conditions that: (1) employ low ionic strength and high temperature for washing, for example 15 mM sodium chloride/1.5 mM sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 in 5×SSC (0.75M NaCl, 75 mM sodium citrate) at 42° C.; or (3) employ during hybridization 50% formamide in 5×SSC, 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC and 50% formamide, followed by a high-stringency wash consisting of 0.1×SSC containing EDTA at 55° C.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variations thereof. In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 40-60 residues, at least about 60-80 residues in length or any integral value therebetween. In some embodiments, identity exists over a longer region than 60-80 residues, such as at least about 80-100 residues, and in some embodiments the sequences are substantially identical over the full length of the sequences being compared, such as the coding region of a nucleotide sequence.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Preferably, conservative substitutions in the sequences of the polypeptides and antibodies of the invention do not abrogate the binding of the polypeptide or antibody containing the amino acid sequence, to the antigen to which the polypeptide or antibody binds. Methods of identifying nucleotide and amino acid conservative substitutions which do not eliminate antigen binding are well-known in the art.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

As used herein the term “soluble receptor” refers to an extracellular domain (or a fragment thereof) of a receptor protein preceding the first transmembrane domain of the receptor that can be secreted from a cell in soluble form. Generally this is the N-terminal portion of the receptor protein.

As used herein the term “FZD soluble receptor” or “soluble FZD receptor” refers to an N-terminal extracellular fragment of a FZD receptor protein preceding the first transmembrane domain of the receptor that can be secreted from a cell in soluble form. FZD soluble receptors comprising the entire N-terminal extracellular domain (ECD) as well as smaller fragments are encompassed by the term. Thus, FZD soluble receptors comprising a FZD Fri domain are also included in this term.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells, or compositions include those which have been purified to a degree that they are no longer in a form in which they are found in nature. In some embodiments, a polypeptide, antibody, polynucleotide, vector, cell, or composition which is isolated is substantially pure.

The term “substantially pure” as used herein refers to material which is at least 50% pure (i.e., free from contaminants), at least 90% pure, at least 95% pure, at least 98% pure, or at least 99% pure.

The terms “cancer” and “cancerous” as used herein refer to or describe the physiological condition in mammals in which a population of cells are characterized by unregulated cell growth. Examples of cancer include, but are not limited to, carcinoma, blastoma, sarcoma, and hematologic cancers such as lymphoma and leukemia.

The terms “tumor” and “neoplasm” as used herein refer to any mass of tissue that results from excessive cell growth or proliferation, either benign (non-cancerous) or malignant (cancerous) including pre-cancerous lesions.

The term “metastasis” as used herein refers to the process by which a cancer spreads or transfers from the site of origin to other regions of the body with the development of a similar cancerous lesion at a new location. A “metastatic” or “metastasizing” cell is one that loses adhesive contacts with neighboring cells and migrates (e.g., via the bloodstream or lymph) from the primary site of disease to secondary sites.

The terms “cancer stem cell” and “CSC” and “tumor stein cell” and “tumor initiating cell” are used interchangeably herein and refer to cells from a cancer or tumor that: (1) have extensive proliferative capacity; 2) are capable of asymmetric cell division to generate one or more types of differentiated cell progeny wherein the differentiated cells have reduced and/or limited proliferative or developmental potential; and (3) are capable of symmetric cell divisions for self-renewal or self-maintenance. These properties confer on the cancer stein cells the ability to form or establish a tumor or cancer upon serial transplantation into an immunocompromised host (e.g., a mouse) compared to the majority of tumor cells that fail to form tumors. Cancer stem cells undergo self-renewal versus differentiation in a chaotic manner to form tumors with abnormal cell types that can change over time as mutations occur.

The terms “cancer cell” and “tumor cell” refer to the total population of cells derived from a cancer or tumor or pre-cancerous lesion, including both non-tumorigenic cells, which comprise the bulk of the cancer cell population, and tumorigenic stem cells (cancer stem cells). As used herein, the terms “cancer cell” or “tumor cell” will be modified by the term “non-tumorigenic” when referring solely to those cells lacking the capacity to renew and differentiate to distinguish those tumor cells from cancer stem cells.

The term “tumorigenic” as used herein refers to the functional features of a cancer stem cell including the properties of self-renewal (giving rise to additional tumorigenic cancer stem cells) and proliferation to generate all other tumor cells (giving rise to differentiated and thus non-tumorigenic tumor cells).

The term “tumorigenicity” as used herein refers to the ability of a random sample of cells from the tumor to form palpable tumors upon serial transplantation into immunocompromised hosts (e.g., mice). This definition also includes enriched and/or isolated populations of cancer stem cells that form palpable tumors upon serial transplantation into immunocompromised hosts (e.g., mice).

The term “subject” refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The term “pharmaceutically acceptable” refers to a product or compound approved (or approvable) by a regulatory agency of the Federal government or a state government or listed in the U.S. Pharmacopeia or other generally recognized pharmacopeia for use in animals, including humans.

The terms “pharmaceutically acceptable excipient, carrier or adjuvant” or “acceptable pharmaceutical carrier” refer to an excipient, carrier, or adjuvant that can be administered to a subject, together with at least one binding agent of the present disclosure, and which does not destroy the activity of the binding agent. The excipient, carrier, or adjuvant should be non-toxic when administered with a binding agent in doses sufficient to deliver a therapeutic effect.

The terms “effective amount” or “therapeutically effective amount” or “therapeutic effect” refer to an amount of a binding agent, an antibody, polypeptide, polynucleotide, small organic molecule, or other drug effective to “treat” a disease or disorder in a subject or mammal. In the case of cancer, the therapeutically effective amount of a drug (e.g., an antibody) has a therapeutic effect and as such can reduce the number of cancer cells; decrease tumorigenicity, tumorigenic frequency or tumorigenic capacity; reduce the number or frequency of cancer stem cells; reduce the tumor size; reduce the cancer cell population; inhibit and/or stop cancer cell infiltration into peripheral organs including, for example, the spread of cancer into soft tissue and bone; inhibit and/or stop tumor or cancer cell metastasis; inhibit and/or stop tumor or cancer cell growth; relieve to some extent one or more of the symptoms associated with the cancer; reduce morbidity and mortality; improve quality of life; or a combination of such effects. To the extent the agent, for example an antibody, prevents growth and/or kills existing cancer cells, it can be referred to as cytostatic and/or cytotoxic.

The terms “treating” or “treatment” or “to treat” or “alleviating” or “to alleviate” refer to both 1) therapeutic measures that cure, slow down, lessen symptoms of, and/or halt progression of a diagnosed pathologic condition or disorder and 2) prophylactic or preventative measures that prevent or slow the development of a targeted pathologic condition or disorder. Thus those in need of treatment include those already with the disorder; those prone to have the disorder; and those in whom the disorder is to be prevented. In some embodiments, a subject is successfully “treated” according to the methods of the present invention if the patient shows one or more of the following: a reduction in the number of or complete absence of cancer cells; a reduction in the tumor size; an inhibition of tumor growth; inhibition of or an absence of cancer cell infiltration into peripheral organs including the spread of cancer cells into soft tissue and bone; inhibition of or an absence of tumor or cancer cell metastasis; inhibition or an absence of cancer growth; relief of one or more symptoms associated with the specific cancer; reduced morbidity and mortality; improvement in quality of life; reduction in tumorigenicity; reduction in the number or frequency of cancer stem cells; or some combination of effects.

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. MET-BINDING AGENTS

The present invention provides agents that specifically bind human MET. The agents are referred to herein as “MET-binding agents”. The phrase “MET-binding agent” encompasses agents that bind only MET and bispecific agents that bind both MET and at least one additional target or antigen. Thus, in some embodiments, the MET-binding agent specifically binds human MET. In some embodiments, the MET-binding agent specifically binds both MET and at least one additional target or antigen. In some embodiments, the MET-binding agent binds both MET and one or more components of the WNT pathway. In some embodiments, the MET-binding agent binds both MET and one or more WNT proteins. In some embodiments, the MET-binding agent binds both MET and one or more FZD proteins. In some embodiments, the MET-binding agent is a polypeptide. In some embodiments, the MET-binding agent is an antibody. In some embodiments, the MET-binding agent is a monovalent antibody. In some embodiments, the MET-binding agent is a heterodimer. In certain embodiments, the MET-binding agent is a bispecific antibody. In certain embodiments, the MET-binding agent is a bispecific agent. In certain embodiments, the MET-binding agent is a bispecific agent comprising a soluble receptor. In certain embodiments, the MET-binding agent is a bispecific agent comprising a monovalent antibody that specifically binds MET. In certain embodiments, the MET-binding agent is a bispecific agent comprising a monovalent antibody that specifically binds MET and a monovalent antibody that specifically binds one or more components of the WNT pathway. In certain embodiments, the MET-binding agent is a bispecific agent (e.g., a heterodimeric agent) comprising a monovalent antibody that specifically binds MET and a soluble receptor that specifically binds one or more WNT proteins.

In certain embodiments, the MET-binding agent specifically binds the extracellular domain of human MET. In some embodiments, the MET-binding agent specifically binds the Sema domain of human MET. In some embodiments, the MET-binding agent specifically binds within the Sema domain of human MET. In some embodiments, the MET-binding agent specifically binds the alpha-chain of the Sema domain of human MET. In some embodiments, the MET-binding agent specifically binds the beta-chain of the Sema domain of human MET. In some embodiments, the binding agent specifically binds the PSI (plexin-semaphorin-integrin) domain of human MET. In some embodiments, the binding agent specifically binds an IPT (immunoglobulin-like fold, plexin, transcription factor) domain of human MET. In some embodiments, the binding agent specifically binds one or more of the IPT repeat domains of human MET. In some embodiments, the binding agent specifically binds the Sema domain, the PSI domain, and/or one or more of the IPT repeat domains of human MET. In some embodiments, the MET-binding agent specifically binds the Sema domain of human MET and does not specifically bind the Sema domain of mouse MET.

In some embodiments, the MET-binding agent specifically binds within amino acids 25-932 of human MET (SEQ ID NO:93). In some embodiments, the MET-binding agent specifically binds within amino acids 25-836 of human MET (SEQ ID NO:93). In some embodiments, the MET-binding agent specifically binds within amino acids 25-515 of human MET (SEQ ID NO:93). In some embodiments, the MET-binding agent specifically binds within amino acids 25-307 of human MET (SEQ ID NO:93). In some embodiments, the MET-binding agent specifically binds within amino acids 308-515 of human MET (SEQ ID NO:93). In some embodiments, the MET-binding agent specifically binds within amino acids 50-130 of human MET (SEQ ID NO:93). In some embodiments, the MET-binding agent specifically binds within amino acids 70-110 of human MET (SEQ ID NO:93). In some embodiments, the MET-binding agent specifically binds within amino acids 90-110 of human MET (SEQ ID NO:93). In some embodiments, the MET-binding agent specifically binds an epitope that comprises amino acids 97-101 of human MET (SEQ ID NO:93). In some embodiments, the MET-binding agent specifically binds an epitope that comprises the glutamine corresponding to position 99 of SEQ ID NO:93. In some embodiments, the MET-binding agent specifically binds an epitope that comprises amino acids PCQDC (SEQ ID NO:113). In some embodiments, the MET-binding agent specifically binds within amino acids 519-562 of human MET (SEQ ID NO:93). In some embodiments, the MET-binding agent specifically binds within amino acids 563-950 of human MET (SEQ ID NO:93). In some embodiments, the MET-binding agent specifically binds within amino acids 563-836 of human MET (SEQ ID NO:93). In some embodiments, the MET-binding agent specifically binds within amino acids 563-656 of human MET (SEQ ID NO:93). In some embodiments, the MET-binding agent specifically binds within amino acids 657-740 of human MET (SEQ ID NO:93). In some embodiments, the MET-binding agent specifically binds within amino acids 741-855 of human MET (SEQ ID NO:93). In some embodiments, the MET-binding agent specifically binds within amino acids 856-950 of human MET (SEQ ID NO:93).

In certain embodiments, the invention provides a MET-binding agent that specifically binds human MET, wherein the MET-binding agent comprises a heavy chain CDR1 comprising ASYAWS (SEQ ID NO:1), a heavy chain CDR2 comprising YISYSGGTDYNPSLKS (SEQ ID NO:2), and a heavy chain CDR3 comprising KGAY (SEQ ID NO:3). In some embodiments, the MET-binding agent further comprises a light chain CDR1 comprising SASSSVSSSYLY (SEQ ID NO:4), a light chain CDR2 comprising STSNLAS (SEQ ID NO:5), and a light chain CDR3 comprising HQWSSYPYT (SEQ ID NO:6). In certain embodiments, the MET-binding agent comprises: (a) a heavy chain CDR1 comprising ASYAWS (SEQ ID NO:1), a heavy chain CDR2 comprising YISYSGGTDYNPSLKS (SEQ ID NO:2), and a heavy chain CDR3 comprising KGAY (SEQ ID NO:3), and (b) a light chain CDR1 comprising SASSSVSSSYLY (SEQ ID NO:4), a light chain CDR2 comprising STSNLAS (SEQ ID NO:5), and a light chain CDR3 comprising HQWSSYPYT (SEQ ID NO:6).

In certain embodiments, the invention provides a MET-binding agent that specifically binds human MET, wherein the MET-binding agent comprises: (a) a heavy chain CDR1 comprising ASYAWS (SEQ ID NO:1), or a variant thereof comprising 1, 2, 3, or 4 amino acid substitutions; (b) a heavy chain CDR2 comprising YISYSGGTDYNPSLKS (SEQ ID NO:2), or a variant thereof comprising 1, 2, 3, or 4 amino acid substitutions; (c) a heavy chain CDR3 comprising KGAY (SEQ ID NO:3), or a variant thereof comprising 1, 2, 3, or 4 amino acid substitutions; (d) a light chain CDR1 comprising SASSSVSSSYLY (SEQ ID NO:4), or a variant thereof comprising 1, 2, 3, or 4 amino acid substitutions; (e) a light chain CDR2 comprising STSNLAS (SEQ ID NO:5), or a variant thereof comprising 1, 2, 3, or 4 amino acid substitutions; and (f) a light chain CDR3 comprising HQWSSYPYT (SEQ ID NO:6), or a variant thereof comprising 1, 2, 3, or 4 amino acid substitutions. In certain embodiments, the amino acid substitutions are conservative substitutions. In some embodiments, the substitutions are identified and/or made as part of a germline humanization process.

In certain embodiments, the invention provides a MET-binding agent that specifically binds MET, wherein the MET-binding agent comprises a heavy chain variable region having at least about 80% sequence identity to SEQ ID NO:7 or SEQ ID NO:94, and a light chain variable region having at least about 80% sequence identity to SEQ ID NO:8 or SEQ ID NO:95. In certain embodiments, the MET-binding agent comprises a heavy chain variable region having at least about 85%, at least about 90%, at least about 95%, at least about 97%, or at least about 99% sequence identity to SEQ ID NO:7 or SEQ ID NO:94. In certain embodiments, the MET-binding agent comprises a light chain variable region having at least about 85%, at least about 90%, at least about 95%, at least about 97%, or at least about 99% sequence identity to SEQ ID NO:8 or SEQ ID NO:95. In certain embodiments, the MET-binding agent comprises a heavy chain variable region having at least about 95% sequence identity to SEQ ID NO:7 and a light chain variable region having at least about 95% sequence identity to SEQ ID NO:8. In certain embodiments, the MET-binding agent comprises a heavy chain variable region having at least about 95% sequence identity to SEQ ID NO:94 and a light chain variable region having at least about 95% sequence identity to SEQ ID NO:95, In certain embodiments, the MET-binding agent comprises a heavy chain variable region comprising SEQ ID NO:7 and a light chain variable region comprising SEQ ID NO:8. In certain embodiments, the MET-binding agent comprises a heavy chain variable region comprising SEQ. ID NO:94 and a light chain variable region comprising SEQ ID NO:95. In certain embodiments, the MET-binding agent comprises a heavy chain variable region consisting essentially of SEQ ID NO:7 and a light chain variable region consisting essentially of SEQ ID NO:8. In certain embodiments, the MET-binding agent comprises a heavy chain variable region consisting essentially of SEQ ID NO:94 and a light chain variable region consisting essentially of SEQ ID NO:95, In certain embodiments, the MET-binding agent comprises a heavy chain variable region of SEQ ID NO:7 and a light chain variable region of SEQ ID NO:8. In certain embodiments, the MET-binding agent comprises a heavy chain variable region of SEQ ID NO:94 and a light chain variable region of SEQ. ID NO:95.

In some embodiments, the invention provides a MET-binding agent that specifically binds MET, wherein the MET-binding agent comprises a heavy chain comprising SEQ ID NO:12 and a light chain comprising SEQ ID NO:14. In some embodiments, the MET-binding agent comprises a heavy chain of SEQ ID NO:12 and a light chain of SEQ ID NO:14. In some embodiments, the MET-binding agent comprises a heavy chain comprising SEQ ID NO:13 and a light chain comprising SEQ ID NO:14. In some embodiments, the MET-binding agent comprises a heavy chain of SEQ ID NO:13 and a light chain of SEQ ID NO:14. In some embodiments, the MET-binding agent comprises a heavy chain comprising SEQ ID NO:88 and a light chain comprising SEQ ID NO:14. In some embodiments, the MET-binding agent comprises a heavy chain of SEQ ID NO:88 and a light chain of SEQ ID NO:14.

In some embodiments, the invention provides a MET-binding agent that specifically binds MET, wherein the MET-binding agent comprises a heavy chain comprising SEQ ID NO:99 and a light chain comprising SEQ ID NO:101. In some embodiments, the MET-binding agent comprises a heavy chain of SEQ ID NO:99 and a light chain of SEQ ID NO:101. In some embodiments, the MET-binding agent comprises a heavy chain comprising SEQ ID NO:100 and a light chain comprising SEQ ID NO:101. In some embodiments, the MET-binding agent comprises a heavy chain of SEQ ID NO:100 and a light chain of SEQ ID NO:101. In some embodiments, the MET-binding agent comprises a heavy chain comprising SEQ ID NO:108 and a light chain comprising SEQ ID NO:101. In some embodiments, the MET-binding agent comprises a heavy chain of SEQ ID NO:108 and a light chain of SEQ ID NO:101. In some embodiments, the MET-binding agent comprises a heavy chain comprising SEQ ID NO:112 and a light chain comprising SEQ ID NO:101. In some embodiments, the MET-binding agent comprises a heavy chain of SEQ ID NO:112 and a light chain of SEQ ID NO:101. In some embodiments, the MET-binding agent comprises a heavy chain comprising SEQ ID NO:111 and a light chain comprising SEQ ID NO:101. In some embodiments, the MET-binding agent comprises a heavy chain of SEQ ID NO:111 and a light chain of SEQ ID NO:101.

In certain embodiments, the invention provides a MET-binding agent that specifically binds human MET, wherein the MET-binding agent comprises one, two, three, four, five, and/or six of the CDRs of antibody 73R009 (see Table 1) or a humanized version of 73R009 (i.e., 73R010 or variants thereof). In some embodiments, the MET-binding agent comprises one or more of the CDRs of 73R009 or a humanized version of 73R009, two or more of the CDRs of 73R009 or a humanized version of 73R009, three or more of the CDRs of 73R009 or a humanized version of 73R009, four or more of the CDRs of 73R009 or a humanized version of 73R009, five or more of the CDRs of 73R009 or a humanized version of 73R009, or all six of the CDRs of 73R009 or a humanized version of 73R009.

TABLE 1

| | 73R009 and 73R010 |
|---|---|
| HC CDR1 | ASYAWS |
| | (SEQ ID NO: 1) |
| HC CDR2 | YISYSGGTDYNPSLKS |
| | (SEQ ID NO: 2) |
| HC CDR3 | KGAY |
| | (SEQ ID NO: 3) |
| LC CDR1 | SASSSVSSSYLY |
| | (SEQ ID NO: 4) |
| LC CDR2 | STSNLAS |
| | (SEQ ID NO: 5) |
| LC CDR3 | HQWSSYPYT |
| | (SEQ ID NO: 6) |

In certain embodiments, a MET-binding agent comprises the heavy chain variable region and the light chain variable region of antibody 73R009. In certain embodiments, a MET-binding agent comprises the heavy chain variable region and the light chain variable region of a humanized version of antibody 73R009. In certain embodiments, a MET-binding agent comprises the heavy chain variable region and the light chain variable region of the antibody 738010. In certain embodiments, a MET-binding agent comprises the heavy chain and the light chain of antibody 73R009 or antibody 73R010 (with or without the leader sequence). In certain embodiments, a MET-binding agent comprises the heavy chain and the light chain of antibody 73R009 or antibody 73R010 (with or without the leader sequence) wherein the heavy chain is modified to promote formation of heterodimers (e.g., bispecific agents) or heteromultimers. In certain embodiments, a MET-binding agent is antibody 73R009. In certain embodiments, a MET-binding agent is antibody 73R010 (73R009 H12L7). In certain embodiments, a MET-binding agent is a variant of antibody 73R010. In some embodiments, the MET-binding agent comprises a heavy chain variable region encoded by the plasmid deposited with American Type Culture Collection (ATCC), and designated PTA-13609. In some embodiments, the MET-binding agent comprises a heavy chain variable region encoded by the plasmid deposited with ATCC and designated PTA-120387. In some embodiments, the MET-binding agent comprises a heavy chain variable region encoded by the plasmid deposited with ATCC and designated PTA-120695. In some embodiments, the MET-binding agent comprises a light chain variable region encoded by the plasmid deposited with ATCC and designated PTA-13610. In some embodiments, the MET-binding agent comprises a light chain variable region encoded by the plasmid deposited with ATCC and designated PTA-120388.

In certain embodiments, a MET-binding agent comprises, consists essentially of, or consists of, antibody 73R009. In certain embodiments, a MET-binding agent comprises, consists essentially of, or consists of, a humanized version of antibody 73R009. In certain embodiments, a MET-binding agent comprises, consists essentially of, or consists of, antibody 73R010.

In certain embodiments, a MET-binding agent binds the same epitope or essentially the same epitope on MET as a binding agent of the invention. In another embodiment, a MET-binding agent is an antibody or a bispecific agent that binds an epitope on MET that overlaps with the epitope on MET bound by a binding agent of the invention. In certain embodiments, a MET-binding agent binds the same epitope, or essentially the same epitope, on MET as antibody 73R009 or a humanized version of antibody 73R009. In certain embodiments, a MET-binding agent binds the same epitope, or essentially the same epitope, on MET as antibody 73R010. In another embodiment, a MET-binding agent is an antibody or a bispecific agent that binds an epitope on MET that overlaps with the epitope on MET bound by antibody 73R009 or a humanized version of antibody 73R009. In another embodiment, a MET-binding agent is an antibody or a bispecific agent that binds an epitope on MET that overlaps with the epitope on MET bound by antibody 73R010.

In certain embodiments, the MET-binding agent is an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In certain embodiments, the antibody is an IgA, IgD, IgE, IgG, or IgM antibody. In certain embodiments, the antibody is an IgG1 antibody. In certain embodiments, the antibody is an IgG2 antibody. In certain embodiments, the antibody is an antibody fragment comprising an antigen-binding site. In some embodiments, the antibody is a bispecific antibody. In some embodiments, the antibody is a monovalent antibody. In some embodiments, the antibody is monospecific. In some embodiment, the antibody is multispecific.

In some embodiments, the MET-binding agent inhibits binding of MET to hepatocyte growth factor. In some embodiments, the MET-binding agent blocks binding of MET to hepatocyte growth factor. In some embodiments, the MET-binding agent specifically binds MET and facilitates internalization of MET. In some embodiments, the MET-binding agent specifically binds MET and stimulates degradation of MET. In some embodiments, the MET-binding agent specifically binds MET and inhibits dimerization of MET. In some embodiments, the MET-binding agent specifically binds MET and inhibits activation of MET. In some embodiments, the MET-binding agent specifically binds MET and inhibits and/or reduces phosphorylation of MET. In some embodiments, the MET-binding agent specifically binds MET and inhibits tumor growth.

In some embodiments, the MET-binding agent binds MET with a K_{D} of about 100 nM or less. In some embodiments, the MET-binding agent binds MET with a K_{D} of about 10 nM or less. In some embodiments, the MET-binding agent binds MET with a K_{D} of about 1 nM or less. In some embodiments, the MET-binding agent binds MET with a K_{D} of about 0.1 nM or less. In some embodiments, the MET-binding agent binds MET with a K_{D} of about 0.01 nM or less. In some embodiments, at least one amino acid residue in at least one CDR of the MET-binding agent is substituted with a different amino acid so that the affinity of the MET-binding agent for MET is altered. In some embodiments, the affinity of the MET-binding agent for MET is increased. In some embodiments, the affinity of the MET-binding agent for MET is decreased. In some embodiments, the MST-binding agent binds human MET. In some embodiments, the MET-binding agent binds human MET and mouse MET. In some embodiments, the MET-binding agent binds human MET and does not bind mouse MET.

In certain embodiments, the invention provides a MET-binding agent that is a bispecific agent. In some embodiments, the MET-binding agent is a bispecific agent comprising a first arm and a second arm. In some embodiments, the MET-binding agent is a bispecific agent comprising a first arm and a second arm, wherein the first arm comprises a first binding site that specifically binds MET. In some embodiments, the MET-binding agent is a bispecific agent comprising a first arm and a second arm, wherein the first arm comprises a first binding site that specifically binds MET and the second arm comprises a second binding site that specifically binds a second target or antigen. In some embodiments, the first binding site comprises an antigen-binding site. In some embodiments, the second binding site comprises an antigen-binding site. In some embodiments, the MET-binding agent is a bispecific agent wherein the first arm comprises a first binding site that specifically binds human MET and the second arm comprises a second binding site that binds one or more components of the WNT pathway.

In certain embodiments, the MET-binding agent is a bispecific agent that specifically binds human MET and one or more human FZD proteins. In certain embodiments, the bispecific agent is a bispecific antibody that specifically binds both human MET and one or more human FZD proteins. In some embodiments, the bispecific antibody specifically binds one, two, three, four, five, six, seven, eight, nine, or ten FZD proteins. In some embodiments, the bispecific antibody binds one or more FZD proteins selected from the group consisting of FZD1, FZD2, FZD3, FZD4, FZD5, FZD6, FZD7, FZD8, FZD9, and FZD10. In some embodiments, the bispecific antibody binds one or more FZD proteins comprising FZD1, FZD2, FZD5, FZD7, and/or FZD8. In certain embodiments, the bispecific antibody binds FZD7. In certain embodiments, the bispecific antibody binds FZD5 and/or FZD8. In certain embodiments, the bispecific antibody specifically binds FZD1, FZD2, FZD5, FZD7, and FZD8. Non-limiting examples of FZD-binding agents can be found in U.S. Pat. No. 7,982,013.

In certain embodiments, the bispecific antibody specifically binds MET and the extracellular domain (ECD) of one or more human FZD proteins. In certain embodiments, the bispecific antibody specifically binds MET and a fragment of the extracellular domain (ECD) of one or more human FZD proteins. In certain embodiments, the bispecific antibody specifically binds within the Fri domain (also known as the cysteine-rich domain (CRD)) of one or more human FZD proteins. Sequences of the Fri domain of each of the human FZD proteins are known in the art and are provided as SEQ ID NO:21 (FZD1), SEQ ID NO:22 (FZD2), SEQ ID NO:23 (FZD3), SEQ ID NO:24 (FZD4), SEQ ID NO:25 (FZD5), SEQ ID NO:26 (FZD6), SEQ ID NO:27 (FZD7), SEQ ID NO:28 (FZD8), SEQ ID NO:29 (FZD8), SEQ ID NO:30 (FZD9) and SEQ ID NO:31 (FZD10). Sequences of the predicted minimal Fri domains are provided as SEQ ID NO:32 (FZD1), SEQ ID NO:33 (FZD2), SEQ ID NO:34 (FZD3), SEQ ID NO:35 (FZD4), SEQ ID NO:36 (FZD5), SEQ ID NO:37 (FZD6), SEQ ID NO:38 (FZD7), SEQ ID NO:39 (FZD8), SEQ ID NO:40 (FZD9) and SEQ ID NO:41 (FZD10). In certain embodiments, the bispecific antibody specifically binds to the Biological Binding Site (BBS) of a human FZD protein. The BBS of FZD proteins has been described in U.S. Pat. No. 7,982,013. In some embodiments, the bispecific antibody specifically binds to at least part of the BBS of a human FZD protein. In some embodiments, the bispecific antibody specifically binds to at least part of the BBS of human FZD1, FZD2, FZD5, FZD7, and/or FZD8.

In certain embodiments, the bispecific antibody binds human MET and binds one, two, three, four, five, or more FZD proteins. In some embodiments, the bispecific antibody specifically binds human MET and binds one, two, three, four, or five FZD proteins selected from the group consisting of FZD1, FZD2, FZD5, FZD7, and FZD8. In some embodiments, the bispecific antibody specifically binds MET and binds at least FZD5 and FZD8.

In certain embodiments, the bispecific antibody that binds human MET and one or more human FZD proteins is a FZD antagonist. In certain embodiments, the bispecific antibody is a Wnt pathway antagonist. In certain embodiments, the bispecific antibody inhibits Wnt signaling. In some embodiments, the bispecific antibody inhibits canonical Wnt signaling.

In certain embodiments, the MET-binding agent is a bispecific agent that specifically binds human MET and one or more human WNT proteins. In certain embodiments, the bispecific agent is a bispecific antibody that specifically binds human MET and one or more human WNT proteins. In certain embodiments, the bispecific antibody specifically binds human MET and binds one, two, three, four, five, six, seven, eight, nine, ten, or more WNT proteins. In some embodiments, the bispecific antibody binds human MET and binds one or more human WNT proteins selected from the group consisting of WNT1, WNT2, WNT2b, WNT3, WNT3a, WNT4, WNT5a, WNT5b, WNT6, WNT7a, WNT7b, WNT8a, WNT8b, WNT9a, WNT9b, WNT10a, WNT10b, WNT11, and WNT16. In certain embodiments, the bispecific antibody binds human MET and binds one or more (or two or more, three or more, four or more, five or more, etc.) WNT proteins selected from the group consisting of WNT1, WNT2, WNT2b, WNT3, WNT3a, WNT7a, WNT7b, WNT8a, WNT8b, WNT10a, and WNT10b. In certain embodiments, the one or more (or two or more, three or more, four or more, five or more, etc.) WNT proteins are selected from the group consisting of WNT1, WNT2, WNT2b, WNT3, WNT3a, WNT8a, WNT8b, WNT10a, and WNT10b. Non-limiting examples of WNT-binding agents can be found in International Publication WO 2011/088127.

In certain embodiments, the bispecific antibody specifically binds MET and the C-terminal cysteine rich domain (CRD) of one or more human WNT proteins. In certain embodiments, the bispecific antibody binds a domain within one or more WNT proteins selected from the group consisting of: SEQ ID NO:57 (WNT1), SEQ ID NO:58 (WNT2), SEQ ID NO:59 (WNT2b), SEQ ID NO:60 (WNT3), SEQ ID NO:61 (WNT3a), SEQ ID NO:62 (WNT7a), SEQ ID NO:63 (WNT7b), SEQ ID NO:64 (WNT8a), SEQ ID NO:65 (WNT8b), SEQ ID NO:66 (WNT10a), and SEQ ID NO:67 (WNT10b).

In certain embodiments, the bispecific antibody that binds human MET and one or more WNT proteins is a WNT antagonist. In certain embodiments, the bispecific antibody is a WNT pathway antagonist. In certain embodiments, the bispecific antibody inhibits WNT signaling. In some embodiments, the bispecific antibody inhibits canonical WNT signaling.

In certain embodiments, the MET-binding agent is a bispecific agent that specifically binds human MET and one or more human WNT proteins. In certain embodiments, the bispecific agent that specifically binds human MET and one or more human WNT proteins is a heterodimeric agent. In certain embodiments, the bispecific agent that specifically binds human MET and one or more human WNT proteins is a heterodimeric agent comprising a soluble receptor. In certain embodiments, the bispecific agent that specifically binds human MET and one or more human WNT proteins is a heterodimeric agent comprising a fusion protein. In certain embodiments, the bispecific agent that specifically binds human MET and one or more human WNT proteins is a heterodimeric agent comprising a first arm comprising a monovalent antibody and a second arm comprising a soluble receptor. In certain embodiments, the bispecific agent that specifically binds human MET and one or more human WNT proteins is a heterodimeric agent comprising a first arm comprising a monovalent antibody and a second arm comprising a fusion protein. In some embodiments, the fusion protein is an immunoadhesin.

In certain embodiments, the MET-binding agent is a bispecific agent that specifically binds human MET and one or more human WNT proteins, wherein the bispecific agent comprises the extracellular domain (ECD) of a FZD receptor protein (e.g., a soluble receptor), In certain embodiments, the FZD protein is a human FZD protein. In certain embodiments, the human FZD protein is FZD1, FZD2, FZD3, FZD4, FZD5, FZD6, FZD7, FZD8, FZD9, or FZD10. In certain embodiments, the human FZD protein is FZD8. Non-limiting examples of soluble FZD receptors can be found in U.S. Pat. Nos. 7,723,477 and 7,947,277; and U.S. Patent Publication No. 2011/0305695.

In some embodiments, the bispecific agent comprises a Fri domain of an ECD of a FZD protein. The Fri domains for each of the human FZD1-10 proteins are provided as SEQ ID NOs:21-31. The minimal (or core) Fri domains for each of the human FZD1-10 proteins are provided as SEQ ID NOs:32-41. Those of skill in the art may differ in their understanding of the exact amino acids corresponding to the various Fri domains, Thus, the N-terminus and/or C-terminus of the domains outlined above and herein may extend or be shortened by 1, 2, 3, 4, 5, 6, 7, 8, 9, or even 10 amino acids.

In some embodiments, a soluble receptor comprising a FZD Fri domain can demonstrate altered biological activity (e.g., increased protein half-life) compared to a soluble receptor comprising the entire FZD ECD. In some embodiments, protein half-life can be further increased by covalent modification with polyethylene glycol (PEG) or polyethylene oxide (PEO).

In certain embodiments, the bispecific agent comprises a Fri domain of a human FZD protein, or a fragment or variant of the Fri domain that binds one or more human WNT proteins. In certain embodiments, the human FZD protein is FZD1, FZD2, FZD3, FZD4, FZD5, FZD6, FZD7, FZD8, FZD9, or FZD10. In certain embodiments, the human FZD protein is FZD8. In certain embodiments, the human FZD protein is FZD4. In certain embodiments, the human FZD protein is FZD5. In certain embodiments, the human FZD protein is FZD10. In certain embodiments the FZD protein is FZD4 and the bispecific agent comprises SEQ ID NO:24. In certain embodiments, the FZD protein is FZD5 and the bispecific agent comprises SEQ ID NO:25. In certain embodiments, the FZD protein is FZD7 and the bispecific agent comprises SEQ ID NO:27. In certain embodiments, the FZD protein is FZD8 and the bispecific agent comprises SEQ ID NO:28 or SEQ ID NO:29. In certain embodiments, the FZD protein is FZD10 and the bispecific agent comprises SEQ ID NO:31.

In some embodiments, the bispecific agent comprises a Fri domain comprising the minimal Fri domain of FZD1 (SEQ ID NO:32), the minimal Fri domain of FZD2 (SEQ ID NO:33), the minimal Fri domain of FZD3 (SEQ ID NO:34), the minimal Fri domain of FZD4 (SEQ ID NO:35), the minimal Fri domain of FZD5 (SEQ ID NO:36), the minimal Fri domain of FZD6 (SEQ. ID NO:37), the minimal Fri domain of FZD7 (SEQ ID NO:38), the minimal Fri domain of FZD8 (SEQ ID NO:39), the minimal Fri domain of FZD9 (SEQ ID NO:40), or the minimal Fri domain of FZD10 (SEQ ID NO:41). In some embodiments, the bispecific agent comprises a Fri domain comprising the minimal Fri domain of FZD8 (SEQ ID NO:39).

In some embodiments, the bispecific agent comprises a Fri domain consisting essentially of the Fri domain of FZD1, the Fri domain of FZD2, the Fri domain of FZD3, the Fri domain of FZD4, the Fri domain of FZD5, the Fri domain of FZD6, the Fri domain of FZD7, the Fri domain of FZD8, the Fri domain of FZD9, or the Fri domain of FZD10. In some embodiments, the bispecific agent comprises a Fri domain consisting essentially of the Fri domain of FZD8.

In some embodiments, the bispecific agent comprises a sequence selected from the group consisting of: SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ NO:38, SEQ ID NO:39, SEQ ID NO:40, and SEQ ID NO:41. In some embodiments, the bispecific agent comprises a Fri domain comprising SEQ ID NO:39. In some embodiments, the bispecific agent comprises a Fri domain of SEQ ID NO:39. In some embodiments, the bispecific agent comprises a Fri domain comprising SEQ ID NO:28. In some embodiments, the bispecific agent comprises a Fri domain of SEQ ID NO:28. In some embodiments, the bispecific agent comprises a Fri domain comprising SEQ ID NO:29. In some embodiments, the bispecific agent comprises a Fri domain of SEQ ID NO:29.

In certain embodiments, the bispecific agent comprises a variant of any one of the aforementioned FZD Fri domain sequences that comprises one or more (e.g., one, two, three, four, five, six, seven, eight, nine, ten, etc.) conservative substitutions and is capable of binding WNT protein(s).

In certain embodiments, a bispecific agent, such as an agent comprising a soluble FZD receptor, further comprises a heterologous polypeptide (i.e., a non-FZD polypeptide). In some embodiments, a soluble FZD receptor may include FZD ECD or Fri domains linked to other heterologous functional and structural polypeptides including, but not limited to a human Fe region, protein tags (e.g., myc, FLAG, GST), other endogenous proteins or protein fragments, or any other useful protein sequence including any linker region between a FZD ECD or Fri domain and a second polypeptide. In certain embodiments, the heterologous polypeptide comprises a human Fe region. The Fe region can be obtained from any of the classes of immunoglobulin, IgG, IgA, IgM, IgD and IgE. In some embodiments, the Fe region is a human IgG1 Fe region. In some embodiments, the Fe region is a human IgG2 Fe region. In some embodiments, the Fe region is a wild-type Fe region (including Fe region variants found in nature). In some embodiments, the Fe region is a mutated Fe region. In some embodiments, the Fc region is truncated at the N-terminal end by 1, 2, 3, 4, 5, 6, 7, 8, 9, 10, or more amino acids, (e.g., in the hinge domain). In some embodiments, an amino acid in the hinge domain is changed to hinder undesirable disulfide bond formation. In some embodiments, a cysteine is replaced with a serine to hinder or block undesirable disulfide bond formation. In some embodiments, the Fc region is truncated at the C-terminal end by 1, 2, 3, or more amino acids. In some embodiments, the Fc region is truncated at the C-terminal end by 1 amino acid. In certain embodiments, the heterologous polypeptide comprises SEQ ID NO:42, SEQ ID NO:43, SEQ ID NO:44, SEQ ID NO:45, SEQ ID NO:46, SEQ ID NO: 47, SEQ ID NO:48, SEQ ID NO:49, SEQ ID NO:50, SEQ ID NO:51, SEQ ID NO:52, SEQ ID NO:91, or SEQ ID NO:92. In certain embodiments, the heterologous polypeptide is SEQ ID NO:42, SEQ ID NO:43, SEQ ID NO:44, SEQ ID NO:45, SEQ ID NO:46, SEQ ID NO: 47, SEQ ID NO:48, SEQ ID NO:49, SEQ ID NO:50, SEQ ID NO:51, SEQ ID NO:52, SEQ ID NO:91, or SEQ ID NO:92. In certain embodiments, the heterologous polypeptide comprises SEQ ID NO:48, SEQ ID NO:51, or SEQ ID NO:52. In certain embodiments, the heterologous polypeptide is SEQ ID NO:48, SEQ ID NO:51, or SEQ ID NO:52.

In certain embodiments, a bispecific agent comprises a fusion protein comprising at least a minimal Fri domain of a FZD receptor and a Fc region. As used herein, a “fusion protein” is a hybrid protein expressed by a nucleic acid molecule comprising nucleotide sequences of at least two genes. In some embodiments, the C-terminus of the first polypeptide is linked to the N-terminus of the immunoglobulin Fc region. In some embodiments, the first polypeptide (e.g., a FZD Fri domain) is directly linked to the Fc region (i.e. without an intervening linker). In some embodiments, the first polypeptide is linked to the Fc region via a linker.

As used herein, the term “linker” refers to a linker inserted between a first polypeptide (e.g., a FZD component) and a second polypeptide (e.g., a Fc region). In some embodiments, the linker is a peptide linker. Linkers should not adversely affect the expression, secretion, or bioactivity of the polypeptide. Linkers should not be antigenic and should not elicit an immune response. Suitable linkers are known to those of skill in the art and often include mixtures of glycine and serine residues and often include amino acids that are sterically unhindered. Other amino acids that can be incorporated into useful linkers include threonine and alanine residues. Linkers can range in length, for example from 1-50 amino acids in length, 1-22 amino acids in length, 1-10 amino acids in length, 1-5 amino acids in length, or 1-3 amino acids in length. Linkers may include, but are not limited to, SerGly, GGSG, GSGS, GGGS, S(GGS)n where n is 1-7, GRA, poly(Gly), poly(Ala), ESGGGGVT (SEQ ID NO:68), LESGGGGVT (SEQ ID NO:69), GRAQVT (SEQ ID NO:70), WRAQVT (SEQ ID NO:71), and ARGRAQVT (SEQ ID NO:72). As used herein, a linker is an intervening peptide sequence that does not include amino acid residues from either the C-terminus of the first polypeptide (e.g., a FZD Fri domain) or the N-terminus of the second polypeptide (e.g., the Fc region).

In some embodiments, the bispecific agent comprises a FZD Fri domain, a Fc region and a linker connecting the FZD Fri domain to the Fc region. In some embodiments, the FZD Fri domain comprises SEQ ID NO:28, SEQ ID NO:29, or SEQ ID NO:39. In some embodiments, the linker comprises ESGGGGVT (SEQ ID NO:68) or LESGGGGVT (SEQ ID NO:69).

FZD receptors and immunoglobulin proteins contain signal sequences that direct the transport of the proteins. Signal sequences (also referred to as signal peptides or leader sequences) are located at the N-terminus of nascent polypeptides. They target the polypeptide to the endoplasmic reticulum and the proteins are sorted to their destinations, for example, to the inner space of an organelle, to an interior membrane, to the cell's outer membrane, or to the cell exterior via secretion. Most signal sequences are cleaved from the protein by a signal peptidase after the proteins are transported to the endoplasmic reticulum. The cleavage of the signal sequence from the polypeptide usually occurs at a specific site in the amino acid sequence and is dependent upon amino acid residues within the signal sequence. Although there is usually one specific cleavage site, more than one cleavage site may be recognized and/or used by a signal peptidase resulting in a non-homogenous N-terminus of the polypeptide. For example, the use of different cleavage sites within a signal sequence can result in a polypeptide expressed with different N-terminal amino acids. Accordingly, in some embodiments, the polypeptides as described herein may comprise a mixture of polypeptides with different N-termini. In some embodiments, the N-termini differ in length by 1, 2, 3, 4, or 5 amino acids. In some embodiments, the polypeptide is substantially homogeneous, i.e., the polypeptides have the same N-terminus. In some embodiments, the signal sequence of the polypeptide comprises one or more (e.g., one, two, three, four, five, six, seven, eight, nine, ten, etc.) amino acid substitutions and/or deletions. In some embodiments, the signal sequence of the polypeptide comprises amino acid substitutions and/or deletions that allow one cleavage site to be dominant, thereby resulting in a substantially homogeneous polypeptide with one N-terminus.

In some embodiments, the bispecific agent that specifically binds MET and one or more WNT proteins comprises: a first polypeptide comprising SEQ ID NO:28 and a second polypeptide comprising SEQ ID NO:47, SEQ ID NO:48, SEQ ID NO:49, SEQ ID NO:50, SEQ ID NO:51, or SEQ ID NO:52. In some embodiments, the bispecific agent comprises: a first polypeptide comprising SEQ ID NO:28 and a second polypeptide comprising SEQ ID NO:47 or SEQ ID NO:48. In some embodiments, the bispecific agent comprises: a first polypeptide comprising SEQ ID NO:28 and a second polypeptide comprising SEQ ID NO:49 or SEQ ID NO:51. In some embodiments, the bispecific agent comprises: a first polypeptide comprising SEQ ID NO:28 and a second polypeptide comprising SEQ ID NO:50 or SEQ ID NO:52. In some embodiments, the bispecific agent comprises: a first polypeptide comprising SEQ ID NO:28 and a second polypeptide comprising SEQ ID NO:52. In some embodiments, the bispecific agent that specifically binds MET and one or more WNT proteins comprises: a first polypeptide comprising SEQ ID NO:29 and a second polypeptide comprising SEQ ID NO:47, SEQ ID NO:48, SEQ ID NO:49, SEQ ID NO:50, SEQ ID NO:51, or SEQ ID NO:52. In some embodiments, the bispecific agent comprises: a first polypeptide comprising SEQ ID NO:29 and a second polypeptide comprising SEQ ID NO:47 or SEQ ID NO:48. In some embodiments, the bispecific agent comprises: a first polypeptide comprising SEQ ID NO:29 and a second polypeptide comprising SEQ ID NO:49 or SEQ ID NO:51. In some embodiments, the bispecific agent comprises: a first polypeptide comprising SEQ ID NO:29 and a second polypeptide comprising SEQ ID NO:50 or SEQ ID NO:52. In some embodiments, the bispecific agent comprises: a first polypeptide comprising SEQ ID NO:29 and a second polypeptide comprising SEQ ID NO:52. In some embodiments, the bispecific agent that specifically binds MET and one or more WNT proteins comprises: a first polypeptide comprising SEQ ID NO:39 and a second polypeptide comprising SEQ ID NO:47, SEQ ID NO:48, SEQ ID NO:49, SEQ ID NO:50, SEQ ID NO:51, or SEQ ID NO:52. In some embodiments, the bispecific agent comprises: a first polypeptide comprising SEQ ID NO:39 and a second polypeptide comprising SEQ ID NO:47 or SEQ ID NO:48. In some embodiments, the bispecific agent comprises: a first polypeptide comprising SEQ ID NO:39 and a second polypeptide comprising SEQ ID NO:49 or SEQ ID NO:51. In some embodiments, the bispecific agent comprises: a first polypeptide comprising SEQ ID NO:39 and a second polypeptide comprising SEQ ID NO:50 or SEQ ID NO:52. In some embodiments, the bispecific agent comprises: a first polypeptide comprising SEQ ID NO:39 and a second polypeptide comprising SEQ ID NO:52.

In some embodiments, the bispecific agent comprises SEQ ID NO:55 or SEQ ID NO:56. In some embodiments, the bispecific agent comprises SEQ ID NO:56. In some embodiments, the bispecific agent comprises SEQ ID NO:87.

In some embodiments, the MET-binding agent is a bispecific agent comprising: (a) a first binding site that specifically binds human MET, and (b) a second binding site that binds one or more components of the WNT pathway, wherein the first binding site comprises (a) a heavy chain CDR1 comprising ASYAWS (SEQ ID NO:1), a heavy chain CDR2 comprising YISYSGGTDYNPSLKS (SEQ ID NO:2), and a heavy chain CDR3 comprising KGAY (SEQ ID NO:3), and (b) a light chain CDR1 comprising SASSSVSSSYLY (SEQ ID NO:4), a light chain CDR2 comprising STSNLAS (SEQ ID NO:5), and a light chain CDR3 comprising HQWSSYPYT (SEQ ID NO:6). In some embodiments, the MET-binding agent is a bispecific agent comprising: (a) a first binding site that specifically binds human MET, and (b) a second binding site that binds one or more WNT proteins, wherein the first binding site comprises (a) a heavy chain CDR1 comprising ASYAWS (SEQ ID NO:1), a heavy chain CDR2 comprising YISYSGGTDYNPSLKS (SEQ ID NO:2), and a heavy chain CDR3 comprising KGAY (SEQ ID NO:3), and (b) a light chain CDR1 comprising SASSSVSSSYLY (SEQ ID NO:4), a light chain CDR2 comprising STSNLAS (SEQ ID NO:5), and a light chain CDR3 comprising HQWSSYPYT (SEQ ID NO:6).

In some embodiments, the MET-binding agent is a bispecific agent comprising (a) a first binding site that specifically binds human MET and (b) a second binding site that binds one or more components of the WNT pathway, wherein the first binding site comprises a heavy chain CDR1 comprising GYTFTSYWLH (SEQ ID NO:78), a heavy chain CDR2 comprising GMIDPSNSDTRFNPNFKD (SEQ ID NO:79), and a heavy chain CDR3 comprising TYGSYVSPLDY (SEQ ID NO:81), SYGSYVSPLDY (SEQ ID NO:82), ATYGSYVSPLDY (SEQ ID NO:83), or XYGSYVSPLDY (SEQ ID NO:80), wherein X is not R; and a light chain CDR1 comprising KSSQSLLYTSSQKNYLA (SEQ ID NO:84), a light chain CDR2 comprising WASTRES (SEQ ID NO:85), and a light chain CDR3 comprising QQYYAYPWT (SEQ ID NO:86).

In some embodiments, the MET-binding agent is a bispecific agent comprising: (a) a first binding site that specifically binds human MET, and (b) a second binding site that binds one or more components of the WNT pathway, wherein the first binding site comprises a heavy chain variable region having at least about 80% sequence identity to SEQ ID NO:7 or SEQ ID NO:94. In some embodiments, the first binding site further comprises a light chain variable region having at least about 80% sequence identity to SEQ ID NO:8 or SEQ ID NO:95. In certain embodiments, the first binding site comprises a heavy chain variable region having at least about 85%, at least about 90%, at least about 95%, at least about 97%, or at least about 99% sequence identity to SEQ ID NO:7 or SEQ ID NO:94, and a light chain variable region having at least about 85%, at least about 90%, at least about 95%, at least about 97%, or at least about 99% sequence identity to SEQ ID NO:8 or SEQ ID NO:95.

In some embodiments, the MET-binding agent is a bispecific agent that comprises (a) a first arm comprising a first binding site that specifically binds human MET, and (b) a second arm comprising a second binding site that binds one or more WNT proteins, wherein the first arm comprises a heavy chain CDR1 comprising ASYAWS (SEQ ID NO:1), a heavy chain CDR2 comprising YISYSGGTDYNPSLKS (SEQ ID NO:2), and a heavy chain CDR3 comprising KGAY (SEQ ID NO:3), and a light chain CDR1 comprising SASSSVSSSYLY (SEQ ID NO:4), a light chain CDR2 comprising STSNLAS (SEQ ID NO:5), and a light chain CDR3 comprising HQWSSYPYT (SEQ ID NO:6); and the second arm comprises a FZD8 Fri domain. In some embodiments, the second arm comprises SEQ ID NO:28, SEQ ID NO:29, or SEQ ID NO:39.

In some embodiments, the MET-binding agent is a bispecific agent that specifically binds human MET and binds one or more components of the WNT pathway, wherein the first arm of the bispecific agent comprises a heavy chain of SEQ ID NO:12, SEQ ID NO:13, SEQ ID NO:88, SEQ ID NO:99, SEQ ID NO:100, SEQ ID NO:108, SEQ ID NO:111, or SEQ ID NO:112, and/or a light chain of SEQ ID NO:14 or SEQ ID NO:101. In some embodiments, the first arm of the bispecific agent comprises a heavy chain of SEQ ID NO:13 and a light chain of SEQ ID NO:14. In some embodiments, the first arm of the bispecific agent comprises a heavy chain of SEQ ID NO:100 and a light chain of SEQ ID NO:101. In some embodiments, the first arm of the bispecific agent comprises a heavy chain of SEQ ID NO:111 and a light chain of SEQ ID NO:101.

In some embodiments, the MET-binding agent is a bispecific agent that specifically binds human MET and binds one or more WNT proteins, wherein the first arm of the bispecific agent comprises a heavy chain of SEQ ID NO:12, SEQ ID NO:13, SEQ ID NO:88, SEQ ID NO:99, SEQ ID NO:100, SEQ ID NO:108, SEQ ID NO:111, or SEQ ID NO:112, and a light chain of SEQ ID NO:14 or SEQ ID NO:101, and wherein the second arm of the bispecific agent comprises a first polypeptide comprising a FZD8 Fri domain. In some embodiments, the second arm of the bispecific agent comprises a first polypeptide comprising a FZD8 Fri domain and a second polypeptide comprising a human Fc region. In some embodiments, the second arm of the bispecific agent comprises a first polypeptide comprising a FZD8 Fri domain and a second polypeptide comprising a human IgG1 Fc region. In some embodiments, the second arm of the bispecific agent comprises a first polypeptide comprising a FZD8 Fri domain and a second polypeptide comprising a human IgG2 Fc region. In some embodiments, the second arm of the bispecific agent comprises SEQ ID NO:28, SEQ ID NO:29, or SEQ ID NO:39. In some embodiments, the second arm of the bispecific agent comprises a first polypeptide comprising SEQ ID NO:28, SEQ ID NO:29, or SEQ ID NO:39 and a second polypeptide comprising SEQ ID NO:49, SEQ ID NO:50, SEQ ID NO:51, or SEQ ID NO:52.

In some embodiments, the MET-binding agent is a bispecific agent that specifically binds human MET and binds one or more WNT proteins, wherein the first arm of the bispecific agent comprises a heavy chain of SEQ ID NO:13 and a light chain of SEQ ID NO:14, and the second arm of the bispecific agent comprises a first polypeptide of SEQ ID NO:28 and a second polypeptide of SEQ ID NO:52. In some embodiments, the MET-binding agent is a bispecific agent that specifically binds human MET and binds one or more WNT proteins, wherein the first arm of the bispecific agent comprises a heavy chain of SEQ ID NO:100 and a light chain of SEQ ID NO:101, and the second arm of the bispecific agent comprises a first polypeptide of SEQ ID NO:28 and a second polypeptide of SEQ ID NO:52. In some embodiments, the MET-binding agent is a bispecific agent that specifically binds human MET and binds one or more WNT proteins, wherein the first arm of the bispecific agent comprises a heavy chain of SEQ ID NO:111 and a light chain of SEQ ID NO:101, and the second arm of the bispecific agent comprises a first polypeptide of SEQ ID NO:28 and a second polypeptide of SEQ ID NO:52. In some embodiments, the MET-binding agent is a bispecific agent that specifically binds human MET and binds one or more WNT proteins, wherein the first arm of the bispecific agent comprises a heavy chain of SEQ ID NO:13 and a light chain of SEQ ID NO:14, and the second arm of the bispecific agent comprises SEQ ID NO:56. In some embodiments, the MET-binding agent is a bispecific agent that specifically binds human MET and binds one or more WNT proteins, wherein the first arm of the bispecific agent comprises a heavy chain of SEQ ID NO:100 and a light chain of SEQ ID NO:101, and the second arm of the bispecific agent comprises SEQ ID NO:56. In some embodiments, the MET-binding agent is a bispecific agent that specifically binds human MET and binds one or more WNT proteins, wherein the first arm of the bispecific agent comprises a heavy chain of SEQ ID NO:111 and a light chain of SEQ ID NO:101, and the second arm of the bispecific agent comprises SEQ ID NO:56.

In some embodiments, the bispecific agent is referred to as bispecific agent 315B06. Bispecific agent 315B06 comprises (a) a heavy chain encoded by the plasmid comprising SEQ ID NO:16 deposited with ATCC, 10801 University Boulevard, Manassas, Va., USA, under the conditions of the Budapest Treaty on Mar. 12, 2013 and assigned designation number PTA-13609; (b) a light chain encoded by the plasmid comprising SEQ ID NO:19 deposited with ATCC under the conditions of the Budapest Treaty on Mar. 12, 2013 and assigned designation number PTA-13610; and (c) a polypeptide encoded by the plasmid comprising SEQ ID NO:89 deposited with ATCC under the conditions of the Budapest Treaty on Mar. 12, 2013 and assigned designation number PTA-13611. Bispecific agent 315B06 comprises (a) a heavy chain comprising SEQ ID NO:13 encoded by the plasmid deposited with ATCC and assigned designation number PTA-13609; (b) a light chain comprising SEQ ID NO:14 encoded by the plasmid deposited with ATCC and assigned designation number PTA-13610; and (c) a polypeptide comprising SEQ ID NO:56 encoded by the plasmid deposited with ATCC and assigned designation number PTA-13611.

In some embodiments, the bispecific agent is referred to as bispecific agent 315B07. In some embodiments, the bispecific agent 315B07 comprises (a) a heavy chain encoded by the plasmid comprising SEQ ID NO:103 deposited with ATCC, 10801 University Boulevard, Manassas, Va., USA, under the conditions of the Budapest Treaty on May 29, 2013 and assigned designation number PTA-120387; (b) a light chain encoded by the plasmid comprising SEQ ID NO:106 deposited with ATCC under the conditions of the Budapest Treaty on May 29, 2013 and assigned designation number PTA-120388; and (c) a polypeptide encoded by the plasmid comprising SEQ ID NO:89 deposited with ATCC under the conditions of the Budapest Treaty on Mar. 12, 2013 and assigned designation number PTA-13611. In some embodiments, bispecific agent 315B07 comprises (a) a heavy chain comprising SEQ ID NO:100 encoded by the plasmid deposited with ATCC and assigned designation number PTA-120387; (b) alight chain comprising SEQ ID NO:101 encoded by the plasmid deposited with ATCC and assigned designation number PTA-120388; and (c) a polypeptide comprising SEQ ID NO:56 encoded by the plasmid deposited with ATCC and assigned designation number PTA-13611.

In some embodiments, the bispecific agent is referred to as bispecific agent 315B09. In some embodiments, the bispecific agent 315B09 comprises (a) a heavy chain encoded by the plasmid comprising SEQ ID NO:109 deposited with ATCC, 10801 University Boulevard, Manassas, Va., USA, under the conditions of the Budapest Treaty on Nov. 6, 2013 and assigned designation number PTA-120695; (b) a light chain encoded by the plasmid comprising SEQ ID NO:106 deposited with ATCC under the conditions of the Budapest Treaty on May 29, 2013 and assigned designation number PTA-120388; and (c) a polypeptide encoded by the plasmid comprising SEQ ID NO:89 deposited with ATCC under the conditions of the Budapest Treaty on Mar. 12, 2013 and assigned designation number PTA-13611. In some embodiments, bispecific agent 315B09 comprises (a) a heavy chain comprising SEQ ID NO:111 encoded by the plasmid deposited with ATCC and assigned designation number PTA-120695; (b) a light chain comprising SEQ ID NO:101 encoded by the plasmid deposited with ATCC and assigned designation number PTA-120388; and (c) a polypeptide comprising SEQ ID NO:56 encoded by the plasmid deposited with ATCC and assigned designation number PTA-13611.

In some embodiments, the bispecific agent comprises a heavy chain comprising the heavy chain variable region encoded by the plasmid deposited with ATCC designated PTA-13609 and a light chain comprising the light chain variable region encoded by the plasmid deposited with ATCC designated PTA-13610. In some embodiments, the bispecific agent comprises a heavy chain comprising the heavy chain variable region encoded by the plasmid deposited with ATCC designated PTA-120387 and a light chain comprising the light chain variable region encoded by the plasmid deposited with ATCC designated PTA-120388. In some embodiments, the bispecific agent comprises a heavy chain comprising the heavy chain variable region encoded by the plasmid deposited with ATCC designated PTA-120695 and a light chain comprising the light chain variable region encoded by the plasmid deposited with ATCC designated PTA-120388. In some embodiments, the bispecific agent comprises a polypeptide encoded by the plasmid deposited with ATCC designated PTA-13611.

In some embodiments, the MET-binding agent is a bispecific agent that specifically binds human MET and binds one or more WNT proteins, wherein the first arm of the bispecific agent comprises a heavy chain of SEQ ID NO:88 and a light chain of SEQ ID NO:14, and wherein the second arm of the bispecific agent comprises a first polypeptide of SEQ ID NO:28 and a second polypeptide of SEQ ID NO:50. In some embodiments, the MET-binding agent is a bispecific agent that specifically binds human MET and binds one or more WNT proteins, wherein the first arm of the bispecific agent comprises a heavy chain of SEQ ID NO:88 and a light chain of SEQ ID NO:14, and wherein the second arm of the bispecific agent comprises SEQ ID NO:87.

In some embodiments, the MET-binding agent is a bispecific agent that specifically binds human MET and binds one or more WNT proteins, wherein the first arm of the bispecific agent comprises a heavy chain of SEQ ID NO:108 and a light chain of SEQ ID NO:101, and wherein the second arm of the bispecific agent comprises a first polypeptide of SEQ ID NO:28 and a second polypeptide of SEQ ID NO:50. In some embodiments, the MET-binding agent is a bispecific agent that specifically binds human MET and binds one or more WNT proteins, wherein the first arm of the bispecific agent comprises a heavy chain of SEQ ID NO:108 and a light chain of SEQ ID NO:101, and wherein the second arm of the bispecific agent comprises SEQ ID NO:87.

In some embodiments, the MET-binding agent is a bispecific agent that specifically binds human MET and binds one or more WNT proteins, wherein the first arm of the bispecific agent comprises a heavy chain of SEQ ID NO:114 and a light chain of SEQ ID NO:101, and wherein the second arm of the bispecific agent comprises a first polypeptide of SEQ ID NO:28 and a second polypeptide of SEQ ID NO:50. In some embodiments, the MET-binding agent is a bispecific agent that specifically binds human MET and binds one or more WNT proteins, wherein the first arm of the bispecific agent comprises a heavy chain of SEQ ID NO:114 and a light chain of SEQ ID NO:101, and wherein the second arm of the bispecific agent comprises SEQ ID NO:87.

In some embodiments, the MET-binding agent is a bispecific agent that specifically binds human MET and binds one or more WNT proteins, wherein the first arm of the bispecific agent comprises a heavy chain variable region having at least about 80% sequence identity to SEQ ID NO:7 or SEQ ID NO:94 and a light chain variable region having at least about 80% sequence identity to SEQ ID NO:8 or SEQ ID NO:95, and the second arm of the bispecific agent comprises a FZD8 Fri domain. In certain embodiments, the first arm, of the bispecific agent comprises a heavy chain variable region having at least about 85%, at least about 90%, at least about 95%, at least about 97%, or at least about 99% sequence identity to SEQ ID NO:7 or SEQ ID NO:94 and a light chain variable region having at least about 85%, at least about 90%, at least about 95%, at least about 97%, or at least about 99% sequence identity to SEQ ID NO:8 or SEQ ID NO:95, and the second arm of the bispecific agent comprises a FZD8 Fri domain. In certain embodiments, the first arm of the bispecific agent comprises a heavy chain variable region having at least about 95% sequence identity to SEQ ID NO:7 and a light chain variable region having at least about 95% sequence identity to SEQ ID NO:8, and the second arm of the bispecific agent comprises a FZD8 Fri domain. In certain embodiments, the first arm of the bispecific agent comprises a heavy chain variable region having at least about 95% sequence identity to SEQ ID NO:94 and a light chain variable region having at least about 95% sequence identity to SEQ ID NO:95, and the second arm of the bispecific agent comprises a FZD8 Fri domain. In certain embodiments, the first arm of the bispecific agent comprises a heavy chain variable region comprising SEQ ID NO:7 and a light chain variable region comprising SEQ ID NO:8, and the second arm of the bispecific agent comprises a FZD8 Fri domain. In certain embodiments, the first arm of the bispecific agent comprises a heavy chain variable region comprising SEQ ID NO:94 and a light chain variable region comprising SEQ ID NO:95, and the second arm of the bispecific agent comprises a FZD8 Fri domain. In certain embodiments, the first arm of the bispecific agent comprises a heavy chain variable region of SEQ ID NO:7 and a light chain variable region of SEQ ID NO:8, and the second arm of the bispecific agent comprises a FZD8 Fri domain. In certain embodiments, the first arm of the bispecific agent comprises a heavy chain variable region of SEQ ID NO:94 and a light chain variable region of SEQ ID NO:95, and the second arm of the bispecific agent comprises a FZD8 Fri domain.

In some embodiments, the MET-binding agent is a bispecific agent, wherein the first arm of the bispecific arm comprises a first CH3 domain and the second arm of the bispecific agent comprises a second CH3 domain, and each of the CH3 domains is modified to promote formation of heterodimers or heteromultimers. In some embodiments, the first and second CH3 domains are modified using a knobs-into-holes technique. In some embodiments, the first and second CH3 domains comprise changes or substitutions in amino acids that result in altered electrostatic interactions. In some embodiments, the first and second CH3 domains comprise changes in amino acids that result in altered hydrophobic/hydrophilic interactions.

In some embodiments, the MET-binding agent is a bispecific agent that comprises two heavy chain constant regions selected from the group consisting of: (a) a first human IgG1 constant region, wherein the amino acids at positions corresponding to positions 253 and 292 of SEQ ID NO:74 are substituted or replaced with glutamate or aspartate, and a second human IgG1 constant region, wherein the amino acids at positions corresponding to positions 240 and 282 of SEQ ID NO:74 are substituted or replaced with lysine; (b) a first human IgG2 constant region, wherein the amino acids at positions corresponding to positions 249 and 288 of SEQ ID NO:75 are substituted or replaced with glutamate or aspartate, and a second human IgG2 constant region wherein the amino acids at positions corresponding to positions 236 and 278 of SEQ ID NO:75 are substituted or replaced with lysine; (c) a first human IgG3 constant region, wherein the amino acids at positions corresponding to positions 300 and 339 of SEQ ID NO:76 are substituted or replaced with glutamate or aspartate, and a second human IgG3 constant region wherein the amino acids at positions corresponding to positions 287 and 329 of SEQ ID NO:76 are substituted or replaced with lysine; and (d) a first human IgG4 constant region, wherein the amino acids at positions corresponding to positions 250 and 289 of SEQ ID NO:77 are substituted or replaced with glutamate or aspartate, and a second IgG4 constant region wherein the amino acids at positions corresponding to positions 237 and 279 of SEQ ID NO:77 are substituted or replaced with lysine.

In some embodiments, the bispecific agent comprises a first human IgG1 constant region with amino acid substitutions at positions corresponding to positions 253 and 292 of SEQ ID NO:74, wherein the amino acids are replaced with glutamate or aspartate, and a second human IgG1 constant region with amino acid substitutions at positions corresponding to positions 240 and 282 of SEQ ID NO:74, wherein the amino acids are replaced with lysine. In some embodiments, the bispecific agent comprises a first human IgG2 constant region with amino acid substitutions at positions corresponding to positions 249 and 288 of SEQ ID NO:75, wherein the amino acids are replaced with glutamate or aspartate, and a second human IgG2 constant region with amino acid substitutions at positions corresponding to positions 236 and 278 of SEQ ID NO:75, wherein the amino acids are replaced with lysine. In some embodiments, the bispecific agent comprises a first human IgG3 constant region with amino acid substitutions at positions corresponding to positions 300 and 339 of SEQ ID NO:76, wherein the amino acids are replaced with glutamate or aspartate, and a second human IgG2 constant region with amino acid substitutions at positions corresponding to positions 287 and 329 of SEQ ID NO:76, wherein the amino acids are replaced with lysine. In some embodiments, the bispecific agent comprises a first human IgG4 constant region with amino acid substitutions at positions corresponding to positions 250 and 289 of SEQ ID NO:77, wherein the amino acids are replaced with glutamate or aspartate, and a second human IgG4 constant region with amino acid substitutions at positions corresponding to positions 237 and 279 of SEQ ID NO:77, wherein the amino acids are replaced with lysine.

In some embodiments, the bispecific agent comprises a first human IgG2 constant region with amino acid substitutions at positions corresponding to positions 249 and 288 of SEQ ID NO:75, wherein the amino acids are replaced with glutamate, and a second human IgG2 constant region with amino acid substitutions at positions corresponding to positions 236 and 278 of SEQ ID NO:75, wherein the amino acids are replaced with lysine. In some embodiments, the bispecific agent comprises a first human IgG2 constant region with amino acid substitutions at positions corresponding to positions 249 and 288 of SEQ ID NO:75, wherein the amino acids are replaced with asparate, and a second human IgG2 constant region with amino acid substitutions at positions corresponding to positions 236 and 278 of SEQ ID NO:75, wherein the amino acids are replaced with lysine.

In certain embodiments, a MET-binding agent binds MET and/or one or more components of the WNT pathway with a dissociation constant (K_{D}) of about 1 μM or less, about 100 nM or less, about 40 nM or less, about 20 nM or less, about 10 nM or less, about 1 nM or less, or about 0.1 nM or less. In some embodiments, a MET-binding agent binds MET and/or one or more components of the WNT pathway with a K_{D} of about 20 nM or less. In some embodiments, a MET-binding agent binds MET and/or one or more components of the WNT pathway with a K_{D} of about 1004 or less. In some embodiments, a MET-binding agent binds MET and/or one or more components of the WNT pathway with a K_{D} of about 1 nM or less. In some embodiments, a MET-binding agent binds MET and/or one or more components of the WNT pathway with a K_{D} of about 0.1 nM or less. In some embodiments, a MET-binding agent binds both human MET and mouse MET with a K_{D} of about 100 nM or less. In some embodiments, a MET-binding agent binds both human MET and mouse MET with a K_{D} of about 50 nM or less. In some embodiments, a MET-binding agent binds human MET and does not bind mouse MET. In some embodiments, a MET-binding agent binds one or more human WNT proteins with a K_{D} of about 100 nM or less. In some embodiments, a MET-binding agent binds one or more human WNT proteins with a K_{D} of about 50 nM or less. In some embodiments, a MET-binding agent binds one or more human WNT proteins with a K_{D} of about 20 nM or less. In some embodiments, the dissociation constant of the binding agent (e.g., an antibody or bispecific agent) to MET is the dissociation constant determined using a MET fusion protein comprising at least a portion of MET immobilized on a Biacore chip. In some embodiments, the dissociation constant of the binding agent (e.g., an antibody or bispecific agent) to a WNT protein is the dissociation constant determined using a WNT-fusion protein comprising at least a portion of a WNT protein immobilized on a Biacore chip.

In some embodiments, the MET-binding agent is a bispecific agent that comprises a first binding site that specifically binds MET and a second binding site that specifically binds one or more components of the WNT pathway. In some embodiments, a MET-binding agent binds both MET and one or more components of the WNT pathway (e.g., WNT proteins or FZD proteins) with a K_{D} of about 100 nM or less. In some embodiments, a MET-binding agent binds both MET and one or more components of the WNT pathway with a K_{D} of about 50 nM or less. In some embodiments, a MET-binding agent binds both MET and one or more components of the WNT pathway with a K_{D} of about 20 nM or less. In some embodiments, a MET-binding agent binds both MET and one or more components of the WNT pathway with a K_{D} of about 10 nM or less. In some embodiments, a MET-binding agent or antibody binds both MET and one or more components of the WNT pathway with a K_{D} of about 1 nM or less.

In some embodiments, the MET-binding agent is a bispecific agent that comprises a first binding site with a binding affinity that is weaker than the binding affinity of the second binding site. For example, in some embodiments, the bispecific agent may bind MET with a K_{D} ranging from about 0.1 nM to 1 nM and may bind one or more components of the WNT pathway with a K_{D} ranging from about 1 nM to 10 nM. Or the bispecific agent may bind MET with a K_{D} ranging from about 1 nM to 10 nM and may bind one or more components of the WNT pathway with a K_{D} ranging from about 0.1 nM to 1 nM. In some embodiments, the bispecific agent may bind one or more components of the WNT pathway with a K_{D} ranging from about 0.1 nM to 1 nM and may bind MET with a K_{D} ranging from about 1 nM to 10 nM. Or the bispecific agent may bind one or more components of the WNT pathway with a K_{D} ranging from about 1 nM to 10 nM and may bind MET with a K_{D} ranging from about 0.1 nM to 1 nM. In some embodiments, the difference in affinity between the two binding sites may be about 2-fold or more, about 3-fold or more, about 5-fold or more, about 8-fold or more, about 10-fold or more, about 15-fold or more, about 30-fold or more, about 50-fold or more, or about 100-fold or more. In some embodiments, at least one amino acid residue in at least one CDR of the antigen-binding site for MET is substituted with a different amino acid so that the affinity of the MET-binding site is altered. In some embodiments, the affinity of the MET-binding site is increased. In some embodiments, the affinity of the MET-binding site is decreased. In some embodiments, the affinities of both the MET and one or more components of the WNT pathway binding sites are altered. Modulation of the affinities of the two binding sites may affect the biological activity of the bispecific agent. For example, decreasing the affinity of the binding site for MET or one or more components of the WNT pathway may have a desirable effect, for example decreased toxicity of the binding agent or an increased therapeutic index of the binding agent.

By way of non-limiting example, the bispecific agent may comprise (a) a first binding site that binds human MET with a K_{D} between about 0.1 nM and about 10 nM, and (b) a second binding site that specifically binds one or more human WNT proteins with a K_{D} between about 0.1 nM and about 20 nM, between about 0.5 nM and about 20 nM, between about 1.0 nM and 10 nM.

In certain embodiments, a MET-binding agent binds MET and one or more components of the WNT pathway (e.g., WNT proteins or FZD proteins) with a half maximal effective concentration (EC₅₀) of about 1 μM or less, about 100 nM or less, about 40 nM or less, about 20 nM or less, about 10 nM or less, about 1 nM or less, or about 0.1 nM or less. In certain embodiments, a MET-binding agent binds MET and one or more components of the WNT pathway (e.g., WNT proteins or FZD proteins) with a half maximal effective concentration (EC₅₀) of about 1 μM or less, about 100 nM or less, about 40 nM or less, about 20 nM or less, about 10 nM or less, about 1 nM or less, or about 0.1 nM or less.

In certain embodiments, the MET-binding agent comprises an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In certain embodiments, the antibody is an IgA, IgD, IgE, IgG, or IgM antibody. In certain embodiments, the antibody is an IgG1 antibody. In certain embodiments, the antibody is an IgG2 antibody. In certain embodiments, the antibody is an antibody fragment comprising an antigen-binding site. In some embodiments, the antibody is a bispecific antibody. In some embodiments, the antibody is a monovalent antibody. In some embodiments, the antibody is a monospecific antibody. In some embodiments, the antibody is a multispecific antibody. In some embodiments, the antibody is conjugated to a cytotoxic moiety. In some embodiments, the antibody is isolated. In some embodiments, the antibody is substantially pure.

The binding agents of the present invention can be assayed for specific binding by any method known in the art. The immunoassays which can be used include, but are not limited to, competitive and non-competitive assay systems using techniques such as Biacore analysis, FACS analysis, immunofluorescence, immunocytochemistry, Western blot analysis, radioimmunoassay, ELISA, “sandwich” immunoassay, immunoprecipitation assay, precipitation reaction, gel diffusion precipitin reaction, immunodiffusion assay, agglutination assay, complement-fixation assay, immunoradiometric assay, fluorescent immunoassay, homogeneous time-resolved fluorescence assay (HTRF), and protein A immunoassay. Such assays are routine and well-known in the art (see, e.g., Ausubel et al., Editors, 1994-present, Current Protocols in Molecular Biology, John Wiley & Sons, Inc., New York, N.Y.).

For example, the specific binding of an agent to human MET and/or to a component of the WNT pathway (e.g., FZD proteins or WNT proteins) may be determined using ELISA. An ELISA assay comprises preparing antigen, coating wells of a 96 well microtiter plate with antigen, adding the binding agent conjugated to a detectable compound such as an enzymatic substrate (e.g. horseradish peroxidase or alkaline phosphatase) to the well, incubating for a period of time, and detecting the presence of the binding agent bound to the antigen. In some embodiments, the binding agent is not conjugated to a detectable compound, but instead a secondary antibody that recognizes the binding agent (e.g., an anti-Fc antibody) and is conjugated to a detectable compound is added to the well. In some embodiments, instead of coating the well with the antigen, the binding agent can be coated to the well and a secondary antibody conjugated to a detectable compound can be added following the addition of the antigen to the coated well. One of skill in the art would be knowledgeable as to the parameters that can be modified to increase the signal detected as well as other variations of ELISAs known in the art.

In another example, the specific binding of an agent to human MET and/or to a component of the WNT pathway (e.g., FZD proteins or WNT proteins) may be determined using FACS. A FACS screening assay may comprise generating a cDNA construct that expresses an antigen as a fusion protein, transfecting the construct into cells, expressing the antigen on the surface of the cells, mixing the binding agent with the transfected cells, and incubating for a period of time. The cells bound by the binding agent may be identified by using a secondary antibody conjugated to a detectable compound (e.g., PE-conjugated anti-Fc antibody) and a flow cytometer. One of skill in the art would be knowledgeable as to the parameters that can be modified to optimize the signal detected as well as other variations of FACS that may enhance screening (e.g., screening for blocking antibodies).

The binding affinity of a binding agent to an antigen (e.g., MET or a component of the WNT pathway) and the off-rate of a binding agent-target interaction can be determined by competitive binding assays. One example of a competitive binding assay is a radioimmunoassay comprising the incubation of labeled antigen/target (e.g., ³H or ¹²⁵I), or fragment or variant thereof, with the binding agent of interest in the presence of increasing amounts of unlabeled antigen followed by the detection of the antibody bound to the labeled antigen/target. The affinity of the binding agent for the antigen/target and the binding off-rates can be determined from the data by Scatchard plot analysis. In some embodiments, Biacore kinetic analysis is used to determine the binding on and off rates of binding agents that bind an antigen (e.g., MET or a component of the WNT pathway). In some embodiments, Biacore kinetic analysis comprises analyzing the binding and dissociation of binding agents from chips with immobilized antigen/target (e.g., MET or a component of the WNT pathway) on their surface. In some embodiments, Biacore kinetic analysis comprises analyzing the binding and dissociation of an antigen or target (e.g., MET or a component of the WNT pathway) from chips with immobilized binding agent on their surface.

The invention provides polypeptides that specifically bind MET, bind at least one component of the WNT pathway, or bind MET and at least one component of the WNT pathway. In some embodiments, a polypeptide binds human MET. In some embodiments, a polypeptide binds human MET and mouse MET. In some embodiments, a polypeptide binds human MET and does not bind mouse MET. In some embodiments, a polypeptide binds one or more components of the human WNT pathway. In some embodiments, a polypeptide binds one or more human FZD proteins. In some embodiments, a polypeptide binds one or more human WNT proteins. In some embodiments, a polypeptide multimer binds MET and one or more components of the human WNT pathway. In some embodiments, a polypeptide multimer binds MET and one or more human FZD proteins. In some embodiments, a polypeptide multimer binds MET and one or more human WNT proteins.

In some embodiments, a MET-binding agent comprises a polypeptide comprising a sequence selected from the group consisting of: SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:28, SEQ ID NO:29, SEQ ID NO:39, SEQ ID NO:55, SEQ ID NO:56, SEQ ID NO:87, SEQ ID NO:88, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:108, SEQ ID NO:110, SEQ ID NO:111, and SEQ ID NO:114. In some embodiments, the MET-binding agent further comprises a polypeptide comprising a sequence selected from the group consisting of: SEQ ID NO:47, SEQ ID NO:48, SEQ ID NO:49, SEQ ID NO:50, SEQ ID NO:51, and SEQ ID NO:52.

In certain embodiments, a MET-binding agent competes for specific binding to MET with an antibody or a bispecific agent that comprises a heavy chain variable region comprising SEQ ID NO:7 and a light chain variable region comprising SEQ ID NO:8. In certain embodiments, a MET-binding agent competes for specific binding to MET with an antibody or a bispecific agent that comprises a heavy chain variable region comprising SEQ ID NO:94 and a light chain variable region comprising SEQ ID NO:95. In certain embodiments, a MET-binding agent competes with antibody 73R009 for specific binding to human MET. In certain embodiments, a MET-binding agent competes with a humanized version of antibody 73R009 for specific binding to human MET. In certain embodiments, a MET-binding agent competes with antibody 73R010 (73R009 H12L7) for specific binding to human MET. In certain embodiments, a MET-binding agent competes with a monovalent version of antibody 73R009 for specific binding to human MET. In some embodiments, a MET-binding agent competes with a bispecific agent comprising the heavy chain variable region and the light chain variable region of antibody 73R009 for specific binding to human MET. In some embodiments, a MET-binding agent competes with a bispecific agent comprising the heavy chain variable region and the light chain variable region of a humanized version of antibody 73R009 for specific binding to human MET. In some embodiments, a MET-binding agent competes with a bispecific agent comprising the heavy chain variable region and the light chain variable region of antibody 73R010 (73R009 H12L7) for specific binding to human MET. In some embodiments, a MET-binding agent competes for specific binding to MET with a MET-binding agent described herein in an in vitro competitive binding assay. In some embodiments, the MET is human MET. In some embodiments, the MET is mouse MET.

In certain embodiments, a MET-binding agent binds the same epitope, or essentially the same epitope, on MET as an antibody or bispecific agent of the invention. In another embodiment, a MET-binding agent is an antibody that binds an epitope on MET that overlaps with the epitope on MET bound by an antibody or bispecific agent of the invention. In certain embodiments, a MET-binding agent binds the same epitope, or essentially the same epitope, on MET as antibody 73R009, a humanized version of 73R009, or antibody 73R010 (73R009 H12L7). In another embodiment, the MET-binding agent is an antibody or binding agent that binds an epitope on MET that overlaps with the epitope on MET bound by antibody 73R009, a humanized version of 73R009, or antibody 73R010 (73R009 H12L7). In certain embodiments, a MET-binding agent binds the same epitope, or essentially the same epitope, on MET as bispecific agent 315B06, 315B07, or 315B09. In another embodiment, the MET-binding agent is an antibody or binding agent that binds an epitope on MET that overlaps with the epitope on MET bound by bispecific agent 315B06, 315B07, or 315B09.

In certain embodiments, the MET-binding agent is an agent that competes for specific binding to MET with the antibody 73R009, a monovalent version of 73R009, a humanized version of antibody 73R009, or antibody 73R010 (73R009 H12L7) (e.g., in a competitive binding assay). In certain embodiments, the MET-binding agent is an agent that competes for specific binding to MET with bispecific agent 315B06, 315B07, or 315B09 (e.g., in a competitive binding assay).

In certain embodiments, a binding agent competes with bispecific agent 315B06, 315B07, or 315B09 for specific binding to one or more WNT proteins. In some embodiments, a binding agent or antibody competes for specific binding to one or more WNT proteins with an agent described herein in an in vitro competitive binding assay. In some embodiments, the one or more WNT proteins are human WNT proteins.

In certain embodiments, a binding agent (e.g., an antibody) binds the same target, or essentially the same target, on one or more WNT proteins as a bispecific agent of the invention. In some embodiments, a binding agent binds a target on one or more WNT proteins that overlaps with the target on one or more WNT proteins bound by a bispecific agent of the invention. In certain embodiments, a binding agent binds the same target, or essentially the same target, on one or more WNT proteins as bispecific agent 315B06, 315B07, or 315B09. In another embodiment, the binding agent binds a target on one or more WNT proteins that overlaps with the target on WNT bound by bispecific agent 315B06, 315B07, or 315B09.

In certain embodiments, the binding agent is an agent that competes for specific binding to one or more WNT proteins with the bispecific agent 315B06, 315B07, or 315B09 (e.g., in a competitive binding assay).

In certain embodiments, the binding agent is an agent that competes for specific binding to MET and/or one or more WNT proteins with the bispecific agent 315B06, 315B07, or 315B09 (e.g., in a competitive binding assay).

In certain embodiments, the MET-binding agent (e.g., an antibody or bispecific agent) described herein binds MET and modulates MET activity. In some embodiments, the MET-binding agent is a MET antagonist and inhibits MET activity. In some embodiments, the MET-binding agent inhibits MET activity. MET activity may be inhibited by several different mechanisms, including but not limited to, inhibition or blockage of the MET/HGF interaction, inhibition or blockage of MET dimerization, increase in MET shedding, increase in MET internalization, and/or increase in MET degradation. In some embodiments, the MET-binding agent inhibits MET activation. In some embodiments, the MET-binding agent inhibits and/or reduces MET phosphorylation. In some embodiments, the MET-binding agent is a MET antagonist and inhibits tumor growth. In some embodiments, the MET-binding agent inhibits tumor growth. In some embodiments, the MET-binding agent is a MET antagonist and inhibits angiogenesis. In some embodiments, the MET-binding agent inhibits angiogenesis. In some embodiments, the MET-binding agent is a MET antagonist and inhibits EMT. In some embodiments, the MET-binding agent inhibits EMT.

In certain embodiments, a MET-binding agent (e.g., an antibody or bispecific agent) described herein binds one or more human WNT proteins and modulates WNT pathway activity. In some embodiments, a MET-binding agent is a WNT pathway antagonist and inhibits WNT pathway activity. In some embodiments, a MET-binding agent inhibits WNT pathway activity. In some embodiments, a MET-binding agent is a WNT pathway antagonist and inhibits β-catenin activity. In some embodiments, a MET-binding agent inhibits β-catenin activity. In some embodiments, a MET-binding agent is a WNT pathway antagonist and inhibits tumor growth. In some embodiments, a MET-binding agent is a WNT pathway antagonist and induces differentiation of tumor cells. In some embodiments, a MET-binding agent induces differentiation of tumor cells. In some embodiments, a MET-binding agent is a WNT pathway antagonist and induces differentiation of cancer stem cells. In some embodiments, a MET-binding agent induces differentiation of cancer stem cells. In some embodiments, a MET-binding agent is a WNT pathway antagonist and induces expression of differentiation markers on tumor cells. In some embodiments, a MET-binding agent induces expression of differentiation markers on tumor cells. In some embodiments, a MET-binding agent is a WNT pathway antagonist and induces expression of differentiation markers on cancer stem cells. In some embodiments, a MET-binding agent induces expression of differentiation markers on cancer stem cells.

In certain embodiments, a MET-binding agent (e.g., an antibody or bispecific agent) described herein is a bispecific agent that binds human MET and modulates MET activity. In certain embodiments, a MET-binding agent described herein is a bispecific agent that binds one or more components of the human WNT pathway and modulates WNT activity. In certain embodiments, a MET-binding agent described herein is a bispecific agent that binds human MET and one or more components of the human WNT pathway and modulates both MET activity and WNT pathway activity. In some embodiments, the bispecific agent is a MET antagonist and a WNT pathway antagonist and inhibits both MET activity and WNT pathway activity. In some embodiments, the bispecific agent is a MET antagonist and a WNT pathway antagonist and inhibits MET signaling and WNT pathway signaling. In some embodiments, the bispecific agent is a MET antagonist and a WNT pathway antagonist and inhibits tumor growth.

In certain embodiments, the MET-binding agent (e.g., an antibody or a bispecific agent) is an antagonist of MET. In some embodiments, the MET-binding agent is an antagonist of MET and inhibits MET activity. In certain embodiments, the MET-binding agent inhibits MET activity by at least about 10%, at least about 20%, at least about 30%, at least about 50%, at least about 75%, at least about 90%, or about 100%. In certain embodiments, a MET-binding agent that inhibits human MET activity comprises antibody 73R009, a humanized version of antibody 73R009, or antibody 73R010 (73R009 H12L7). In certain embodiments, a MET-binding agent that inhibits human MET activity comprises a monovalent version of antibody 73R009 or antibody 73R010 (73R009 H12L7). In certain embodiments, a MET-binding agent that inhibits human MET activity comprises the heavy chain variable region and the light chain variable region of antibody 73R009, the heavy chain variable region and the light chain variable region of a humanized version of antibody 73R009, or the heavy chain variable region and the light chain variable region of antibody 73R010 (73R009 H12L7). In certain embodiments, a MET-binding agent that inhibits human MET activity is bispecific agent 315B06, 315B07, or 315B09.

In certain embodiments, the MET-binding agent is an antagonist of the WNT pathway. In some embodiments, the MET-binding agent is an antagonist of the WNT pathway and inhibits WNT pathway activity. In certain embodiments, the MET-binding agent inhibits WNT pathway activity by at least about 10%, at least about 20%, at least about 30%, at least about 50%, at least about 75%, at least about 90%, or about 100%. In certain embodiments, a MET-binding agent that inhibits human WNT pathway activity comprises antibody 73R009, a humanized version of antibody 73R009, or antibody 73R010 (73R009 H12L7). In certain embodiments, a MET-binding agent that inhibits human WNT pathway activity comprises a monovalent version of antibody 73R009 or antibody 73R010 (73R009 H12L7). In certain embodiments, a MET-binding agent that inhibits human WNT pathway activity comprises the heavy chain variable region and the light chain variable region of antibody 73R009, the heavy chain variable region and the light chain variable region of a humanized version of antibody 73R009, or the heavy chain variable region and the light chain variable region of antibody 73R010 (73R009 H12L7). In certain embodiments, a MET-binding agent that inhibits human WNT pathway activity is a bispecific agent comprising the antigen-binding site of antibody 73R009, the antigen-binding site of a humanized version of antibody 73R009, or the antigen-binding site of antibody 73R010 (73R009 H12L7). In certain embodiments, a MET-binding agent that inhibits human WNT pathway activity is bispecific agent 315B06, 315B07, or 315B09.

In certain embodiments, the MET-binding agent inhibits binding of MET to hepatocyte growth factor (HGF). In certain embodiments, the MET-binding agent inhibits binding of MET to HGF by at least about 10%, at least about 25%, at least about 50%, at least about 75%, at least about 90%, or at least about 95%. In certain embodiments, a MET-binding agent that inhibits binding of human MET to HGF is antibody 73R009, a humanized version of antibody 73R009, or antibody 73R010 (73R009 H12L7). In certain embodiments, a MET-binding agent that inhibits binding of human MET to HGF is a monovalent version of antibody 73R009 or antibody 73R010 (73R009 H12L7). In certain embodiments, a MET-binding agent that inhibits binding of human MET to HGF is a bispecific agent comprising the antigen-binding site of antibody 73R009, the antigen-binding site of a humanized version of antibody 73R009, or the antigen-binding site of antibody 73R010 (73R009 H12L7). In certain embodiments, a MET-binding agent that inhibits binding of human MET to HGF is a bispecific agent comprising the heavy chain variable region and the light chain variable region of antibody 73R009, the heavy chain variable region and the light chain variable region of a humanized version of antibody 73R009, or the heavy chain variable region and the light chain variable region of antibody 73R010 (73R009 H12L7). In certain embodiments, a MET-binding agent that inhibits binding of human MET to HGF is bispecific agent 315B06, 315B07, or 315B09.

In certain embodiments, the MET-binding agent (e.g., a bispecific agent) inhibits binding of one or more WNT proteins to one or more FZD proteins. In some embodiments, the MET-binding agent (e.g., a bispecific agent) inhibits binding of one or more WNT proteins to FZD1, FZD2, FZD3, FZD4, FZD5, FZD6, FZD7, FZD8, FZD9, and/or FZD10. In some embodiments, the MET-binding agent (e.g., a bispecific agent) inhibits binding of one or more WNT proteins to FZD8. In certain embodiments, the MET-binding agent inhibits binding of one or more WNT proteins to at least one FZD receptor by at least about 10%, at least about 25%, at least about 50%, at least about 75%, at least about 90%, or at least about 95%. In certain embodiments, a MET-binding agent that inhibits binding of one or more human WNT proteins to at least one FZD receptor is bispecific agent 315B06, 315B07, or 315B09.

In vivo and in vitro assays for determining whether a MET-binding agent (or candidate MET-binding agent) inhibits MET activation are known in the art. For example, binding of human HGF to MET results in tyrosine phosphorylation of MET and activation of the MET signaling pathway. Therefore, human cells that are responsive to HGF may be used to assess the inhibition of HGF-induced MET activation by analyzing phosphorylation of MET and phosphorylation of downstream MET pathway components such as mitogen activate protein kinase (MAPK) and AKT. Assays to determine whether a MET-binding agent (or candidate MET-binding agent) inhibits MET dimerization, promotes MET degradation, and/or promotes MET “shedding” are also known in the art.

In vivo and in vitro assays for determining whether a MET-binding agent (or candidate MET-binding agent) inhibits WNT pathway activation or signaling are known in the art. For example, cell-based, luciferase reporter assays utilizing a TCF/Luc reporter vector containing multiple copies of the TCF-binding domain upstream of a firefly luciferase reporter gene may be used to measure β-catenin signaling levels in vitro (Gazit et al., 1999, Oncogene, 18; 5959-66; TOPflash, Millipore, Billerica Mass.). The level of β-catenin signaling in the presence of one or more WNT proteins (e.g., WNT(s) expressed by transfected cells or provided by WNT-conditioned media) in the presence of a binding agent is compared to the level of signaling without the binding agent present. In addition to the TCF/Luc reporter assay, the effect of a binding agent (or candidate agent) on β-catenin signaling may be measured in vitro or in vivo by measuring the effect of the agent on the level of expression of β-catenin-regulated genes, such as c-myc (He et al., 1998, Science, 281:1509-12), cyclin D1 (Tetsu et al., 1999, Nature, 398:422-6), and/or fibronectin (Gradl et al. 1999, Mol. Cell Biol., (9:5576-87). In certain embodiments, the effect of a binding agent on β-catenin signaling may also be assessed by measuring the effect of the agent on the phosphorylation state of Dishevelled-1, Dishevelled-2, Dishevelled-3, LRP5, LRP6, and/or β-catenin.

In certain embodiments, the MET-binding agents have one or more of the following effects: inhibit proliferation of tumor cells, inhibit tumor growth, reduce the tumorigenicity of a tumor, reduce the frequency of cancer stem cells in a tumor, reduce the tumorigenicity of a tumor by reducing the frequency of cancer stem cells in the tumor, trigger cell death of tumor cells, induce cells in a tumor to differentiate, differentiate tumorigenic cells to a non-tumorigenic state, differentiate cancer stem cells, induce expression of differentiation markers in the tumor cells, induce expression of differentiation markers in the cancer stem cells, prevent metastasis of tumor cells, inhibit angiogenesis, decrease survival of tumor cells, or any combination of the above.

In certain embodiments, the MET-binding agents are capable of inhibiting tumor growth. In certain embodiments, the MET-binding agents are capable of inhibiting tumor growth in vivo (e.g., in a xenograft mouse model, and/or in a human having cancer). In certain embodiments, tumor growth is inhibited at least about two-fold, about three-fold, about five-fold, about ten-fold, about 50-fold, about 100-fold, or about 1000-fold as compared to an untreated tumor.

In certain embodiments, the MET-binding agents are capable of reducing the tumorigenicity of a tumor. In certain embodiments, the MET-binding agent is capable of reducing the tumorigenicity of a tumor comprising cancer stem cells in an animal model, such as a mouse xenograft model. In certain embodiments, the MET-binding agent is capable of reducing the tumorigenicity of a tumor by decreasing the number or frequency of cancer stem cells in the tumor. In certain embodiments, the number or frequency of cancer stem cells in a tumor is reduced by at least about two-fold, about three-fold, about five-fold, about ten-fold, about 50-fold, about 100-fold, or about 1000-fold. In certain embodiments, the reduction in the number or frequency of cancer stem cells is determined by limiting dilution assay using an animal model. Additional examples and guidance regarding the use of limiting dilution assays to determine a reduction in the number or frequency of cancer stem cells in a tumor can be found, e.g., in International Publication Number WO 2008/042236; U.S. Patent Publication No. 2008/0064049; and U.S. Patent Publication No. 2008/0178305.

In certain embodiments, the MET-binding agents described herein have a circulating half-life in mice, cynomolgus monkeys, or humans of at least about 2 hours, at least about 5 hours, at least about 10 hours, at least about 24 hours, at least about 3 days, at least about 1 week, or at least about 2 weeks. In certain embodiments, the MET-binding agent is an IgG (e.g., IgG1 or IgG2) antibody that has a circulating half-life in mice, cynomolgus monkeys, or humans of at least about 2 hours, at least about 5 hours, at least about 10 hours, at least about 24 hours, at least about 3 days, at least about 1 week, or at least about 2 weeks. In certain embodiments, the MET-binding agent is an agent comprising at least one IgG (e.g., IgG1 or IgG2) constant region that has a circulating half-life in mice, cynomolgus monkeys, or humans of at least about 2 hours, at least about 5 hours, at least about 10 hours, at least about 24 hours, at least about 3 days, at least about 1 week, or at least about 2 weeks. Methods of increasing (or decreasing) the half-life of agents such as polypeptides, soluble receptors, and/or antibodies are known in the art. For example, known methods of increasing the circulating half-life of IgG antibodies include the introduction of mutations in the Fc region which increase the pH-dependent binding of the antibody to the neonatal Fc receptor (FcRn) at pH 6.0 (see, e.g., U.S. Patent Publication Nos. 2005/0276799, 2007/0148164, and 2007/0122403). Known methods of increasing the circulating half-life of antibody fragments lacking the Fc region include such techniques as PEGylation.

In some embodiments, the binding agents described herein are antibodies. Polyclonal antibodies can be prepared by any known method. In some embodiments, polyclonal antibodies are produced by immunizing an animal (e.g., a rabbit, rat, mouse, goat, or donkey) with an antigen of interest (e.g., a purified peptide fragment, full-length recombinant protein, or fusion protein) by multiple subcutaneous or intraperitoneal injections. The antigen can be optionally conjugated to a carrier such as keyhole limpet hemocyanin (KLH) or serum albumin. The antigen (with or without a carrier protein) is diluted in sterile saline and usually combined with an adjuvant (e.g., Complete or Incomplete Freund's Adjuvant) to form a stable emulsion. After a sufficient period of time, polyclonal antibodies are recovered from the immunized animal, usually from blood or ascites. The polyclonal antibodies can be purified from serum or ascites according to standard methods in the art including, but not limited to, affinity chromatography, ion-exchange chromatography, gel electrophoresis, and dialysis.

In some embodiments, the binding agents are monoclonal antibodies. Monoclonal antibodies can be prepared using hybridoma methods known to one of skill in the art (see e.g., Kohler and Milstein, 1975, Nature, 256:495-497). In some embodiments, using the hybridoma method, a mouse, hamster, or other appropriate host animal, is immunized as described above to elicit from lymphocytes the production of antibodies that specifically bind the immunizing antigen. In some embodiments, lymphocytes can be immunized in vitro. In some embodiments, the immunizing antigen can be a human protein or a portion thereof. In some embodiments, the immunizing antigen can be a mouse protein or a portion thereof.

Following immunization, lymphocytes are isolated and fused with a suitable myeloma cell line using, for example, polyethylene glycol. The hybridoma cells are selected using specialized media as known in the art and unfused lymphocytes and myeloma cells do not survive the selection process. Hybridomas that produce monoclonal antibodies directed specifically against a chosen antigen may be identified by a variety of methods including, but not limited to, immunoprecipitation, immunoblotting, and in vitro binding assays (e.g., flow cytometry, FACS, ELISA, and radioimmunoassay). The hybridomas can be propagated either in in vitro culture using standard methods (J. W. Goding, 1996, Monoclonal Antibodies: Principles and Practice, 3^{rd} Edition, Academic Press, San Diego, Calif.) or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In certain embodiments, monoclonal antibodies can be made using recombinant DNA techniques as known to one skilled in the art. The polynucleotides encoding a monoclonal antibody are isolated from mature B-cells or hybridoma cells, such as by RT-PCR using oligonucleotide primers that specifically amplify the genes encoding the heavy and light chains of the antibody, and their sequence is determined using standard techniques. The isolated polynucleotides encoding the heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins.

In certain other embodiments, recombinant monoclonal antibodies, or fragments thereof, can be isolated from phage display libraries expressing variable domains or CDRs of a desired species (see e.g., McCafferty et al., 1990, Nature, 348:552-554; Clackson et al., 1991, Nature, 352:624-628; and Marks et al., 1991, J. Mol. Biol., 222:581-597). In some embodiments, recombinant monoclonal antibodies, or fragments thereof, can be isolated from mammalian cell display libraries expressing variable domains or CDRs of a desired species (see e.g., U.S. patent publication No. 2011/0287979).

The polynucleotide(s) encoding a monoclonal antibody can be modified, for example, by using recombinant DNA technology to generate alternative antibodies or alternative bispecific agents. In some embodiments, the constant domains of the light and heavy chains of, for example, a mouse monoclonal antibody can be substituted for those regions of, for example, a human antibody to generate a chimeric antibody, or for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, etc. of a monoclonal antibody.

In some embodiments, the binding agent is a humanized antibody. Typically, humanized antibodies are human immunoglobulins in which residues from the CDRs are replaced by residues from a CDR of a non-human species (e.g., mouse, rat, rabbit, hamster, etc.) that have the desired specificity, affinity, and/or binding capability using methods known to one skilled in the art. In some embodiments, the Fv framework region residues of a human immunoglobulin are replaced with the corresponding residues in an antibody from a non-human species that has the desired specificity, affinity, and/or binding capability. In some embodiments, a humanized antibody can be further modified by the substitution of additional residues either in the Fv framework region and/or within the replaced non-human residues to refine and optimize antibody specificity, affinity, and/or capability. In general, a humanized antibody will comprise substantially all of at least one, and typically two or three, variable domain regions containing all, or substantially all, of the CDRs that correspond to the non-human immunoglobulin whereas all, or substantially all, of the framework regions are those of a human immunoglobulin consensus sequence. In some embodiments, a humanized antibody can also comprise at least a portion of an immunoglobulin constant region or domain (Fc), typically that of a human immunoglobulin. In certain embodiments, such humanized antibodies are used therapeutically because they may reduce antigenicity and HAMA (human anti-mouse antibody) responses when administered to a human subject. One skilled in the art would be able to obtain a functional humanized antibody with reduced immunogenicity following known techniques (see e.g., U.S. Pat. Nos. 5,225,539; 5,585,089; 5,693,761; and 5,693,762).

In certain embodiments, the binding agent is a human antibody. Human antibodies can be directly prepared using various techniques known in the art. In some embodiments, human antibodies may be generated from immortalized human B lymphocytes immunized in vitro or from lymphocytes isolated from an immunized individual. In either case, cells that produce an antibody directed against a target antigen can be generated and isolated (see, e.g., Cole et al., 1985, Monoclonal Antibodies and Cancer Therapy, Alan R. Liss, p. 77; Boemer et al., 1991, J. Immunol., 147:86-95; and U.S. Pat. Nos. 5,750,373; 5,567,610; and 5,229,275). In some embodiments, the human antibody can be selected from a phage library, where that phage library expresses human antibodies (Vaughan et al., 1996, Nature Biotechnology, 14:309-314; Sheets et al., 1998, PNAS, 95:6157-6162; Hoogenboom and Winter, 1991, J. Mol. Biol., 227:381; Marks et al., 1991, J. Mol. Biol., 222:581). Alternatively, phage display technology can be used to produce human antibodies and antibody fragments in vitro, from immunoglobulin variable domain gene repertoires from unimmunized donors. Techniques for the generation and use of antibody phage libraries are also described in U.S. Pat. Nos. 5,969,108; 6,172,197; 5,885,793; 6,521,404; 6,544,731; 6,555,313; 6,582,915; 6,593,081; 6,300,064; 6,653,068; 6,706,484; and 7,264,963; and Rothe et al., 2008, J. Mol. Bio., 376:1182-1200. Once antibodies are identified, affinity maturation strategies known in the art, including but not limited to, chain shuffling (Marks et al., 1992, Bio/Technology, 10:779-783) and site-directed mutagenesis, may be employed to generate high affinity human antibodies.

In some embodiments, human antibodies can be made in transgenic mice that contain human immunoglobulin loci. Upon immunization these mice are capable of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

This invention also encompasses bispecific agents and bispecific antibodies. Bispecific agents are capable of specifically recognizing and binding at least two different targets or epitopes. The different targets can either be within the same molecule (e.g., two targets on a single protein) or on different molecules (e.g., one target on a protein and a second target on a second protein). In some embodiments, a bispecific agent or bispecific antibody has enhanced potency as compared to an individual agent or antibody or to a mixture of two agents. In some embodiments, a bispecific agent or bispecific antibody has reduced toxicity as compared to an individual agent or to a combination of more than one agent. It is known to those of skill in the art that any binding agent may have unique pharmacokinetics (PK) (e.g., circulating half-life). In some embodiments, a bispecific agent or bispecific antibody has the ability to synchronize the PK of two active binding agents wherein the two individual binding agents have different PK profiles. In some embodiments, a bispecific agent or bispecific antibody has the ability to concentrate the actions of two binding agents in a common area (e.g., a tumor and/or tumor environment). In some embodiments, a bispecific agent or bispecific antibody has the ability to concentrate the actions of two binding agents to a common target (e.g., a tumor or a tumor cell). In some embodiments, a bispecific agent or bispecific antibody has the ability to target the actions of two binding agents to more than one biological pathway or function.

In certain embodiments, a bispecific antibody specifically binds MET and a second target. In certain embodiments, a bispecific antibody specifically binds MET and one or more components of the WNT pathway. In some embodiments, a bispecific antibody specifically binds human MET and one or more human WNT proteins. In some embodiments, a bispecific antibody specifically binds human MET and one or more human FZD proteins. In some embodiments, the bispecific antibody is a monoclonal antibody. In some embodiments, the bispecific antibody is a humanized antibody. In some embodiments, the bispecific antibody is a human antibody. In some embodiments, the bispecific antibody is a chimeric antibody. In some embodiments, the bispecific antibody reduces cancer stem cell number or frequency. In some embodiments, the bispecific antibody has decreased toxicity and/or side effects. In some embodiments, the bispecific antibody has decreased toxicity and/or side effects as compared to a mixture of the two individual antibodies or the antibodies as single agents. In some embodiments, the bispecific antibody has an increased therapeutic index. In some embodiments, the bispecific antibody has an increased therapeutic index as compared to a mixture of the two individual antibodies or the antibodies as single agents.

In some embodiments, a bispecific antibody can specifically recognize and bind human MET as well as a second antigen target, such as an effector molecule on a leukocyte (e.g., CD2, CD3, CD28, CD80, or CD86) or a Fc receptor (e.g., CD64, CD32, or CD16) so as to focus cellular defense mechanisms to the cell expressing MET. In some embodiments, a bispecific antibody can be used to direct cytotoxic agents to cells which express a particular target antigen. These antibodies possess an antigen-binding site (e.g., to human MET) and a second site which binds a cytotoxic agent or a radionuclide chelator, such as EOTUBE, DPTA, DOTA, or TETA.

Techniques for making bispecific antibodies are known by those skilled in the art, see for example, Millstein et al., 1983, Nature, 305:537-539; Brennan et al., 1985, Science, 229:81; Suresh et al., 1986, Methods in Enzymol., 121:120; Traunecker et al., 1991, EMBO J., 10:3655-3659; Shalaby et al., 1992, J. Exp. Med., 175:217-225; Kostelny et al., 1992, J. Immunol., 148:1547-1553; Gruber et al., 1994, J. Immunol., 152:5368; U.S. Pat. No. 5,731,168; International Publication No. WO 2009/089004; and U.S. Patent Publication No. 2011/0123532. In some embodiments, the bispecific antibodies comprise heavy chain constant regions with modifications in the amino acids which are part of the interface between the two heavy chains. In some embodiments, the bispecific antibodies can be generated using a “knobs-into-holes” strategy (see, e.g., U.S. Pat. No. 5,731,168; Ridgway et. al., 1996, Prot. Engin., 9:617-621). At times the “knobs” and “holes” terminology is replaced with the terms “protuberances” and “cavities”. In some embodiments, the bispecific antibodies may comprise variant hinge regions incapable of forming disulfide linkages between the heavy chains (see, e.g., WO 2006/028936). In some embodiments, the modifications may comprise changes in amino acids that result in altered electrostatic interactions. In some embodiments, the modifications may comprise changes in amino acids that result in altered hydrophobic/hydrophilic interactions (see, e.g., U.S. Patent Application No. 2011/0123532).

Bispecific antibodies can be intact antibodies or antibody fragments comprising antigen-binding sites. Antibodies with more than two valencies are also contemplated. For example, trispecific antibodies can be prepared (Tuft et al., 1991, J. Immunol., 147:60). Thus, in certain embodiments the antibodies to MET and/or one or more components of the WNT pathway are multispecific.

In certain embodiments, the antibodies (or other polypeptides) described herein may be monospecific. In certain embodiments, each of the one or more antigen-binding sites that an antibody contains is capable of binding (or binds) a homologous epitope on different proteins.

In certain embodiments, the binding agent comprises an antibody fragment. Antibody fragments may have different functions or capabilities than intact antibodies; for example, antibody fragments can have increased tumor penetration. Various techniques are known for the production of antibody fragments including, but not limited to, proteolytic digestion of intact antibodies. In some embodiments, antibody fragments include a F(ab′)2 fragment produced by pepsin digestion of an antibody molecule. In some embodiments, antibody fragments include a Fab fragment generated by reducing the disulfide bridges of an F(ab′)2 fragment. In other embodiments, antibody fragments include a Fab fragment generated by the treatment of the antibody molecule with papain and a reducing agent. In certain embodiments, antibody fragments are produced using recombinant techniques. In some embodiments, antibody fragments include Fv or single chain Fv (scFv) fragments. Fab, Fv, and scFv antibody fragments can be expressed in and secreted from E. coli or other host cells, allowing for the production of large amounts of these fragments. In some embodiments, antibody fragments are isolated from antibody phage libraries as discussed herein. For example, methods can be used for the construction of Fab expression libraries (Huse et al 1989, Science, 246:1275-1281) to allow rapid and effective identification of monoclonal Fab fragments with the desired specificity for MET and/or one or more components of the WNT pathway or derivatives, fragments, analogs or homologs thereof. In some embodiments, antibody fragments are linear antibody fragments. In certain embodiments, antibody fragments are monospecific or bispecific. In certain embodiments, the binding agent is a scFv. Various techniques can be used for the production of single-chain antibodies specific to MET or one or more components of the WNT pathway.

It can further be desirable, especially in the case of antibody fragments, to modify an antibody in order to alter (e.g., increase or decrease) its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

Heteroconjugate antibodies are also within the scope of the present invention. Heteroconjugate antibodies are composed of two covalently joined antibodies. Such antibodies have, for example, been proposed to target immune cells to unwanted cells (see, e.g., U.S. Pat. No. 4,676,980). It is also contemplated that the heteroconjugate antibodies can be prepared in vitro using known methods in synthetic protein chemistry, including those involving cross-linking agents. For example, immunotoxins can be constructed using a disulfide exchange reaction or by forming a thioether bond. Examples of suitable reagents for this purpose include iminothiolate and methyl-4-mercaptobutyrimidate.

For the purposes of the present invention, it should be appreciated that modified agents can comprise any type of region that provides for the association of the agent with the target (i.e., human MET or a human WNT protein). In some embodiments, the region is a variable region that may comprise or be derived from any type of mammal that can be induced to mount a humoral response and generate immunoglobulins against the desired antigen. As such, a variable region of modified antibodies can be, for example, of human, murine, non-human primate (e.g. cynomolgus monkeys, macaques, etc.) or rabbit origin. In some embodiments, both a variable and a constant region of a modified immunoglobulin are human. In other embodiments, variable regions of compatible antibodies (usually derived from a non-human source) can be engineered or specifically tailored to improve the binding properties or reduce the immunogenicity of the molecule. In this respect, variable regions useful in the present invention can be humanized or otherwise altered through the inclusion of imported amino acid sequences.

In certain embodiments, variable domains in both the heavy and light chains are altered by at least partial replacement of one or more CDRs and, if necessary, by partial framework region replacement and sequence modification and/or alteration. Although the CDRs may be derived from an antibody of the same class or even subclass as the antibody from which the framework regions are derived, it is envisaged that the CDRs may be derived from an antibody of different class and often from an antibody from a different species. It may not be necessary to replace all of the CDRs with all of the CDRs from the donor variable region to transfer the antigen binding capacity of one variable domain to another. Rather, it may only be necessary to transfer those residues that are required to maintain the activity of the antigen-binding site.

Alterations to a variable region notwithstanding, those skilled in the art will appreciate that the modified antibodies of this invention will comprise antibodies (e.g., full-length antibodies or immunoreactive fragments thereof) or bispecific agents in which at least a fraction of one or more of the constant region domains has been deleted or otherwise altered so as to provide desired biochemical characteristics such as increased tumor localization or increased serum half-life when compared with an antibody of approximately the same immunogenicity comprising a native or unaltered constant region. In some embodiments, the constant region of the modified antibodies will comprise a human constant region. Modifications to the constant region compatible with this invention comprise additions, deletions or substitutions of one or more amino acids in one or more domains. The modified antibodies and/or bispecific agents disclosed herein may comprise alterations or modifications to one or more of the three heavy chain constant domains (CH1, CH2 or CH3) and/or to the light chain constant domain (CL). In some embodiments, one or more domains are partially or entirely deleted from the constant regions of the modified antibodies. In some embodiments, the modified antibodies will comprise domain-deleted constructs or variants wherein the entire CH2 domain has been removed (ΔCH2 constructs). In some embodiments, the omitted constant region domain is replaced by a short amino acid spacer (e.g., 10 amino acid residues) that provides some of the molecular flexibility typically imparted by the absent constant region.

In some embodiments, the modified antibodies or bispecific agents are engineered to fuse the CH3 domain directly to the hinge region of the antibody. In other embodiments, a peptide spacer is inserted between the hinge region and the modified CH2 and/or CH3 domains. For example, constructs may be expressed wherein the CH2 domain has been deleted and the remaining CH3 domain (modified or unmodified) is joined to the hinge region with a 5-20 amino acid spacer. Such a spacer may be added to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, in certain embodiments, any spacer added to the construct will be relatively non-immunogenic so as to maintain the desired biological qualities of the modified antibodies.

In some embodiments, the modified antibodies or bispecific agents may have only a partial deletion of a constant domain or substitution of a few or even a single amino acid. For example, the mutation of a single amino acid in selected areas of the CH2 domain may be enough to substantially reduce Fc binding and thereby increase cancer cell localization and/or tumor penetration. Similarly, it may be desirable to simply delete the part of one or more constant region domains that control a specific effector function (e.g. complement C1q binding) to be modulated. Such partial deletions of the constant regions may improve selected characteristics of the antibody (serum half-life) while leaving other desirable functions associated with the subject constant region domain intact. Moreover, as alluded to above, the constant regions of the disclosed antibodies and/or bispecific agents may be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified antibody. In certain embodiments, the modified antibodies and/or bispecific agents comprise the addition of one or more amino acids to the constant region to enhance desirable characteristics such as decreasing or increasing effector function or provide for more cytotoxin or carbohydrate attachment sites.

It is known in the art that the constant region mediates several effector functions. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region of an antibody or a Fc-fusion proteins can bind a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell cytotoxicity or ADCC), release of inflammatory mediators, placental transfer, and control of immunoglobulin production.

In certain embodiments, the modified antibodies and/or bispecific agents provide for altered effector functions that, in turn, affect the biological profile of the administered antibody. For example, in some embodiments, the deletion or inactivation (through point mutations or other means) of a constant region domain may reduce Fc receptor binding of the circulating modified antibody thereby increasing cancer cell localization and/or tumor penetration. In other embodiments, the constant region modifications increase the serum half-life of the antibody and/or bispecific agent. In other embodiments, the constant region modifications reduce the serum half-life of the antibody and/or bispecific agent. In some embodiments, the constant region is modified to eliminate disulfide linkages or oligosaccharide moieties. Modifications to the constant region in accordance with this invention may easily be made using well known biochemical or molecular engineering techniques known to those of skill in the art.

In certain embodiments, an antibody and/or bispecific agent does not have one or more effector functions. For instance, in some embodiments, the antibody or bispecific agent has no ADCC activity, and/or no complement-dependent cytotoxicity (CDC) activity. In certain embodiments, the antibody and/or bispecific agent does not bind an Fc receptor, and/or complement factors. In certain embodiments, the antibody and/or bispecific agent has no effector function.

The present invention further embraces variants and equivalents which are substantially homologous to the chimeric, humanized, and human antibodies, or antibody fragments thereof, or bispecific agents, described herein. These can contain, for example, conservative substitution mutations, i.e. the substitution of one or more amino acids by similar amino acids. For example, conservative substitution refers to the substitution of an amino acid with another amino acid within the same general class such as, for example, one acidic amino acid with another acidic amino acid, one basic amino acid with another basic amino acid or one neutral amino acid by another neutral amino acid. What is intended by a conservative amino acid substitution is well known in the art and described herein.

Thus, the present invention provides methods for producing an antibody or bispecific agent that binds MET and/or one or more components of the WNT pathway, including bispecific agents that specifically bind both MET and one or more WNT proteins. In some embodiments, the method for producing an antibody that binds MET or one or more components of the WNT pathway comprises using hybridoma techniques. In some embodiments, the method of generating an agent that binds MET or one or more components of the WNT pathway or a bispecific agent that binds MET and one or more components of the WNT pathway comprises screening a human phage display library. In some embodiments, the method of generating an agent that binds MET or one or more components of the WNT pathway or a bispecific agent that binds MET and one or more components of the WNT pathway comprises screening a mammalian cell display library. The present invention further provides methods of identifying an agent that binds MET and/or one or more components of the WNT pathway. In some embodiments, the agent is identified by FACS screening for binding to MET or a fragment thereof. In some embodiments, the agent is identified by FACS screening for binding to one or more components of the WNT pathway or a fragment thereof. In some embodiments, the agent is identified by FACS screening for binding to both MET and one or more components of the WNT pathway or a fragment thereof. In some embodiments, the agent is identified by screening using ELISA for binding to MET. In some embodiments, the agent is identified by screening using ELISA for binding to one or more components of the WNT pathway. In some embodiments, the agent is identified by screening using ELISA for binding to MET and one or more components of the WNT pathway. In some embodiments, the agent is identified by FACS screening for blocking of binding of human MET to human hepatocyte growth factor. In some embodiments, the agent is identified by FACS screening for blocking of binding of one or more WNT proteins to a human FZD protein. In some embodiments, the agent is identified by screening for inhibition or blocking of WNT pathway signaling. In some embodiments, the agent is identified by screening for inhibition or blocking of MET activity.

In certain embodiments, the antibodies and/or bispecific agents described herein are isolated. In certain embodiments, the antibodies and/or bispecific agents described herein are substantially pure.

In some embodiments of the present invention, the MET-binding agents are polypeptides. The polypeptides can be recombinant polypeptides, natural polypeptides, or synthetic polypeptides comprising an antibody, or fragment thereof, that bind MET and/or one or more components of the WNT pathway. In some embodiments, the polypeptides are multimeric. In some embodiments, the polypeptides are dimeric. The polypeptides can be recombinant polypeptides, natural polypeptides, or synthetic polypeptides comprising a soluble receptor, or fragment thereof, that bind one or more components of the WNT pathway. It will be recognized in the art that some amino acid sequences of the binding agents described herein can be varied without significant effect on the structure or function of the protein. Thus, the invention further includes variations of the polypeptides which show substantial activity or which include regions of an antibody, or fragment thereof, against human MET and/or one or more components of the WNT pathway. In some embodiments, amino acid sequence variations of MET-binding polypeptides include deletions, insertions, inversions, repeats, and/or other types of substitutions.

In some embodiments, the polypeptides described herein are isolated. In some embodiments, the polypeptides described herein are substantially pure.

The polypeptides, analogs and variants thereof, can be further modified to contain additional chemical moieties not normally part of the polypeptide. The derivatized moieties can improve or otherwise modulate the solubility, the biological half-life, and/or absorption of the polypeptide. The moieties can also reduce or eliminate undesirable side effects of the polypeptides and variants. An overview for chemical moieties can be found in Remington: The Science and Practice of Pharmacy, 22^{st} Edition, 2012, Pharmaceutical Press, London.

The polypeptides described herein can be produced by any suitable method known in the art. Such methods range from direct protein synthesis methods to constructing a DNA sequence encoding polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g., Zoeller et al., 1984, PNAS, 81:5662-5066 and U.S. Pat. No. 4,588,585.

In some embodiments, a DNA sequence encoding a polypeptide of interest may be constructed by chemical synthesis using an oligonucleotide synthesizer. Oligonucleotides can be designed based on the amino acid sequence of the desired polypeptide and selecting those codons that are favored in the host cell in which the recombinant polypeptide of interest will be produced. Standard methods can be applied to synthesize a polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis, or another method), the polynucleotide sequences encoding a particular polypeptide of interest can be inserted into an expression vector and operatively linked to an expression control sequence appropriate for expression of the protein in a desired host. Proper assembly can be confirmed by nucleotide sequencing, restriction enzyme mapping, and/or expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

In certain embodiments, recombinant expression vectors are used to amplify and express DNA encoding antibodies or fragments thereof or bispecific agents that bind human MET and/or one or more components of the WNT pathway. For example, recombinant expression vectors can be replicable DNA constructs which have synthetic or cDNA-derived DNA fragments encoding a polypeptide chain of a MET-binding agent, such as an anti-MET antibody or bispecific agent comprising an anti-MET antibody and a FZD soluble receptor, or fragment thereof, operatively linked to suitable transcriptional and/or translational regulatory elements derived from mammalian, microbial, viral, or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can additionally be incorporated. DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In other embodiments, in situations where recombinant protein is expressed without a leader or transport sequence, it can include an N-terminal methionine residue. This residue can optionally be subsequently cleaved from the expressed recombinant protein to provide a final product.

The choice of an expression control sequence and an expression vector depends upon the choice of host. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9, and their derivatives, and wider host range plasmids, such as M13 and other filamentous single-stranded DNA phages.

The binding agents (e.g., polypeptides) of the present invention can be expressed from one or more vectors. For example, in some embodiments, a heavy chain polypeptide is expressed by one vector and a light chain polypeptide is expressed by a second vector. In some embodiments, a heavy chain polypeptide and a light chain polypeptide are expressed by one vector. In some embodiments, a heavy chain polypeptide is expressed by one vector, a light chain polypeptide is expressed by a second vector and a polypeptide comprising a soluble receptor is expressed by a third vector. In some embodiments, a heavy chain polypeptide and a light chain polypeptide are expressed by one vector and a polypeptide comprising a soluble receptor is expressed by a second vector. In some embodiments, three polypeptides are expressed from one vector. Thus, in some embodiments, a heavy chain polypeptide, a light chain polypeptide, and a polypeptide comprising a soluble receptor are expressed by a single vector.

Suitable host cells for expression of a MET-binding polypeptide or agent (or a MET, WNT, or FZD protein to use as an antigen) include prokaryotes, yeast cells, insect cells, or higher eukaryotic cells under the control of appropriate promoters. Prokaryotes include gram-negative or gram-positive organisms, for example E. coli or Bacillus. Higher eukaryotic cells include established cell lines of mammalian origin as described below. Cell-free translation systems may also be employed. Appropriate cloning and expression vectors for use with bacterial, fungal, yeast, and mammalian cellular hosts are described in Pouwels et al., 1985, Cloning Vectors: A Laboratory Manual, Elsevier, New York, N.Y. Additional information regarding methods of protein production, including antibody production, can be found, e.g., in U.S. Patent Publication No. 2008/0187954; U.S. Pat. Nos. 6,413,746; 6,660,501; and International Patent Publication No. WO 04/009823.

Various mammalian cell culture systems may be used to express recombinant polypeptides. Expression of recombinant proteins in mammalian cells may be desirable because these proteins are generally correctly folded, appropriately modified, and biologically functional. Examples of suitable mammalian host cell lines include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants of these cell lines. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art (see, e.g., Luckow and Summers, 1988, Bio/Technology. 6:47).

Thus, the present invention provides cells comprising the binding agents described herein. In some embodiments, the cells produce the binding agents described herein. In certain embodiments, the cells produce an antibody. In some embodiments, the cells produce a MET-binding agent, such as an anti-MET antibody. In some embodiments, the cells produce a bispecific agent that binds MET. In some embodiments, the cells produce a bispecific agent that binds MET and one or more components of the WNT pathway. In some embodiments, the cells produce a bispecific agent that binds MET and one or more FZD proteins. In some embodiments, the cells produce a bispecific agent that binds MET and one or more WNT proteins. In certain embodiments, the cells produce antibody 73R009. In certain embodiments, the cells produce a humanized version of antibody 73R009. In certain embodiments, the cells produce antibody 73R010 (73R009 H12L7). In certain embodiments, the cells produce a bispecific agent which comprises an antigen-binding site from antibody 73R009. In certain embodiments, the cells produce a bispecific agent which comprises an antigen-binding site from a humanized version of antibody 73R009. In certain embodiments, the cells produce a bispecific agent which comprises an antigen-binding site from antibody 73R010 (73R009 H12L7). In certain embodiments, the cells produce a bispecific agent which comprises an antigen-binding site from antibody 73R009 and a FZD Fri domain. In certain embodiments, the cells produce a bispecific agent which comprises an antigen-binding site from a humanized version of antibody 73R009 and a FZD Fri domain. In certain embodiments, the cells produce a bispecific agent which comprises an antigen-binding site from antibody 73R010 (73R009 H12L7) and a FZD Fri domain. In certain embodiments, the cells produce a bispecific agent which comprises an antigen-binding site from antibody 73R009 and a FZD8 Fri domain. In certain embodiments, the cells produce a bispecific agent which comprises an antigen-binding site from a humanized version of antibody 73R009 and a FZD8 Fri domain. In certain embodiments, the cells produce a bispecific agent which comprises an antigen-binding site from antibody 73R010 (73R009 H12L7) and a FZD8 Fri domain. In certain embodiments, the cells produce the bispecific agent 315B06. In certain embodiments, the cells produce the bispecific agent 315B07. In certain embodiments, the cells produce the bispecific agent 315B09.

The proteins, including bispecific agents, produced by a transformed host can be purified according to any suitable method. Standard methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence, and glutathione-S-transferase can be attached to the protein to allow easy purification by passage over an appropriate affinity column. Affinity chromatography used for purifying immunoglobulins can include Protein A, Protein G, and Protein L chromatography. Isolated proteins can be physically characterized using such techniques as proteolysis, size exclusion chromatography (SEC), mass spectrometry (MS), nuclear magnetic resonance (NMR), isoelectric focusing (IEF), high performance liquid chromatography (HPLC), and x-ray crystallography. The purity of isolated proteins can be determined using techniques known to those of skill in the art, including but not limited to, SDS-PAGE, SEC, capillary gel electrophoresis, IEF, and capillary isoelectric focusing (cIEF).

In some embodiments, supernatants from expression systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. In some embodiments, an anion exchange resin can be employed, for example, a matrix or substrate having pendant diethylaminoethyl (DEAE) groups. The matrices can be acrylamide, agarose, dextran, cellulose, or other types commonly employed in protein purification. In some embodiments, a cation exchange step can be employed. Suitable cation exchangers include various insoluble matrices comprising sulfopropyl or carboxymethyl groups. In some embodiments, a hydroxyapatite media can be employed, including but not limited to, ceramic hydroxyapatite (CHT). In certain embodiments, one or more reverse-phase HPLC steps employing hydrophobic RP-HPLC media, e.g., silica gel having pendant methyl or other aliphatic groups, can be employed to further purify a recombinant protein (e.g., a MET-binding agent). Some or all of the foregoing purification steps, in various combinations, can be employed to provide a homogeneous recombinant protein.

In some embodiments, heterodimeric proteins such as bispecific agents described herein are purified according the any of the methods described herein. In some embodiments, bispecific agents are isolated and/or purified using at least one chromatography step. In some embodiments, the at least one chromatography step comprises affinity chromatography. In some embodiments, the at least one chromatography step further comprises anion exchange chromatography. In some embodiments, the isolated and/or purified antibody product comprises at least 90% heterodimeric agent. In some embodiments, the isolated and/or purified product comprises at least 95%, 96%, 97%, 98% or 99% heterodimeric agent. In some embodiments, the isolated and/or purified product comprises about 100% heterodimeric agent.

In some embodiments, recombinant protein produced in bacterial culture can be isolated, for example, by initial extraction from cell pellets, followed by one or more concentration, salting-out, aqueous ion exchange, or size exclusion chromatography steps. HPLC can be employed for final purification steps. Microbial cells employed in expression of a recombinant protein can be disrupted by any convenient method, including freeze-thaw cycling, sonication, mechanical disruption, or use of cell lysing agents.

Methods known in the art for purifying antibodies and other proteins also include, for example, those described in U.S. Patent Publication Nos. 2008/0312425; 2008/0177048, and 2009/0187005.

In certain embodiments, a MET-binding agent is a polypeptide that is not an antibody. A variety of methods for identifying and producing non-antibody polypeptides that bind with high affinity to a protein target are known in the art. See, e.g., Skerra, 2007, Curr. Opin. Biotechnol., 18:295-304; Hosse et al., 2006, Protein Science, 15:14-27; Gill et al., 2006, Curr. Opin. Biotechnol., 17:653-658; Nygren, 2008, FEBS J., 275:2668-76; and Skerra, 2008, FEBS J., 275:2677-83, In certain embodiments, phage or mammalian cell display technology may be used to produce and/or identify a MET-binding polypeptide that is not an antibody. In certain embodiments, the polypeptide comprises a protein scaffold of a type selected from the group consisting of protein A, protein G, a lipocalin, fibronectin domain, an ankyrin consensus repeat domain, and thioredoxin.

In certain embodiments, a MET-binding agent can be used in any one of a number of conjugated (i.e. an immunoconjugate or radioconjugate) or non-conjugated forms. In certain embodiments, the agent can be used in a non-conjugated form to harness the subject's natural defense mechanisms including complement-dependent cytotoxicity and antibody-dependent cellular toxicity to eliminate malignant or cancer cells.

In some embodiments, a MET-binding agent (e.g., an antibody or bispecific agent) is conjugated to a cytotoxic agent. In some embodiments, the cytotoxic agent is a chemotherapeutic agent including, but not limited to, methotrexate, adriamycin, doxorubicin, melphalan, mitomycin C, chlorambucil, daunorubicin or other intercalating agents. In some embodiments, the cytotoxic agent is an enzymatically active toxin of bacterial, fungal, plant, or animal origin, or fragments thereof, including, but not limited to, diphtheria A chain, non-binding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, the cytotoxic agent is a radioisotope to produce a radioconjugate or a radioconjugated antibody. A variety of radionuclides are available for the production of radioconjugated antibodies including, but not limited to, ⁹⁰Y, ¹²⁵I, ¹³¹I, ¹²³I, ¹¹¹In, ¹³¹In, ¹⁰⁵Rh, ¹⁵³Sm, ⁶⁷Cu, ⁶⁷Ga, ¹⁶⁶Ho, ¹⁷⁷Lu, ¹⁸⁶Re, ¹⁸⁸Re and ²¹²Bi. In some embodiments, conjugates of a binding agent described herein and one or more small molecule toxins, such as calicheamicins, maytansinoids, trichothecenes, and CC1065, and the derivatives of these toxins that have toxin activity, can also be used. In some embodiments, a binding agent described herein is conjugated to a maytansinoid. In some embodiments, a binding agent described herein is conjugated to mertansine (DM1). Conjugates of a binding agent described herein and a cytotoxic agent can be made using a variety of bifunctional protein-coupling agents including, but not limited to, N-succinimidyl-3-(2-pyridyidithiol) propionate (SPDP), iminothiolane (IT), bifunctional derivatives of imidoesters (such as dimethyl adipimidate HCl), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

### III. POLYNUCLEOTIDES

In certain embodiments, the invention encompasses polynucleotides comprising polynucleotides that encode a polypeptide (or a fragment of a polypeptide) that specifically binds MET, one or more components of the WNT pathway, or both MET and one or more components of the WNT pathway. The term “polynucleotides that encode a polypeptide” encompasses a polynucleotide which includes only coding sequences for the polypeptide, as well as a polynucleotide which includes additional coding and/or non-coding sequences. For example, in some embodiments, the invention provides a polynucleotide comprising a polynucleotide sequence that encodes an antibody to human MET or encodes a fragment of such an antibody (e.g., a fragment comprising the antigen-binding site). In some embodiments, the invention provides a polynucleotide comprising a polynucleotide sequence that encodes a polypeptide that binds one or more human FZD proteins or encodes a fragment of such a polypeptide (e.g., a fragment comprising the binding site). In some embodiments, the invention provides a polynucleotide comprising a polynucleotide sequence that encodes a polypeptide that binds one or more human WNT proteins or encodes a fragment of such a polypeptide (e.g., a fragment comprising the binding site). The polynucleotides of the invention can be in the form of RNA or in the form of DNA. DNA includes cDNA, genomic DNA, and synthetic DNA; and can be double-stranded or single-stranded, and if single-stranded can be the coding strand or non-coding (anti-sense) strand.

In certain embodiments, the polynucleotide comprises a polynucleotide encoding a polypeptide comprising a sequence selected from the group consisting of: SEQ ID NO:12, SEQ ID NO:13, SEQ ID NO:14, SEQ ID NO:55, SEQ ID NO:56, SEQ ID NO:87, SEQ ID NO:88, SEQ ID NO:96, SEQ ID NO:97, SEQ ID NO:98, SEQ ID NO:99, SEQ ID NO:100, SEQ ID NO:101, SEQ ID NO:108, SEQ ID NO:110, SEQ ID NO:111, and SEQ ID NO:114. In some embodiments, the polynucleotide comprises a polynucleotide sequence selected from the group consisting of: SEQ ID NO:15, SEQ ID NO:16, SEQ ID NO:17, SEQ ID NO:18, SEQ ID NO:19, SEQ ID NO:20, SEQ ID NO:89, SEQ ID NO:90, SEQ ID NO:102, SEQ ID NO:103, SEQ ID NO:104, SEQ ID NO:105, SEQ ID NO:106, SEQ ID NO:107, and SEQ ID NO:109. In some embodiments, the polynucleotide comprises the complement of a polynucleotide sequence selected from the group consisting of: SEQ ID NO:15, SEQ ID NO:16, SEQ ID NO:17, SEQ ID NO:18, SEQ ID NO:19, SEQ ID NO:20, SEQ ID NO:89, SEQ ID NO:90, SEQ ID NO:102, SEQ ID NO:103, SEQ ID NO:104, SEQ ID NO:105, SEQ ID NO:106, SEQ ID NO:107, and SEQ ID NO:109.

In certain embodiments, the polynucleotide comprises a polynucleotide having a nucleotide sequence at least about 80% identical, at least about 85% identical, at least about 90% identical, at least about 95% identical, and in some embodiments, at least about 96%, 97%, 98% or 99% identical to a polynucleotide comprising a sequence selected from the group consisting of: SEQ ID NO:15, SEQ ID NO:16, SEQ ID NO:17, SEQ ID NO:18, SEQ ID NO:19, SEQ ID NO:20, SEQ ID NO:89, SEQ ID NO:90, SEQ ID NO:102, SEQ ID NO:103, SEQ ID NO:104, SEQ ID NO:105, SEQ ID NO:106, SEQ ID NO:107, and SEQ ID NO:109. Also provided is a polynucleotide that comprises a polynucleotide that hybridizes to SEQ ID NO:15, SEQ ID NO:16, SEQ ID NO:17, SEQ ID NO:18, SEQ ID NO:19, SEQ ID NO:20, SEQ ID NO:89, SEQ ID NO:90, SEQ ID NO:102, SEQ ID NO:103, SEQ ID NO:104, SEQ ID NO:105, SEQ ID NO:106, SEQ ID NO:107, or SEQ ID NO:109. Also provided is a polynucleotide that comprises a polynucleotide that hybridizes to a complement of SEQ ID NO:15, SEQ ID NO:16, SEQ ID NO:17, SEQ ID NO:18, SEQ ID NO:19, SEQ ID NO:20, SEQ ID NO:89, SEQ ID NO:90, SEQ ID NO:102, SEQ ID NO:103, SEQ ID NO:104, SEQ ID NO:105, SEQ ID NO:106, SEQ ID NO:107, or SEQ ID NO:109, In certain embodiments, the hybridization is under conditions of high stringency.

The binding agents of the present invention can be encoded by one or more polynucleotides. For example, in some embodiments, a heavy chain polypeptide is encoded by one polynucleotide and a light chain polypeptide is encoded by a second polynucleotide. In some embodiments, a heavy chain polypeptide and a light chain polypeptide are encoded by one polynucleotide. In some embodiments, a heavy chain polypeptide is encoded by one polynucleotide, a light chain polypeptide is encoded by a second polynucleotide and a polypeptide comprising a soluble receptor is encoded by a third polynucleotide. In some embodiments, a heavy chain polypeptide and a light chain polypeptide are encoded by one polynucleotide and a polypeptide comprising a soluble receptor is encoded by a second polynucleotide. In some embodiments, three polypeptides are encoded from one polynucleotide. Thus, in some embodiments, a heavy chain polypeptide, a light chain polypeptide, and a polypeptide comprising a soluble receptor are encoded by a single polynucleotide.

In certain embodiments, the polynucleotides comprise the coding sequence for the mature polypeptide fused in the same reading frame to a polynucleotide which aids, for example, in expression and secretion of a polypeptide from a host cell (e.g., a leader sequence which functions as a secretory sequence for controlling transport of a polypeptide from the cell). The polypeptide having a leader sequence is a pre-protein and can have the leader sequence cleaved by the host cell to form the mature form of the polypeptide. The polynucleotides can also encode for a pro-protein which is the mature protein plus additional 5′ amino acid residues. A mature protein having a pro-sequence is a pro-protein and is an inactive form of the protein. Once the pro-sequence is cleaved an active mature protein remains.

In certain embodiments, the polynucleotides comprise the coding sequence for the mature polypeptide fused in the same reading frame to a marker sequence that allows, for example, for purification of the encoded polypeptide. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. In some embodiments, the marker sequence is a FLAG tag, a peptide of sequence DYKDDDDK (SEQ ID NO:73) which can be used in conjunction with other affinity tags.

The present invention further relates to variants of the hereinabove described polynucleotides encoding, for example, fragments, analogs, and/or derivatives.

In certain embodiments, the present invention provides polynucleotides comprising polynucleotides having a nucleotide sequence at least about 80% identical, at least about 85% identical, at least about 90% identical, at least about 95% identical, and in some embodiments, at least about 96%, 97%, 98% or 99% identical to a polynucleotide encoding a polypeptide comprising a MET-binding agent (e.g., an antibody or bispecific agent), or fragment thereof, described herein.

As used herein, the phrase a polynucleotide having a nucleotide sequence at least, for example, 95% “identical” to a reference nucleotide sequence is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In some embodiments, at least one polynucleotide variant is produced (without changing the amino acid sequence of the encoded polypeptide) to increase production of a heterodimeric or heteromultimeric molecule. In some embodiments, at least one polynucleotide variant is produced (without changing the amino acid sequence of the encoded polypeptide) to increase production of a bispecific agent.

In certain embodiments, the polynucleotides are isolated. In certain embodiments, the polynucleotides are substantially pure.

Vectors and cells comprising the polynucleotides described herein are also provided. In some embodiments, an expression vector comprises a polynucleotide. In some embodiments, a host cell comprises an expression vector comprising the polynucleotide. In some embodiments, a host cell comprises a polynucleotide.

### IV. METHODS OF USE AND PHARMACEUTICAL COMPOSITIONS

The MET-binding agents (including antibodies and bispecific agents) of the invention that bind MET or MET and one or more components of the WNT pathway are useful in a variety of applications including, but not limited to, therapeutic treatment methods, such as the treatment of cancer. In certain embodiments, the agents are useful for inhibiting MET activity, inhibiting WNT pathway activity, inhibiting tumor growth, reducing tumor volume, reducing the frequency of cancer stem cells in a tumor, reducing the tumorigenicity of a tumor, inducing differentiation of tumor cells, inducing differentiation of cancer stem cells, inducing expression of differentiation markers on tumor cells, inducing expression of differentiation markers on cancer stem cells, inhibiting angiogenesis, and/or inhibiting EMT. The methods of use may be in vitro, ex vivo, or in vivo. In certain embodiments, a MET-binding agent is an antagonist of human MET. In certain embodiments, a MET-binding agent is an antagonist of one or more components of the WNT pathway. In certain embodiments, a MET-binding agent is an antagonist of both MET and one or more components of the WNT pathway.

The present invention provides methods for inhibiting growth of a tumor using the MET-binding agents described herein. In certain embodiments, the method of inhibiting growth of a tumor comprises contacting a tumor cell with a MET-binding agent (e.g., an antibody or a bispecific agent) in vitro. For example, an immortalized cell line or a cancer cell line is cultured in medium to which is added an antibody or a bispecific agent described herein to inhibit tumor cell growth. In some embodiments, tumor cells are isolated from a patient sample such as, for example, a tissue biopsy, pleural effusion, or blood sample and cultured in medium to which is added a MET-binding agent (e.g., an antibody or a bispecific agent) to inhibit tumor cell growth.

In some embodiments, the method of inhibiting growth of a tumor comprises contacting a tumor or tumor cells with a MET-binding agent (e.g., an antibody or a bispecific agent) in vivo. In certain embodiments, contacting a tumor or tumor cell with a MET-binding agent is undertaken in an animal model. For example, an antibody or bispecific agent described herein may be administered to an immunocompromised host animal (e.g., NOD/SCID mice) that has a tumor xenograft. In some embodiments, tumor cells and/or cancer stem cells are isolated from a patient sample such as, for example, a tissue biopsy, pleural effusion, or blood sample and injected into an immunocompromised host animal (e.g., NOD/SCID mice) that is then administered a binding agent to inhibit tumor cell growth. In some embodiments, the MET-binding agent is administered at the same time or shortly after introduction of tumorigenic cells into the animal to prevent tumor growth (“preventative model”). In some embodiments, the MET-binding agent is administered as a therapeutic after tumors have gown to a specified size (“therapeutic model”). In certain embodiments, the MET-binding agent is a bispecific agent described herein that specifically binds human MET and one or more components of the WNT pathway. In certain embodiments, the MET-binding agent is a bispecific agent described herein that specifically binds human MET and one or more WNT proteins.

In certain embodiments, the method of inhibiting growth of a tumor in a subject comprises administering to the subject a therapeutically effective amount of a MET-binding agent described herein. In certain embodiments, the subject is a human. In certain embodiments, the subject has a tumor or had a tumor that was removed. In certain embodiments, the tumor comprises cancer stem cells. In certain embodiments, the frequency of cancer stem cells in the tumor is reduced by administration of the MET-binding agent. The invention also provides a method of reducing the frequency of cancer stem cells in a tumor, comprising contacting the tumor with an effective amount of a MET-binding agent (e.g., an antibody or a bispecific agent) described herein. In some embodiments, a method of reducing the frequency of cancer stem cells in a tumor in a subject, comprises administering to the subject a therapeutically effective amount of a MET-binding agent described herein. In certain embodiments, the MET-binding agent is a bispecific agent described herein that specifically binds human MET and one or more components of the WNT pathway. In certain embodiments, the MET-binding agent is a bispecific agent described herein that specifically binds human MET and one or more WNT proteins.

The present invention further provides methods for inhibiting angiogenesis in a subject comprising administering a therapeutically effective amount of a MET-binding agent described herein to the subject. In some embodiments, the angiogenesis is tumor angiogenesis.

The present invention further provides methods for inhibiting epithelial-mesenchymal transition (EMT) of tumor cells comprising contacting tumor cells with an effective amount of a MET-binding agent described herein. The present invention further provides methods for inhibiting EMT of tumor cells in a subject comprising administering a therapeutically effective amount of a MET-binding, agent described herein to the subject.

The present invention further provides methods for inducing differentiation of tumor cells comprising contacting tumor cells with an effective amount of a MET-binding agent described herein. In some embodiments, the method comprises inducing differentiation of cancer stem cells by contacting tumor cells comprising cancer stem cells with an effective amount of a MET-binding agent described herein. The present invention further provides methods for inducing differentiation of tumor cells in a subject comprising administering a therapeutically effective amount of a MET-binding agent described herein to the subject. In some embodiments, the method comprises inducing differentiation of cancer stem cells by administering a therapeutically effective amount of a MET-binding agent described herein to the subject.

In some embodiments, the tumor is a solid tumor. In certain embodiments, the tumor is a tumor selected from the group consisting of colorectal tumor, colon tumor, pancreatic tumor, lung tumor, ovarian tumor, liver tumor, breast tumor, kidney tumor, prostate tumor, gastrointestinal tumor, melanoma, cervical tumor, bladder tumor, glioblastoma, and head and neck tumor. In certain embodiments, the tumor is a colorectal tumor or a colon tumor. In certain embodiments, the tumor is an ovarian tumor. In some embodiments, the tumor is a lung tumor. In certain embodiments, the tumor is a pancreatic tumor. In certain embodiments, the tumor is a breast tumor, including triple negative breast tumors. In some embodiments, the tumor is a glioblastoma.

The present invention further provides methods for treating cancer in a subject comprising administering a therapeutically effective amount of a MET-binding agent described herein to the subject. In some embodiments, the MET-binding agent binds MET, and inhibits or reduces cancer growth. In some embodiments, the MET-binding agent binds one or more components of the WNT pathway, and inhibits or reduces cancer growth. In some embodiments, the MET-binding agent is a bispecific agent that binds MET and one or more components of the WNT pathway, and inhibits or reduces cancer growth. In some embodiments, the MET-binding agent is a bispecific agent that binds MET and one or more components of the WNT pathway and provides dual inhibition of cancer-involved signaling pathways. In some embodiments, the MET-binding agent binds MET, interferes with MET/HGF interactions, and inhibits or reduces cancer growth. In some embodiments, the MET-binding agent binds MET, blocks binding of MET to HGF, and inhibits or reduces cancer growth. In some embodiments, the MET-binding agent binds MET, inhibits angiogenesis, and inhibits or reduces cancer growth. In some embodiments, the MET-binding agent binds one or more components of the WNT pathway, interferes with WNT/FZD interactions, and inhibits or reduces cancer growth. In some embodiments, the MET-binding agent binds both MET and one or more components of the WNT pathway, interferes with MET/HGF interactions and with WNT/FZD interactions, and inhibits or reduces cancer growth. In some embodiments, the MET-binding agent binds one or more WNT proteins and reduces the frequency of cancer stem cells in the cancer.

The present invention provides methods of treating cancer in a subject (e.g., a subject in need of treatment) comprising administering a therapeutically effective amount of a MET-binding agent described herein to the subject. In certain embodiments, the subject is a human. In certain embodiments, the subject has a cancerous tumor. In certain embodiments, the subject has had a tumor removed. The invention also provides a bispecific agent or antibody for use in a method of treating cancer, wherein the bispecific agent or antibody is an agent or antibody described herein. The invention also provides the use of a bispecific agent or antibody described herein for the manufacture of a medicament for the treatment of cancer.

In certain embodiments, the cancer is a cancer selected from the group consisting of colorectal cancer, pancreatic cancer, lung cancer, ovarian cancer, liver cancer, breast cancer, kidney cancer, prostate cancer, gastrointestinal cancer, melanoma, cervical cancer, bladder cancer, glioblastoma, and head and neck cancer. In certain embodiments, the cancer is ovarian cancer. In certain embodiments, the cancer is colorectal cancer or colon cancer. In certain embodiments, the cancer is pancreatic cancer. In certain embodiments, the cancer is breast cancer, including triple negative breast cancer. In certain embodiments, the cancer is prostate cancer. In certain embodiments, the cancer is lung cancer, including non-small cell lung cancer and small cell lung cancer.

In some embodiments, the subject's cancer/tumor may be refractory to certain treatment(s). As a non-limiting example, the subject's cancer (or tumor) may be chemorefractory. In some embodiments, the subject's cancer may be resistant to EGFR inhibitors.

Methods of treating a disease or disorder in a subject, wherein the disease or disorder is characterized by an increased level of stem cells and/or progenitor cells are further provided. In some embodiments, the treatment methods comprise administering a therapeutically effective amount of a MET-binding agent, polypeptide, antibody, or bispecific agent described herein to the subject.

In certain embodiments of any of the methods described herein, the MET-binding agent is a bispecific agent that specifically binds human MET and one or more components of the WNT pathway. In some embodiments, the bispecific agent comprises a first binding site that specifically binds human MET and a second binding site that specifically binds one or more components of the human WNT pathway, wherein the first binding site comprises a heavy chain CDR1 comprising ASYAWS (SEQ ID NO:1), a heavy chain CDR2 comprising YISYSGGTDYNPSLKS (SEQ ID NO:2), and a heavy chain CDR3 comprising KGAY (SEQ ID NO:3), and a light chain CDR1 comprising SASSSVSSSYLY (SEQ ID NO:4), a light chain CDR2 comprising STSNLAS (SEQ ID NO:5), and a light chain CDR3 comprising HQWSSYPYT (SEQ ID NO:6). In some embodiments, the bispecific agent comprises a first binding site that specifically binds human MET and a second binding site that specifically binds one or more components of the human WNT pathway, wherein the first antigen-binding site comprises a heavy chain CDR1 comprising GYTFTSYWLH (SEQ ID NO:78), a heavy chain CDR2 comprising GMIDPSNSDTRFNPNFKD (SEQ ID NO:79), and a heavy chain CDR3 comprising TYGSYVSPLDY (SEQ ID NO:81), SYGSYVSPLDY (SEQ ID NO:82), ATYGSYVSPLDY (SEQ ID NO:83), or XYGSYVSPLDY (SEQ ID NO:80), wherein X is not R; and a light chain CDR1 comprising KSSQSLLYTSSQKNYLA (SEQ ID NO:84), a light chain CDR2 comprising WASTRES (SEQ ID NO:85), and a light chain CDR3 comprising QQYYAYPWT (SEQ ID NO:86).

In certain embodiments of any of the methods described herein, the MET-binding agent is a bispecific agent that comprises a heavy chain variable region having at least about 80% sequence identity to SEQ ID NO:7 or SEQ ID NO:94, and a light chain variable region having at least about 80% sequence identity to SEQ ID NO:8 or SEQ ID NO:95.

In some embodiments of any of the methods described herein, the MET-binding agent is an antibody. In some embodiments, the anti-MET antibody comprises the heavy chain variable region and the light chain variable region of antibody 73R009, a humanized version of antibody 73R009, or antibody 73R010 (73R009 H12L7). In some embodiments, the anti-MET antibody is antibody 73R009. In some embodiments, the anti-MET antibody is a humanized version of antibody 73R009. In some embodiments, the anti-MET antibody is antibody 738010 (73R009 H12L7) or variants thereof. In some embodiments, the anti-MET antibody is a monovalent version of antibody 73R009 or antibody 73R010 (73R007 H12L7). In some embodiments, the anti-MET antibody is an antibody comprising a heavy chain variable region encoded by the plasmid deposited with ATCC as PTA-13609 and a light chain variable region encoded by the plasmid deposited with ATCC as PTA-13610. In some embodiments, the anti-MET antibody is an antibody comprising a heavy chain variable region encoded by the plasmid deposited with ATCC as PTA-120387 and a light chain variable region encoded by the plasmid deposited with ATCC as PTA-120388. In some embodiments, the anti-MET antibody is an antibody comprising a heavy chain variable region encoded by the plasmid deposited with ATCC as PTA-120695 and a light chain variable region encoded by the plasmid deposited with ATCC as PTA-120388. In some embodiments, the MET-binding agent is a bispecific agent comprising an antigen-binding site from antibody 73R009, an antigen-binding site from a humanized version of antibody 73R009, or an antigen-binding site from antibody 73R010 (73R009 H12L7). In some embodiments, the MET-binding agent is a bispecific agent comprising a heavy chain variable region encoded by the plasmid deposited with ATCC as PTA-13609 and a light chain variable region encoded by the plasmid deposited with ATCC as PTA-13610. In some embodiments, the MET-binding agent is a bispecific agent comprising a heavy chain variable region encoded by the plasmid deposited with ATCC as PTA-120387 and a light chain variable region encoded by the plasmid deposited with ATCC as PTA-120388. In some embodiments, the MET-binding agent is a bispecific agent comprising a heavy chain variable region encoded by the plasmid deposited with ATCC as PTA-120695 and a light chain variable region encoded by the plasmid deposited with ATCC as PTA-120388. In some embodiments, the MET-binding agent is a bispecific agent comprising a first arm comprising the heavy chain variable region and the light chain variable region of antibody 73R009, the heavy chain variable region and the light chain variable region of a humanized version of antibody 73R009, or the heavy chain variable region and the light chain variable region of antibody 73R010 (73R009 H12L7), and a second arm comprising a FZD8 Fri domain. In some embodiments, the MET-binding agent is a bispecific agent comprising a first arm comprising the heavy chain variable region and the light chain variable region of antibody 73R009, the heavy chain variable region and the light chain variable region of a humanized version of antibody 73R009, or the heavy chain variable region and the light chain variable region of antibody 73R010 (73R009 H12L7), and a second arm comprising a FZD8 Fri domain and a human Fc region. In some embodiments, the MET-binding agent is bispecific agent 315B06. In some embodiments, the MET-binding agent is bispecific agent 315B07. In some embodiments, the MET-binding agent is bispecific agent 315B09.

In some embodiments, the MET-binding agent is a bispecific agent comprising SEQ ID NO:7, SEQ ID NO:8, and SEQ ID NO:28. In some embodiments, the MET-binding agent is a bispecific agent comprising SEQ ID NO:7, SEQ ID NO:8, and SEQ ID NO:29. In some embodiments, the MET-binding agent is a bispecific agent comprising SEQ ID NO:7, SEQ ID NO:8, and SEQ ID NO:39. In some embodiments, the MET-binding agent is a bispecific agent comprising SEQ ID NO:13, SEQ ID NO:14, and SEQ ID NO:56. In some embodiments, the MET-binding agent is a bispecific agent, wherein a first arm of the bispecific agent comprises SEQ ID NO:13 and SEQ ID NO:14; and a second arm of the bispecific agent comprises SEQ ID NO:56. In some embodiments, the MET-binding agent is a bispecific agent comprising SEQ ID NO:94, SEQ ID NO:95, and SEQ ID NO:28. In some embodiments, the MET-binding agent is a bispecific agent comprising SEQ ID NO:94, SEQ ID NO:95, and SEQ ID NO:29. In some embodiments, the MET-binding agent is a bispecific agent comprising SEQ ID NO:94, SEQ ID NO:95, and SEQ ID NO:39. In some embodiments, the MET-binding agent is a bispecific agent comprising SEQ ID NO:100, SEQ ID NO:101, and SEQ ID NO:56. In some embodiments, the MET-binding agent is a bispecific agent, wherein a first arm of the bispecific agent comprises SEQ ID NO:100 and SEQ ID NO:101; and a second arm of the bispecific agent comprises SEQ ID NO:56. In some embodiments, the MET-binding agent is a bispecific agent comprising SEQ ID NO:111, SEQ ID NO:101, and SEQ ID NO:56. In some embodiments, the MET-binding agent is a bispecific agent, wherein a first arm of the bispecific agent comprises SEQ ID NO:111 and SEQ ID NO:101; and a second arm of the bispecific agent comprises SEQ ID NO:56.

In certain embodiments, the methods further comprise a step of determining the level of MET expression in the tumor or cancer. In some embodiments, the “level of MET expression” is at the protein level. In some embodiments, the “level of MET expression” is at the nucleic acid level (DNA or RNA). In some embodiments, the level of expression of MET in a tumor or cancer is compared to the level of expression of MET in a reference sample. As used herein, a “reference sample” includes but is not limited to, normal tissue, non-cancerous tissue of the same tissue type, tumor tissue of the same tissue type, and tumor tissue of a different tissue type. Thus, in some embodiments, the level of expression of MET in a tumor or cancer is compared to the level of expression of MET in normal tissue. In some embodiments, the level of expression of MET in a tumor or cancer is compared to the level of expression of MET in non-cancerous tissue of the same tissue type. In some embodiments, the level of expression of MET in a tumor or cancer is compared to the level of expression of MET in tumors or cancers of the same tissue type. In some embodiments, the level of expression of MET in a tumor or cancer is compared to the level of expression of MET in tumors or cancers of a different tissue type. In some embodiments, the level of expression of MET in a tumor or cancer is compared to a pre-determined level of MET. In some embodiments, determining the level of MET expression is done prior to treatment. In some embodiments, determining the level of MET expression is by immunohistochemistry. In some embodiments, the subject is administered a MET-binding agent described herein if the tumor or cancer has an elevated level of MET expression as compared to the expression of MET in normal tissue or non-cancerous tissue of the same tissue type. For example, in some embodiments, the subject is administered a MET-binding agent (e.g., bispecific agent 315B06, 315B07, or 315B09) if the tumor or cancer has an elevated level of MET expression as compared to the level of MET expression in a reference sample. In some embodiments, the subject is administered a MET-binding agent described herein if the tumor or cancer has an elevated level of MET expression as compared to the pre-determined level of MET.

In addition, the present invention provides methods of identifying a human subject for treatment with a MET-binding agent, comprising determining if the subject has a tumor that has an elevated level of MET expression as compared to expression of MET in a reference sample. In some embodiments, the reference sample is normal tissue or non-cancerous tissue of the same tissue type. In some embodiments, the reference sample is tumor/cancer tissue of the same tissue type. In some embodiments, the reference sample is tumor/cancer tissue of a different tissue type. In some embodiments, the level of expression of MET in a tumor or cancer is compared to a pre-determined level of MET. In some embodiments, if the tumor has an elevated level of MET expression the subject is selected for treatment with an agent that specifically binds MET. In some embodiments, if selected for treatment, the subject is administered a MET-binding agent described herein. For example, in some embodiments, the expression level of MET in a tumor is determined, if the tumor has an elevated level of MET expression as compared to the level of MET in a reference sample or a pre-determined level, the subject is selected for treatment with an agent that specifically binds MET. If selected for treatment, the subject is administered a MET-binding agent described herein. In certain embodiments, the subject has had a tumor removed. In some embodiments, the MET-binding agent is antibody 73R009 or a monovalent version thereof. In some embodiments, the MET-binding agent is a humanized version of antibody 73R009 or a monovalent version thereof. In some embodiments, the MET-binding agent is antibody 73R010 (73R009 H12L7) or a monovalent version thereof. In some embodiments, the MET-binding agent is an anti-MET/FZD-Fc bispecific agent. In some embodiments, the MET-binding agent is an anti-MET/FZD8-Fc bispecific agent. In some embodiments, the MET-binding agent is bispecific agent 315B06. In some embodiments, the MET-binding agent is bispecific agent 315B07. In some embodiments, the MET-binding agent is bispecific agent 315B09.

The present invention provides methods of selecting a human subject for treatment with a MET-binding agent, comprising determining if the subject has a tumor that has an elevated expression level of MET. In some embodiments, the methods of selecting a human subject for treatment with a MET-binding agent comprise determining if the subject has a tumor that has an elevated expression level of MET, wherein if the tumor has an elevated expression level of MET, the subject is selected for treatment with an agent that specifically binds MET. The present invention provides methods of selecting a human subject for treatment with a MET-binding agent, comprising determining if the subject has a tumor that has a high expression level of MET. In some embodiments, the methods of selecting a human subject for treatment with a MET-binding agent comprise determining if the subject has a tumor that has a high expression level of MET, wherein if the tumor has a high expression level of MET the subject is selected for treatment with an agent that specifically binds MET. In some embodiments, the “elevated” or “high” expression level is in comparison to the expression level of MET in normal tissue of the same tissue type. In some embodiments, the “elevated” or “high” expression level is in comparison to the expression level of MET in other tumors of the same tissue type. In some embodiments, the “elevated” or “high” expression level is in comparison to the expression level of MET in a reference sample. In some embodiments, the “elevated” or “high” expression level is in comparison to a pre-determined level of MET. In some embodiments, if selected for treatment, the subject is administered a MET-binding agent described herein. In certain embodiments, the subject has had a tumor removed. In some embodiments, the MET-binding agent is an anti-MET antibody. In some embodiments, the anti-MET antibody is antibody 73R009 or a monovalent version thereof. In some embodiments, the anti-MET antibody is a humanized version of antibody 73R009 or a monovalent version thereof. In some embodiments, the anti-MET antibody is antibody 73R010 (73R009 H12L7) or a monovalent version thereof. In some embodiments, the MET-binding agent is an anti-MET/FZD-Fc bispecific agent. In some embodiments, the MET-binding agent is an anti-MET/FZD8-Fc bispecific agent. In some embodiments, the anti-MET/FZD-Fc bispecific agent is 315B06. In some embodiments, the anti-MET/FZD-Fc bispecific agent is 315B07. In some embodiments, the MET-binding agent is bispecific agent 315B09.

The present invention also provides methods of treating cancer in a human subject, comprising: (a) selecting a subject for treatment based, at least in part, on the subject having a cancer that has an elevated or high expression level of MET, and (b) administering to the subject a therapeutically effective amount of a MET-binding agent described herein.

Methods for determining the level of MET expression in a cell, tumor, or cancer are known by those of skill in the art. For nucleic acid expression these methods include, but are not limited to, PCR-based assays, microarray analyses, and nucleotide sequencing (e.g., NextGen sequencing). For protein expression, these methods include, but are not limited to, Western blot analysis, protein arrays, ELISAs, immunohistochemistry (IHC) assays, and FACS analysis.

Methods for determining whether a tumor or cancer has an elevated or high level of MET expression can use a variety of samples. In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is processed to a cell lysate. In some embodiments, the sample is processed to DNA. In some embodiments, the sample is processed to RNA.

The present invention further provides pharmaceutical compositions comprising the binding agents described herein. In certain embodiments, the pharmaceutical compositions further comprise a pharmaceutically acceptable vehicle. These pharmaceutical compositions find use in inhibiting tumor growth and/or treating cancer in a subject (e.g., a human patient).

In certain embodiments, the invention provides pharmaceutical compositions comprising bispecific agents, wherein at least about 90%, at least about 95%, at least about 98%, at least about 99% of the agents in the composition are bispecific agents or heterodimeric agents. In certain embodiments, the bispecific agents are IgG (e.g., IgG2 or IgG1) based agents. In certain embodiments, the bispecific agents are IgG2-based agents. In certain embodiments, less than about 10%, less than about 5%, less than about 2%, or less than about 1% of the total agents in the composition are monospecific agents or homodimeric agents. In certain embodiments, the agents in the composition are at least about 98% heterodimeric.

In certain embodiments, formulations are prepared for storage and use by combining a purified antibody or agent of the present invention with a pharmaceutically acceptable vehicle (e.g., a carrier or excipient). Suitable pharmaceutically acceptable vehicles include, but are not limited to, non-toxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose, or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 22^{st} Edition, 2012, Pharmaceutical Press, London).

The pharmaceutical compositions of the present invention can be administered in any number of ways for either local or systemic treatment. Administration can be topical by epidermal or transdermal patches, ointments, lotions, creams, gels, drops, suppositories, sprays, liquids, and powders; pulmonary by inhalation or insufflation of powders or aerosols, including by nebulizer, intratracheal, and intranasal; oral; or parenteral including intravenous, intraarterial, intratumoral, subcutaneous, intraperitoneal, intramuscular (e.g., injection or infusion), or intracranial (e.g., intrathecal or intraventricular).

The therapeutic formulation can be in unit dosage form. Such formulations include tablets, pills, capsules, powders, granules, solutions or suspensions in water or non-aqueous media, or suppositories. In solid compositions such as tablets the principal active ingredient is mixed with a pharmaceutical carrier. Conventional tableting ingredients include corn starch, lactose, sucrose, sorbitol, talc, stearic acid, magnesium stearate, dicalcium phosphate, or gums, and diluents (e.g., water). These can be used to form a solid pre-formulation composition containing a homogeneous mixture of a compound of the present invention, or a non-toxic pharmaceutically acceptable salt thereof. The solid pre-formulation composition is then subdivided into unit dosage forms of a type described above. The tablets, pills, etc. of the formulation or composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials include a number of polymeric acids and mixtures of polymeric acids with such materials as shellac, cetyl alcohol and cellulose acetate.

The MET-binding agents described herein can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22^{st} Edition, 2012, Pharmaceutical Press, London.

In certain embodiments, pharmaceutical formulations include a MET-binding agent (e.g., an antibody or a bispecific agent) of the present invention complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In certain embodiments, sustained-release preparations can be produced. Suitable examples of sustained-release preparations include semi-permeable matrices of solid hydrophobic polymers containing a MET-binding agent (e.g., an antibody or a bispecific agent), where the matrices are in the form of shaped articles (e.g., films or microcapsules). Additional examples of sustained-release matrices include polyesters, hydrogels such as poly(2-hydroxyethyl-methacrylate) or polyvinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

In certain embodiments, in addition to administering a MET-binding agent described herein (e.g., an antibody or bispecific agent), a method or treatment further comprises administering at least one additional therapeutic agent. An additional therapeutic agent can be administered prior to, concurrently with, and/or subsequently to, administration of the MET-binding agent. Pharmaceutical compositions comprising a MET-binding agent and the additional therapeutic agent(s) are also provided. In some embodiments, the at least one additional therapeutic agent comprises 1, 2, 3, or more additional therapeutic agents.

Combination therapy with at least two therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of at least one of the agents. Combination therapy may decrease the likelihood that resistant cancer cells will develop. In some embodiments, combination therapy comprises a therapeutic agent that primarily affects (e.g., inhibits or kills) non-tumorigenic cells and a therapeutic agent that primarily affects (e.g., inhibits or kills) tumorigenic CSCs.

Useful classes of therapeutic agents include, for example, anti-tubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, antifolates, antimetabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the second therapeutic agent is an alkylating agent, an anti-metabolite, an anti-mitotic, a topoisomerase inhibitor, or an angiogenesis inhibitor. In some embodiments, the second therapeutic agent is a platinum complex such as carboplatin or cisplatin. In some embodiments, the additional therapeutic agent is a platinum complex in combination with a taxane.

Therapeutic agents that may be administered in combination with the MET-binding agents include chemotherapeutic agents. Thus, in some embodiments, the method or treatment involves the administration of a MET-binding agent of the present invention in combination with a chemotherapeutic agent or cocktail of multiple different chemotherapeutic agents. In some embodiments, the method or treatment involves the administration of a bispecific agent of the present invention that binds MET and one or more WNT proteins in combination with a chemotherapeutic agent or cocktail of multiple different chemotherapeutic agents.

Chemotherapeutic agents useful in the instant invention include, but are not limited to, alkylating agents such as thiotepa and cyclophosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin: vinblastine; platinum; etoposide (VP-16); ifosfamide; mitomycin C; mitoxantrone; vincristine; vinorelbine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA); and pharmaceutically acceptable salts, acids or derivatives of any of the above. Chemotherapeutic agents also include anti-hormonal agents that act to regulate or inhibit hormone action on tumors such as anti estrogens including, for example, tamoxifen, raloxifene, aromatase inhibiting 4(5)-imidazoles, 4-hydroxytamoxifen, trioxifene, keoxifene, LY117018, onapristone, and toremifene (FARESTON); and anti-androgens such as flutamide, nilutamide, bicalutamide, leuprolide, and goserelin; and pharmaceutically acceptable salts, acids or derivatives of any of the above. In certain embodiments, the second therapeutic agent is cisplatin. In certain embodiments, the second therapeutic agent is carboplatin. In certain embodiments, the second therapeutic agent is paclitaxel.

In certain embodiments, the chemotherapeutic agent is a topoisomerase inhibitor. Topoisomerase inhibitors are chemotherapeutic agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan, as well as pharmaceutically acceptable salts, acids, or derivatives of any of these. In certain embodiments, the second therapeutic agent is irinotecan.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these. In certain embodiments, the second therapeutic agent is gemcitabine.

In certain embodiments, the chemotherapeutic agent is an anti-mitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (ABRAXANE), DHA-paclitaxel, or PG-paclitaxel. In certain alternative embodiments, the anti-mitotic agent comprises a vinca alkaloid, such as vincristine, binblastine, vinorelbine, or vindesine, or pharmaceutically acceptable salts, acids, or derivatives thereof. In some embodiments, the anti-mitotic agent is an inhibitor of kinesin Eg5 or an inhibitor of a mitotic kinase such as Aurora A or Plk1. In certain embodiments, where the chemotherapeutic agent administered in combination with a MET-binding agent is an anti-mitotic agent, the cancer or tumor being treated is breast cancer or a breast tumor.

In some embodiments, an additional therapeutic agent comprises an agent such as a small molecule. For example, treatment can involve the combined administration of a MET-binding agent (e.g. an antibody or bispecific agent) of the present invention with a small molecule that acts as an inhibitor against additional tumor-associated proteins including, but not limited to, EGFR, ErbB2, HER2, and/or MET. In certain embodiments, the additional therapeutic agent is a small molecule that inhibits a cancer stem cell pathway. In some embodiments, the additional therapeutic agent is a small molecule inhibitor of the NOTCH pathway. In some embodiments, the additional therapeutic agent is a small molecule inhibitor of the WNT pathway. In some embodiments, the additional therapeutic agent is a small molecule inhibitor of the BMP pathway. In some embodiments, the additional therapeutic agent is a small molecule that inhibits β-catenin signaling.

In some embodiments, an additional therapeutic agent comprises a biological molecule, such as an antibody. For example, treatment can involve the combined administration of a MET-binding agent (e.g. an antibody or bispecific agent) of the present invention with other antibodies against additional tumor-associated proteins including, but not limited to, antibodies that bind EGFR, ErbB2, and/or HER2. In certain embodiments, the additional therapeutic agent is an antibody that is an anti-cancer stem cell marker antibody. In some embodiments, the additional therapeutic agent is an antibody that binds a component of the NOTCH pathway. In some embodiments, the additional therapeutic agent is an antibody that binds a component of the WNT pathway. In certain embodiments, the additional therapeutic agent is an antibody that inhibits a cancer stem cell pathway. In some embodiments, the additional therapeutic agent is an antibody inhibitor of the NOTCH pathway. In some embodiments, the additional therapeutic agent is an antibody inhibitor of the WNT pathway. In some embodiments, the additional therapeutic agent is an antibody inhibitor of the BMF pathway. In some embodiments, the additional therapeutic agent is an antibody that inhibits β-catenin signaling. In certain embodiments, the additional therapeutic agent is an antibody that is an angiogenesis inhibitor or modulator (e.g., an anti-VEGF or VEGF receptor antibody). In certain embodiments, the additional therapeutic agent is bevacizumab (AVASTIN), trastuzumab (HERCEPTIN), panitumumab (VECTIBIX), or cetuximab (ERBITUX).

Furthermore, treatment with a MET-binding agent described herein can include combination treatment with other biologic molecules, such as one or more cytokines (e.g., lymphokines, interleukins, tumor necrosis factors, and/or growth factors) or can be accompanied by surgical removal of tumors, cancer cells, or any other therapy deemed necessary by a treating physician.

In certain embodiments, the treatment involves the administration of a MET-binding agent (e.g. an antibody or bispecific agent) of the present invention in combination with radiation therapy. Treatment with a MET-binding agent can occur prior to, concurrently with, or subsequent to administration of radiation therapy. Dosing schedules for such radiation therapy can be determined by the skilled medical practitioner.

It will be appreciated that the combination of a MET-binding agent and an additional therapeutic agent may be administered in any order or concurrently. Treatment with a MET-binding agent (e.g., an antibody or a bispecific agent) can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in The Chemotherapy Source Book, 4^{th} Edition, 2008, M. C. Perry, Editor, Lippincott, Williams & Wilkins, Philadelphia, Pa.

In some embodiments, the MET-binding agent will be administered to patients that have previously undergone treatment with therapeutic agents. In certain other embodiments, the MET-binding agent and an additional therapeutic agent will be administered substantially simultaneously or concurrently. For example, a subject may be given a MET-binding agent (e.g., an antibody or bispecific agent) while undergoing a course of treatment with a second therapeutic agent (e.g., chemotherapy). In certain embodiments, a MET-binding agent will be administered within 1 year of the treatment with a second therapeutic agent. In certain alternative embodiments, a MET-binding agent will be administered within 10, 8, 6, 4, or 2 months of any treatment with a second therapeutic agent. In certain other embodiments, a MET-binding agent will be administered within 4, 3, 2, or 1 weeks of any treatment with a second therapeutic agent. In some embodiments, a MET-binding agent will be administered within 5, 4, 3, 2, or 1 days of any treatment with a second therapeutic agent. It will further be appreciated that the two (or more) agents or treatments may be administered to the subject within a matter of hours or minutes (i.e., substantially simultaneously).

For the treatment of a disease, the appropriate dosage of a MET-binding agent (e.g., an antibody or bispecific agent) of the present invention depends on the type of disease to be treated, the severity and course of the disease, the responsiveness of the disease, whether the MET-binding agent is administered for therapeutic or preventative purposes, previous therapy, the patient's clinical history, and so on, all at the discretion of the treating physician. The MET-binding agent can be administered one time or as a series of treatments spread over several days to several months, or until a cure is effected or a diminution of the disease state is achieved (e.g., reduction in tumor size), or a stable disease state is achieved. Optimal dosing schedules can be calculated from measurements of drug accumulation in the body of the patient and will vary depending on the relative potency of an individual antibody or agent. The administering physician can determine optimum dosages, dosing methodologies, and repetition rates. In certain embodiments, dosage of a MET-binding agent is from about 0.01 μg to about 100 mg/kg of body weight, from about 0.1 μg to about 100 mg/kg of body weight, from about 1 μg to about 100 mg/kg of body weight, from about 1 mg to about 100 mg/kg of body weight, about 1 mg to about 80 mg/kg of body weight from about 10 mg to about 100 mg/kg of body weight, from about 10 mg to about 75 mg/kg of body weight, or from about 10 mg to about 50 mg/kg of body weight. In certain embodiments, the dosage of the MET-binding agent is from about 0.1 mg to about 20 mg/kg of body weight. In certain embodiments, dosage can be given once or more daily, weekly, monthly, or yearly. In certain embodiments, the MET-binding agent is given once every week, once every two weeks, once every three weeks, or once every month.

In some embodiments, a MET-binding agent (e.g., an antibody or bispecific agent) may be administered at an initial higher “loading” dose, followed by one or more lower doses. In some embodiments, the frequency of administration may also change. In some embodiments, a dosing regimen may comprise administering an initial dose, followed by additional doses (or “maintenance” doses) once a week, once every two weeks, once every three weeks, or once every month. For example, a dosing regimen may comprise administering an initial loading dose, followed by a weekly maintenance dose of for example, one-half of the initial dose. Or a dosing regimen may comprise administering an initial loading dose, followed by maintenance doses of, for example one-half of the initial dose every other week. Or a dosing regimen may comprise administering three initial doses for 3 weeks, followed by maintenance doses of, for example, the same amount every other week. Or a dosing regimen may comprise administering an initial dose followed by additional doses every 3 weeks or once a month. The treating physician can estimate repetition rates for dosing based on measured residence times and concentrations of the drug in bodily fluids or tissues. The progress of therapy can be monitored by conventional techniques and assays.

As is known to those of skill in the art, administration of any therapeutic agent may lead to side effects and/or toxicities. In some cases, the side effects and/or toxicities are so severe as to preclude administration of the particular agent at a therapeutically effective dose. In some cases, drug therapy must be discontinued, and other agents may be tried. However, many agents in the same therapeutic class often display similar side effects and/or toxicities, meaning that the patient either has to stop therapy, or if possible, suffer from the unpleasant side effects associated with the therapeutic agent.

Side effects from therapeutic agents may include, but are not limited to, hives, skin rashes, itching, nausea, vomiting, decreased appetite, diarrhea, chills, fever, fatigue, muscle aches and pain, headaches, low blood pressure, high blood pressure, hypokalemia, bone effects, low blood counts, bleeding, and cardiovascular problems.

Thus, one aspect of the present invention is directed to methods of treating cancer in a patient comprising administering a MET-binding agent described herein using an intermittent dosing regimen, which may reduce side effects and/or toxicities associated with administration of the agent. As used herein, “intermittent dosing” refers to a dosing regimen using a dosing interval of more than once a week, e.g., dosing once every 2 weeks, once every 3 weeks, once every 4 weeks, etc. In some embodiments, a method for treating cancer in a human patient comprises administering to the patient an effective dose of a MET-binding agent (e.g., an antibody or a bispecific agent) described herein according to an intermittent dosing regimen. In some embodiments, a method for treating cancer in a human patient comprises administering to the patient an effective dose of a MET-binding agent (e.g., an antibody or a bispecific agent) according to an intermittent dosing regimen, and increasing the therapeutic index of the MET-binding agent. In some embodiments, the intermittent dosing regimen comprises administering an initial dose of a MET-binding agent (e.g., an antibody or a bispecific agent) to the patient, and administering subsequent doses of the MET-binding agent about once every 2 weeks. In some embodiments, the intermittent dosing regimen comprises administering an initial dose of a MET-binding agent (e.g., an antibody or a bispecific agent) to the patient, and administering subsequent doses of the MET-binding agent about once every 3 weeks. In some embodiments, the intermittent dosing regimen comprises administering an initial dose of a MET-binding agent (e.g., an antibody or a bispecific agent) to the patient, and administering subsequent doses of the MET-binding agent about once every 4 weeks.

In some embodiments, the subsequent doses in an intermittent dosing regimen are about the same amount or less than the initial dose. In other embodiments, the subsequent doses are a greater amount than the initial dose. As is known by those of skill in the art, doses used will vary depending on the clinical goals to be achieved. In some embodiments, the initial dose is about 0.25 mg/kg to about 20 mg/kg. In some embodiments, the initial dose is about 0.5, 1, 2, 3, 4, 5, 6, 7, 8, 9, 10, 11, 12, 13, 14, 15, 16, 17, 18, 19, or 20 mg/kg. In certain embodiments, the initial dose is about 0.5 mg/kg. In certain embodiments, the initial dose is about 1 mg/kg. In certain embodiments, the initial dose is about 2.5 mg/kg. In certain embodiments, the initial dose is about 5 mg/kg. In certain embodiments, the initial dose is about 7.5 mg/kg. In certain embodiments, the initial dose is about 10 mg/kg. In certain embodiments, the initial dose is about 12.5 mg/kg. In certain embodiments, the initial dose is about 15 mg/kg. In certain embodiments, the initial dose is about 20 mg/kg. In some embodiments, the subsequent doses are about 0.25 mg/kg to about 15 mg/kg. In certain embodiments, the subsequent doses are about 0.5, 1, 2, 3, 4, 5, 6, 7, 8, 9, 10, 11, 12, 13, 14, or 15 mg/kg. In certain embodiments, the subsequent doses are about 0.5 mg/kg. In certain embodiments, the subsequent doses are about 1 mg/kg. In certain embodiments, the subsequent doses are about 2.5 mg/kg. In certain embodiments, the subsequent doses are about 5 mg/kg. In some embodiments, the subsequent doses are about 7.5 mg/kg. In some embodiments, the subsequent doses are about 10 mg/kg. In some embodiments, the subsequent doses are about 12.5 mg/kg.

In some embodiments, the dosing schedule may be limited to a specific number of administrations or “cycles”. In some embodiments, the MET-binding agent is administered for 3, 4, 5, 6, 7, 8, or more cycles. For example, the MET-binding agent is administered every 2 weeks for 6 cycles, the MET-binding agent is administered every 3 weeks for 6 cycles, the MET-binding agent is administered every 2 weeks for 4 cycles, the MET-binding agent is administered every 3 weeks for 4 cycles, etc. Dosing schedules can be decided upon and subsequently modified by those skilled in the art.

Thus the present invention provides methods for reducing toxicity of a MET-binding agent (e.g., an antibody or a bispecific agent) described herein in a human patient that comprise administering to the patient the MET-binding agent using an intermittent dosing regimen. Also provided are methods for reducing side effects of a MET-binding agent (e.g., an antibody or a bispecific agent) in a human patient which comprise administering to the patient the MET-binding agent using an intermittent dosing regimen. Also provided are methods for increasing the therapeutic index of a MET-binding agent (e.g., an antibody or a bispecific agent) in a human patient, wherein the methods comprise administering to the patient the MET-binding agent using an intermittent dosing regimen.

The choice of delivery method for the initial and subsequent doses is made according to the ability of the animal or human patient to tolerate introduction of the MET-binding agent into the body. Thus, in any of the aspects and/or embodiments described herein, the administration of the MET-binding agent (e.g., an antibody or a bispecific agent) may be by intravenous injection or intravenously. In some embodiments, the administration is by intravenous infusion. In any of the aspects and/or embodiments described herein, the administration of the MET-binding agent may be by a non-intravenous route.

### V. KITS COMPRISING MET/WNT-BINDING AGENTS

The present invention provides kits that comprise the MET-binding agents (e.g., antibodies or bispecific agents) described herein and that can be used to perform the methods described herein. In certain embodiments, a kit comprises at least one purified antibody against MET or at least one purified bispecific agent that binds MET and one or more components of the WNT pathway in one or more containers. In some embodiments, the kits contain all of the components necessary and/or sufficient to perform a detection assay, including all controls, directions for performing assays, and any necessary software for analysis and presentation of results. One skilled in the art will readily recognize that the disclosed MET-binding agents of the present invention can be readily incorporated into one of the established kit formats which are well known in the art.

Further provided are kits comprising a MET-binding agent (e.g., an antibody or bispecific agent), as well as at least one additional therapeutic agent. In certain embodiments, the second (or more) therapeutic agent is a chemotherapeutic agent. In certain embodiments, the second (or more) therapeutic agent is an angiogenesis inhibitor.

Embodiments of the present disclosure can be further defined by reference to the following non-limiting examples, which describe in detail preparation of certain antibodies of the present disclosure and methods for using antibodies of the present disclosure. It will be apparent to those skilled in the art that many modifications, both to materials and methods, may be practiced without departing from the scope of the present disclosure.

### EXAMPLES

### Example 1

### Binding Affinities of MET-Binding Agents

The K_{D} of monovalent version of anti-MET antibody 73R009, monovalent anti-MET antibody 5D5, and anti-MET/FZD8-Fc bispecific agent 315B06 were determined using a Biacore 2000 system from Biacore Life Sciences (GE Healthcare). A goat anti-human antibody (Invitrogen H10500) was coupled to a carboxymethyl-dextran (CM5) SPR chip using standard amine-based chemistry (NHS/EDC) and blocked with ethanolamine. Antibodies were diluted to a concentration of 10 μg/ml in HBS-P-BSA (0.01M HEPES pH7.4, 0.15M NaCl, 0.005% v/v Polysorbate 20, 10 μg/ml BSA) and captured onto the chip via the anti-human antibody. Human MET was serially diluted 2-fold from 300 nM to 37.5 nM in HBS-P-BSA and injected sequentially over the captured anti-MET antibodies. MET association and dissociation was measured at each concentration. After each antigen injection 5 μl of 100 mM H₃P0₄ was injected to remove the antigen-antibody complex and a subsequent injection performed. Kinetic data were collected over time and were fit using the simultaneous global fit equation to yield affinity constants (K_{D} values) for each agent.

Bivalent “parental” antibody 73R009 had an affinity constant (K_{D}) for human MET of 1.1 nM, the monovalent version of 73R009 had a K_{D} for human MET of 1.4 nM, monovalent antibody 5D5 had a K_{D} for human MET of 7.2 nM, and bispecific agent 315B06 had a K_{D} for human MET of 1.8 nM. Thus, the monovalent anti-MET antibody 73R009 and the bispecific agent 315B06 both demonstrated binding affinity very similar to the parental antibody despite the fact the parental antibody is bivalent. In addition, the bispecific agent 315B06 appeared to have stronger affinity for human MET than anti-MET antibody 5D5.

The anti-MET/FZD8-Fc bispecific agent 315B06 has been shown to not bind mouse MET.

Anti-MET/FZD8-Fc bispecific agent 315B06 comprises (a) a heavy chain encoded by the plasmid deposited with ATCC, 10801 University Boulevard, Manassas, Va., USA, under the conditions of the Budapest Treaty on Mar. 12, 2013 and assigned designation number PTA-13609, (b) a light chain encoded by the plasmid deposited with ATCC under the conditions of the Budapest Treaty on Mar. 12, 2013 and assigned designation number PTA-13610, and (c) a fusion protein encoded by the plasmid deposited with ATCC under the conditions of the Budapest Treaty on Mar. 12, 2013 and assigned designation number PTA-13611.

### Example 2

### Inhibition of Binding of Hepatocyte Growth Factor to MET

A full-length human MET (FL-MET) construct was generated using standard recombinant DNA techniques. HEK-293T cells were transiently transfected with the MET construct and a GFP plasmid at a plasmid MET:GFP ratio of 2:1. After 24 hours, transfected cells were harvested and suspended in ice cold PBS containing 2% FBS. The transfected cells were incubated on ice in the presence of 10, 5, 2.5, 1.25, 0.625, 0.3, or 0.16 μg/ml of monovalent anti-MET antibody 5D5, a monovalent version of anti-MET antibody 73R009, or anti-MET/FZD8-Fc bispecific agent 315B06 for 1 hour. 30 ng of hepatocyte growth factor (HGF) conjugated to biotin was added to each sample and incubated on ice for an additional 40 minutes. Cells were washed with PBS containing 2% FBS, PE-conjugated streptavidin was added, and the cells were incubated for 1 hour. Transfected cells were incubated with no HGF as a negative control and with HGF but no antibody or binding agent as a positive control. After final incubation, cells were stained with 5 μg/ml DAPI and analyzed on a FACSCanto II instrument (BD Biosciences, San Jose, Calif.) and the data was processed using FlowJo software.

As shown in FIG. 1, approximately 20% of the transfected cells expressed MET and were bound by human HGF (FIG. 1A). Inhibition of HGF binding to MET by the binding agents was compared to the positive control of 20% binding. The monovalent anti-MET antibody 5D5 reduced binding of HGF to MET by approximately 70% at the highest concentration of 10 μg/ml with a dose-dependent response down to a reduction of 28% at the lowest concentration of 0.16 μg/ml (FIG. 1B). In contrast, the monovalent version of anti-MET antibody 73R009 reduced binding of HGF to MET by approximately 72% at the highest concentration of 10 μg/ml with a dose-dependent response down to a reduction of approximately 56% at the lowest concentration of 0.16 μg/ml (FIG. 1C). Similarly, the bispecific anti-MET/FZD8-Fc agent 315B06 reduced binding of HGF to MET by approximately 80% at the highest concentration of 10 μg/ml with a dose-dependent response down to a reduction of approximately 56% at the lowest concentration of 0.16 μg/ml (FIG. 1D).

These results showed that both the monovalent version of anti-MET antibody 73R009 and the bispecific anti-MET/FZD8-Fc agent 351B06 were strong blockers of HGF binding to MET. In addition, both appeared to have a greater ability to block binding of HGF to MET than anti-MET antibody 5D5 and were able to block binding at lower concentrations.

### Example 3

### Inhibition of HGF-Induced MET Activity

MET activation in human cells can be characterized by analyzing MET phosphorylation and downstream activation of mitogen-activated protein kinase (MAPK) and AKT after HGF stimulation.

A549 cells were seeded into 12-well plates at 1.5×10⁵ cells/well in DMEM medium containing 10% FBS and grown overnight. Cells were transferred to serum-free medium and after approximately 18 hours the cells were pre-treated for one hour with a monovalent version of anti-MET antibody 73R009, bispecific anti-MET/FZD8-Fc agent 5D5/FZD, and bispecific anti-MET/FZD8-Fc agent 315B06 at concentrations of 50, 10, 2, and 0.4 μg/ml. Subsequently the cells were stimulated with 50 ng/ml human HGF (EMD Millipore, Billerica Mass.) for 15 minutes. Cells were lysed and cell lysate supernatants were collected. Cell lysates were resolved by SDS-PAGE using 4-12% NuPAGE Novex gels (Invitrogen/Life Technologies, Grand Island, N.Y.), transferred to nitrocellulose membranes, and analyzed by Western blot techniques. Antibodies used were anti-human MET (anti-Met mAb, Cell Signaling Technology, Danvers, Mass.); anti-phospho-MET (anti-phospho-MET (Tyr1234/1235) mAb, Cell Signaling Technology, Danvers, Mass.); anti-phospo-AKT (anti-phospho-AKT (Ser473) mAb, Cell Signaling Technology, Danvers, Mass.); anti-phospho-MAPK (anti-phospho-p44/42 MAPK (Erk1/2) (Thr202/Tyr204), Cell Signaling Technology, Danvers, Mass.); and anti-actin (anti-beta actin antibody, Abcam, Cambridge, Mass.).

As shown in FIG. 2, bispecific anti-MET/FZD8-Fc agent 315B06 reduced the amount of phosphorylated MET to a greater extent than the bispecific anti-MET/FZD agent 5D5/FZD or the monovalent version of anti-MET antibody 73R009. At the highest concentration, it appeared that 315B06 reduced the amount of phosphorylated AKT to a greater extent than the other agents also. These studies demonstrated that the bispecific anti-MET/FZD8-Fc agent 315B06 was able to inhibit and/or block HGF-induced MET activation and was able to inhibit and/or block MET activation to a greater extent than the bispecific anti-MET/FZD agent 5D5/FZD or the monovalent version of anti-MET antibody 73R009.

### Example 4

### Inhibition of WNT Signaling

STF-293 cells were cultured in DMEM supplemented with antibiotics and 10% FCS. The STF-293 cells are HEK-293 cells stably integrated with an 8×TCF Luc reporter vector and a Renilla luciferase reporter. The 8×TCF Luc reporter contains seven copies of the TCF binding site linked to a promoter upstream of a firefly luciferase reporter gene to measure canonical WNT signaling levels (Gazit et al., 1999, Oncogene, 18:5959-66). The Renilla luciferase reporter (Promega; Madison, Wis.) was used as an internal control for transfection efficiency. Anti-MET/FZD bispecific agent 315B06 and control agents anti-MET monovalent agent 5D5/FLAG and monovalent agent FZD8/FLAG were serially diluted 5-fold from 20 μg/ml to 0.0064 μg/ml, added to the appropriate wells, and incubated overnight. The cells were then incubated in the presence or absence of WNT3a-conditioned medium that had been prepared from L cells that stably express WNT3a or control conditioned media from L cells not over-expressing WNT3a. After overnight incubation, luciferase levels were measured using a dual luciferase assay kit (Promega; Madison, Wis.) with firefly luciferase activity normalized to Renilla luciferase activity.

As shown in FIG. 3, anti-MET/FZD8-Fc bispecific agent 315B06 inhibited WNT pathway signaling. The inhibition was similar to the monovalent FZD8/FLAG agent and as expected the anti-MET 5D5/FLAG agent had no ability to inhibit WNT pathway signaling. Thus, in combination with the results presented in Example 3, the anti-MET/FZD8-Fc bispecific agent 315B06 has demonstrated the ability to inhibit both MET-induced and WNT-induced signaling and/or activation.

### Example 5

### Inhibition of Lung Tumor Growth In Vivo

OMP-LU45 tumors were selected based on the high level of MET expression observed in microarray analysis. Dissociated OMP-LU45 lung tumor cells (1×10⁵ cells) were injected into 6-8 week old NOD/SCID mice. Tumors were allowed to grow for 26 days until they reached an average volume of 90 mm³. The mice were randomized (n=10 per group) and treated with a monovalent anti-MET antibody (5D5/FLAG), a control antibody, a monovalent FZD8-Fc (FZD8Fc/FLAG), a bivalent FZD8-Fc (54F28), or an anti-MET/FZD8Fc bispecific agent, either as single agents or in combination with taxol. Protein agents were dosed at 25 mg/kg once a week, and taxol was dosed at 15 mg/kg once a week. Administration of the protein agents and taxol was performed via injection into the intraperitoneal cavity. Tumor growth was monitored and tumor volumes were measured with electronic calipers at the indicated time points. Data are expressed as mean±S.E.M.

When used as a monotherapy, all of the agents had minimal or no detectable effect on LU45 tumor growth as compared to the control antibody (FIG. 4A). In contrast, the MET/FZD8-Fc bispecific agent in combination with taxol significantly inhibited OMP-LU45 tumor growth and this inhibition of tumor growth was greater than inhibition observed with any of the other agents in combination with taxol (FIG. 4B).

### Example 6

### Humanization of MET Antibodies

Humanized versions of the “parental” antibody 73R009 were generated. The best combinations of humanized heavy and light chains were chosen that contained minimal murine sequence content and retained the binding characteristics of the parental antibody. Humanized antibody 73R009 H12L7, also referred to as antibody 73R010, comprises a heavy chain encoded by the plasmid deposited with ATCC, 10801 University Boulevard, Manassas, Va., USA, under the conditions of the Budapest Treaty on May 29, 2013 and assigned designation number PTA-120387 and a light chain encoded by the plasmid deposited with ATCC under the conditions of the Budapest Treaty on May 29, 2013 and assigned designation number PTA-120388.

Antibody 73R010 was used in a variation of the anti-MET/FZD8-Fc bispecific agent, and this variant bispecific agent is referred to as 315B07. The anti-MET/FZD8-Fc bispecific agent 3151307 was determined to have a K_{D} of 126.4 pM using a Biacore 2000 system as described in Example 1.

A variant of anti-MET antibody 73R010 was generated by substituting the cysteine residue corresponding to position 234 of SEQ ID NO:97 with a serine residue. A plasmid encoding this variant heavy chain was deposited with ATCC, 10801 University Boulevard, Manassas, Va., USA, under the conditions of the Budapest Treaty on Nov. 6, 2013 and assigned designation number PTA-120695. This 73R010 variant was used in a variation of the anti-MET/FZD8-Fc bispecific agent and this bispecific agent is referred to as 315B09.

### Example 7

### Epitope Mapping

To identify the MET epitope recognized by the anti-MET/FZD8-Fc bispecific agent, epitope mapping was performed. Although the amino acid sequences of the human and mouse Sema domains are approximately 88% identical, bispecific agent 315B07 does not bind mouse MET. Therefore, site-directed mutagenesis was performed to create a series of human Sema mutants. Kunkel mutagenesis was used to replace specific amino acids in the human Sema sequence with the corresponding amino acids found in the mouse Sema sequence at predicted antibody-binding sites. The human Sema mutants were: Mut96,99 (F96L and Q99R), Mut96 (F96L), Mut99 (Q99R), Mut328 (Q328N), Mut331 (R331K), and Mut337,338 (L337P and N338S).

Mammalian expression plasmid vectors encoding a wild-type human Sema, a wild-type mouse Sema, a mouse/human Sema mutant wherein the 5′ end is mouse (aa 1-331) and the 3′ end is human (aa 332-515), and the human Sema mutants outlined above were generated by standard recombinant technology. These constructs comprise the transmembrane domain of a human CD4 protein and GFP (for example, Sema-hCD4TM-GFP). These expression constructs allow for expression of the Sema domain on the cell surface, as well as expression of GFP to monitor expression. I-K-293T cells were transfected with expression vectors encoding the Sema domains using a calcium phosphate transfection kit (Clontech, Mountain View, Calif.). Sixteen hours post-transfection, cells were detached with Versene solution (Life Technologies, Grand Island, N.Y.), and incubated with bispecific agent 315B07 or bispecific agent anti-MET antibody 5D5/FZD8-Fc for one hour at 4° C. The cells were washed and bound bispecific agents were detected with a secondary anti-human Fc antibody conjugated to allophycocyanin (APC). After incubation, cells were washed and 1 ug/ml DAPI was added to the cells. Labeled cells were analyzed by flow cytometry to measure the binding of 315B07 and 5D5/FZD8-Fc to the series of Sema mutants.

As shown in FIG. 5, bispecific agent 315B07 binds the wild-type human Sema domain but does not bind the mouse Sema domain or the mouse/human chimeric mutant. When amino acid residue 96 is changed from phenylalanine to leucine and amino acid residue 99 is changed from glutamine to arginine, binding of 315B07 to the human Sema domain is not detected. When only amino acid residue 96 is changed from phenylalanine to leucine, binding of 315B07 to the human Sema domain is unaffected. In contrast, when only amino acid residue 99 is changed from glutamine to arginine, binding of 315B07 to the Sema domain is not detected. When amino acid 331 is changed from arginine to lysine binding of 315B07 is unaffected. In addition, when amino acid 328 is changed from glutamine to asparagine binding of 315B07 is unaffected. Similarly when amino acid 337 is changed from leucine to proline and amino acid 338 is changed from asparagine to serine, binding of 315B07 is unaffected (data not shown). In contrast, when amino acid residue 99 is changed from glutamine to arginine, the binding of 5D5/FZD8-Fc is unaffected. In addition, when amino acid 331 is changed from arginine to lysine binding of 5D5/FZD8-Fc is not detected. When amino acid 328 is changed from glutamine to asparagine binding of 5D5/FZD8-Fc is not detected and when amino acid 337 is changed from leucine to proline and amino acid 338 is changed from asparagine to serine, binding of 5D5/FZD8-Fc is greatly reduced.

These results show that the glutamine at amino acid 99 of the human SEMA domain is a key amino acid of the epitope for the anti-MET arm of 315B07. Furthermore, these results clearly show that the epitope on c-MET for bispecific agent 315B07 is different than the epitope on c-MET for anti-MET antibody 5D5, which comprises at least amino acids 328, 331, 337, and 338 of human MET. The positions of the 315B07 epitope and the 5D5 epitope are depicted in the ribbon diagrams of FIG. 6.

### Example 8

### Inhibition of MMTV-Wnt1 Tumor Growth In Vivo

The MMTV-Wnt1 breast cancer model was chosen for study because this tumor model has previously been shown to be highly sensitive to WNT pathway inhibition using anti-FZD antibody OMP-18R5 (vantictumab) or OMP-54F28 (FZD8-Fc). MMTV-Wnt1 tumor cells (25,000 cells) were injected subcutaneously into NOD/SCID mice. Tumors were allowed to grow 17 days until they reached an average volume of 110 mm³. Tumor-bearing mice were randomized (n=12 per group) and treated with either control antibody (30 mg/kg), OMP-54F28 (5 mg/kg), or 315B07 (anti-MET/FZD8-Fc bispecific agent) (30 or 60 mg/kg) once a week. Tumor growth was monitored and tumor volumes were measured with electronic calipers at the indicated time points. Data are expressed as mean±S.E.M.

As shown in FIG. 7, anti-MET/FZD8-Fc bispecific agent 315B07 significantly inhibited MMTV-Wnt tumor growth as compared to control antibody indicating that it is an effective Wnt antagonist in vivo.

### Example 9

### Inhibition of KP4 Pancreatic Tumor Growth In Vivo

The KP4 pancreatic cell line expresses high levels of both human growth factor (HGF) and MET in an autocrine manner and has been shown to be sensitive to anti-MET antibody (5D5) treatment in vivo (Jin et al., 2008, Cancer Res. 68:4360-4368). KP4 tumor cells (1×10⁶) were injected subcutaneously into NOD/SCID mice. Tumors were allowed to grow 15 days until they reached an average volume of 125 mm³. Tumor-bearing mice were randomized into five groups (n=10 per group) and treated with control antibody (25 mg/kg, weekly), OMP-18R5 (25 mg/kg, weekly), FZD8-Fc/FLAG (30 mg/kg), anti-MET 5D5/FLAG (30 mg/kg) or anti-MET/FZD8-Fc (5D5/FZD8-Fc, 30 mg/kg). The latter 3 proteins were administered twice weekly. Tumor growth was monitored and tumor volumes were measured with electronic calipers at the indicated time points. Data are expressed as mean±S.E.M.

As shown in FIG. 8, the anti-MET/FZD8-Fc bispecific inhibited KP4 tumor growth indicating that it is an effective MET antagonist in vivo.

### Example 10

### Inhibition of OMP-LIV1 HCC Tumor Growth In Vivo

OMP-LIV1 is a patient-derived hepatocellular carcinoma (HCC) tumor. OMP-LIV1 tumor cells (50,000) were injected subcutaneously into NOD/SCID mice. Tumors were allowed to grow approximately 50 days until they reached an average volume of 220 mm³. Tumor-bearing mice were randomized into four groups (n=9-11 per group) and treated with sorafenib alone, sorafenib in combination with a one-armed MET antibody, sorafenib in combination with FZD8-Fc/FLAG, or sorafenib in combination with anti-MET/FZD8-Fc bispecific agent OMP-315B07. Antibodies or bispecific agents were dosed weekly at 30 mg/kg, twice per week. Sorafenib was dosed at 60 mg/kg, administered daily, 5 times per week. Tumor growth was monitored and tumor volumes were measured with electronic calipers at the indicated time points. Data are expressed as mean±S.E.M.

The anti-MET/FZD8-Fc bispecific agent OMP-315B07 inhibited OMP-LIV1 HCC tumor growth in combination with sorafenib to a greater extent than sorafenib alone (p=0.02) and to a greater extent than the sorafenib in combination with either the one-armed anti-MET or the one-armed FZD8-Fc (FIG. 9).

It is understood that the examples and embodiments described herein are for illustrative purposes only and that various modifications or changes in light thereof will be suggested to persons skilled in the art and are to be included within the spirit and purview of this application.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence was specifically and individually indicated to be so incorporated by reference.

Following are the sequences disclosed in the application:

73R009 Heavy chain CDR1
(SEQ ID NO: 1)
ASYAWS
73R009 Heavy chain CDR2
(SEQ ID NO: 2)
YISYSGGTDYNPSLKS
73R009 Heavy chain CDR3
(SEQ ID NO: 3)
KGAY
73R009 Light chain CDR1
(SEQ ID NO: 4)
SASSSVSSSYLY
73R009 Light chain CDR2
(SEQ ID NO: 5)
STSNLAS
73R009 Light chain CDR3
(SEQ ID NO: 6)
HQWSSYPYT
73R009 Heavy chain variable region amino acid sequence
(SEQ ID NO: 7)
QVQLQESGPGLVKPSETLSLTCTVTGTITASYAWSWIRQPPGKGLEWMGYISYSGGTDY
NPSLKSRITISRDTFKNQFSLKLSSVTAADTATYYCARKGAYWGQGTLVTVSS
73R009 Light chain variable region amino acid sequence
(SEQ ID NO: 8)
DIVLTQSPATLSASPGEKVTLTCSASSSVSSSYLYWYQQKPGQAPKLLIYSTSNLASGVP
ARFSGSGSGTSYSLTISSLEPEDFATYYCHQWSSYPYTFGGGTKLEIK
73R009 Heavy chain amino acid sequence with predicted
signal sequence underlined
(SEQ ID NO: 9)
MKHLWFFLLLVAAPRWVLSQVQLQESGPGLVKPSETLSLTCTVTGTTITASYAWSWIRQP
PGKGLEWMGYISYSGGTDYNPSLKSRITISRDTFKNQFSLKLSSVTAADTATYYCARKGA
YWGQGTLVTVSSASTKGPSVFPLAPCSRSTSESTAALGCLVKDYFPEPVTVSWNSGALTS
GVHTFPAVLQSSGLYSLSSVVTVPSSNFGTQTYTCNVDHKPSNTKVDKTVERKCCVECPP
CPAPPVAGPSVFLFPPKPKDTLMISRTPEVTCVVVDVSHEDPEVQFNWYVDGVEVHNAKT
KPREEQFNSTFRVVSVLTVVHQDWLNGKEYKCKVSNKGLPAPIEKTISKTKGQPREPQVY
TLPPSREEMTKNQVSLTCLVKGFYPSDIAVEWESNGQPENNYKTTPPMLDSDGSFFLYSK
LTVDKSRWQQGNVFSCSVMHEALHNHYTQKSLSLSPGK
73R009 (13A variant) Heavy chain amino acid sequence
with predicted signal sequence underlined
(SEQ ID NO: 10)
MKHLWFFLLLVAAPRWVLSQVQLQESGPGLVKPSETLSLTCTVTGTTITASYAWSWIRQP
PGKGLEWMGYISYSGGTDYNPSLKSRITISRDTFKNQFSLKLSSVTAADTATYYCARKGA
YWGQGTLVTVSSASTKGPSVFPLAPCSRSTSESTAALGCLVKDYFPEPVTVSWNSGALTS
GVHTFPAVLQSSGLYSLSSVVTVPSSNFGTQTYTCNVDHKPSNTKVDKTVERKCCVECPP
CPAPPVAGPSVFLFPPKPKDTLMISRTPEVTCVVVDVSHEDPEVQFNWYVDGVEVHNAKT
KPREEQFNSTFRVVSVLTVVHQDWLNGKEYKCKVSNKGLPAPIEKTISKTKGQPREPQVY
TLPPSREKMTKNQVSLTCLVKGFYPSDIAVEWESNGQPENNYKTTPPMLKSDGSFFLYSK
LTVDKSRWQQGNVFSCSVMHEALHNHYTQKSLSLSPGK
73R009 Light chain amino acid sequence with predicted
signal sequence underlined
(SEQ ID NO: 11)
MKHLWFFLLLVAAPRWVLSDIVLTQSPATLSASPGEKVTLTCSASSSVSSSYLYWYQQKP
GQAPKLLIYSTSNLASGVPARFSGSGSGTSYSLTISSLEPEDFATYYCHQWSSYPYTFGG
GTKLEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
73R009 Heavy chain amino acid sequence without
predicted signal sequence
(SEQ ID NO: 12)
QVQLQESGPGLVKPSETLSLTCTVTGTTITASYAWSWIRQPPGKGLEWMGYISYSGGTDY
NPSLKSRITISRDTFKNQFSLKLSSVTAADTATYYCARKGAYWGQGTLVTVSSASTKGPS
VFPLAPCSRSTSESTAALGCLVKDYFPEPVTVSWNSGALTSGVHTFPAVLQSSGLYSLSS
VVTVPSSNFGTQTYTCNVDHKPSNTKVDKTVERKCCVECPPCPAPPVAGPSVFLFPPKPK
DTLMISRTPEVTCVVVDVSHEDPEVQFNWYVDGVEVHNAKTKPREEQFNSTFRVVSVLTV
VHQDWLNGKEYKCKVSNKGLPAPIEKTISKTKGQPREPQVYTLPPSREEMTKNQVSLTCL
VKGFYPSDIAVEWESNGQPENNYKTTPPMLDSDGSFFLYSKLTVDKSRWQQGNVFSCSVM
HEALHNHYTQKSLSLSPGK
73R009 (13A variant) Heavy chain amino acid sequence
without predicted signal sequence
(SEQ ID NO: 13)
QVQLQESGPGLVKPSETLSLTCTVTGTTITASYAWSWIRQPPGKGLEWMGYISYSGGTDY
NPSLKSRITISRDTFKNQFSLKLSSVTAADTATYYCARKGAYWGQGTLVTVSSASTKGPS
VFPLAPCSRSTSESTAALGCLVKDYFPEPVTVSWNSGALTSGVHTFPAVLQSSGLYSLSS
VVTVPSSNFGTQTYTCNVDHKPSNTKVDKTVERKCCVECPPCPAPPVAGPSVFLFPPKPK
DTLMISRTPEVTCVVVDVSHEDPEVQFNWYVDGVEVHNAKTKPREEQFNSTFRVVSVLTV
VHQDWLNGKEYKCKVSNKGLPAPIEKTISKTKGQPREPQVYTLPPSREKMTKNQVSLTCL
VKGFYPSDIAVEWESNGQPENNYKTTPPMLKSDGSFFLYSKLTVDKSRWQQGNVFSCSVM
HEALHNHYTQKSLSLSPGK
73R009 Light chain amino acid sequence without
predicted signal sequence
(SEQ ID NO: 14)
DIVLTQSPATLSASPGEKVTLTCSASSSVSSSYLYWYQQKPGQAPKLLIYSTSNLASGVP
ARFSGSGSGTSYSLTISSLEPEDFATYYCHQWSSYPYTFGGGTKLEIKRTVAAPSVFIFP
PSDEQLKSGTASVVCLLNNFYPREAKVQWKVDNALQSGNSQESVTEQDSKDSTYSLSSTL
TLSKADYEKHKVYACEVTHQGLSSPVTKSFNRGEC
73R009 Heavy chain nucleotide sequence
(SEQ ID NO: 15)
ATGAAGCATCTGTGGTTTTTCCTGCTGCTCGTGGCTGCTCCCCGGTGGGTCCTGTCTCAG
GTCCAATTGCAAGAGTCAGGACCAGGGCTTGTGAAGCCCTCAGAGACTCTGTCACTCACT
TGTACCGTGACCGGAACTACCATCACTGCCTCCTACGCCTGGAGCTGGATCAGGCAGCCT
CCGGGAAAAGGCCTGGAATGGATGGGTTACATCTCCTATTCAGGCGGAACCGACTACAAT
CCTAGCCTGAAGTCTCGCATCACCATTTCACGCGATACCTTCAAGAACCAATTCAGCCTT
AAACTCTCCAGCGTGACCGCTGCAGACACTGCCACCTACTACTGCGCAAGAAAGGGAGCC
TATTGGGGTCAGGGGACCCTTGTGACCGTGAGCTCAGCCTCTACCAAGGGCCCTAGCGTC
TTCCCTCTGGCCCCCTGCTCCCGGTCCACCAGCGAGAGCACAGCCGCCCTGGGCTGCCTG
GTCAAGGACTACTTCCCCGAACCTGTGACAGTGTCCTGGAACTCCGGCGCTCTGACCAGC
GGCGTGCACACCTTCCCAGCTGTCCTCCAGTCCTCCGGACTCTACTCCCTCTCCTCCGTG
GTGACAGTGCCCTCCTCCAACTTCGGCACCCAGACCTACACCTGCAACGTCGATCACAAG
CCCAGCAACACCAAGGTTGATAAGACAGTTGAGCGCAAATGTTGTGTCGAGTGCCCTCCT
TGCCCAGCCCCTCCTGTGGCTGGACCTTCCGTCTTCCTCTTCCCCCCTAAACCCAAAGAC
ACCCTCATGATCTCCCGGACCCCTGAGGTCACATGCGTGGTGGTGGACGTGAGCCACGAA
GACCCCGAGGTCCAGTTCAACTGGTATGTGGACGGCGTGGAGGTGCATAATGCCAAGACA
AAGCCACGGGAGGAGCAGTTCAACAGCACATTCCGGGTGGTCAGCGTCCTCACCGTTGTG
CACCAGGACTGGCTGAACGGCAAGGAGTACAAGTGCAAAGTCTCCAACAAAGGCCTCCCT
GCCCCCATCGAGAAAACCATCTCCAAAACCAAAGGGCAGCCCAGGGAACCACAGGTGTAC
ACCCTGCCCCCTTCCCGGGAGGARATGACCAAGAACCAAGTCAGCCTGACCTGCCTGGTC
AAAGGCTTCTACCCCTCCGACATCGCCGTGGAGTGGGAGAGCAATGGGCAGCCTGAGAAC
AACTACAAGACCACACCTCCCATGCTGGAYTCCGACGGCTCCTTCTTCCTCTACTCCAAA
CTCACCGTGGACAAGAGCAGGTGGCAGCAGGGGAACGTCTTCTCCTGCTCCGTGATGCAT
GAGGCTCTGCACAACCACTACACACAGAAGTCCCTCTCCCTGTCTCCTGGAAAA
Wherein R = A or G
Wherein Y = C or T
73R009 (13A variant) Heavy chain nucleotide sequence
(SEQ ID NO: 16)
ATGAAGCATCTGTGGTTTTTCCTGCTGCTCGTGGCTGCTCCCCGGTGGGTCCTGTCTCAG
GTCCAATTGCAAGAGTCAGGACCAGGGCTTGTGAAGCCCTCAGAGACTCTGTCACTCACT
TGTACCGTGACCGGAACTACCATCACTGCCTCCTACGCCTGGAGCTGGATCAGGCAGCCT
CCGGGAAAAGGCCTGGAATGGATGGGTTACATCTCCTATTCAGGCGGAACCGACTACAAT
CCTAGCCTGAAGTCTCGCATCACCATTTCACGCGATACCTTCAAGAACCAATTCAGCCTT
AAACTCTCCAGCGTGACCGCTGCAGACACTGCCACCTACTACTGCGCAAGAAAGGGAGCC
TATTGGGGTCAGGGGACCCTTGTGACCGTGAGCTCAGCCTCTACCAAGGGCCCTAGCGTC
TTCCCTCTGGCCCCCTGCTCCCGGTCCACCAGCGAGAGCACAGCCGCCCTGGGCTGCCTG
GTCAAGGACTACTTCCCCGAACCTGTGACAGTGTCCTGGAACTCCGGCGCTCTGACCAGC
GGCGTGCACACCTTCCCAGCTGTCCTCCAGTCCTCCGGACTCTACTCCCTCTCCTCCGTG
GTGACAGTGCCCTCCTCCAACTTCGGCACCCAGACCTACACCTGCAACGTCGATCACAAG
CCCAGCAACACCAAGGTTGATAAGACAGTTGAGCGCAAATGTTGTGTCGAGTGCCCTCCT
TGCCCAGCCCCTCCTGTGGCTGGACCTTCCGTCTTCCTCTTCCCCCCTAAACCCAAAGAC
ACCCTCATGATCTCCCGGACCCCTGAGGTCACATGCGTGGTGGTGGACGTGAGCCACGAA
GACCCCGAGGTCCAGTTCAACTGGTATGTGGACGGCGTGGAGGTGCATAATGCCAAGACA
AAGCCACGGGAGGAGCAGTTCAACAGCACATTCCGGGTGGTCAGCGTCCTCACCGTTGTG
CACCAGGACTGGCTGAACGGCAAGGAGTACAAGTGCAAAGTCTCCAACAAAGGCCTCCCT
GCCCCCATCGAGAAAACCATCTCCAAAACCAAAGGGCAGCCCAGGGAACCACAGGTGTAC
ACCCTGCCCCCTTCCCGGGAGAAGATGACCAAGAACCAAGTCAGCCTGACCTGCCTGGTC
AAAGGCTTCTACCCCTCCGACATCGCCGTGGAGTGGGAGAGCAATGGGCAGCCTGAGAAC
AACTACAAGACCACACCTCCCATGCTGAAGTCCGACGGCTCCTTCTTCCTCTACTCCAAA
CTCACCGTGGACAAGAGCAGGTGGCAGCAGGGGAACGTCTTCTCCTGCTCCGTGATGCAT
GAGGCTCTGCACAACCACTACACACAGAAGTCCCTCTCCCTGTCTCCTGGAAAA
73R009 Heavy chain nucleotide sequence without
predicted signal sequence
(SEQ ID NO: 17)
CAGGTCCAATTGCAAGAGTCAGGACCAGGGCTTGTGAAGCCCTCAGAGACTCTGTCACTC
ACTTGTACCGTGACCGGAACTACCATCACTGCCTCCTACGCCTGGAGCTGGATCAGGCAG
CCTCCGGGAAAAGGCCTGGAATGGATGGGTTACATCTCCTATTCAGGCGGAACCGACTAC
AATCCTAGCCTGAAGTCTCGCATCACCATTTCACGCGATACCTTCAAGAACCAATTCAGC
CTTAAACTCTCCAGCGTGACCGCTGCAGACACTGCCACCTACTACTGCGCAAGAAAGGGA
GCCTATTGGGGTCAGGGGACCCTTGTGACCGTGAGCTCAGCCTCTACCAAGGGCCCTAGC
GTCTTCCCTCTGGCCCCCTGCTCCCGGTCCACCAGCGAGAGCACAGCCGCCCTGGGCTGC
CTGGTCAAGGACTACTTCCCCGAACCTGTGACAGTGTCCTGGAACTCCGGCGCTCTGACC
AGCGGCGTGCACACCTTCCCAGCTGTCCTCCAGTCCTCCGGACTCTACTCCCTCTCCTCC
GTGGTGACAGTGCCCTCCTCCAACTTCGGCACCCAGACCTACACCTGCAACGTCGATCAC
AAGCCCAGCAACACCAAGGTTGATAAGACAGTTGAGCGCAAATGTTGTGTCGAGTGCCCT
CCTTGCCCAGCCCCTCCTGTGGCTGGACCTTCCGTCTTCCTCTTCCCCCCTAAACCCAAA
GACACCCTCATGATCTCCCGGACCCCTGAGGTCACATGCGTGGTGGTGGACGTGAGCCAC
GAAGACCCCGAGGTCCAGTTCAACTGGTATGTGGACGGCGTGGAGGTGCATAATGCCAAG
ACAAAGCCACGGGAGGAGCAGTTCAACAGCACATTCCGGGTGGTCAGCGTCCTCACCGTT
GTGCACCAGGACTGGCTGAACGGCAAGGAGTACAAGTGCAAAGTCTCCAACAAAGGCCTC
CCTGCCCCCATCGAGAAAACCATCTCCAAAACCAAAGGGCAGCCCAGGGAACCACAGGTG
TACACCCTGCCCCCTTCCCGGGAGGARATGACCAAGAACCAAGTCAGCCTGACCTGCCTG
GTCAAAGGCTTCTACCCCTCCGACATCGCCGTGGAGTGGGAGAGCAATGGGCAGCCTGAG
AACAACTACAAGACCACACCTCCCATGCTGGAYTCCGACGGCTCCTTCTTCCTCTACTCC
AAACTCACCGTGGACAAGAGCAGGTGGCAGCAGGGGAACGTCTTCTCCTGCTCCGTGATG
CATGAGGCTCTGCACAACCACTACACACAGAAGTCCCTCTCCCTGTCTCCTGGAAAA
Wherein R = A or G
Wherein Y = C or T
73R009 (13A variant) Heavy chain nucleotide sequence
without predicted signal sequence
(SEQ ID NO: 18)
CAGGTCCAATTGCAAGAGTCAGGACCAGGGCTTGTGAAGCCCTCAGAGACTCTGTCACTC
ACTTGTACCGTGACCGGAACTACCATCACTGCCTCCTACGCCTGGAGCTGGATCAGGCAG
CCTCCGGGAAAAGGCCTGGAATGGATGGGTTACATCTCCTATTCAGGCGGAACCGACTAC
AATCCTAGCCTGAAGTCTCGCATCACCATTTCACGCGATACCTTCAAGAACCAATTCAGC
CTTAAACTCTCCAGCGTGACCGCTGCAGACACTGCCACCTACTACTGCGCAAGAAAGGGA
GCCTATTGGGGTCAGGGGACCCTTGTGACCGTGAGCTCAGCCTCTACCAAGGGCCCTAGC
GTCTTCCCTCTGGCCCCCTGCTCCCGGTCCACCAGCGAGAGCACAGCCGCCCTGGGCTGC
CTGGTCAAGGACTACTTCCCCGAACCTGTGACAGTGTCCTGGAACTCCGGCGCTCTGACC
AGCGGCGTGCACACCTTCCCAGCTGTCCTCCAGTCCTCCGGACTCTACTCCCTCTCCTCC
GTGGTGACAGTGCCCTCCTCCAACTTCGGCACCCAGACCTACACCTGCAACGTCGATCAC
AAGCCCAGCAACACCAAGGTTGATAAGACAGTTGAGCGCAAATGTTGTGTCGAGTGCCCT
CCTTGCCCAGCCCCTCCTGTGGCTGGACCTTCCGTCTTCCTOTTCCCCCCTAAACCCAAA
GACACCCTCATGATCTCCCGGACCCCTGAGGTCACATGCGTGGTGGTGGACGTGAGCCAC
GAAGACCCCGAGGTCCAGTTCAACTGGTATGTGGACGGCGTGGAGGTGCATAATGCCAAG
ACAAAGCCACGGGAGGAGCAGTTCAACAGCACATTCCGGGTGGTCAGCGTCCTCACCGTT
GTGCACCAGGACTGGCTGAACGGCAAGGAGTACAAGTGCAAAGTCTCCAACAAAGGCCTC
CCTGCCCCCATCGAGAAAACCATCTCCAAAACCAAAGGGCAGCCCAGGGAACCACAGGTG
TACACCCTGCCCCCTTCCCGGGAGAAGATGACCAAGAACCAAGTCAGCCTGACCTGCCTG
GTCAAAGGCTTCTACCCCTCCGACATCGCCGTGGAGTGGGAGAGCAATGGGCAGCCTGAG
AACAACTACAAGACCACACCTCCCATGCTGAAGTCCGACGGCTCCTTCTTCCTCTACTCC
AAACTCACCGTGGACAAGAGCAGGTGGCAGCAGGGGAACGTOTTCTCCTGCTCCGTGATG
CATGAGGCTCTGCACAACCACTACACACAGAAGTCCCTCTCCCTGTCTCCTGGAAAA
73R009 Light chain nucleotide sequence
(SEQ ID NO: 19)
ATGAAGCACCTCTGGTTCTTCCTTCTTCTTGTGGCCGCTCCCCGCTGGGTCCTCAGCGAT
ATCGTGCTGACCCAGTCACCCGCCACCCTCTCAGCTTCACCTGGCGAGAAGGTCACTCTG
ACTTGCTCTGCCTCATCTAGCGTGTCATCTTCATATCTGTACTGGTATCAGCAAAAACCG
GGACAAGCCCCGAAGCTCCTGATCTACAGCACCAGCAACCTTGCATCCGGAGTGCCTGCC
AGGTTTAGCGGGTCCGGGTCCGGTACCTCATATTCACTGACCATTTCTTCTCTTGAACCC
GAAGATTTCGCTACCTACTACTGTCATCAGTGGTCTAGCTACCCATACACTTTCGGCGGA
GGAACCAAACTGGAGATTAAGCGTACGGTGGCAGCCCCTTCTGTCTTTATCTTCCCTCCA
TCCGACGAGCAGCTCAAATCAGGAACCGCTTCTGTCGTGTGCCTGCTTAACAATTTCTAC
CCACGGGAAGCCAAGGTGCAGTGGAAGGTGGACAATGCCCTGCAATCAGGTAATTCCCAA
GAGTCAGTGACTGAACAGGATAGCAAGGACAGCACCTATTCACTCTCCAGCACTCTGACC
CTGTCCAAGGCTGACTACGAAAAGCATAAGGTGTACGCATGCGAGGTGACCCACCAGGGT
CTGAGCAGCCCCGTCACCAAGTCTTTCAACAGAGGGGAGTGT
73R009 Light chain nucleotide sequence without
predicted signal sequence
(SEQ ID NO: 20)
GATATCGTGCTGACCCAGTCACCCGCCACCCTCTCAGCTTCACCTGGCGAGAAGGTCACT
CTGACTTGCTCTGCCTCATCTAGCGTGTCATCTTCATATCTGTACTGGTATCAGCAAAAA
CCGGGACAAGCCCCGAAGCTCCTGATCTACAGCACCAGCAACCTTGCATCCGGAGTGCCT
GCCAGGTTTAGCGGGTCCGGGTCCGGTACCTCATATTCACTGACCATTTCTTCTCTTGAA
CCCGAAGATTTCGCTACCTACTACTGTCATCAGTGGTCTAGCTACCCATACACTTTCGGC
GGAGGAACCAAACTGGAGATTAAGCGTACGGTGGCAGCCCCTTCTGTCTTTATCTTCCCT
CCATCCGACGAGCAGCTCAAATCAGGAACCGCTTCTGTCGTGTGCCTGCTTAACAATTTC
TACCCACGGGAAGCCAAGGTGCAGTGGAAGGTGGACAATGCCCTGCAATCAGGTAATTCC
CAAGAGTCAGTGACTGAACAGGATAGCAAGGACAGCACCTATTCACTCTCCAGCACTCTG
ACCCTGTCCAAGGCTGACTACGAAAAGCATAAGGTGTACGCATGCGAGGTGACCCACCAG
GGTCTGAGCAGCCCCGTCACCAAGTCTTTCAACAGAGGGGAGTGT
Human FZD1 Fri domain amino acid sequence
(SEQ ID NO: 21)
QQPPPPPQQQQSGQQYNGERGISVPDHGYCQPISIPLCTDIAYNQTIMPNLLGHTNQEDA
GLEVHQFYPLVKVQCSAELKFFLCSMYAPVCTVLEQALPPCRSLCERARQGCEALMNKFG
FQWPDTLKCEKFPVHGAGELCVGQNTSDKGT
Human FZD2 Fri domain amino acid sequence
(SEQ ID NO: 22)
QFHGEKGISIPDHGFCQPISIPLCTDIAYNQTIMPNLLGHTNQEDAGLEVHQFYPLVKVQ
CSPELRFFLCSMYAPVCTVLEQAIPPCRSICERARQGCEALMNKFGFQWPERLRCEHFPR
HGAEQICVGQNHSEDG
Human FZD3 Fri domain amino acid sequence
(SEQ ID NO: 23)
HSLFSCEPITLRMCQDLPYNTTFMPNLLNHYDQQTAALAMEPFHPMVNLDCSRDF
RPFLCALYAPICMEYGRVTLPCRRLCQRAYSECSKLMEMFGVPWPEDMECSRFPDCDEPY
PRLVDL
Human FZD4 Fri domain amino acid sequence
(SEQ ID NO: 24)
FGDEEERRCDPIRISMCQNLGYNVTKMPNLVGHELQTDAELQLTTFTPLIQYGCSSQLQF
FLCSVYVPMCTEKINIPIGPCGGMCLSVKRRCEPVLKEFGFAWPESLNCSKFPPQNDHNH
MCMEGPGDEEV
Human FZD5 Fri domain amino acid sequence
(SEQ ID NO: 25)
ASKAPVCQEITVPMCRGIGYNLTHMPNQFNHDTQDEAGLEVHQFWPLVEIQCSPDLRFFL
CSMYTPICLPDYHKPLPPCRSVCERAKAGCSPLMRQYGFAWPERMSCDRLPVLGRDAEVL
CMDYNRSEATT
Human FZD6 Fri domain amino acid sequence
(SEQ ID NO: 26)
HSLFTCEPITVPRCMKMAYNMTFFPNLMGHYDQSIAAVEMEHFLPLANLECSPNIETFLC
KAFVPTCIEQIHVVPPCRKLCEKVYSDCKKLIDTFGIRWPEELECDRLQYCDETVPVTFD
PHTEFLG
Human FZD7 Fri domain amino acid sequence
(SEQ ID NO: 27)
QPYHGEKGISVPDHGFCQPISIPLCTDIAYNQTILPNLLGHTNQEDAGLEVHQFYPLVKV
QCSPELRFFLCSMYAPVCTVLDQAIPPCRSLCERARQGCEALMNKFGFQWPERLRCENFP
VHGAGEICVGQNTSDGSG
Human FZD8 Fri domain amino acid sequence
(SEQ ID NO: 28)
ASAKELACQEITVPLCKGIGYNYTMPNQFNHDTQDEAGLEVHQFWPLVEIQCSPDLYKFF
LCSMYTPICLEDYKKPLPPCRSVCERAKAGCAPLMRQYGFAWPDRMRCDRLPEQGNPDTL
CMDYNRTDLTT
Human FZD8 Fri domain amino acid sequence (variant)
(SEQ ID NO: 29)
ASAKELACQEITVPLCKGIGYNYTYMPNQFNHDTQDEAGLEVHQFWPLVEIQCSPDLKFF
LCSMYTPICLEDYKKPLPPCRSVCERAKAGCAPLMRQYGFAWPDRMRCDRLPEQGNPDTL
CMDYNRTDL
Human FZD9 Fri domain amino acid sequence
(SEQ ID NO: 30)
LEIGRFDPERGRGAAPCQAVEIPMCRGIGYNLTRMPNLLGHTSQGEAAAELAEFAPLVQY
GCHSHLRFFLCSLYAPMCTDQVSTPIPACRPMCEQARLRCAPIMEQFNFGWPDSLDCARL
PTRNDPHALCMEAPENA
Human FZD10 Fri domain amino acid sequence
(SEQ ID NO: 31)
ISSMDMERPGDGKCQPIEIPMCKDIGYNMTRMPNLMGHENQREAAIQLHEFAPLVEYGCH
GHLRFFLCSLYAPMCTEQVSTPIPACRVMCEQARLKCSPIMEQFNFKWPDSLDCRKLPNK
NDPNYLCMEAPNNG
Human FZD1 amino acids 116-227
(SEQ ID NO: 32)
CQPISIPLCTDIAYNQTIMPNLLGHTNQEDAGLEVHQFYPLVKVQCSAELKFFLCSMYAP
VCTVLEQALPPCRSLCERARQGCEALMNKFGFQWPDTLKCEKFPVHGAGELC
Human FZD2 amino acids 39-150
(SEQ ID NO: 33)
CQPISIPLCTDIAYNQTIMPNLLGHTNQEDAGLEVHQFYPLVKVQCSPELRFFLCSMYAP
VCTVLEQAIPPCRSICERARQGCEALMNKFGFQWPERLRCEHFPRHGAEQIC
Human FZD3 amino acids 28-133
(SEQ ID NO: 34)
CEPITLRMCQDLPYNTTFMPNLLNHYDQQTAALAMEPFHPMVNLDCSRDFRPFLCALYAP
ICMEYGRVTLPCRRLCQRAYSECSKLMEMFGVPWPEDMECSRFPDC
Human FZD4 amino acids 48-161
(SEQ ID NO: 35)
CDPIRISMCQNLGYNVTKMPNLVGHELQTDAELQLTTFTPLIQYGCSSQLQFFLCSVYVP
MCTEKINIPIGPCGGMCLSVKRRCEPVLKEFGFAWPESLNCSKFPPQNDHNHMC
Human FZD5 amino acids 33-147
(SEQ ID NO: 36)
CQEITVPMCRGIGYNLTHMPNQFNHDTQDEAGLEVHQFWPLVEIQCSPDLRFFLCSMYTP
ICLPDYHKPLPPCRSVCERAKAGCSPLMRQYGFAWPERMSCDRLPVLGRDAEVLC
Human FZD6 amino acids 24-129
(SEQ ID NO: 37)
CEPITVPRCMKMAYNMTFFPNLMGHYDQSIAAVEMEHFLPLANLECSPNIETFLCKAFVP
TCIEQIHVVPPCRKLCEKVYSDCKKLIDTFGIRWPEELECDRLQYC
Human FZD7 amino acids 49-160
(SEQ ID NO: 38)
CQPISIPLCTDIAYNQTILPNLLGHTNQEDAGLEVHQFYPLVKVQCSPELRFFLCSMYAP
VCTVLDQAIPPCRSLCERARQGCEALMNKFGFQWPERLRCENFPVHGAGEIC
Human FZD8 amino acids 35-148
(SEQ ID NO: 39)
CQEITVPLCKGIGYNYTYMPNQFNHDTQDEAGLEVHQFWPLVEIQCSPDLKFFLCSMYTP
ICLEDYKKPLPPCRSVCERAKAGCAPLMRQYGFAWPDRMRCDRLPEQGNPDTLC
Human FZD9 amino acids 39-152
(SEQ ID NO: 40)
CQAVEIPMCRGIGYNLTRMPNLLGHTSQGEAAAELAEFAPLVQYGCHSHLRFFLCSLYAP
MCTDQVSTPIPACRPMCEQARLRCAPIMEQFNFGWPDSLDCARLPTRNDPHALC
Human FZD10 amino acids 34-147
(SEQ ID NO: 41)
CQPIEIPMCKDIGYNMTRMPNLMGHENQREAAIQLHEFAPLVEYGCHGHLRFFLCSLYAP
MCTEQVSTPIPACRVMCEQARLKCSPIMEQFNFKWPDSLDCRKLPNKNDPNYLC
Human IgG₁ Fc region
(SEQ ID NO: 42)
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
Human IgG₁ Fc region (variant)
(SEQ ID NO: 43)
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
Human IgG₂ Fc region
(SEQ ID NO: 44)
CVECPPCPAPPVAGPSVFLFPPKPKDTLMISRTPEVTCVVVDVSHEDPEVQFNWYVDGVE
VHNAKTKPREEQFNSTFRVVSVLTVVHQDWLNGKEYKCKVSNKGLPAPIEKTISKTKGQP
REPQVYTLPPSREEMTKNQVSLTCLVKGFYPSDIAVEWESNGQPENNYKTTPPMLDSDGS
FFLYSKLTVDKSRWQQGNVFSCSVMHEALHNHYTQKSLSLSPGK
Human IgG₂ Fc region
(SEQ ID NO: 45)
TKVDKTVERKCCVECPPCPAPPVAGPSVFLFPPKPKDTLMISRTPEVTCVVVDVSHEDPE
VQFNWYVDGVEVHNAKTKPREEQFNSTFRVVSVLTVVHQDWLNGKEYKCKVSNKGLPAPI
EKTISKTKGQPREPQVYTLPPSREEMTKNQVSLTCLVKGFYPSDIAVEWESNGQPENNYK
TTPPMLDSDGSFFLYSKLTVDKSRWQQGNVFSCSVMHEALHNHYTQKSLSLSPGK
Human IgG₂ Fc region variant
(SEQ ID NO: 46)
TKVDKTVERKSCVECPPCPAPPVAGPSVFLFPPKPKDTLMISRTPEVTCVVVDVSHEDPE
VQFNWYVDGVEVHNAKTKPREEQFNSTFRVVSVLTVVHQDWLNGKEYKCKVSNKGLPAPI
EKTISKTKGQPREPQVYTLPPSREEMTKNQVSLTCLVKGFYPSDIAVEWESNGQPENNYK
TTPPMLDSDGSFFLYSKLTVDKSRWQQGNVFSCSVMHEALHNHYTQKSLSLSPGK
Human IgG₂ Fc region (Variant 13A)
(SEQ ID NO: 47)
CVECPPCPAPPVAGPSVFLFPPKPKDTLMISRTPEVTCVVVDVSHEDPEVQFNWYVDGVE
VHNAKTKPREEQFNSTFRVVSVLTVVHQDWLNGKEYKCKVSNKGLPAPIEKTISKTKGQP
REPQVYTLPPSREKMTKNQVSLTCLVKGFYPSDIAVEWESNGQPENNYKTTPPMLKSDGS
FFLYSKLTVDKSRWQQGNVFSCSVMHEALHNHYTQKSLSLSPGK
Human IgG₂ Fc region (Variant 13B)
(SEQ ID NO: 48)
CVECPPCPAPPVAGPSVFLFPPKPKDTLMISRTPEVTCVVVDVSHEDPEVQFNWYVDGVE
VHNAKTKPREEQFNSTFRVVSVLTVVHQDWLNGKEYKCKVSNKGLPAPIEKTISKTKGQP
REPQVYTLPPSREEMTKNQVSLTCLVEGFYPSDIAVEWESNGQPENNYKTTPPMLDSDGS
FFLYSELTVDKSRWQQGNVFSCSVMHEALHNHYTQKSLSLSPGK
Human IgG₂ Fc region (Variant 13A)
(SEQ ID NO: 49)
TKVDKTVERKCCVECPPCPAPPVAGPSVFLFPPKPKDTLMISRTPEVTCVVVDVSHEDPE
VQFNWYVDGVEVHNAKTKPREEQFNSTFRVVSVLTVVHQDWLNGKEYKCKVSNKGLPAPI
EKTISKTKGQPREPQVYTLPPSREKMTKNQVSLTCLVKGFYPSDIAVEWESNGQPENNYK
TTPPMLKSDGSFFLYSKLTVDKSRWQQGNVFSCSVMHEALHNHYTQKSLSLSPGK
Human IgG₂ Fc region variant (Variant 13A)
(SEQ ID NO: 50)
TKVDKTVERKSCVECPPCPAPPVAGPSVFLFPPKPKDTLMISRTPEVTCVVVDVSHEDPE
VQFNWYVDGVEVHNAKTKPREEQFNSTFRVVSVLTVVHQDWLNGKEYKCKVSNKGLPAPI
EKTISKTKGQPREPQVYTLPPSREKMTKNQVSLTCLVKGFYPSDIAVEWESNGQPENNYK
TTPPMLKSDGSFFLYSKLTVDKSRWQQGNVFSCSVMHEALHNHYTQKSLSLSPGK
Human IgG₂ Fc region (Variant 13B)
(SEQ ID NO: 51)
TKVDKTVERKCCVECPPCPAPPVAGPSVFLFPPKPKDTLMISRTPEVTCVVVDVSHEDPE
VQFNWYVDGVEVHNAKTKPREEQFNSTFRVVSVLTVVHQDWLNGKEYKCKVSNKGLPAPI
EKTISKTKGQPREPQVYTLPPSREEMTKNQVSLTCLVEGFYPSDIAVEWESNGQPENNYK
TTPPMLDSDGSFFLYSELTVDKSRWQQGNVFSCSVMHEALHNHYTQKSLSLSPGK
Human IgG₂ Fc region variant (Variant 13B)
(SEQ ID NO: 52)
TKVDKTVERKSCVECPPCPAPPVAGPSVFLFPPKPKDTLMISRTPEVTCVVVDVSHEDPE
VQFNWYVDGVEVHNAKTKPREEQFNSTFRVVSVLTVVHQDWLNGKEYKCKVSNKGLPAPI
EKTISKTKGQPREPQVYTLPPSREEMTKNQVSLTCLVEGFYPSDIAVEWESNGQPENNYK
TTPPMLDSDGSFFLYSELTVDKSRWQQGNVFSCSVMHEALHNHYTQKSLSLSPGK
FZD8-Fc variant 54F28 amino acid sequence
(without predicted signal sequence)
(SEQ ID NO: 53)
ASAKELACQEITVPLCKGIGYNYTYMPNQFNHDTQDEAGLEVHQFWPLVEIQCSPDLKFF
LCSMYTPICLEDYKKPLPPCRSVCERAKAGCAPLMRQYGFAWPDRMRCDRLPEQGNPDTL
CMDYNRTDLTTEPKSSDKTHTCPPCPAPELLGGPSVFIFPPKPKDTLMISRTPEVTCVVV
DVSHEDPEVKFNWYVDGVEVHNAKTKPREEQYNSTYRVVSVLTVLHQDWLNGKEYKCKVS
NKALPAPIEKTISKAKGQPREPQVYTLppsRDELTKNQVSLTCLVKGFYPSDIAVEWESN
GQPENNYKTTPPVLDSDGSFFLYSKLTVDKSRWQQGNVFSCSVMHEALHNHYTQKSLSLS
PGK
FZD8-Fc variant 54F28 amino acid sequence with
signal sequence
(SEQ ID NO: 54)
MEWGYLLEVTSLLAALLLLQRSPFVHAASAKELACQETTVPLCKGIGYNYTYMPNQFNHD
TQDEAGLEVHQFWPLVEIQCSPDLKFFLCSMYTPICLEDYKKPLPPCRSVCERAKAGCAP
LMRQYGFAWPDRMRCDRLPEQGNPDTLCMDYNRTDLTTEPKSSDKTHTCPPCPAPELLGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPGK
FZD84c variant (13B variant) amino acid sequence
with signal sequence
(SEQ ID NO: 55)
MEWGYLLEVTSLLAALLLLQRSPIVHAASAKELACQEITVPLCKGIGYNYTYMPNQFNHD
TQDEAGLEVHQFWPLVEIQCSPDLKFFLCSMYTPICLEDYKKPLPPCRSVCERAKAGCAP
LMRQYGFAWPDRMRCDRLPEQGNPDTLCMDYNRTDLTTTKVDKTVERKSCVECPPCPAPP
VAGPSVFLFPPKPKDTLMISRTPEVTCVVVDVSHEDPEVQFNWYVDGVEVHNAKTKPREE
QFNSTFRVVSVLTVVHQDWLNGKEYKCKVSNKGLPAPIEKTISKTKGQPREPQVYTLPPS
REEMTKNQVSLTCLVEGFYPSDIAVEWESNGQPENNYKTTPPMLDSDGSFFLYSELTVDK
SRWQQGNVESCSVMHEALHNHYTQKSLSLSPGK
FZD8-Fc variant (13B variant) amino acid sequence
without signal sequence
(SEQ ID NO: 56)
ASAKELACQEITVPLCKGIGYNYTYMPNQFNHDTQDEAGLEVHQFWPLVEIQCSPDLKFF
LCSMYTPICLEDYKKPLPPCRSVCERAKAGCAPLMRQYGFAWPDRMRCDRLPEQGNPDTL
CMDYNRTDLTTTKVDKTVERKSCVECPPCPAPPVAGPSVFLFPPKPKDTLMISRTPEVTC
VVVDVSHEDPEVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVEGFYPSDIAVEW
ESNGQPENNYKTTPPMLDSDGSFFLYSELTVDKSRWQQGNVFSCSVMHEALHNHYTQKSL
SLSPGK
Human WNT1 C-terminal cysteine rich domain (aa 288-370)
(SEQ ID NO: 57)
DLVYFEKSPNFCTYSGRLGTAGTAGRACNSSSPALDGCELLCCGRGHRTRTQRVTERCNC
TFHWCCHVSCRNCTHTRVLHECL
Human WNT2 C-terminal cysteine rich domain (aa 267-360)
(SEQ ID NO: 58)
DLVYFENSPDYCIRDREAGSLGTAGRVCNLTSRGMDSCEVMCCGRGYDTSHVTRMTKCGC
KFHWCCAVRCQDCLEALDVHTCKAPKNADWTTAT
Human Wnt2b C-terminal cysteine rich domain (aa 298-391)
(SEQ ID NO: 59)
DLVYFDNSPDYCVLDKAAGSLGTAGRVCSKTSKGTDGCEIMCCGRGYDTTRVTRVTQCEC
KFHWCCAVRCKECRNTVDVHTCKAPKKAEWLDQT
Human WNT3 C-terminal cysteine rich domain (aa 273-355)
(SEQ ID NO: 60)
DLVYYENSPNFCEPNPETGSFGTRDRTCNVTSHGIDGCDLLCCGRGHNTRTEKRKEKCHC
IFHWCCYVSCQECIRIYDVHTCK
Human WNT3a C-terminal cysteine rich domain (aa 270-352)
(SEQ ID NO: 61)
DLVYYEASPNFCEPNPETGSFGTRDRTCNVSSHGIDGCDLLCCGRGHNARAERRREKCRC
VFHWCCYVSCQECTRVYDVHTCK
Human WNT7a C-terminal cysteine rich domain (aa 267-359)
(SEQ ID NO: 62)
DLVYIEKSPNYCEEDPVTGSVGTQGRACNKTAPQASGCDLMCCGRGYNTHQYARVWQCNC
KFHWCCYVKCNTCSERTEMYTCK
Human WNT7b C-terminal cysteine rich domain (aa 267-349)
(SEQ ID NO: 63)
DLVYIEKSPNYCEEDAATGSVGTQGRLCNRTSPGADGCDTMCCGRGYNTHQYTKVWQCNC
KFHWCCFVKCNTCSERTEVFTCK
Human WNT8a C-terminal cysteine rich domain (aa 248-355)
(SEQ ID NO: 64)
ELIFLEESPDYCTCNSSLGIYGTEGRECLQNSHNTSRWERRSCGRLCTECGLQVEERKTE
VISSCNCKFQWCCTVKCDQCRHVVSKYYCARSPGSAQSLGRVWFGVYI
Human WNT8b C-terminal cysteine rich domain (aa 245-351)
(SEQ ID NO: 65)
ELVHLEDSPDYCLENKTLGLLGTEGRECLRRGRALGRWELRSCRRLCGDCGLAVEERRAE
TVSSCNCKFHWCCAVRCEQCRRRVTKYFCSRAERPRGGAAHKPGRKP
Human WNT10a C-terminal cysteine rich domain (aa 335-417)
(SEQ ID NO: 66)
DLVYFEKSPDFCEREPRLDSAGTVGRLCNKSSAGSDGCGSMCCGRGHNILRQTRSERCHC
RFHWCCFVVCEECRITEWVSVCK
Human WNT10b C-terminal cysteine rich domain (aa 307-389)
(SEQ ID NO: 67)
ELVYFEKSPDFCERDPTMGSPGTRGRACNKTSRLLDGCGSLCCGRGHNVLRQTRVERCHC
RFHWCCYVLCDECKVTEWVNVCK
Linker
(SEQ ID NO: 68)
ESGGGGVT
Linker
(SEQ ID NO: 69)
LESGGGGVT
Linker
(SEQ ID NO: 70)
GRAQVT
Linker
(SEQ ID NO: 71)
WRAQVT
Linker
(SEQ ID NO: 72)
ARGRAQVT
FLAG peptide
(SEQ ID NO: 73)
DYKDDDDK
Human IgG1 Heavy chain constant region
(SEQ ID NO: 74)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPGK
Human IgG2 Heavy chain constant region
(SEQ ID NO: 75)
ASTKGPSVFPLAPCSRSTSESTAALGCLVKDYFPEPVTVSWNSGALTSGVHTFPAVLQSS
GLYSLSSVVTVPSSNFGTQTYTCNVDHKPSNTKVDKTVERKCCVECPPCPAPPVAGPSVF
LFPPKPKDTLMISRTPEVTCVVVDVSHEDPEVQFNWYVDGVEVHNAKTKPREEQFNSTFR
VVSVLTVVHQDWLNGKEYKCKVSNKGLPAPIEKTISKTKGQPREPQVYTLPPSREEMTKN
QVSLTCLVKGFYPSDIAVEWESNGQPENNYKTTPPMLDSDGSFFLYSKLTVDKSRWQQGN
VFSCSVMHEALHNHYTQKSLSLSPGK
Human IgG3 Heavy chain constant region
(SEQ ID NO:76)
ASTKGPSVFPLAPCSRSTSGGTAALGCLVKDYFPEPVTVSWNSGALTSGVHTFPAVLQSS
GLYSLSSVVTVPSSSLGTQTYTCNVNHKPSNTKVDKRVELKTPLGDTTHTCPRCPEPKSC
DTPPPCPRCPEPKSCDTPPPCPRCPEPKSCDTPPPCPRCPAPELLGGPSVFLFPPKPKDT
LMISRTPEVTCVVVDVSHEDPEVQFKWYVDGVEVHNAKTKPREEQYNSTFRVVSVLTVLH
QDWLNGKEYKCKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQGNIFSCSVMHE
ALHNRFTQKSLSLSPGK
Human IgG4 Heavy chain constant region
(SEQ ID NO: 77)
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPSCPAPEFLGGPSV
FLFPPKPKDTLMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSRLTVDKSRWQEG
NVFSCSVMHEALHNHYTQKSLSLSLGK
MET antibody Heavy chain CDR1
(SEQ ID NO: 78)
GYTFTSYWLH
MET antibody Heavy chain CDR2
(SEQ ID NO: 79)
GMIDPSNSDTRFNPNFKD
MET Heavy chain CDR3
(SEQ ID NO: 80)
XYGSYVSPLDY
wherein X is not R
MET Heavy chain CDR3
(SEQ ID NO: 81)
TYGSYVSPLDY
MET Heavy chain CDR3
(SEQ ID NO: 82)
SYGSYVSPLDY
MET Heavy chain CDR3
(SEQ ID NO: 83)
ATYGSYVSPLDY
MET Light chain CDR1
(SEQ ID NO: 84)
KSSQSLLYTSSQKNYLA
MET Light chain CDR2
(SEQ ID NO: 85)
WASTRES
MET Light chain CDR3
(SEQ ID NO: 86)
QQYYAYPWT
FZD8-Fc variant (13A variant) amino acid sequence
without signal sequence
(SEQ ID NO: 87)
ASAKELACQEITVPLCKGIGYNYTYMPNQFNHDTQDEAGLEVHQFWPLVEIQCSPDLKFF
LCSMYTPICLEDYKKPLPPCRSVCERAKAGCAPLMRQYGFAWPDRMRCDRLPEQGNPDTL
CMDYNRTDLTTTKVDKTVERKSCVECPPCPAPPVAGPSVFLFPPKPKDTLMISRTPEVTC
VVVDVSHEDPEVQFNWYVDGVEVHNAKTKPREEQFNSTFRVVSVLTVVHQDWLNGKEYKC
KVSNKGLPAPIEKTISKTKGQPREPQVYTLPPSREKMTKNQVSLTCLVKGFYPSDIAVEW
ESNGQPENNYKTTPPMLKSDGSFFLYSKLTVDKSRWQQGNVFSCSVMHEALHNHYTQKSL
SLSPGK
73R009 (13B variant) Heavy chain amino acid sequence
without predicted signal sequence
(SEQ ID NO: 88)
QVQLQESGPGLVKPSETLSLTCTVTGTTITASYAWSWIRQPPGKGLEWMGYISYSGGTDY
NPSLKSRITISRDTFKNQFSLKLSSVTAADTATYYCARKGAYWGQGTLVTVSSASTKGPS
VFPLAPCSRSTSESTAALGCLVKDYFPEPVTVSWNSGALTSGVHTFPAVLQSSGLYSLSS
VVTVPSSNFGTQTYTCNVDHKPSNTKVDKTVERKCCVECPPCPAPPVAGPSVFLFPPKPK
DTLMISRTPEVTCVVVDVSHEDPEVQFNWYVDGVEVHNAKTKPREEQFNSTFRVVSVLTV
VHQDWLNGKEYKCKVSNKGLPAPIEKTISKTKGQPREPQVYTLPPSREEMTKNQVSLTCL
VEGFYPSDIAVEWESNGQPENNYKTTPPMLDSDGSFFLYSELTVDKSRWQQGNVFSCSVM
HEALHNHYTQKSLSLSPGK
FZDS-Fc variant (13B variant) nucleotide sequence
with signal sequence
(SEQ ID NO: 89)
ATGGAGTGGGGTTATCTTTTAGAAGTGACCTCGCTGCTAGCCGCCTTGCTACTGCTGCAG
CGCTCTCCGATCGTGCACGCCGCCTCGGCCAAGGAGCTGGCATGCCAAGAGATCACCGTG
CCGCTATGCAAGGGCATCGGCTACAACTACACCTACATGCCCAATCAATTCAACCACGAC
ACGCAAGACGAGGCGGGCCTGGAGGTGCACCAGTTCTGGCCGCTGGTGGAGATCCAGTGC
TCGCCCGATCTCAAGTTCTTCCTGTGCAGCATGTACACGCCCATCTGCCTAGAGGACTAC
AAGAAGCCGCTGCCGCCCTGCCGCTCGGTGTGCGAGCGCGCCAAGGCCGGCTGCGCGCCG
CTCATGCGCCAGTACGGCTTCGCCTGGCCCGACCGCATGCGCTGCGACCGGCTGCCCGAG
CAAGGCAACCCTGACACGCTGTGCATGGACTACAACCGCACCGACCTAACCACCACCAAA
GTTGACAAGACTGTTGAGCGAAAGAGCTGCGTTGAGTGCCCTCCATGTCCTGCACCTCCT
GTGGCTGGCCCTTCTGTGTTCCTGTTCCCTCCAAAACCTAAAGACACTCTAATGATCTCT
CGGACTCCTGAGGTGACTTGCGTGGTTGTGGACGTGTCCCACGAGGACCCTGAGGTGCAG
TTTAATTGGTACGTGGACGGAGTCGAGGTGCACAATGCAAAGACCAAGCCTCGGGAGGAA
CAGTTCAACTCCACCTTCCGGGTGGTTTCTGTGTTGACCGTTGTGCACCAAGACTGGCTG
AACGGCAAAGAATACAAGTGCAAGGTGTCCAACAAGGGCCTGCCTGCCCCTATCGAAAAG
ACCATCAGCAAGACCAAGGGCCAGCCTCGCGAGCCTCAGGTGTACACCCTGCCTCCCAGC
CGGGAAGAAATGACCAAGAACCAGGTGTCCCTGACCTGTCTGGTGGAGGGCTTCTACCCT
TCCGACATCGCCGTTGAGTGGGAGTCTAACGGACAGCCGGAGAACAACTACAAGACTACG
CCTCCAATGCTGGACTCCGACGGCTCCTTCTTCCTGTACTCCGAACTGACCGTGGACAAG
TCCCGGTGGCAGCAGGGCAACGTGTTCTCATGCTCCGTAATGCACGAAGCCTTACACAAT
CACTACACTCAAAAGTCCCTATCCTTATCTCCTGGCAAGTAG
FZD8-Fc variant (13B variant) nucleotide sequence
without signal sequence
(SEQ ID No: 90)
CGCTCTCCGATCGTGCACGCCGCCTCGGCCAAGGAGCTGGCATGCCAAGAGATCACCGTG
CCGCTATGCAAGGGCATCGGCTACAACTACACCTACATGCCCAATCAATTCAACCACGAC
ACGCAAGACGAGGCGGGCCTGGAGGTGCACCAGTTCTGGCCGCTGGTGGAGATCCAGTGC
TCGCCCGATCTCAAGTTCTTCCTGTGCAGCATGTACACGCCCATCTGCCTAGAGGACTAC
AAGAAGCCGCTGCCGCCCTGCCGCTCGGTGTGCGAGCGCGCCAAGGCCGGCTGCGCGCCG
CTCATGCGCCAGTACGGCTTCGCCTGGCCCGACCGCATGCGCTGCGACCGGCTGCCCGAG
CAAGGCAACCCTGACACGCTGTGCATGGACTACAACCGCACCGACCTAACCACCACCAAA
GTTGACAAGACTGTTGAGCGAAAGAGCTGCGTTGAGTGCCCTCCATGTCCTGCACCTCCT
GTGGCTGGCCCTTCTGTGTTCCTGTTCCCTCCAAAACCTAAAGACACTCTAATGATCTCT
CGGACTCCTGAGGTGACTTGCGTGGTTGTGGACGTGTCCCACGAGGACCCTGAGGTGCAG
TTTAATTGGTACGTGGACGGAGTCGAGGTGCACAATGCAAAGACCAAGCCTCGGGAGGAA
CAGTTCAACTCCACCTTCCGGGTGGTTTCTGTGTTGACCGTTGTGCACCAAGACTGGCTG
AACGGCAAAGAATACAAGTGCAAGGTGTCCAACAAGGGCCTGCCTGCCCCTATCGAAAAG
ACCATCAGCAAGACCAAGGGCCAGCCTCGCGAGCCTCAGGTGTACACCCTGCCTCCCAGC
CGGGAAGAAATGACCAAGAACCAGGTGTCCCTGACCTGTCTGGTGGAGGGCTTCTACCCT
TCCGACATCGCCGTTGAGTGGGAGTCTAACGGACAGCCGGAGAACAACTACAAGACTACG
CCTCCAATGCTGGACTCCGACGGCTCCTTCTTCCTGTACTCCGAACTGACCGTGGACAAG
TCCCGGTGGCAGCAGGGCAACGTGTTCTCATGCTCCGTAATGCACGAAGCCTTACACAAT
CACTACACTCAAAAGTCCCTATCCTTATCTCCTGGCAAGTAG
Human IgG₁ Fc region
(SEQ ID NO: 91)
KSSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
Human IgG₁ Fc region
(SEQ ID NO: 92)
EPKSSDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSPGK
Human MET
(SEQ ID NO: 93)
MKAPAVLAPGILVLLFTLVQRSNGECKEALAKSEMNVNMKYQLPNFTAETPIQNVILHEH
HIFLGATNYIYVLNEEDLQKVAEYKTGPVLEHPDCFPCQDCSSKANLSGGVWKDNINMAL
VVDTYYDDQLISCGSVNRGTCQRHVFPHNHTADIQSEVHCIFSPQIEEPSQCPDCVVSAL
GAKVLSSVKDRFINFFVGNTINSSYFPDHPLHSISVRRLKETKDGFMFLTDQSYIDVLPE
FRDSYPIKYVHAFESNNFIYFLTVQRETLDAQTFHTRIIRFCSINSGLHSYMEMPLECIL
TEKRKKRSTKKEVFNILQAAYVSKPGAQLARQIGASLNDDILFGVFAQSKPDSAEPMDRS
AMCAFPIKYVNDFFNKIVNKNNVRCLQHFYGPNHEHCFNRTLLRNSSGCEARRDEYRTEF
TTALQRVDLFMGQFSEVLLTSISTFIKGDLTIANLGTSEGRFMQVVVSRSGPSTPHVNFL
LDSHPVSPEVIVEHTLNQNGYTLVITGKKITKIPLNGLGCRHFQSCSQCLSAPPFVQCGW
CHDKCVRSEECLSGTWTQQICLPAIYKVFPNSAPLEGGTRLTICGWDFGFRRNNKFDLKK
TRVLLGNESCTLTLSESTMNTLKCTVGPAMNKHFNMSIIISNGHGTTQYSTFSYVDPVIT
SISPKYGPMAGGTLLTLTGNYLNSGNSRHISIGGKTCTLKSVSNSILECYTPAQTISTEF
AVKLKIDLANRETSIFSYREDPIVYEIHPTKSFISGGSTITGVGKNLNSVSVPRMVINVH
EAGRNFTVACQHRSNSEIICCTTPSLQQLNLQLPLKTKAFFMLDGILSKYFDLIYVHNPV
FKPFEKPVMISMGNENVLEIKGNDIDPEAVKGEVLKVGNKSCENIHLHSEAVLCTVPNDL
LKLNSELNIEWKQAISSTVLGKVIVQPDQNFTGLIAGVVSISTALLLLLGFFLWLKKRKQ
IKDLGSELVRYDARVHTPHLDRLVSARSVSPTTEMVSNESVDYRATFPEDQFPNSSQNGS
CRQVQYPLTDMSPILTSGDSDISSPLLQNTVHIDLSALNPELVQAVQHVVIGPSSLIVHF
NEVIGRGHFGCVYHGTLLDNDGKKIHCAVKSLNRITDIGEVSQFLTEGIIMKDFSHPNVL
SLLGICLRSEGSPLVVLPYMKHGDLRNFIRNETHNPTVKDLIGFGLQVAKGMKYLASKKF
VHRDLAARNCMLDEKFTVKVADFGLARDMYDKEYYSVHNKTGAKLPVKWMALESLQTQKF
TTKSDVWSFGVLLWELMTRGAPPYPDVNTFDITVYLLQGRRLLQPEYCPDPLYEVMIKCW
HPKAEMRPSFSELVSRISAIFSTFIGEHYVHVNATYVNVKCVAPYPSLLSSEDNADDEVD
TRPASFWETS
Humanized Antibody
73R010 (73R009 H12) Heavy chain variable region
amino acid sequence
(SEQ ID NO: 94)
QVQLQESGPGLVKPSETLSLTCTVSGTTITASYAWSWIRQPPGKGLEWMGYISYSGGTDY
NPSLKSRVTISRDTFKNQFSLKLSSVTAADTAVYYCARKGAYWGQGTLVTVSS
73R010 (73R009 L7) Light chain variable region
amino acid sequence
(SEQ ID NO: 95)
EIVLTQSPATLSLSPGERATLSCSASSSVSSSYLYWYQQKPGQAPRLLIYSTSNLASGIP
ARFSGSGSGTDFTLTISRLEPEDFAVYYCHQWSSYPYTFGQGTKLEIK
73R010 (73R009 H12) Heavy chain amino acid sequence
with predicted signal sequence underlined
(SEQ ID NO: 96)
MKHLWFFLLLVAAPRWVLSQVQLQESGPGLVKPSETLSLTCTVSGTTITASYAWSWIRQP
PGKGLEWMGYISYSGGTDYNPSLKSRVTISRDTFKNQFSLKLSSVTAADTAVYYCARKGA
YWGQGTLVTVSSASTKGPSVFPLAPCSRSTSESTAALGCLVKDYFPEPVTVSWNSGALTS
GVHTFPAVLQSSGLYSLSSVVTVPSSNFGTQTYTCNVDHKPSNTKVDKTVERKCCVECPP
CPAPPVAGPSVFLFPPKPKDTLMISRTPEVTCVVVDVSHEDPEVQFNWYVDGVEVHNAKT
KPREEQFNSTFRVVSVLTVVHQDWLNGKEYKCKVSNKGLPAPIEKTISKTKGQPREPQVY
TLPPSREEMTKNQVSLTCLVKGFYPSDIAVEWESNGQPENNYKTTPPMLDSDGSFFLYSK
LTVDKSRWQQGNVFSCSVMHEALHNHYTQKSLSLSPGK
73R010 13A variant (73R009 H12) Heavy chain amino acid
sequence with predicted signal sequence underlined
(SEQ ID NO: 97)
MKHLWFFLLLVAAPRWVLSQVQLQESGPGLVKPSETLSLTCTVSGTTITASYAWSWIRQP
PGKGLEWMGYISYSGGTDYNPSLKSRVTISRDTFKNQFSLKLSSVTAADTAVYYCARKGA
YWGQGTLVTVSSASTKGPSVFPLAPCSRSTSESTAALGCLVKDYFPEPVTVSWNSGALTS
GVHTFPAVLQSSGLYSLSSVVTVPSSNFGTQTYTCNVDHKPSNTKVDKTVERKCCVECPP
CPAPPVAGPSVFLFPPKPKDTLMISRTPEVTCVVVDVSHEDPEVQFNWYVDGVEVHNAKT
KPREEQFNSTFRVVSVLTVVHQDWLNGKEYKCKVSNKGLPAPIEKTISKTKGQPREPQVY
TLPPSREKMTKNQVSLTCLVKGFYPSDIAVEWESNGQPENNYKTTPPMLKSDGSFFLYSK
LTVDKSRWQQGNVFSCSVMHEALHNHYTQKSLSLSPGK
73R010 (73R009 L7) Light chain amino acid sequence
with predicted signal sequence underlined
(SEQ ID NO: 98)
METPAQLLFLLLLWLPDTTGEIVLTQSPATLSLSPGERATLSCSASSSVSSSYLYWYQQK
PGQAPRLLIYSTSNLASGIPARFSGSGSGTDFTLTISRLEPEDFAVYYCHQWSSYPYTFG
QGTKLEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC
73R010 (73R009 H12) Heavy chain amino acid sequence
without predicted signal sequence
(SEQ ID NO: 99)
QVQLQESGPGLVKPSETLSLTCTVSGTTITASYAWSWIRQPPGKGLEWMGYISYSGGTDY
NPSLKSRVTISRDTFKNQFSLKLSSVTAADTAVYYCARKGAYWGQGTLVTVSSASTKGPS
VFPLAPCSRSTSESTAALGCLVKDYFPEPVTVSWNSGALTSGVHTFPAVLQSSGLYSLSS
VVTVPSSNFGTQTYTCNVDHKPSNTKVDKTVERKCCVECPPCPAPPVAGPSVFLFPPKPK
DTLMISRTPEVTCVVVDVSHEDPEVQFNWYVDGVEVHNAKTKPREEQFNSTFRVVSVLTV
VHQDWLNGKEYKCKVSNKGLPAPIEKTISKTKGQPREPQVYTLPPSREEMTKNQVSLTCL
VKGFYPSDIAVEWESNGQPENNYKTTPPMLDSDGSFFLYSKLTVDKSRWQQGNVFSCSVM
HEALHNHYTQKSLSLSPGK
73R010 13A Variant (73R009 H12) Heavy chain amino
acid sequence without predicted signal sequence
(SEQ ID NO: 100)
QVQLQESGPGLVKPSETLSLTCTVSGTTITASYAWSWIRQPPGKGLEWMGYISYSGGTDY
NPSLKSRVTISRDTFKNQFSLKLSSVTAADTAVYYCARKGAYWGQGTLVTVSSASTKGPS
VFPLAPCSRSTSESTAALGCLVKDYFPEPVTVSWNSGALTSGVHTFPAVLQSSGLYSLSS
VVTVPSSNFGTQTYTCNVDHKPSNTKVDKTVERKCCVECPPCPAPPVAGPSVFLFPPKPK
DTLMISRTPEVTCVVVQVSHEDPEVQFNWYVDGVEVHNAKTKPREEQFNSTFRVVSVLTV
VHQDWLNGKEYKCKVSNKGLPAPIEKTISKTKGQPREPQVYTLPPSREKMTKNQVSLTCL
VKGFYPSDIAVEWESNGQPENNYKTTPPMLKSDGSFFLYSKLTVDKSRWQQGNVFSCSVM
HEALHNHYTQKSLSLSPGK
73R010 (73R009 L7) Light chain amino acid sequence
without predicted signal sequence
(SEQ ID NO: 101)
EIVLTQSPATLSLSPGERATLSCSASSSVSSSYLYWYQQKPGQAPRLLIYSTSNLASGIP
ARFSGSGSGTDFTLTISRLEPEDFAVYYCHQWSSYPYTFGQGTKLEIKRTVAAPSVFIFP
PSDEQLKSGTASVVCLLNNFYPREAKVQWKVDNALQSGNSQESVTEQDSKDSTYSLSSTL
TLSKADYFKHKVYACEVTHQGLSSPVTKSFNRGEC
73R010 (73R009 H12) Heavy chain nucleotide sequence
(SEQ ID NO: 102)
ATGAAACATCTGTGGTTCTTCCTCCTCCTGGTGGCTGCTCCCCGCTGGGTCCTGTCCCAA
GTGCAGCTGCAGGAGTCTGGCCCAGGACTGGTGAAGCCTTCCGAGACCCTGTCCCTCACC
TGCACCGTCTCTGGAACTACCATCACTGCCTCCTATGCCTGGAGCTGGATTCGGCAGCCC
CCTGGCAAGGGACTGGAGTGGATGGGGTACATAAGCTACTCCGGGGGCACTGACTACAAC
CCATCTCTCAAATCCCGGGTCACAATATCACGGGACACATTCAAGAACCAGTTCTCCCTG
AAACTCTCCTCTGTGACCGCTGCTGACACCGCCGTCTATTACTGTGCAAGAAAGGGGGCT
TACTGGGGCCAAGGGACTCTGGTCACTGTCAGCTCAGCCTCTACCAAGGGCCCTAGCGTC
TTCCCTCTGGCCCCCTGCTCCCGGTCCACCAGCGAGAGCACAGCCGCCCTGGGCTGCCTG
GTCAAGGACTACTTCCCCGAACCTGTGACAGTGTCCTGGAACTCCGGCGCTCTGACCAGC
GGCGTGCACACCTTCCCAGCTGTCCTCCAGTCCTCCGGACTCTACTCCCTCTCCTCCGTG
GTGACAGTGCCCTCCTCCAACTTCGGCACCCAGACCTACACCTGCAACGTCGATCACAAG
CCCAGCAACACCAAGGTTGATAAGACAGTTGAGCGCAAATGTTGTGTCGAGTGCCCTCCT
TGCCCAGCCCCTCCTGTGGCTGGACCTTCCGTCTTCCTCTTCCCCCCTAAACCCAAAGAC
ACCCTCATGATCTCCCGGACCCCTGAGGTCACATGCGTGGTGGTGGACGTGAGCCACGAA
GACCCCGAGGTCCAGTTCAACTGGTATGTGGACGGCGTGGAGGTGCATAATGCCAAGACA
AAGCCACGGGAGGAGCAGTTCAACAGCACATTCCGGGTGGTCAGCGTCCTCACCGTTGTG
CACCAGGACTGGCTGAACGGCAAGGAGTACAAGTGCAAAGTCTCCAACAAAGGCCTCCCT
GCCCCCATCGAGAAAACCATCTCCAAAACCAAAGGGCAGCCCAGGGAACCACAGGTGTAC
ACCCTGCCCCCTTCCCGGGAGGARATGACCAAGAACCAAGTCAGCCTGACCTGCCTGGTC
AAAGGCTTCTACCCCTCCGACATCGCCGTGGAGTGGGAGAGCAATGGGCAGCCTGAGAAC
AACTACAAGACCACACCTCCCATGCTGGAYTCCGACGGCTCCTTCTTCCTCTACTCCAAA
CTCACCGTGGACAAGAGCAGGTGGCAGCAGGGGAACGTCTTCTCCTGCTCCGTGATGCAT
GAGGCTCTGCACAACCACTACACACAGAAGTCCCTCTCCCTGTCTCCTGGAAAA
Wherein R = A or G
Wherein Y = C or T
73R010 13A variant (73R009 H12) Heavy chain nucleotide
sequence
(SEQ ID NO: 103)
ATGAAACATCTGTGGTTCTTCCTCCTCCTGGTGGCTGCTCCCCGCTGGGTCCTGTCCCAA
GTGCAGCTGCAGGAGTCTGGCCCAGGACTGGTGAAGCCTTCCGAGACCCTGTCCCTCACC
TGCACCGTCTCTGGAACTACCATCACTGCCTCCTATGCCTGGAGCTGGATTCGGCAGCCC
CCTGGCAAGGGACTGGAGTGGATGGGGTACATAAGCTACTCCGGGGGCACTGACTACAAC
CCATCTCTCAAATCCCGGGTCACAATATCACGGGACACATTCAAGAACCAGTTCTCCCTG
AAACTCTCCTCTGTGACCGCTGCTGACACCGCCGTCTATTACTGTGCAAGAAAGGGGGCT
TACTGGGGCCAAGGGACTCTGGTCACTGTCAGCTCAGCCTCTACCAAGGGCCCTAGCGTC
TTCCCTCTGGCCCCCTGCTCCCGGTCCACCAGCGAGAGCACAGCCGCCCTGGGCTGCCTG
GTCAAGGACTACTTCCCCGAACCTGTGACAGTGTCCTGGAACTCCGGCGCTCTGACCAGC
GGCGTGCACACCTTCCCAGCTGTOCTCCAGTCCTCCGGACTCTACTCCCTCTCCTCCGTG
GTGACAGTGCCCTCCTCCAACTTCGGCACCCAGACCTACACCTGCAACGTCGATCACAAG
CCCAGCAACACCAAGGTTGATAAGACAGTTGAGCGCAAATGTTGTGTCGAGTGCCCTCCT
TGCCCAGCCCCTCCTGTGGCTGGACCTTCCGTCTTCCTCTTCCCCCCTAAACCCAAAGAC
ACCCTCATGATCTCCCGGACCCCTGAGGTCACATGCGTGGTGGTGGACGTGAGCCACGAA
GACCCCGAGGTCCAGTTCAACTGGTATGTGGACGGCGTGGAGGTGCATAATGCCAAGACA
AAGCCACGGGAGGAGCAGTTCAACAGCACATTCCGGGTGGTCAGCGTCCTCACCGTTGTG
CACCAGGACTGGCTGAACGGCAAGGAGTACAAGTGCAAAGTCTCCAACAAAGGCCTCCCT
GCCCCCATCGAGAAAACCATCACCAAAACCAAAGGGCAGCCCAGGGAACCACAGGTGTAC
ACCCTGCCCCCTTCCCGGGAGAAGATGACCAAGAACCAAGTCAGCCTGACCTGCCTGGTC
AAAGGCTTCTACCCCTCCGACATCGCCGTGGAGTGGGAGAGCAATGGGCAGCCTGAGAAC
AACTACAAGACCACACCTCCCATGCTGAAGTCCGACGGCTCCTTCTTCCTCTACTCCAAA
CTCACCGTGGACAAGAGCAGGTGGCAGCAGGGGAACGTCTTCTCCTGCTCCGTGATGCAT
GAGGCTCTGCACAACCACTACACACAGAAGTCCCTCTCCCTGTCTCCTGGAAAA
73R010 (73R009 H12) Heavy chain nucleotide sequence
without predicted signal sequence
(SEQ ID NO: 104)
CAAGTGCAGCTGCAGGAGTCTGGCCCAGGACTGGTGAAGCCTTCCGAGACCCTGTCCCTC
ACCTGCACCGTCTCTGGAACTACCATCACTGCCTCCTATGCCTGGAGCTGGATTCGGCAG
CCCCCTGGCAAGGGACTGGAGTGGATGGGGTACATAAGCTACTCCGGGGGCACTGACTAC
AACCCATCTCTCAAATCCCGGGTCACAATATCACGGGACACATTCAAGAACCAGTTCTCC
CTGAAACTCTCCTCTGTGACCGCTGCTGACACCGCCGTCTATTACTGTGCAAGAAAGGGG
GCTTACTGGGGCCAAGGGACTCTGGTCACTGTCAGCTCAGCCTCTACCAAGGGCCCTAGC
GTCTTCCCTCTGGCCCCCTGCTCCCGGTCCACCAGCGAGAGCACAGCCGCCCTGGGCTGC
CTGGTCAAGGACTACTTCCCCGAACCTGTGACAGTGTCCTGGAACTCCGGCGCTCTGACC
AGCGGCGTGCACACCTTCCCAGCTGTCCTCCAGTCCTCCGGACTCTACTCCCTCTCCTCC
GTGGTGACAGTGCCCTCCTCCAACTTCGGCACCCAGACCTACACCTGCAACGTCGATCAC
AAGCCCAGCAACACCAAGGTTGATAAGACAGTTGAGCGCAAATGTTGTGTCGAGTGCCCT
CCTTGCCCAGCCCCTCCTGTGGCTGGACCTTCCGTCTTCCTCTTCCCCCCTAAACCCAAA
GACACCCTCATGATCTCCCGGACCCCTGAGGTCACATGCGTGGTGGTGGACGTGAGCCAC
GAAGACCCCGAGGTCCAGTTCAACTGGTATGTGGACGGCGTGGAGGTGCATAATGCCAAG
ACAAAGCCACGGGAGGAGCAGTTCAACAGCACATTCCGGGTGGTCAGCGTCCTCACCGTT
GTGCACCAGGACTGGCTGAACGGCAAGGAGTACAAGTGCAAAGTCTCCAACAAAGGCCTC
CCTGCCCCCATCGAGAAAACCATCTCCAAAACCAAAGGGCAGCCCAGGGAACCACAGGTG
TACACCCTGCCCCCTTCCCGGGAGGARATGACCAAGAACCAAGTCAGCCTGACCTGCCTG
GTCAAAGGCTTCTACCCCTCCGACATCGCCGTGGAGTGGGAGAGCAATGGGCAGCCTGAG
AACAACTACAAGACCACACCTCCCATGCTGGAYTCCGACGGCTCCTTCTTCCTCTACTCC
AAACTCACCGTGGACAAGAGCAGGTGGCAGCAGGGGAACGTCTTCTCCTGCTCCGTGATG
CATGAGGCTCTGCACAACCACTACACACAGAAGTCCCTCTCCCTGTCTCCTGGAAAA
Wherein R = A or G
Wherein Y = C or T
73R010 13A variant (73R009 H12) Heavy chain nucleotide
sequence without predicted signal sequence
(SEQ ID NO: 105)
CAAGTGCAGCTGCAGGAGTCTGGCCCAGGACTGGTGAAGCCTTCCGAGACCCTGTCCCTC
ACCTGCACCGTCTCTGGAACTACCATCACTGCCTCCTATGCCTGGAGCTGGATTCGGCAG
CCCCCTGGCAAGGGACTGGAGTGGATGGGGTACATAAGCTACTCCGGGGGCACTGACTAC
AACCCATCTCTCAAATCCCGGGTCACAATATCACGGGACACATTCAAGAACCAGTTCTCC
CTGAAACTCTCCTCTGTGACCGCTGCTGACACCGCCGTCTATTACTGTGCAAGAAAGGGG
GCTTACTGGGGCCAAGGGACTCTGGTCACTGTCAGCTCAGCCTCTACCAAGGGCCCTAGC
GTCTTCCCTCTGGCCCCCTGCTCCCGGTCCACCAGCGAGAGCACAGCCGCCCTGGGCTGC
CTGGTCAAGGACTACTTCCCCGAACCTGTGACAGTGTCCTGGAACTCCGGCGCTCTGACC
AGCGGCGTGCACACCTTCCCAGCTGTCCTCCAGTCCTCCGGACTCTACTCCCTCTCCTCC
GTGGTGACAGTGCCCTCCTCCAACTTCGGCACCCAGACCTACACCTGCAACGTCGATCAC
AAGCCCAGCAACACCAAGGTTGATAAGACAGTTGAGCGCAAATGTTGTGTCGAGTGCCCT
CCTTGCCCAGCCCCTCCTGTGGCTGGACCTTCCGTCTTCCTCTTCCCCCCTAAACCCAAA
GACACCCTCATGATCTCCCGGACCCCTGAGGTCACATGCGTGGTGGTGGACGTGAGCCAC
GAAGACCCCGAGGTCCAGTTCAACTGGTATGTGGACGGCGTGGAGGTGCATAATGCCAAG
ACAAAGCCACGGGAGGAGCAGTTCAACAGCACATTCCGGGTGGTCAGCGTCCTCACCGTT
GTGCACCAGGACTGGCTGAACGGCAAGGAGTACAAGTGCAAAGTCTCCAACAAAGGCCTC
CCTGCCCCCATCGAGAAAACCATCTCCAAAACCAAAGGGCAGCCCAGGGAACCACAGGTG
TACACCCTGCCCCCTTCCCGGGAGAAGATGACCAAGAACCAAGTCAGCCTGACCTGCCTG
GTCAAAGGCTTCTACCCCTCCGACATCGCCGTGGAGTGGGAGAGCAATGGGCAGCCTGAG
AACAACTACAAGACCACACCTCCCATGCTGAAGTCCGACGGCTCCTTCTTCCTCTACTCC
AAACTCACCGTGGACAAGAGCAGGTGGCAGCAGGGGAACGTCTTCTCCTGCTCCGTGATG
CATGAGGCTCTGCACAACCACTACACACAGAAGTCCCTCTCCCTGTCTCCTGGAAAA
73R010 (73R009 L7) Light chain nucleotide sequence
(SEQ ID NO: 106)
ATGGAAACCCCAGCTCAACTCCTCTTCCTCCTGCTCCTCTGGCTCCCCGATACCACCGGA
GAAATTGTGCTCACTCAGTCTCCCGCTACCCTGTCTCTCTCTCCTGGGGAAAGAGCCACC
CTCTCCTGCTCTGCCTCCTCATCCGTTTCTTCCTCCTACCTCTACTGGTACCAGCAGAAA
CCTGGCCAGGCTCCCAGGCTCCTCATCTATAGCACATCCAACCTGGCTTCTGGCATCCCA
GCTAGGTTCTCTGGCTCTGGGTCTGGGACAGACTTCACTCTCACCATCAGCAGACTGGAG
CCTGAAGATTTTGCAGTGTATTACTGTCATCAGTGGTCATCCTACCCATACACATTCGGC
CAGGGAACCAAACTGGAGATTAAGCGTACGGTGGCAGCCCCTTCTGTCTTTATCTTCCCT
CCATCCGACGAGCAGCTCAAATCAGGAACCGCTTCTGTCGTGTGCCTGCTTAACAATTTC
TACCCACGGGAAGCCAAGGTGCAGTGGAAGGTGGACAATGCCCTGCAATCAGGTAATTCC
CAAGAGTCAGTGACTGAACAGGATAGCAAGGACAGCACCTATTCACTCTCCAGCACTCTG
ACCCTGTCCAAGGCTGACTACGAAAAGCATAAGGTGTACGCATGCGAGGTGACCCACCAG
GGTCTGAGCAGCCCCGTCACCAAGTCTTTCAACAGAGGGGAGTGT
73R010 (731009 L7) Light chain nucleotide sequence
without predicted signal sequence
(SEQ ID NO: 107)
GAAATTGTGCTCACTCAGTCTCCCGCTACCCTGTCTCTCTCTCCTGGGGAAAGAGCCACC
CTCTCCTGCTCTGCCTCCTCATCCGTTTCTTCCTCCTACCTCTACTGGTACCAGCAGAAA
CCTGGCCAGGCTCCCAGGCTCCTCATCTATAGCACATCCAACCTGGCTTCTGGCATCCCA
GCTAGGTTCTCTGGCTCTGGGTCTGGGACAGACTTCACTCTCACCATCAGCAGACTGGAG
CCTGAAGATTTTGCAGTGTATTACTGTCATCAGTGGTCATCCTACCCATACACATTCGGC
CAGGGAACCAAACTGGAGATTAAGCGTACGGTGGCAGCCCCTTCTGTCTTTATCTTCCCT
CCATCCGACGAGCAGCTCAAATCAGGAACCGCTTCTGTCGTGTGCCTGCTTAACAATTTC
TACCCACGGGAAGCCAAGGTGCAGTGGAAGGTGGACAATGCCCTGCAATCAGGTAATTCC
CAAGAGTCAGTGACTGAACAGGATAGCAAGGACAGCACCTATTCACTCTCCAGCACTCTG
ACCCTGTCCAAGGCTGACTACGAAAAGCATAAGGTGTACGCATGCGAGGTGACCCACCAG
GGTCTGAGCAGCCCCGTCACCAAGTCTTTCAACAGAGGGGAGTGT
73R010 13B Variant (73R009 H12) Heavy chain amino acid
sequence without predicted signal sequence
(SEQ ID NO: 108)
QVQLQESGPGLVKPSETLSLTCTVSGTTITASYAWSWIRQPPGKGLEWMGYISYSGGTDY
NPSLKSRVTISRDTFKNQFSLKLSSVTAADTAVYYCARKGAYWGQGTLVTVSSASTKGPS
VFPLAPCSRSTSESTAALGCLVKDYFPEPVTVSWNSGALTSGVHTFPAVLQSSGLYSLSS
VVTVPSSNFGTQTYTCNVDHKPSNTKVDKTVERKCCVECPPCPAPPVAGPSVFLFPPKPK
DTLMISRTPEVTCVVVDVSHEDPEVQFNWYVDGVEVHNAKTKPREEQFNSTFRVVSVITV
VHQDWLNGKEYKCKVSNKGLPAPIEKTISKTKGQPREPQVYTLPPSREEMTKNQVSLTCL
VEGFYPSDIAVEWESNGQPENNYKTTPPMLDSDGSFDLYSELTVDKSRWQQGNVFSCSVM
HEALHNHYTQKSLSLSPGK
73R010 13A Variant (Cys/Ser) (73R009 H12) Heavy chain
nucleotide sequence
(SEQ ID NO: 109)
ATGAAACATCTGTGGTTCTTCCTCCTCCTGGTGGCTGCTCCCCGCTGGGTCCTGTCCCAA
GTGCAGCTGCAGGAGTCTGGCCCAGGACTGGTGAAGCCTTCCGAGACCCTGTCCCTCACC
TGCACCGTCTCTGGAACTACCATCACTGCCTCCTATGCCTGGAGCTGGATTCGGCAGCCC
CCTGGCAAGGGACTGGAGTGGATGGGGTACATAAGCTACTCCGGGGGCACTGACTACAAC
CCATCTCTCAAATCCCGGGTCACAATATCACGGGACACATTCAAGAACCAGTTCTCCCTG
AAACTCTCCTCTGTGACCGCTGCTGACACCGCCGTCTATTACTGTGCAAGAAAGGGGGCT
TACTGGGGCCAAGGGACTCTGGTCACTGTCAGCTCAGCCTCAACTAAGGGCCCTAGCGTC
TTCCCTCTGGCCCCCTGCTCCCGGTCCACCAGCGAGAGCACAGCCGCCCTGGGCTGCCTG
GTCAAGGACTACTTCCCCGAACCTGTGACAGTGTCCTGGAACTCCGGCGCTCTGACCAGC
GGCGTGCACACCTTCCCAGCTGTCCTCCAGTCCTCCGGACTCTACTCCCTCTCCTCCGTG
GTGACAGTGCCCTCCTCCAACTTCGGCACCCAGACCTACACCTGCAACGTCGATCACAAG
CCCAGCAACACCAAGGTTGATAAGACAGTTGAGCGCAAATCTTGTGTCGAGTGCCCTCCT
TGCCCAGCCCCTCCTGTGGCTGGACCTTCCGTCTTCCTCTTCCCCCCTAAACCCAAAGAC
ACCCTCATGATCTCCCGGACCCCTGAGGTCACATGCGTGGTGGTGGACGTGAGCCACGAA
GACCCCGAGGTCCAGTTCAACTGGTATGTGGACGGCGTGGAGGTGCATAATGCCAAGACA
AAGCCACGGGAGGAGCAGTTCAACAGCACATTCCGGGTGGTCAGCGTCCTCACCGTTGTG
CACCAGGACTGGCTGAACGGCAAGGAGTACAAGTGCAAAGTCTCCAACAAAGGCCTCCCT
GCCCCCATCGAGAAAACCATCTCCAAAACCAAAGGGCAGCCCAGGGAACCACAGGTGTAC
ACCCTGCCCCCTTCCCGGGAGAAGATGACCAAGAACCAAGTCAGCCTGACCTGCCTGGTC
AAAGGCTTCTACCCCTCCGACATCGCCGTGGAGTGGGAGAGCAATGGGCAGCCTGAGAAC
AACTACAAGACCACACCTCCCATGCTGAAGTCCGACGGCTCCTTCTTCCTCTACTCCAAA
CTCACCGTGGACAAGAGCAGGTGGCAGCAGGGGAACGTCTTCTCCTGCTCCGTGATGCAT
GAGGCTCTGCACAACCACTACACACAGAAGTCCCTCTCCCTGTCTCCTGGAAAATGA
73R010 13A Variant (Cys/Ser) (73R009 H12) Heavy chain
amino acid sequence with predicted signal sequence
underlined
(SEQ ID NO: 110)
MKHLWFFLLLVAAPRWVLSQVQLQESGPGLVKPSETLSLTCTVSGTTITASYAWSWIRQP
PGKGLEWMGYISYSGGTDYNPSLKSRVTISRDTFKNQFSLKLSSVTAADTAVYYCARKGA
YWGQGTLVTVSSASTKGPSVFPLAPCSRSTSESTAALGCLVKDYFPEPVTVSWNSGALTS
GVHTFPAVLQSSGLYSLSSVVTVPSSNFGTQTYTCNVDHKPSNTKVDKTVERKSCVECPP
CPAPPVAGPSVFLFPPKPKDTLMISRTPEVTCVVVDVSHEDPEVQFNWYVDGVEVHNAKT
KPREEQFNSTFRVVSVLTVVHQDWLNGKEYKCKVSNKGLPAPIEKTISKTKGQPREPQVY
TLPPSREKMTKNQVSLTCLVKGFYPSDIAVEWESNGQPENNYKTTPPMLKSDGSFFLYSK
LTVDKSRWQQGNVFSCSVMHEALHNHYTQKSLSLSPGK
73R010 13A Variant (Cys/Ser) (73R009 H12) Heavy chain
amino acid sequence without predicted signal sequence
(SEQ ID NO: 111)
QVQLQESGPGLVKPSETLSLTCTVSGTTITASYAWSWIRQPPGKGLEWMGYISYSGGTDY
NPSLKSRVTISRDTFKNQFSLKLSSVTAADTAVYYCARKGAYWGQGTLVTVSSASTKGPS
VFPLAPCSRSTSESTAALGCLVKDYFPEPVTVSWNSGALTSGVHTFPAVLQSSGLYSLSS
VVTVPSSNFGTQTYTCNVDHKPSNTKVDKTVERKSCVECPPCPAPPVAGPSVFLFPPKPK
DTLMISRTPEVTCVVVDVSHEDPEVQFNWYVDGVEVHNAKTKPREEQFNSTFRVVSVLTV
VHQDWLNGKEYKCKVSNKGLPAPIEKTISKTKGQPREPQVYTLPPSREKMTKNQVSLTCL
VKGFYPSDIAVEWESNGQPENNYKTTPPMLKSDGSFFLYSKLTVDKSRWQQGNVFSCSVM
HEALHNHYTQKSLSLSPGK
73R010 (Cys/Ser) (73R009 H12) Heavy chain amino acid
sequence without predicted signal sequence
(SEQ ID NO: 112)
QVQLQESGPGLVKPSETLSLTCTVSGTTITASYAWSWIRQPPGKGLEWMGYISYSGGTDY
NPSLKSRVTISRDTFKNQFSLKLSSVTAADTAVYYCARKGAYWGQGTLVTVSSASTKGPS
VFPLAPCSRSTSESTAALGCLVKDYFPEPVTVSWNSGALTSGVHTFPAVLQSSGLYSLSS
VVTVpSSNFGTQTYTCNVDHKPSNTKVDKTVERKSCVECPPCPAPPVAGPSVFLFPPKPK
DTLMISRTPEVTCVVVDVSHEDPEVQFNWYVDGVEVHNAKTKPREEQFNSTFRVVSVLTV
VHQDWLNGKEYKCKVSNKGLPAPIEKTISKTKGQPREPQVYTLPPSREEMTKNQVSLTCL
VKGFYPSDIAVEWESNGQPENNYKTTPPMLDSDGSFFLYSKLTVDKSRWQQGNVFSCSVM
HEALHNHYTQKSLSLSPGK
MET (amino acids 97-101 of SEQ ID NO: 93)
(SEQ ID NO: 113)
PCQDC
73R010 13B Variant (Cys/Ser) (73R009 H12) Heavy chain
amino acid sequence without predicted signal sequence
(SEQ ID NO: 114)
QVQLQESGPGLVKPSETLSLTCTVSGTTITASYAWSWIRQPPGKGLEWMGYISYSGGTDY
NPSLKSRVTISRDTFKNQFSLKLSSVTAADTAVYYCARKGAYWGQGTLVTVSSASTKGPS
VFPLAPCSRSTSESTAALGCLVKDYFPEPVTVSWNSGALTSGVHTFPAVLQSSGLYSLSS
VVTVPSSNFGTQTYTCNVDHKPSNTKVDKTVERKSCVECPPCPAPPVAGPSVFLFPPKPK
DTLMISRTPEVTCVVVDVSHEDPEVQFNWYVDGVEVHNAKTKPREEQFNSTFRVVSVLTV
VHQDWLNGKEYKCKVSNKGLPAPIEKTISKTKGQPREPQVYTLPPSREEMTKNQVSLTCL
VEGFYPSDIAVEWESNGQPENNYKTTPPMLDSDGSFFLYSELTVDKSRWQQGNVFSCSVM
HEALHNHYTQKSLSLSPGK

1-112. (canceled)

113. A bispecific agent comprising:
a) a first binding site Comprising an antigen-binding site of an antibody that specifically binds the extracellular domain of human MET; and
b) a second binding site that specifically binds one or more components of the WNT pathway.

114. The bispecific agent of claim 113, wherein the first binding site comprises a heavy chain CDR1 comprising ASYAWS (SEQ ID NO:1), a heavy chain CDR2 comprising YISYSGGTDYNPSLKS (SEQ ID NO:2), and a heavy chain CDR3 comprising KGAY (SEQ ID NO:3); and a light chain CDR1 comprising SASSSVSSSYLY (SEQ ID NO:4), a light chain CDR2 comprising STSNLAS (SEQ ID NO:5), and a light chain CDR3 comprising HQWSSYPYT (SEQ ID NO:6).

115. The bispecific agent of claim 113, wherein the first binding site comprises a heavy chain variable region, having at least 90% sequence identity to SEQ ID NO:94 and a light chain variable region having at least 90% sequence identity to SEQ ID NO:95.

116. The bispecific agent, of claim 115, wherein the first binding site comprises a heavy chain variable region of SEQ ID NO:94 and a light chain variable region of SEQ ID NO:95.

117. The bispecific agent of claim 113, which comprises a heavy chain sequence of SEQ ID NO:100 or SEQ ID NO:111, and a light chain sequence of SEQ ID NO:101.

118. The bispecific agent of claim 113, wherein the second binding site comprises a soluble human frizzled (FZD) receptor.

119. The bispecific agent of claim 118, wherein the soluble FZD receptor comprises a Fri domain of a human FZD protein.

120. The bispecific agent of claim 116, wherein the second binding site comprises a soluble FZD receptor.

121. The bispecific agent of claim 20, wherein the soluble FZD receptor comprises a Fri domain of a human FZD protein.

122. The bispecific agent of claim 121, wherein the Fri domain of a human FZD protein is the Fri domain of human FZD8.

123. The bispecific agent of claim 113, wherein the first binding site comprises a heavy chain variable region comprising SEQ ID NO:94 and a light chain variable region comprising SEQ ID NO 95; and the second binding site comprises a soluble receptor comprising SEQ ID NO:28, SEQ ID NO:29, or SEQ ID NO:39.

124. The bispecific agent of claim 113, wherein the first binding site comprises a heavy chain sequence of SEQ ID NO:100 or SEQ ID NO:111 and a light chain sequence of SEQ ID NO:101; and the second binding site comprises a soluble receptor of SEQ ID NO:56.

125. The bispecific agent of claim 113, which comprises:
(a) a heavy chain encoded by the plasmid deposited with ATCC as designation number PTA-120695;
(b) a light chain encoded by the plasmid deposited with ATCC as designation number PTA-120388; and
(c) a soluble receptor encoded by the plasmid deposited with ATCC as designation number PTA-13611.

126. The bispecific agent of claim 113, which:
(a) binds the Sema domain of human MET;
(b) binds the alpha-chain of the Sema domain;
(c) binds an epitope within the Sema domain that comprises amino acids PCQDC (SEQ ID NO:113);
(d) binds an epitope that comprises amino acids 97-101 of human MET (SEQ ID NO:93); and/or
(e) binds an epitope that comprises the glutamine residue corresponding to position 99 of SEQ ID NO:93.

127. A pharmaceutical composition comprising the bispecific agent of claim 113 and a pharmaceutically acceptable carrier.

128. A cell producing the bispecific agent of claim 113.

129. An isolated polynucleotide molecule comprising a nucleotide sequence that encodes the bispecific agent of claim 113.

130. A method of inhibiting, growth of a tumor, comprising contacting the tumor with an effective amount of the bispecific agent of claim 113.

131. A method of inhibiting growth of a tumor in a subject, comprising administering to the subject a therapeutically effective amount of the bispecific agent of claim 113.

132. A method of treating cancer in a subject, comprising administering to the subject a therapeutically effective amount of the bispecific agent of claim 113.

133. The method of claim 132, which further comprises administering at least one addition therapeutic agent.

134. An isolated antibody that specifically binds human MET, which comprises a heavy chain variable region comprising SEQ ID NO:94 and a light chain variable region comprising SEQ ID NO:95.

135. The antibody of claim 134, which comprises a heavy chain sequence of SEQ ID NO:99 or SEQ ID NO:112 and a light chain sequence of SEQ ID NO: 101.


SEQUENCE LISTING (114 sequences)

SEQ ID NO: 1 - protein, 6 aa, Artificial Sequence
  73R009 Heavy chain CDR1
ASYAWS

SEQ ID NO: 2 - protein, 16 aa, Artificial Sequence
  73R009 Heavy chain CDR2
YISYSGGTDYNPSLKS

SEQ ID NO: 3 - protein, 4 aa, Artificial Sequence
  73R009 Heavy chain CDR3
KGAY

SEQ ID NO: 4 - protein, 12 aa, Artificial Sequence
  73R009 Light chain CDR1
SASSSVSSSYLY

SEQ ID NO: 5 - protein, 7 aa, Artificial Sequence
  73R009 Light chain CDR2
STSNLAS

SEQ ID NO: 6 - protein, 9 aa, Artificial Sequence
  73R009 Light chain CDR3
HQWSSYPYT

SEQ ID NO: 7 - protein, 113 aa, Artificial Sequence
  73R009 Heavy chain variable region amino acid sequence
QVQLQESGPGLVKPSETLSLTCTVTGTTITASYAWSWIRQPPGKGLEWMGYISYSGGTDYNPSLKSRITISRDTFKNQFSLKLSSVTAADTATYYCARKGAYWGQGTLVTVSS

SEQ ID NO: 8 - protein, 108 aa, Artificial Sequence
  73R009 Light chain variable region amino acid sequence
DIVLTQSPATLSASPGEKVTLTCSASSSVSSSYLYWYQQKPGQAPKLLIYSTSNLASGVPARFSGSGSGTSYSLTISSLEPEDFATYYCHQWSSYPYTFGGGTKLEIK

SEQ ID NO: 9 - protein, 458 aa, Artificial Sequence
  73R009 Heavy chain amino acid sequence with predicted signal sequence
MKHLWFFLLLVAAPRWVLSQVQLQESGPGLVKPSETLSLTCTVTGTTITASYAWSWIRQPPGKGLEWMGYISYSGGTDYNPSLKSRITISRDTFKNQFSLKLSSVTAADTATYYCARKGAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 458 aa, Artificial Sequence
  73R009 (13A variant) Heavy chain amino acid sequence with predicted signal sequence
MKHLWFFLLLVAAPRWVLSQVQLQESGPGLVKPSETLSLTCTVTGTTITASYAWSWIRQPPGKGLEWMGYISYSGGTDYNPSLKSRITISRDTFKNQFSLKLSSVTAADTATYYCARKGAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREKMTKNQVSLTCLVKGFYPSDIAVEWESNGQPENNYKTTPPMLKSDGSFFLYSKLTVDKSRWQQGNVFSCSVMHEALHNHYTQKSLSLSPGK

SEQ ID NO: 11 - protein, 234 aa, Artificial Sequence
  73R009 Light chain amino acid sequence with predicted signal sequence
MKHLWFFLLLVAAPRWVLSDIVLTQSPATLSASPGEKVTLTCSASSSVSSSYLYWYQQKPGQAPKLLIYSTSNLASGVPARFSGSGSGTSYSLTISSLEPEDFATYYCHQWSSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 439 aa, Artificial Sequence
  73R009 Heavy chain amino acid sequence without predicted signal sequence
QVQLQESGPGLVKPSETLSLTCTVTGTTITASYAWSWIRQPPGKGLEWMGYISYSGGTDYNPSLKSRITISRDTFKNQFSLKLSSVTAADTATYYCARKGAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 13 - protein, 439 aa, Artificial Sequence
  73R009 (13A variant) Heavy chain amino acid sequence without predicted signal sequence
QVQLQESGPGLVKPSETLSLTCTVTGTTITASYAWSWIRQPPGKGLEWMGYISYSGGTDYNPSLKSRITISRDTFKNQFSLKLSSVTAADTATYYCARKGAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREKMTKNQVSLTCLVKGFYPSDIAVEWESNGQPENNYKTTPPMLKSDGSFFLYSKLTVDKSRWQQGNVFSCSVMHEALHNHYTQKSLSLSPGK

SEQ ID NO: 14 - protein, 215 aa, Artificial Sequence
  73R009 Light chain amino acid sequence without predicted signal sequence
DIVLTQSPATLSASPGEKVTLTCSASSSVSSSYLYWYQQKPGQAPKLLIYSTSNLASGVPARFSGSGSGTSYSLTISSLEPEDFATYYCHQWSSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - DNA, 1374 bp, Artificial Sequence
  73R009 Heavy chain nucleotide sequence
atgaagcatctgtggtttttcctgctgctcgtggctgctccccggtgggtcctgtctcaggtccaattgcaagagtcaggaccagggcttgtgaagccctcagagactctgtcactcacttgtaccgtgaccggaactaccatcactgcctcctacgcctggagctggatcaggcagcctccgggaaaaggcctggaatggatgggttacatctcctattcaggcggaaccgactacaatcctagcctgaagtctcgcatcaccatttcacgcgataccttcaagaaccaattcagccttaaactctccagcgtgaccgctgcagacactgccacctactactgcgcaagaaagggagcctattggggtcaggggacccttgtgaccgtgagctcagcctctaccaagggccctagcgtcttccctctggccccctgctcccggtccaccagcgagagcacagccgccctgggctgcctggtcaaggactacttccccgaacctgtgacagtgtcctggaactccggcgctctgaccagcggcgtgcacaccttcccagctgtcctccagtcctccggactctactccctctcctccgtggtgacagtgccctcctccaacttcggcacccagacctacacctgcaacgtcgatcacaagcccagcaacaccaaggttgataagacagttgagcgcaaatgttgtgtcgagtgccctccttgcccagcccctcctgtggctggaccttccgtcttcctcttcccccctaaacccaaagacaccctcatgatctcccggacccctgaggtcacatgcgtggtggtggacgtgagccacgaagaccccgaggtccagttcaactggtatgtggacggcgtggaggtgcataatgccaagacaaagccacgggaggagcagttcaacagcacattccgggtggtcagcgtcctcaccgttgtgcaccaggactggctgaacggcaaggagtacaagtgcaaagtctccaacaaaggcctccctgcccccatcgagaaaaccatctccaaaaccaaagggcagcccagggaaccacaggtgtacaccctgcccccttcccgggaggaratgaccaagaaccaagtcagcctgacctgcctggtcaaaggcttctacccctccgacatcgccgtggagtgggagagcaatgggcagcctgagaacaactacaagaccacacctcccatgctggaytccgacggctccttcttcctctactccaaactcaccgtggacaagagcaggtggcagcaggggaacgtcttctcctgctccgtgatgcatgaggctctgcacaaccactacacacagaagtccctctccctgtctcctggaaaa

SEQ ID NO: 16 - DNA, 1374 bp, Artificial Sequence
  73R009 (13A variant) Heavy chain nucleotide sequence
atgaagcatctgtggtttttcctgctgctcgtggctgctccccggtgggtcctgtctcaggtccaattgcaagagtcaggaccagggcttgtgaagccctcagagactctgtcactcacttgtaccgtgaccggaactaccatcactgcctcctacgcctggagctggatcaggcagcctccgggaaaaggcctggaatggatgggttacatctcctattcaggcggaaccgactacaatcctagcctgaagtctcgcatcaccatttcacgcgataccttcaagaaccaattcagccttaaactctccagcgtgaccgctgcagacactgccacctactactgcgcaagaaagggagcctattggggtcaggggacccttgtgaccgtgagctcagcctctaccaagggccctagcgtcttccctctggccccctgctcccggtccaccagcgagagcacagccgccctgggctgcctggtcaaggactacttccccgaacctgtgacagtgtcctggaactccggcgctctgaccagcggcgtgcacaccttcccagctgtcctccagtcctccggactctactccctctcctccgtggtgacagtgccctcctccaacttcggcacccagacctacacctgcaacgtcgatcacaagcccagcaacaccaaggttgataagacagttgagcgcaaatgttgtgtcgagtgccctccttgcccagcccctcctgtggctggaccttccgtcttcctcttcccccctaaacccaaagacaccctcatgatctcccggacccctgaggtcacatgcgtggtggtggacgtgagccacgaagaccccgaggtccagttcaactggtatgtggacggcgtggaggtgcataatgccaagacaaagccacgggaggagcagttcaacagcacattccgggtggtcagcgtcctcaccgttgtgcaccaggactggctgaacggcaaggagtacaagtgcaaagtctccaacaaaggcctccctgcccccatcgagaaaaccatctccaaaaccaaagggcagcccagggaaccacaggtgtacaccctgcccccttcccgggagaagatgaccaagaaccaagtcagcctgacctgcctggtcaaaggcttctacccctccgacatcgccgtggagtgggagagcaatgggcagcctgagaacaactacaagaccacacctcccatgctgaagtccgacggctccttcttcctctactccaaactcaccgtggacaagagcaggtggcagcaggggaacgtcttctcctgctccgtgatgcatgaggctctgcacaaccactacacacagaagtccctctccctgtctcctggaaaa

SEQ ID NO: 17 - DNA, 1317 bp, Artificial Sequence
  73R009 Heavy chain nucleotide sequence without predicted signal sequence
caggtccaattgcaagagtcaggaccagggcttgtgaagccctcagagactctgtcactcacttgtaccgtgaccggaactaccatcactgcctcctacgcctggagctggatcaggcagcctccgggaaaaggcctggaatggatgggttacatctcctattcaggcggaaccgactacaatcctagcctgaagtctcgcatcaccatttcacgcgataccttcaagaaccaattcagccttaaactctccagcgtgaccgctgcagacactgccacctactactgcgcaagaaagggagcctattggggtcaggggacccttgtgaccgtgagctcagcctctaccaagggccctagcgtcttccctctggccccctgctcccggtccaccagcgagagcacagccgccctgggctgcctggtcaaggactacttccccgaacctgtgacagtgtcctggaactccggcgctctgaccagcggcgtgcacaccttcccagctgtcctccagtcctccggactctactccctctcctccgtggtgacagtgccctcctccaacttcggcacccagacctacacctgcaacgtcgatcacaagcccagcaacaccaaggttgataagacagttgagcgcaaatgttgtgtcgagtgccctccttgcccagcccctcctgtggctggaccttccgtcttcctcttcccccctaaacccaaagacaccctcatgatctcccggacccctgaggtcacatgcgtggtggtggacgtgagccacgaagaccccgaggtccagttcaactggtatgtggacggcgtggaggtgcataatgccaagacaaagccacgggaggagcagttcaacagcacattccgggtggtcagcgtcctcaccgttgtgcaccaggactggctgaacggcaaggagtacaagtgcaaagtctccaacaaaggcctccctgcccccatcgagaaaaccatctccaaaaccaaagggcagcccagggaaccacaggtgtacaccctgcccccttcccgggaggaratgaccaagaaccaagtcagcctgacctgcctggtcaaaggcttctacccctccgacatcgccgtggagtgggagagcaatgggcagcctgagaacaactacaagaccacacctcccatgctggaytccgacggctccttcttcctctactccaaactcaccgtggacaagagcaggtggcagcaggggaacgtcttctcctgctccgtgatgcatgaggctctgcacaaccactacacacagaagtccctctccctgtctcctggaaaa

SEQ ID NO: 18 - DNA, 1317 bp, Artificial Sequence
  73R009 (13A variant) Heavy chain nucleotide sequence without predicted signal sequence
caggtccaattgcaagagtcaggaccagggcttgtgaagccctcagagactctgtcactcacttgtaccgtgaccggaactaccatcactgcctcctacgcctggagctggatcaggcagcctccgggaaaaggcctggaatggatgggttacatctcctattcaggcggaaccgactacaatcctagcctgaagtctcgcatcaccatttcacgcgataccttcaagaaccaattcagccttaaactctccagcgtgaccgctgcagacactgccacctactactgcgcaagaaagggagcctattggggtcaggggacccttgtgaccgtgagctcagcctctaccaagggccctagcgtcttccctctggccccctgctcccggtccaccagcgagagcacagccgccctgggctgcctggtcaaggactacttccccgaacctgtgacagtgtcctggaactccggcgctctgaccagcggcgtgcacaccttcccagctgtcctccagtcctccggactctactccctctcctccgtggtgacagtgccctcctccaacttcggcacccagacctacacctgcaacgtcgatcacaagcccagcaacaccaaggttgataagacagttgagcgcaaatgttgtgtcgagtgccctccttgcccagcccctcctgtggctggaccttccgtcttcctcttcccccctaaacccaaagacaccctcatgatctcccggacccctgaggtcacatgcgtggtggtggacgtgagccacgaagaccccgaggtccagttcaactggtatgtggacggcgtggaggtgcataatgccaagacaaagccacgggaggagcagttcaacagcacattccgggtggtcagcgtcctcaccgttgtgcaccaggactggctgaacggcaaggagtacaagtgcaaagtctccaacaaaggcctccctgcccccatcgagaaaaccatctccaaaaccaaagggcagcccagggaaccacaggtgtacaccctgcccccttcccgggagaagatgaccaagaaccaagtcagcctgacctgcctggtcaaaggcttctacccctccgacatcgccgtggagtgggagagcaatgggcagcctgagaacaactacaagaccacacctcccatgctgaagtccgacggctccttcttcctctactccaaactcaccgtggacaagagcaggtggcagcaggggaacgtcttctcctgctccgtgatgcatgaggctctgcacaaccactacacacagaagtccctctccctgtctcctggaaaa

SEQ ID NO: 19 - DNA, 702 bp, Artificial Sequence
  73R009 Light chain nucleotide sequence
atgaagcacctctggttcttccttcttcttgtggccgctccccgctgggtcctcagcgatatcgtgctgacccagtcacccgccaccctctcagcttcacctggcgagaaggtcactctgacttgctctgcctcatctagcgtgtcatcttcatatctgtactggtatcagcaaaaaccgggacaagccccgaagctcctgatctacagcaccagcaaccttgcatccggagtgcctgccaggtttagcgggtccgggtccggtacctcatattcactgaccatttcttctcttgaacccgaagatttcgctacctactactgtcatcagtggtctagctacccatacactttcggcggaggaaccaaactggagattaagcgtacggtggcagccccttctgtctttatcttccctccatccgacgagcagctcaaatcaggaaccgcttctgtcgtgtgcctgcttaacaatttctacccacgggaagccaaggtgcagtggaaggtggacaatgccctgcaatcaggtaattcccaagagtcagtgactgaacaggatagcaaggacagcacctattcactctccagcactctgaccctgtccaaggctgactacgaaaagcataaggtgtacgcatgcgaggtgacccaccagggtctgagcagccccgtcaccaagtctttcaacagaggggagtgt

SEQ ID NO: 20 - DNA, 645 bp, Artificial Sequence
  73R009 Light chain nucleotide sequence without predicted signal sequence
gatatcgtgctgacccagtcacccgccaccctctcagcttcacctggcgagaaggtcactctgacttgctctgcctcatctagcgtgtcatcttcatatctgtactggtatcagcaaaaaccgggacaagccccgaagctcctgatctacagcaccagcaaccttgcatccggagtgcctgccaggtttagcgggtccgggtccggtacctcatattcactgaccatttcttctcttgaacccgaagatttcgctacctactactgtcatcagtggtctagctacccatacactttcggcggaggaaccaaactggagattaagcgtacggtggcagccccttctgtctttatcttccctccatccgacgagcagctcaaatcaggaaccgcttctgtcgtgtgcctgcttaacaatttctacccacgggaagccaaggtgcagtggaaggtggacaatgccctgcaatcaggtaattcccaagagtcagtgactgaacaggatagcaaggacagcacctattcactctccagcactctgaccctgtccaaggctgactacgaaaagcataaggtgtacgcatgcgaggtgacccaccagggtctgagcagccccgtcaccaagtctttcaacagaggggagtgt

SEQ ID NO: 21 - protein, 151 aa, Homo sapiens
QQPPPPPQQQQSGQQYNGERGISVPDHGYCQPISIPLCTDIAYNQTIMPNLLGHTNQEDAGLEVHQFYPLVKVQCSAELKFFLCSMYAPVCTVLEQALPPCRSLCERARQGCEALMNKFGFQWPDTLKCEKFPVHGAGELCVGQNTSDKGT

SEQ ID NO: 22 - protein, 136 aa, Homo sapiens
QFHGEKGISIPDHGFCQPISIPLCTDIAYNQTIMPNLLGHTNQEDAGLEVHQFYPLVKVQCSPELRFFLCSMYAPVCTVLEQAIPPCRSICERARQGCEALMNKFGFQWPERLRCEHFPRHGAEQICVGQNHSEDG

SEQ ID NO: 23 - protein, 121 aa, Homo sapiens
HSLFSCEPITLRMCQDLPYNTTFMPNLLNHYDQQTAALAMEPFHPMVNLDCSRDFRPFLCALYAPICMEYGRVTLPCRRLCQRAYSECSKLMEMFGVPWPEDMECSRFPDCDEPYPRLVDL

SEQ ID NO: 24 - protein, 131 aa, Homo sapiens
FGDEEERRCDPIRISMCQNLGYNVTKMPNLVGHELQTDAELQLTTFTPLIQYGCSSQLQFFLCSVYVPMCTEKINIPIGPCGGMCLSVKRRCEPVLKEFGFAWPESLNCSKFPPQNDHNHMCMEGPGDEEV

SEQ ID NO: 25 - protein, 131 aa, Homo sapiens
ASKAPVCQEITVPMCRGIGYNLTHMPNQFNHDTQDEAGLEVHQFWPLVEIQCSPDLRFFLCSMYTPICLPDYHKPLPPCRSVCERAKAGCSPLMRQYGFAWPERMSCDRLPVLGRDAEVLCMDYNRSEATT

SEQ ID NO: 26 - protein, 127 aa, Homo sapiens
HSLFTCEPITVPRCMKMAYNMTFFPNLMGHYDQSIAAVEMEHFLPLANLECSPNIETFLCKAFVPTCIEQIHVVPPCRKLCEKVYSDCKKLIDTFGIRWPEELECDRLQYCDETVPVTFDPHTEFLG

SEQ ID NO: 27 - protein, 138 aa, Homo sapiens
QPYHGEKGISVPDHGFCQPISIPLCTDIAYNQTILPNLLGHTNQEDAGLEVHQFYPLVKVQCSPELRFFLCSMYAPVCTVLDQAIPPCRSLCERARQGCEALMNKFGFQWPERLRCENFPVHGAGEICVGQNTSDGSG

SEQ ID NO: 28 - protein, 131 aa, Homo sapiens
ASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTT

SEQ ID NO: 29 - protein, 129 aa, Homo sapiens
ASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDL

SEQ ID NO: 30 - protein, 137 aa, Homo sapiens
LEIGRFDPERGRGAAPCQAVEIPMCRGIGYNLTRMPNLLGHTSQGEAAAELAEFAPLVQYGCHSHLRFFLCSLYAPMCTDQVSTPIPACRPMCEQARLRCAPIMEQFNFGWPDSLDCARLPTRNDPHALCMEAPENA

SEQ ID NO: 31 - protein, 134 aa, Homo sapiens
ISSMDMERPGDGKCQPIEIPMCKDIGYNMTRMPNLMGHENQREAAIQLHEFAPLVEYGCHGHLRFFLCSLYAPMCTEQVSTPIPACRVMCEQARLKCSPIMEQFNFKWPDSLDCRKLPNKNDPNYLCMEAPNNG

SEQ ID NO: 32 - protein, 112 aa, Homo sapiens
CQPISIPLCTDIAYNQTIMPNLLGHTNQEDAGLEVHQFYPLVKVQCSAELKFFLCSMYAPVCTVLEQALPPCRSLCERARQGCEALMNKFGFQWPDTLKCEKFPVHGAGELC

SEQ ID NO: 33 - protein, 112 aa, Homo sapiens
CQPISIPLCTDIAYNQTIMPNLLGHTNQEDAGLEVHQFYPLVKVQCSPELRFFLCSMYAPVCTVLEQAIPPCRSICERARQGCEALMNKFGFQWPERLRCEHFPRHGAEQIC

SEQ ID NO: 34 - protein, 106 aa, Homo sapiens
CEPITLRMCQDLPYNTTFMPNLLNHYDQQTAALAMEPFHPMVNLDCSRDFRPFLCALYAPICMEYGRVTLPCRRLCQRAYSECSKLMEMFGVPWPEDMECSRFPDC

SEQ ID NO: 35 - protein, 114 aa, Homo sapiens
CDPIRISMCQNLGYNVTKMPNLVGHELQTDAELQLTTFTPLIQYGCSSQLQFFLCSVYVPMCTEKINIPIGPCGGMCLSVKRRCEPVLKEFGFAWPESLNCSKFPPQNDHNHMC

SEQ ID NO: 36 - protein, 115 aa, Homo sapiens
CQEITVPMCRGIGYNLTHMPNQFNHDTQDEAGLEVHQFWPLVEIQCSPDLRFFLCSMYTPICLPDYHKPLPPCRSVCERAKAGCSPLMRQYGFAWPERMSCDRLPVLGRDAEVLC

SEQ ID NO: 37 - protein, 106 aa, Homo sapiens
CEPITVPRCMKMAYNMTFFPNLMGHYDQSIAAVEMEHFLPLANLECSPNIETFLCKAFVPTCIEQIHVVPPCRKLCEKVYSDCKKLIDTFGIRWPEELECDRLQYC

SEQ ID NO: 38 - protein, 112 aa, Homo sapiens
CQPISIPLCTDIAYNQTILPNLLGHTNQEDAGLEVHQFYPLVKVQCSPELRFFLCSMYAPVCTVLDQAIPPCRSLCERARQGCEALMNKFGFQWPERLRCENFPVHGAGEIC

SEQ ID NO: 39 - protein, 114 aa, Homo sapiens
CQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLC

SEQ ID NO: 40 - protein, 114 aa, Homo sapiens
CQAVEIPMCRGIGYNLTRMPNLLGHTSQGEAAAELAEFAPLVQYGCHSHLRFFLCSLYAPMCTDQVSTPIPACRPMCEQARLRCAPIMEQFNFGWPDSLDCARLPTRNDPHALC

SEQ ID NO: 41 - protein, 114 aa, Homo sapiens
CQPIEIPMCKDIGYNMTRMPNLMGHENQREAAIQLHEFAPLVEYGCHGHLRFFLCSLYAPMCTEQVSTPIPACRVMCEQARLKCSPIMEQFNFKWPDSLDCRKLPNKNDPNYLC

SEQ ID NO: 42 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 227 aa, Artificial Sequence
  Human IgG1 Fc region variant
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 224 aa, Homo sapiens
CVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 45 - protein, 235 aa, Homo sapiens
TKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 46 - protein, 235 aa, Artificial Sequence
  Human IgG2 Fc region variant
TKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 47 - protein, 224 aa, Artificial Sequence
  Human IgG2 Fc region (Variant 13A)
CVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREKMTKNQVSLTCLVKGFYPSDIAVEWESNGQPENNYKTTPPMLKSDGSFFLYSKLTVDKSRWQQGNVFSCSVMHEALHNHYTQKSLSLSPGK

SEQ ID NO: 48 - protein, 224 aa, Artificial Sequence
  Human IgG2 Fc region (Variant 13B)
CVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVEGFYPSDIAVEWESNGQPENNYKTTPPMLDSDGSFFLYSELTVDKSRWQQGNVFSCSVMHEALHNHYTQKSLSLSPGK

SEQ ID NO: 49 - protein, 235 aa, Artificial Sequence
  Human IgG2 Fc region (Variant 13A)
TKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREKMTKNQVSLTCLVKGFYPSDIAVEWESNGQPENNYKTTPPMLKSDGSFFLYSKLTVDKSRWQQGNVFSCSVMHEALHNHYTQKSLSLSPGK

SEQ ID NO: 50 - protein, 235 aa, Artificial Sequence
  Human IgG2 Fc region variant (Variant 13A)
TKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREKMTKNQVSLTCLVKGFYPSDIAVEWESNGQPENNYKTTPPMLKSDGSFFLYSKLTVDKSRWQQGNVFSCSVMHEALHNHYTQKSLSLSPGK

SEQ ID NO: 51 - protein, 235 aa, Artificial Sequence
  Human IgG2 Fc region (Variant 13B)
TKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVEGFYPSDIAVEWESNGQPENNYKTTPPMLDSDGSFFLYSELTVDKSRWQQGNVFSCSVMHEALHNHYTQKSLSLSPGK

SEQ ID NO: 52 - protein, 235 aa, Artificial Sequence
  Human IgG2 Fc region variant (Variant 13B)
TKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVEGFYPSDIAVEWESNGQPENNYKTTPPMLDSDGSFFLYSELTVDKSRWQQGNVFSCSVMHEALHNHYTQKSLSLSPGK

SEQ ID NO: 53 - protein, 363 aa, Artificial Sequence
  FZD8-Fc variant 54F28 amino acid sequence without predicted signal sequence
ASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 390 aa, Artificial Sequence
  FZD8-Fc variant 54F28 amino acid sequence with signal sequence
MEWGYLLEVTSLLAALLLLQRSPFVHAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 393 aa, Artificial Sequence
  FZD8-Fc variant (13B variant) amino acid sequence with signal sequence
MEWGYLLEVTSLLAALLLLQRSPIVHAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVEGFYPSDIAVEWESNGQPENNYKTTPPMLDSDGSFFLYSELTVDKSRWQQGNVFSCSVMHEALHNHYTQKSLSLSPGK

SEQ ID NO: 56 - protein, 366 aa, Artificial Sequence
  FZD8-Fc variant (13B variant) amino acid sequence without signal sequence
ASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVEGFYPSDIAVEWESNGQPENNYKTTPPMLDSDGSFFLYSELTVDKSRWQQGNVFSCSVMHEALHNHYTQKSLSLSPGK

SEQ ID NO: 57 - protein, 83 aa, Homo sapiens
DLVYFEKSPNFCTYSGRLGTAGTAGRACNSSSPALDGCELLCCGRGHRTRTQRVTERCNCTFHWCCHVSCRNCTHTRVLHECL

SEQ ID NO: 58 - protein, 94 aa, Homo sapiens
DLVYFENSPDYCIRDREAGSLGTAGRVCNLTSRGMDSCEVMCCGRGYDTSHVTRMTKCGCKFHWCCAVRCQDCLEALDVHTCKAPKNADWTTAT

SEQ ID NO: 59 - protein, 94 aa, Homo sapiens
DLVYFDNSPDYCVLDKAAGSLGTAGRVCSKTSKGTDGCEIMCCGRGYDTTRVTRVTQCECKFHWCCAVRCKECRNTVDVHTCKAPKKAEWLDQT

SEQ ID NO: 60 - protein, 83 aa, Homo sapiens
DLVYYENSPNFCEPNPETGSFGTRDRTCNVTSHGIDGCDLLCCGRGHNTRTEKRKEKCHCIFHWCCYVSCQECIRIYDVHTCK

SEQ ID NO: 61 - protein, 83 aa, Homo sapiens
DLVYYEASPNFCEPNPETGSFGTRDRTCNVSSHGIDGCDLLCCGRGHNARAERRREKCRCVFHWCCYVSCQECTRVYDVHTCK

SEQ ID NO: 62 - protein, 83 aa, Homo sapiens
DLVYIEKSPNYCEEDPVTGSVGTQGRACNKTAPQASGCDLMCCGRGYNTHQYARVWQCNCKFHWCCYVKCNTCSERTEMYTCK

SEQ ID NO: 63 - protein, 83 aa, Homo sapiens
DLVYIEKSPNYCEEDAATGSVGTQGRLCNRTSPGADGCDTMCCGRGYNTHQYTKVWQCNCKFHWCCFVKCNTCSERTEVFTCK

SEQ ID NO: 64 - protein, 108 aa, Homo sapiens
ELIFLEESPDYCTCNSSLGIYGTEGRECLQNSHNTSRWERRSCGRLCTECGLQVEERKTEVISSCNCKFQWCCTVKCDQCRHVVSKYYCARSPGSAQSLGRVWFGVYI

SEQ ID NO: 65 - protein, 107 aa, Homo sapiens
ELVHLEDSPDYCLENKTLGLLGTEGRECLRRGRALGRWELRSCRRLCGDCGLAVEERRAETVSSCNCKFHWCCAVRCEQCRRRVTKYFCSRAERPRGGAAHKPGRKP

SEQ ID NO: 66 - protein, 83 aa, Homo sapiens
DLVYFEKSPDFCEREPRLDSAGTVGRLCNKSSAGSDGCGSMCCGRGHNILRQTRSERCHCRFHWCCFVVCEECRITEWVSVCK

SEQ ID NO: 67 - protein, 83 aa, Homo sapiens
ELVYFEKSPDFCERDPTMGSPGTRGRACNKTSRLLDGCGSLCCGRGHNVLRQTRVERCHCRFHWCCYVLCDECKVTEWVNVCK

SEQ ID NO: 68 - protein, 8 aa, Artificial Sequence
  Linker
ESGGGGVT

SEQ ID NO: 69 - protein, 9 aa, Artificial Sequence
  Linker
LESGGGGVT

SEQ ID NO: 70 - protein, 6 aa, Artificial Sequence
  Linker
GRAQVT

SEQ ID NO: 71 - protein, 6 aa, Artificial Sequence
  Linker
WRAQVT

SEQ ID NO: 72 - protein, 8 aa, Artificial Sequence
  Linker
ARGRAQVT

SEQ ID NO: 73 - protein, 8 aa, Artificial Sequence
  FLAG peptide
DYKDDDDK

SEQ ID NO: 74 - protein, 330 aa, Homo sapiens [sequence omitted]

SEQ ID NO: 75 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 76 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 77 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 78 - protein, 10 aa, Artificial Sequence
  MET antibody Heavy chain CDR1
GYTFTSYWLH

SEQ ID NO: 79 - protein, 18 aa, Artificial Sequence
  MET antibody Heavy chain CDR2
GMIDPSNSDTRFNPNFKD

SEQ ID NO: 80 - protein, 11 aa, Artificial Sequence
  MET Heavy chain CDR3
XYGSYVSPLDY

SEQ ID NO: 81 - protein, 11 aa, Artificial Sequence
  MET Heavy chain CDR3
TYGSYVSPLDY

SEQ ID NO: 82 - protein, 11 aa, Artificial Sequence
  MET Heavy chain CDR3
SYGSYVSPLDY

SEQ ID NO: 83 - protein, 12 aa, Artificial Sequence
  MET Heavy chain CDR3
ATYGSYVSPLDY

SEQ ID NO: 84 - protein, 17 aa, Artificial Sequence
  MET Light chain CDR1
KSSQSLLYTSSQKNYLA

SEQ ID NO: 85 - protein, 7 aa, Artificial Sequence
  MET Light chain CDR2
WASTRES

SEQ ID NO: 86 - protein, 9 aa, Artificial Sequence
  MET Light chain CDR3
QQYYAYPWT

SEQ ID NO: 87 - protein, 366 aa, Artificial Sequence
  FZD8-Fc variant (13A variant) amino acid sequence without signal sequence
ASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREKMTKNQVSLTCLVKGFYPSDIAVEWESNGQPENNYKTTPPMLKSDGSFFLYSKLTVDKSRWQQGNVFSCSVMHEALHNHYTQKSLSLSPGK

SEQ ID NO: 88 - protein, 439 aa, Artificial Sequence
  73R009 (13B variant) Heavy chain amino acid sequence without signal sequence
QVQLQESGPGLVKPSETLSLTCTVTGTTITASYAWSWIRQPPGKGLEWMGYISYSGGTDYNPSLKSRITISRDTFKNQFSLKLSSVTAADTATYYCARKGAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVEGFYPSDIAVEWESNGQPENNYKTTPPMLDSDGSFFLYSELTVDKSRWQQGNVFSCSVMHEALHNHYTQKSLSLSPGK

SEQ ID NO: 89 - DNA, 1182 bp, Artificial Sequence
  FZD8-Fc variant (13B variant) nucleotide sequence with signal sequence
atggagtggggttatcttttagaagtgacctcgctgctagccgccttgctactgctgcagcgctctccgatcgtgcacgccgcctcggccaaggagctggcatgccaagagatcaccgtgccgctatgcaagggcatcggctacaactacacctacatgcccaatcaattcaaccacgacacgcaagacgaggcgggcctggaggtgcaccagttctggccgctggtggagatccagtgctcgcccgatctcaagttcttcctgtgcagcatgtacacgcccatctgcctagaggactacaagaagccgctgccgccctgccgctcggtgtgcgagcgcgccaaggccggctgcgcgccgctcatgcgccagtacggcttcgcctggcccgaccgcatgcgctgcgaccggctgcccgagcaaggcaaccctgacacgctgtgcatggactacaaccgcaccgacctaaccaccaccaaagttgacaagactgttgagcgaaagagctgcgttgagtgccctccatgtcctgcacctcctgtggctggcccttctgtgttcctgttccctccaaaacctaaagacactctaatgatctctcggactcctgaggtgacttgcgtggttgtggacgtgtcccacgaggaccctgaggtgcagtttaattggtacgtggacggagtcgaggtgcacaatgcaaagaccaagcctcgggaggaacagttcaactccaccttccgggtggtttctgtgttgaccgttgtgcaccaagactggctgaacggcaaagaatacaagtgcaaggtgtccaacaagggcctgcctgcccctatcgaaaagaccatcagcaagaccaagggccagcctcgcgagcctcaggtgtacaccctgcctcccagccgggaagaaatgaccaagaaccaggtgtccctgacctgtctggtggagggcttctacccttccgacatcgccgttgagtgggagtctaacggacagccggagaacaactacaagactacgcctccaatgctggactccgacggctccttcttcctgtactccgaactgaccgtggacaagtcccggtggcagcagggcaacgtgttctcatgctccgtaatgcacgaagccttacacaatcactacactcaaaagtccctatccttatctcctggcaagtag

SEQ ID NO: 90 - DNA, 1122 bp, Artificial Sequence
  FZD8-Fc variant (13B variant) nucleotide sequence without signal sequence
cgctctccgatcgtgcacgccgcctcggccaaggagctggcatgccaagagatcaccgtgccgctatgcaagggcatcggctacaactacacctacatgcccaatcaattcaaccacgacacgcaagacgaggcgggcctggaggtgcaccagttctggccgctggtggagatccagtgctcgcccgatctcaagttcttcctgtgcagcatgtacacgcccatctgcctagaggactacaagaagccgctgccgccctgccgctcggtgtgcgagcgcgccaaggccggctgcgcgccgctcatgcgccagtacggcttcgcctggcccgaccgcatgcgctgcgaccggctgcccgagcaaggcaaccctgacacgctgtgcatggactacaaccgcaccgacctaaccaccaccaaagttgacaagactgttgagcgaaagagctgcgttgagtgccctccatgtcctgcacctcctgtggctggcccttctgtgttcctgttccctccaaaacctaaagacactctaatgatctctcggactcctgaggtgacttgcgtggttgtggacgtgtcccacgaggaccctgaggtgcagtttaattggtacgtggacggagtcgaggtgcacaatgcaaagaccaagcctcgggaggaacagttcaactccaccttccgggtggtttctgtgttgaccgttgtgcaccaagactggctgaacggcaaagaatacaagtgcaaggtgtccaacaagggcctgcctgcccctatcgaaaagaccatcagcaagaccaagggccagcctcgcgagcctcaggtgtacaccctgcctcccagccgggaagaaatgaccaagaaccaggtgtccctgacctgtctggtggagggcttctacccttccgacatcgccgttgagtgggagtctaacggacagccggagaacaactacaagactacgcctccaatgctggactccgacggctccttcttcctgtactccgaactgaccgtggacaagtcccggtggcagcagggcaacgtgttctcatgctccgtaatgcacgaagccttacacaatcactacactcaaaagtccctatccttatctcctggcaagtag

SEQ ID NO: 91 - protein, 230 aa, Homo sapiens
KSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 232 aa, Homo sapiens
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 1390 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 94 - protein, 113 aa, Artificial Sequence
  Humanized Antibody 73R010 (73R009 H12) Heavy chain variable region amino acid sequence
QVQLQESGPGLVKPSETLSLTCTVSGTTITASYAWSWIRQPPGKGLEWMGYISYSGGTDYNPSLKSRVTISRDTFKNQFSLKLSSVTAADTAVYYCARKGAYWGQGTLVTVSS

SEQ ID NO: 95 - protein, 108 aa, Artificial Sequence
  73R010 (73R009 L7) Light chain variable region amino acid sequence
EIVLTQSPATLSLSPGERATLSCSASSSVSSSYLYWYQQKPGQAPRLLIYSTSNLASGIPARFSGSGSGTDFTLTISRLEPEDFAVYYCHQWSSYPYTFGQGTKLEIK

SEQ ID NO: 96 - protein, 458 aa, Artificial Sequence
  73R010 (73R009 H12) Heavy chain amino acid sequence with predicted signal sequence
MKHLWFFLLLVAAPRWVLSQVQLQESGPGLVKPSETLSLTCTVSGTTITASYAWSWIRQPPGKGLEWMGYISYSGGTDYNPSLKSRVTISRDTFKNQFSLKLSSVTAADTAVYYCARKGAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 97 - protein, 458 aa, Artificial Sequence
  73R010 13A variant (73R009 H12) Heavy chain amino acid sequence with predicted signal sequence
MKHLWFFLLLVAAPRWVLSQVQLQESGPGLVKPSETLSLTCTVSGTTITASYAWSWIRQPPGKGLEWMGYISYSGGTDYNPSLKSRVTISRDTFKNQFSLKLSSVTAADTAVYYCARKGAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREKMTKNQVSLTCLVKGFYPSDIAVEWESNGQPENNYKTTPPMLKSDGSFFLYSKLTVDKSRWQQGNVFSCSVMHEALHNHYTQKSLSLSPGK

SEQ ID NO: 98 - protein, 235 aa, Artificial Sequence
  73R010 (73R009 L7) Light chain amino acid sequence with predicted signal sequence
METPAQLLFLLLLWLPDTTGEIVLTQSPATLSLSPGERATLSCSASSSVSSSYLYWYQQKPGQAPRLLIYSTSNLASGIPARFSGSGSGTDFTLTISRLEPEDFAVYYCHQWSSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 439 aa, Artificial Sequence
  73R010 (73R009 H12) Heavy chain amino acid sequence without predicted signal sequence
QVQLQESGPGLVKPSETLSLTCTVSGTTITASYAWSWIRQPPGKGLEWMGYISYSGGTDYNPSLKSRVTISRDTFKNQFSLKLSSVTAADTAVYYCARKGAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 100 - protein, 439 aa, Artificial Sequence
  73R010 13A Variant (73R009 H12) Heavy chain amino acid sequence without predicted signal sequence
QVQLQESGPGLVKPSETLSLTCTVSGTTITASYAWSWIRQPPGKGLEWMGYISYSGGTDYNPSLKSRVTISRDTFKNQFSLKLSSVTAADTAVYYCARKGAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREKMTKNQVSLTCLVKGFYPSDIAVEWESNGQPENNYKTTPPMLKSDGSFFLYSKLTVDKSRWQQGNVFSCSVMHEALHNHYTQKSLSLSPGK

SEQ ID NO: 101 - protein, 215 aa, Artificial Sequence
  73R010 (73R009 L7) Light chain amino acid sequence without predicted signal sequence
EIVLTQSPATLSLSPGERATLSCSASSSVSSSYLYWYQQKPGQAPRLLIYSTSNLASGIPARFSGSGSGTDFTLTISRLEPEDFAVYYCHQWSSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - DNA, 1374 bp, Artificial Sequence
  73R010 (73R009 H12) Heavy chain nucleotide sequence
atgaaacatctgtggttcttcctcctcctggtggctgctccccgctgggtcctgtcccaagtgcagctgcaggagtctggcccaggactggtgaagccttccgagaccctgtccctcacctgcaccgtctctggaactaccatcactgcctcctatgcctggagctggattcggcagccccctggcaagggactggagtggatggggtacataagctactccgggggcactgactacaacccatctctcaaatcccgggtcacaatatcacgggacacattcaagaaccagttctccctgaaactctcctctgtgaccgctgctgacaccgccgtctattactgtgcaagaaagggggcttactggggccaagggactctggtcactgtcagctcagcctctaccaagggccctagcgtcttccctctggccccctgctcccggtccaccagcgagagcacagccgccctgggctgcctggtcaaggactacttccccgaacctgtgacagtgtcctggaactccggcgctctgaccagcggcgtgcacaccttcccagctgtcctccagtcctccggactctactccctctcctccgtggtgacagtgccctcctccaacttcggcacccagacctacacctgcaacgtcgatcacaagcccagcaacaccaaggttgataagacagttgagcgcaaatgttgtgtcgagtgccctccttgcccagcccctcctgtggctggaccttccgtcttcctcttcccccctaaacccaaagacaccctcatgatctcccggacccctgaggtcacatgcgtggtggtggacgtgagccacgaagaccccgaggtccagttcaactggtatgtggacggcgtggaggtgcataatgccaagacaaagccacgggaggagcagttcaacagcacattccgggtggtcagcgtcctcaccgttgtgcaccaggactggctgaacggcaaggagtacaagtgcaaagtctccaacaaaggcctccctgcccccatcgagaaaaccatctccaaaaccaaagggcagcccagggaaccacaggtgtacaccctgcccccttcccgggaggaratgaccaagaaccaagtcagcctgacctgcctggtcaaaggcttctacccctccgacatcgccgtggagtgggagagcaatgggcagcctgagaacaactacaagaccacacctcccatgctggaytccgacggctccttcttcctctactccaaactcaccgtggacaagagcaggtggcagcaggggaacgtcttctcctgctccgtgatgcatgaggctctgcacaaccactacacacagaagtccctctccctgtctcctggaaaa

SEQ ID NO: 103 - DNA, 1374 bp, Artificial Sequence
  73R010 13A variant (73R009 H12) Heavy chain nucleotide sequence
atgaaacatctgtggttcttcctcctcctggtggctgctccccgctgggtcctgtcccaagtgcagctgcaggagtctggcccaggactggtgaagccttccgagaccctgtccctcacctgcaccgtctctggaactaccatcactgcctcctatgcctggagctggattcggcagccccctggcaagggactggagtggatggggtacataagctactccgggggcactgactacaacccatctctcaaatcccgggtcacaatatcacgggacacattcaagaaccagttctccctgaaactctcctctgtgaccgctgctgacaccgccgtctattactgtgcaagaaagggggcttactggggccaagggactctggtcactgtcagctcagcctctaccaagggccctagcgtcttccctctggccccctgctcccggtccaccagcgagagcacagccgccctgggctgcctggtcaaggactacttccccgaacctgtgacagtgtcctggaactccggcgctctgaccagcggcgtgcacaccttcccagctgtcctccagtcctccggactctactccctctcctccgtggtgacagtgccctcctccaacttcggcacccagacctacacctgcaacgtcgatcacaagcccagcaacaccaaggttgataagacagttgagcgcaaatgttgtgtcgagtgccctccttgcccagcccctcctgtggctggaccttccgtcttcctcttcccccctaaacccaaagacaccctcatgatctcccggacccctgaggtcacatgcgtggtggtggacgtgagccacgaagaccccgaggtccagttcaactggtatgtggacggcgtggaggtgcataatgccaagacaaagccacgggaggagcagttcaacagcacattccgggtggtcagcgtcctcaccgttgtgcaccaggactggctgaacggcaaggagtacaagtgcaaagtctccaacaaaggcctccctgcccccatcgagaaaaccatctccaaaaccaaagggcagcccagggaaccacaggtgtacaccctgcccccttcccgggagaagatgaccaagaaccaagtcagcctgacctgcctggtcaaaggcttctacccctccgacatcgccgtggagtgggagagcaatgggcagcctgagaacaactacaagaccacacctcccatgctgaagtccgacggctccttcttcctctactccaaactcaccgtggacaagagcaggtggcagcaggggaacgtcttctcctgctccgtgatgcatgaggctctgcacaaccactacacacagaagtccctctccctgtctcctggaaaa

SEQ ID NO: 104 - DNA, 1317 bp, Artificial Sequence
  73R010 (73R009 H12) Heavy chain nucleotide sequence without predicted signal sequence
caagtgcagctgcaggagtctggcccaggactggtgaagccttccgagaccctgtccctcacctgcaccgtctctggaactaccatcactgcctcctatgcctggagctggattcggcagccccctggcaagggactggagtggatggggtacataagctactccgggggcactgactacaacccatctctcaaatcccgggtcacaatatcacgggacacattcaagaaccagttctccctgaaactctcctctgtgaccgctgctgacaccgccgtctattactgtgcaagaaagggggcttactggggccaagggactctggtcactgtcagctcagcctctaccaagggccctagcgtcttccctctggccccctgctcccggtccaccagcgagagcacagccgccctgggctgcctggtcaaggactacttccccgaacctgtgacagtgtcctggaactccggcgctctgaccagcggcgtgcacaccttcccagctgtcctccagtcctccggactctactccctctcctccgtggtgacagtgccctcctccaacttcggcacccagacctacacctgcaacgtcgatcacaagcccagcaacaccaaggttgataagacagttgagcgcaaatgttgtgtcgagtgccctccttgcccagcccctcctgtggctggaccttccgtcttcctcttcccccctaaacccaaagacaccctcatgatctcccggacccctgaggtcacatgcgtggtggtggacgtgagccacgaagaccccgaggtccagttcaactggtatgtggacggcgtggaggtgcataatgccaagacaaagccacgggaggagcagttcaacagcacattccgggtggtcagcgtcctcaccgttgtgcaccaggactggctgaacggcaaggagtacaagtgcaaagtctccaacaaaggcctccctgcccccatcgagaaaaccatctccaaaaccaaagggcagcccagggaaccacaggtgtacaccctgcccccttcccgggaggaratgaccaagaaccaagtcagcctgacctgcctggtcaaaggcttctacccctccgacatcgccgtggagtgggagagcaatgggcagcctgagaacaactacaagaccacacctcccatgctggaytccgacggctccttcttcctctactccaaactcaccgtggacaagagcaggtggcagcaggggaacgtcttctcctgctccgtgatgcatgaggctctgcacaaccactacacacagaagtccctctccctgtctcctggaaaa

SEQ ID NO: 105 - DNA, 1317 bp, Artificial Sequence
  73R010 13A variant (73R009 H12) Heavy chain nucleotide sequence without predicted signal sequence
caagtgcagctgcaggagtctggcccaggactggtgaagccttccgagaccctgtccctcacctgcaccgtctctggaactaccatcactgcctcctatgcctggagctggattcggcagccccctggcaagggactggagtggatggggtacataagctactccgggggcactgactacaacccatctctcaaatcccgggtcacaatatcacgggacacattcaagaaccagttctccctgaaactctcctctgtgaccgctgctgacaccgccgtctattactgtgcaagaaagggggcttactggggccaagggactctggtcactgtcagctcagcctctaccaagggccctagcgtcttccctctggccccctgctcccggtccaccagcgagagcacagccgccctgggctgcctggtcaaggactacttccccgaacctgtgacagtgtcctggaactccggcgctctgaccagcggcgtgcacaccttcccagctgtcctccagtcctccggactctactccctctcctccgtggtgacagtgccctcctccaacttcggcacccagacctacacctgcaacgtcgatcacaagcccagcaacaccaaggttgataagacagttgagcgcaaatgttgtgtcgagtgccctccttgcccagcccctcctgtggctggaccttccgtcttcctcttcccccctaaacccaaagacaccctcatgatctcccggacccctgaggtcacatgcgtggtggtggacgtgagccacgaagaccccgaggtccagttcaactggtatgtggacggcgtggaggtgcataatgccaagacaaagccacgggaggagcagttcaacagcacattccgggtggtcagcgtcctcaccgttgtgcaccaggactggctgaacggcaaggagtacaagtgcaaagtctccaacaaaggcctccctgcccccatcgagaaaaccatctccaaaaccaaagggcagcccagggaaccacaggtgtacaccctgcccccttcccgggagaagatgaccaagaaccaagtcagcctgacctgcctggtcaaaggcttctacccctccgacatcgccgtggagtgggagagcaatgggcagcctgagaacaactacaagaccacacctcccatgctgaagtccgacggctccttcttcctctactccaaactcaccgtggacaagagcaggtggcagcaggggaacgtcttctcctgctccgtgatgcatgaggctctgcacaaccactacacacagaagtccctctccctgtctcctggaaaa

SEQ ID NO: 106 - DNA, 705 bp, Artificial Sequence
  73R010 (73R009 L7) Light chain nucleotide sequence
atggaaaccccagctcaactcctcttcctcctgctcctctggctccccgataccaccggagaaattgtgctcactcagtctcccgctaccctgtctctctctcctggggaaagagccaccctctcctgctctgcctcctcatccgtttcttcctcctacctctactggtaccagcagaaacctggccaggctcccaggctcctcatctatagcacatccaacctggcttctggcatcccagctaggttctctggctctgggtctgggacagacttcactctcaccatcagcagactggagcctgaagattttgcagtgtattactgtcatcagtggtcatcctacccatacacattcggccagggaaccaaactggagattaagcgtacggtggcagccccttctgtctttatcttccctccatccgacgagcagctcaaatcaggaaccgcttctgtcgtgtgcctgcttaacaatttctacccacgggaagccaaggtgcagtggaaggtggacaatgccctgcaatcaggtaattcccaagagtcagtgactgaacaggatagcaaggacagcacctattcactctccagcactctgaccctgtccaaggctgactacgaaaagcataaggtgtacgcatgcgaggtgacccaccagggtctgagcagccccgtcaccaagtctttcaacagaggggagtgt

SEQ ID NO: 107 - DNA, 645 bp, Artificial Sequence
  73R010 (73R009 L7) Light chain nucleotide sequence without predicted signal sequence
gaaattgtgctcactcagtctcccgctaccctgtctctctctcctggggaaagagccaccctctcctgctctgcctcctcatccgtttcttcctcctacctctactggtaccagcagaaacctggccaggctcccaggctcctcatctatagcacatccaacctggcttctggcatcccagctaggttctctggctctgggtctgggacagacttcactctcaccatcagcagactggagcctgaagattttgcagtgtattactgtcatcagtggtcatcctacccatacacattcggccagggaaccaaactggagattaagcgtacggtggcagccccttctgtctttatcttccctccatccgacgagcagctcaaatcaggaaccgcttctgtcgtgtgcctgcttaacaatttctacccacgggaagccaaggtgcagtggaaggtggacaatgccctgcaatcaggtaattcccaagagtcagtgactgaacaggatagcaaggacagcacctattcactctccagcactctgaccctgtccaaggctgactacgaaaagcataaggtgtacgcatgcgaggtgacccaccagggtctgagcagccccgtcaccaagtctttcaacagaggggagtgt

SEQ ID NO: 108 - protein, 439 aa, Artificial Sequence
  3R010 13B Variant (73R009 H12) Heavy chain amino acid sequence without predicted signal sequence
QVQLQESGPGLVKPSETLSLTCTVSGTTITASYAWSWIRQPPGKGLEWMGYISYSGGTDYNPSLKSRVTISRDTFKNQFSLKLSSVTAADTAVYYCARKGAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVEGFYPSDIAVEWESNGQPENNYKTTPPMLDSDGSFFLYSELTVDKSRWQQGNVFSCSVMHEALHNHYTQKSLSLSPGK

SEQ ID NO: 109 - DNA, 1377 bp, Artificial Sequence
  73R010 13A Variant (Cys/Ser) (73R009 H12) Heavy chain nucleotide sequence
atgaaacatctgtggttcttcctcctcctggtggctgctccccgctgggtcctgtcccaagtgcagctgcaggagtctggcccaggactggtgaagccttccgagaccctgtccctcacctgcaccgtctctggaactaccatcactgcctcctatgcctggagctggattcggcagccccctggcaagggactggagtggatggggtacataagctactccgggggcactgactacaacccatctctcaaatcccgggtcacaatatcacgggacacattcaagaaccagttctccctgaaactctcctctgtgaccgctgctgacaccgccgtctattactgtgcaagaaagggggcttactggggccaagggactctggtcactgtcagctcagcctcaactaagggccctagcgtcttccctctggccccctgctcccggtccaccagcgagagcacagccgccctgggctgcctggtcaaggactacttccccgaacctgtgacagtgtcctggaactccggcgctctgaccagcggcgtgcacaccttcccagctgtcctccagtcctccggactctactccctctcctccgtggtgacagtgccctcctccaacttcggcacccagacctacacctgcaacgtcgatcacaagcccagcaacaccaaggttgataagacagttgagcgcaaatcttgtgtcgagtgccctccttgcccagcccctcctgtggctggaccttccgtcttcctcttcccccctaaacccaaagacaccctcatgatctcccggacccctgaggtcacatgcgtggtggtggacgtgagccacgaagaccccgaggtccagttcaactggtatgtggacggcgtggaggtgcataatgccaagacaaagccacgggaggagcagttcaacagcacattccgggtggtcagcgtcctcaccgttgtgcaccaggactggctgaacggcaaggagtacaagtgcaaagtctccaacaaaggcctccctgcccccatcgagaaaaccatctccaaaaccaaagggcagcccagggaaccacaggtgtacaccctgcccccttcccgggagaagatgaccaagaaccaagtcagcctgacctgcctggtcaaaggcttctacccctccgacatcgccgtggagtgggagagcaatgggcagcctgagaacaactacaagaccacacctcccatgctgaagtccgacggctccttcttcctctactccaaactcaccgtggacaagagcaggtggcagcaggggaacgtcttctcctgctccgtgatgcatgaggctctgcacaaccactacacacagaagtccctctccctgtctcctggaaaatga

SEQ ID NO: 110 - protein, 458 aa, Artificial Sequence
  73R010 13A Variant (Cys/Ser) (73R009 H12) Heavy chain amino acid sequence with predicted signal sequence
MKHLWFFLLLVAAPRWVLSQVQLQESGPGLVKPSETLSLTCTVSGTTITASYAWSWIRQPPGKGLEWMGYISYSGGTDYNPSLKSRVTISRDTFKNQFSLKLSSVTAADTAVYYCARKGAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREKMTKNQVSLTCLVKGFYPSDIAVEWESNGQPENNYKTTPPMLKSDGSFFLYSKLTVDKSRWQQGNVFSCSVMHEALHNHYTQKSLSLSPGK

SEQ ID NO: 111 - protein, 439 aa, Artificial Sequence
  73R010 13A Variant (Cys/Ser) (73R009 H12) Heavy chain amino acid sequence without predicted signal sequence
QVQLQESGPGLVKPSETLSLTCTVSGTTITASYAWSWIRQPPGKGLEWMGYISYSGGTDYNPSLKSRVTISRDTFKNQFSLKLSSVTAADTAVYYCARKGAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREKMTKNQVSLTCLVKGFYPSDIAVEWESNGQPENNYKTTPPMLKSDGSFFLYSKLTVDKSRWQQGNVFSCSVMHEALHNHYTQKSLSLSPGK

SEQ ID NO: 112 - protein, 439 aa, Artificial Sequence
  73R010 (Cys/Ser) (73R009 H12) Heavy chain amino acid sequence without predicted signal sequence
QVQLQESGPGLVKPSETLSLTCTVSGTTITASYAWSWIRQPPGKGLEWMGYISYSGGTDYNPSLKSRVTISRDTFKNQFSLKLSSVTAADTAVYYCARKGAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 113 - protein, 5 aa, Artificial Sequence
  MET
PCQDC

SEQ ID NO: 114 - protein, 439 aa, Artificial Sequence
  73R010 13B Variant (Cys/Ser) (73R009 H12) Heavy chain amino acid sequence without predicted signal sequence
QVQLQESGPGLVKPSETLSLTCTVSGTTITASYAWSWIRQPPGKGLEWMGYISYSGGTDYNPSLKSRVTISRDTFKNQFSLKLSSVTAADTAVYYCARKGAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVEGFYPSDIAVEWESNGQPENNYKTTPPMLDSDGSFFLYSELTVDKSRWQQGNVFSCSVMHEALHNHYTQKSLSLSPGK